(19) United States
(12) Patent Application Publication
(10) Pub. No.: US 2026/0209793 A1
(43) Pub. Date: Jul. 23, 2026

(54) RECOMBINANT REGULATORY SEQUENCES AND THERAPEUTIC REGULATING OF SPLICING

(71) Applicant: Emugen Therapeutics LLC, Woburn, MA (US)

(72) Inventors: Jonathan J. Wilde, Waltham, MA (US); Tobias Kaiser, Boston, MA (US); Xian Gao, Belmont, MA (US); Olga Brevnova, Somerville, MA (US); Tomomi Aida, Arlington, MA (US); Yuanyuan Hou, Cambridge, MA (US); Tyler Brown, Somerville, MA (US)

(21) Appl. No.: 19/636,499

(22) Filed: Apr. 1, 2026

Related U.S. Application Data
(63) Continuation of application No. PCT/US24/50737, filed on Oct. 10, 2024.
(60) Provisional application No. 63/589,511, filed on Oct. 11, 2023.

(51) Int. Cl. C12N 15/85; C12N 15/113; C12N 15/67

(52) U.S. Cl. CPC C12N 15/85; C12N 15/113; C12N 15/67; C12N 2320/33; C12N 2830/36

(57) ABSTRACT

Disclosed herein are systems for modifying nucleic acid splicing of a target RNA. The system may be useful in a method such as a method for treating a genetic disorder such as epilepsy. The system may include an expression system, or a modified small nuclear RNA (snRNA) that includes an exonic splicing silencer (ESS) sequence, and a target nucleic acid sequence that targets the target RNA. Described herein are recombinant regulatory elements for expression systems. Also described are nucleic acid expression systems with recombinant regulatory elements.

### CROSS-REFERENCE

This application is a Continuation application of International Patent Application No. PCT/US2024/050737 filed Oct. 10, 2024, which claims the benefit of U.S. Provisional Application No. 63/589,511, filed Oct. 11, 2023, which application is incorporated herein by reference.

### INCORPORATION BY REFERENCE OF SEQUENCE LISTING

The present application is being filed along with a Sequence Listing in electronic format. The Sequence Listing is provided as a file entitled 062692-506001WO.xml, created Oct. 9, 2024, which is 1,488,898 bytes in size. The information in the electronic format of the Sequence Listing is incorporated by reference in its entirety.

### BACKGROUND

Improved expression systems are needed for expressing recombinant genes. Additionally, epileptic disorders and genetic disorders such as Dravet syndrome are a concern for many individuals, and improved treatments are needed.

### SUMMARY

Described herein are recombinant regulatory elements for expression systems. Also described are nucleic acid expression systems with recombinant regulatory elements. The regulatory elements and systems described herein address needs for improved expression systems, such as for gene editing or for transgenic expression.

Described herein, in some embodiments, are nucleic acid expression systems, comprising: a regulatory sequence operably coupled to a transcribable region of a nucleic acid, the regulatory sequence comprising a proximal regulatory sequence element (PSE) and a distal regulatory sequence element (DSE); wherein the PSE comprises a PSE sequence of a first small nuclear RNA (snRNA) of a first organism species, wherein the DSE comprises a DSE sequence of a second small nuclear RNA (snRNA) of a second organism species, and wherein the first and second snRNAs are different or wherein the first and second organism species are different. In some embodiments, the first snRNA is selected from the group consisting of snRNA U1, snRNA U2, snRNA U3, snRNA U4, snRNA U5, snRNA U6, snRNA U7, snRNA U11, snRNA U12 and snRNA 7SK. In some embodiments, the second snRNA is selected from the group consisting of snRNA U7, snRNA U2, snRNA U3, snRNA U4, snRNA U5, snRNA U6, snRNA U1, snRNA U11, snRNA U12 and snRNA 7SK. In some embodiments, the first organism species is selected from the group consisting of human and mouse. In some embodiments, the second organism species is selected from the group consisting of mouse and human. In some embodiments, the first snRNA is selected from the group consisting of snRNA U1, snRNA U2, snRNA U3, snRNA U4, snRNA U5, snRNA U6, snRNA U7, snRNA U11, snRNA U12 and snRNA 7SK; the second snRNA is selected from the group consisting of snRNA U7, snRNA U2, snRNA U3, snRNA U4, snRNA U5, snRNA U6, snRNA U1, snRNA U11, snRNA U12 and snRNA 7SK; the first organism species is selected from the group consisting of human and mouse; and the second organism species is selected from the group consisting of mouse and human. In some embodiments, the regulatory sequence comprises a promoter sequence. In some embodiments, the regulatory sequence comprises a promoter sequence. In some embodiments, the regulatory sequence comprises a terminator sequence. In some embodiments, the regulatory sequence comprises a terminator sequence. In some embodiments, the expression system produces a greater amount of RNA from the transcribable region than an expression system having a wild-type promoter and wild-type terminator operably coupled to the transcribable region. In some embodiments, the amount of RNA produced is at least 10% greater. Described herein, in some embodiments, are methods of producing a ribonucleic acid (RNA), comprising contacting a cell with the expression system.

Described herein, in some embodiments, are nucleic acid expression systems, comprising: a regulatory sequence operably coupled to a transcribable region of a nucleic acid, the regulatory sequence comprising a proximal regulatory sequence element (PSE) and a distal regulatory sequence element (DSE); wherein the PSE comprises a mouse U1 snRNA (“MmU1”) PSE, a mouse U2 snRNA (“MmU2”) PSE, a mouse U3 snRNA (“MmU3”) PSE, a mouse U4 snRNA (“MmU4”) PSE, a mouse U5 snRNA (“MmU5”) PSE, a mouse U6 snRNA (“MmU6”) PSE, a mouse U7 snRNA (“MmU7”) PSE, a mouse U11 snRNA (“MmU11”) PSE, a mouse U12 snRNA (“MmU12”) PSE, a mouse U7SK snRNA (“MmU7SK”) PSE, a human U1 snRNA (“HsU1”) PSE, a human U2 snRNA (“HsU2”) PSE, a human U3 snRNA (“HsU3”) PSE, a human U4 snRNA (“HsU4”) PSE, a human U5 snRNA (“HsU5”) PSE, a human U6 snRNA (“HsU6”) PSE, a human U7 snRNA (“HsU7”) PSE, a human U11 snRNA (“HsU11”) PSE, a human U12 snRNA (“HsU12”) PSE, or a human U7SK snRNA (“HsU7SK”) PSE; wherein the DSE comprises a mouse U1 snRNA (“MmU1”) DSE, a mouse U2 snRNA (“MmU2”) DSE, a mouse U3 snRNA (“MmU3”) DSE, a mouse U4 snRNA (“MmU4”) DSE, a mouse U5 snRNA (“MmU5”) DSE, a mouse U6 snRNA (“MmU6”) DSE, a mouse U7 snRNA (“MmU7”) DSE, a mouse U11 snRNA (“MmU11”) DSE, a mouse U12 snRNA (“MmU12”) DSE, a mouse U7SK snRNA (“MmU7SK”) DSE, a human U1 snRNA (“HsU1”) DSE, a human U2 snRNA (“HsU2”) DSE, a human U3 snRNA (“HsU3”) DSE, a human U4 snRNA (“HsU4”) DSE, a human U5 snRNA (“HsU5”) DSE, a human U6 snRNA (“HsU6”) DSE, a human U7 snRNA (“HsU7”) DSE, a human U11 snRNA (“HsU11”) DSE, a human U12 snRNA (“HsU12”) DSE, or a human U7SK snRNA (“HsU7SK”) DSE; and wherein the PSE and the DSE are from a different species as each other, or from a different snRNA as each other. In some embodiments, the PSE and the DSE are from a different species as each other, and from a different snRNA as each other. In some embodiments, the regulatory sequence comprises a promoter sequence. In some embodiments, the regulatory sequence comprises a terminator sequence. In some embodiments, the expression system produces a greater amount of RNA from the transcribable region than an expression system having a wild-type promoter and wild-type terminator operably coupled to the transcribable region. In some embodiments, the amount of RNA produced is at least 10% greater. Described herein, in some embodiments, are methods of producing a ribonucleic acid (RNA), comprising contacting a cell with the expression system.

Described herein, in some embodiments, are nucleic acid expression systems, comprising: a promoter sequence comprising a promoter proximal sequence element (PSE) and a promoter distal sequence element (DSE); wherein the PSE comprises a mouse U1 snRNA (“MmU1”) promoter PSE, a mouse U2 snRNA (“MmU2”) promoter PSE, a mouse U3 snRNA (“MmU3”) promoter PSE, a mouse U4 snRNA (“MmU4”) promoter PSE, a mouse U5 snRNA (“MmU5”) promoter PSE, a mouse U6 snRNA (“MmU6”) promoter PSE, a mouse U7 snRNA (“MmU7”) promoter PSE, a mouse U11 snRNA (“MmU11”) promoter PSE, a mouse U12 snRNA (“MmU12”) promoter PSE, a mouse U7SK snRNA (“MmU7SK”) promoter PSE, a human U1 snRNA (“HsU1”) promoter PSE, a human U2 snRNA (“HsU2”) promoter PSE, a human U3 snRNA (“HsU3”) promoter PSE, a human U4 snRNA (“HsU4”) promoter PSE, a human U5 snRNA (“HsU5”) promoter PSE, a human U6 snRNA (“HsU6”) promoter PSE, a human U7 snRNA (“HsU7”) promoter PSE, a human U11 snRNA (“HsU11”) promoter PSE, a human U12 snRNA (“HsU12”) promoter PSE, or a human U7SK snRNA (“HsU7SK”) promoter PSE; wherein the DSE comprises a mouse U1 snRNA (“MmU1”) promoter DSE, a mouse U2 snRNA (“MmU2”) promoter DSE, a mouse U3 snRNA (“MmU3”) promoter DSE, a mouse U4 snRNA (“MmU4”) promoter DSE, a mouse U5 snRNA (“MmU5”) promoter DSE, a mouse U6 snRNA (“MmU6”) promoter DSE, a mouse U7 snRNA (“MmU7”) promoter DSE, a mouse U11 snRNA (“MmU11”) promoter DSE, a mouse U12 snRNA (“MmU12”) promoter DSE, a mouse U7SK snRNA (“MmU7SK”) promoter DSE, a human U1 snRNA (“HsU1”) promoter DSE, a human U2 snRNA (“HsU2”) promoter DSE, a human U3 snRNA (“HsU3”) promoter DSE, a human U4 snRNA (“HsU4”) promoter DSE, a human U5 snRNA (“HsU5”) promoter DSE, a human U6 snRNA (“HsU6”) promoter DSE, a human U7 snRNA (“HsU7”) promoter DSE, a human U11 snRNA (“HsU11”) promoter DSE, a human U12 snRNA (“HsU12”) promoter DSE, or a human U7SK snRNA (“HsU7SK”) promoter DSE; and wherein the promoter PSE and the promoter DSE are from a different species as each other, or from a different snRNA as each other. In some embodiments, the promoter PSE and the promoter DSE are from a different species as each other, and from a different snRNA as each other. Some embodiments include a transcribable region operably coupled to the promoter sequence. Some embodiments include a terminator sequence 3′ to the transcribable region. In some embodiments, the expression system produces a greater amount of RNA from the transcribable region than an expression system having a wild-type promoter and wild-type terminator operably coupled to the transcribable region. In some embodiments, the amount of RNA produced is at least 10% greater. Described herein, in some embodiments, are methods of producing a ribonucleic acid (RNA), comprising contacting a cell with the expression system.

Described herein, in some embodiments, are nucleic acid expression systems, comprising, comprising: a terminator sequence 3′ to a transcribable region, the terminator sequence comprising a terminator proximal sequence element (PSE) and a terminator distal sequence element (DSE); wherein the PSE comprises a mouse U1 snRNA (“MmU1”) terminator PSE, a mouse U2 snRNA (“MmU2”) terminator PSE, a mouse U3 snRNA (“MmU3”) terminator PSE, a mouse U4 snRNA (“MmU4”) terminator PSE, a mouse U5 snRNA (“MmU5”) terminator PSE, a mouse U6 snRNA (“MmU6”) terminator PSE, a mouse U7 snRNA (“MmU7”) terminator PSE, a mouse U11 snRNA (“MmU11”) terminator PSE, a mouse U12 snRNA (“MmU12”) terminator PSE, a mouse U7SK snRNA (“MmU7SK”) terminator PSE, a human U1 snRNA (“HsU1”) terminator PSE, a human U2 snRNA (“HsU2”) terminator PSE, a human U3 snRNA (“HsU3”) terminator PSE, a human U4 snRNA (“HsU4”) terminator PSE, a human U5 snRNA (“HsU5”) terminator PSE, a human U6 snRNA (“HsU6”) terminator PSE, a human U7 snRNA (“HsU7”) terminator PSE, a human U11 snRNA (“HsU11”) terminator PSE, a human U12 snRNA (“HsU12”) terminator PSE, or a human U7SK snRNA (“HsU7SK”) terminator PSE; wherein the DSE comprises a mouse U1 snRNA (“MmU1”) terminator DSE, a mouse U2 snRNA (“MmU2”) terminator DSE, a mouse U3 snRNA (“MmU3”) terminator DSE, a mouse U4 snRNA (“MmU4”) terminator DSE, a mouse U5 snRNA (“MmU5”) terminator DSE, a mouse U6 snRNA (“MmU6”) terminator DSE, a mouse U7 snRNA (“MmU7”) terminator DSE, a mouse U11 snRNA (“MmU11”) terminator DSE, a mouse U12 snRNA (“MmU12”) terminator DSE, a mouse U7SK snRNA (“MmU7SK”) terminator DSE, a human U1 snRNA (“HsU1”) terminator DSE, a human U2 snRNA (“HsU2”) terminator DSE, a human U3 snRNA (“HsU3”) terminator DSE, a human U4 snRNA (“HsU4”) terminator DSE, a human U5 snRNA (“HsU5”) terminator DSE, a human U6 snRNA (“HsU6”) terminator DSE, a human U7 snRNA (“HsU7”) terminator DSE, a human U11 snRNA (“HsU11”) terminator DSE, a human U12 snRNA (“HsU12”) terminator DSE, or a human U7SK snRNA (“HsU7SK”) terminator DSE; and wherein the terminator PSE and the terminator DSE are from a different species as each other, or from a different snRNA as each other. In some embodiments, the terminator PSE and the terminator DSE are from a different species as each other, and from a different snRNA as each other. Some embodiments include a promoter sequence, wherein the transcribable region is operably coupled to the promoter sequence. In some embodiments, the expression system produces a greater amount of functional RNA from the transcribable region than an expression system having a wild-type U7 promoter and wild-type U7 terminator operably coupled to the transcribable region. In some embodiments, the amount of functional RNA produced is at least 10% greater. Described herein, in some embodiments, are methods of producing a ribonucleic acid (RNA), comprising contacting a cell with the expression system.

Described herein, in some embodiments, are nucleic acid expression systems, comprising, comprising: a promoter sequence operably coupled to a transcribable region of a nucleic acid, and a terminator sequence coupled to the transcribable region; wherein the promoter sequence comprises a promoter sequence of a first small nuclear RNA (snRNA) of a first organism species, wherein the terminator sequence comprises a terminator sequence of a second small nuclear RNA (snRNA) of a second organism species, and wherein the first and second snRNAs are different or wherein the first and second organism species are different. In some embodiments, the first snRNA is selected from the group consisting of snRNA U1, snRNA U2, snRNA U3, snRNA U4, snRNA U5, snRNA U6, snRNA U7, snRNA U11, snRNA U12 and snRNA 7SK. In some embodiments, the second snRNA is selected from the group consisting of snRNA U7, snRNA U2, snRNA U3, snRNA U4, snRNA U5, snRNA U6, snRNA U1, snRNA U11, snRNA U12 and snRNA 7SK. In some embodiments, the first snRNA is selected from the group consisting of snRNA U1, snRNA U2, snRNA U3, snRNA U4, snRNA U5, snRNA U6, snRNA U7, snRNA U11, snRNA U12 and snRNA 7SK; the second snRNA is selected from the group consisting of snRNA U7, snRNA U2, snRNA U3, snRNA U4, snRNA U5, snRNA U6, snRNA U1, snRNA U11, snRNA U12 and snRNA 7SK; the first organism species is selected from the group consisting of human and mouse; and the second organism species is selected from the group consisting of mouse and human. In some embodiments, the promoter comprises a promoter PSE sequence and a promoter DSE sequence, and wherein the either the promoter PSE or the promoter DSE is of a different organism species than the first organism species. In some embodiments, the promoter comprises a promoter PSE sequence and a promoter DSE sequence, and wherein the either the promoter PSE or the promoter DSE is of a different snRNA than the first snRNA. In some embodiments, the promoter comprises a promoter PSE sequence and a promoter DSE sequence, and wherein the either the promoter PSE or the promoter DSE is of a different organism species than the first organism species; and wherein the promoter comprises a promoter PSE sequence and a promoter DSE sequence, and wherein the either the promoter PSE or the promoter DSE is of a different snRNA than the first snRNA. In some embodiments, the terminator comprises a terminator PSE sequence and a terminator DSE sequence, and wherein the either the terminator PSE or the terminator DSE is of a different organism species than the second organism species. In some embodiments, the terminator comprises a terminator PSE sequence and a terminator DSE sequence, and wherein the either the terminator PSE or the terminator DSE is of a different snRNA than the second snRNA. In some embodiments, the terminator comprises a terminator PSE sequence and a terminator DSE sequence, and wherein the either the terminator PSE or the terminator DSE is of a different organism species than the second organism species; and wherein the terminator comprises a terminator PSE sequence and a terminator DSE sequence, and wherein the either the terminator PSE or the terminator DSE is of a different snRNA than the second snRNA. In some embodiments, the promoter comprises a promoter PSE sequence and a promoter DSE sequence, and wherein the either the promoter PSE or the promoter DSE is of a different organism species than the first organism species; and wherein the promoter comprises a promoter PSE sequence and a promoter DSE sequence, and wherein the either the promoter PSE or the promoter DSE is of a different snRNA than the first snRNA; wherein the terminator comprises a terminator PSE sequence and a terminator DSE sequence, and wherein the either the terminator PSE or the terminator DSE is of a different organism species than the second organism species; and wherein the terminator comprises a terminator PSE sequence and a terminator DSE sequence, and wherein the either the terminator PSE or the terminator DSE is of a different snRNA than the second snRNA. In some embodiments, the expression system produces a greater amount of RNA from the transcribable region than an expression system having a wild-type promoter and wild-type terminator operably coupled to the transcribable region. In some embodiments, the amount of RNA produced is at least 10% greater. Described herein, in some embodiments, are methods of producing a ribonucleic acid (RNA), comprising contacting a cell with the expression system.

Described herein, in some embodiments, are nucleic acid expression systems, comprising: a promoter sequence comprising a mouse U1 snRNA (“MmU1”) promoter sequence, a mouse U2 snRNA (“MmU2”) promoter sequence, a mouse U3 snRNA (“MmU3”) promoter sequence, a mouse U4 snRNA (“MmU4”) promoter sequence, a mouse U5 snRNA (“MmU5”) promoter sequence, a mouse U6 snRNA (“MmU6”) promoter sequence, a mouse U7 snRNA (“MmU7”) promoter sequence, a mouse U11 snRNA (“MmU11”) promoter sequence, a mouse U12 snRNA (“MmU12”) promoter sequence, a mouse U7SK snRNA (“MmU7SK”) promoter sequence, a human U1 snRNA (“HsU1”) promoter sequence, a human U2 snRNA (“HsU2”) promoter sequence, a human U3 snRNA (“HsU3”) promoter sequence, a human U4 snRNA (“HsU4”) promoter sequence, a human U5 snRNA (“HsU5”) promoter sequence, a human U6 snRNA (“HsU6”) promoter sequence, a human U7 snRNA (“HsU7”) promoter sequence, a human U11 snRNA (“HsU11”) promoter sequence, a human U12 snRNA (“HsU12”) promoter sequence, or a human U7SK snRNA (“HsU7SK”) promoter sequence, or a fragment or combination of fragments thereof, and a transcribable region operably coupled to the promoter sequence and to a terminator sequence comprising a mouse U1 snRNA (“MmU1”) terminator sequence, a mouse U2 snRNA (“MmU2”) terminator sequence, a mouse U3 snRNA (“MmU3”) terminator sequence, a mouse U4 snRNA (“MmU4”) terminator sequence, a mouse U5 snRNA (“MmU5”) terminator sequence, a mouse U6 snRNA (“MmU6”) terminator sequence, a mouse U7 snRNA (“MmU7”) terminator sequence, a mouse U11 snRNA (“MmU11”) terminator sequence, a mouse U12 snRNA (“MmU12”) terminator sequence, a mouse U7SK snRNA (“MmU7SK”) terminator sequence, a human U1 snRNA (“HsU1”) terminator sequence, a human U2 snRNA (“HsU2”) terminator sequence, a human U3 snRNA (“HsU3”) terminator sequence, a human U4 snRNA (“HsU4”) terminator sequence, a human U5 snRNA (“HsU5”) terminator sequence, a human U6 snRNA (“HsU6”) terminator sequence, a human U7 snRNA (“HsU7”) terminator sequence, a human U11 snRNA (“HsU11”) terminator sequence, a human U12 snRNA (“HsU12”) terminator sequence, or a human U7SK snRNA (“HsU7SK”) terminator sequence, or a fragment or combination of fragments thereof, wherein the promoter sequence and the terminator sequence are at least partially of a different organism species as each other, or are at least partially of a different snRNA as each other. In some embodiments, the promoter sequence and the terminator sequence are at least partially of a different organism species as each other, and are at least partially of a different snRNA as each other. In some embodiments, the expression system produces a greater amount of RNA from the transcribable region than an expression system having a wild-type promoter and wild-type terminator operably coupled to the transcribable region. In some embodiments, the amount of RNA produced is at least 10% greater. Described herein, in some embodiments, are methods of producing a ribonucleic acid (RNA), comprising contacting a cell with the expression system.

Disclosed herein, in some embodiments, are pharmaceutical compositions comprising a system described herein and a pharmaceutically acceptable carrier. Disclosed herein, in some embodiments, are engineered viruses comprising a system described herein. Disclosed herein, in some embodiments, are methods, comprising: administering a pharmaceutical composition or virus disclosed herein to a subject. In some embodiments, the virus comprises a parvovirus. In some embodiments, the virus is an adeno-associated virus (AAV). Disclosed herein, in some embodiments, are methods, comprising: administering the engineered virus to a subject. Disclosed herein, in some embodiments, are cells comprising a system described herein. In some embodiments, the cell is a neural cell.

### BRIEF DESCRIPTION OF THE DRAWINGS

FIG. 1 shows a pairwise alignment of mouse Scn1A and human SCN1A genomic regions including an NMD exon referred to as exon 20N.

FIG. 2 includes results of an initial U7 screen showing the rank of 96 U7 constructs based on the percent of productive Scn1A isoform expressed.

FIG. 3 includes an analysis of relative NMD transcript levels from Neuro-2a cells transfected with constructs expressing U7 cassettes containing single targeting sequences complementary to regions at both the 5′ and 3′ ends of mouse Scn1A exon 21N.

FIG. 4 includes an analysis of relative NMD transcript levels from Neuro-2a cells transfected with constructs expressing either single U7 cassettes or two U7 cassettes, with one targeting the 5′ region of exon 21N and one targeting the 3′ region of exon 21N (N=3 per construct).

FIG. 5 shows results of end-point RT-PCR for NMD (upper band) and productive (lower band) Scn1A isoform expression in brain tissue samples collected from animals transduced with AAV expressing either Scramble (Ctrl), Candidate 1, or Candidate 2 with 1×U7 cassettes. Each lane represents an individual animal.

FIG. 6 shows end-point RT-PCR for NMD (upper band) and productive (lower band) Scn1A isoform expression in samples collected from primary mouse cortical neurons transduced with AAVs expressing either Scramble (Ctrl), Candidate 1, or Candidate 2 with 4×U7 cassettes. Each pair of lanes shows independent biological replicates.

FIG. 7 shows end-point RT-PCR for NMD and productive Scn1A isoform expression in wildtype mice injected with increasing doses of AAVs expressing U7 candidates targeting mouse exon 21N.

FIG. 8 shows a dose-dependent increase in NaV1.1 protein expression in wildtype mice injected with AAVs expressing candidate U7 constructs targeting the 5′ end of mouse exon 21N. NaV1.1 levels are normalized to total protein levels in each sample.

FIG. 9 shows an analysis of relative NMD transcript levels in Neuro-2a cells transfected with constructs expressing the core U7 structural sequence with Candidate 1 targeting sequence under the control of different promoters and 3′ elements.

FIG. 10 shows a summary of end-point RT-PCR for relative amounts of productive Scn1A splice isoform in primary mouse cortical neurons transduced with increasing doses of AAVs expressing U7 candidate-1 driven by different combinations of mouse and human U1- and U7-related promoter and 3′ terminator elements.

FIG. 11 shows NaV1.1 levels in whole-brain lysates from mice injected with AAVs expressing U7 candidate 1 driven by different combinations of mouse and human U1-related promoter and 3′ terminator elements. NaV1.1 levels are normalized to total protein levels in each sample.

FIG. 12 shows survival curves of Scn1A control and heterozygous mutant mice injected with either AAV-mu1a1-Scramble-HU1 or AAV-mu1a1-Candidate 1-HU1.

FIG. 13 shows relative NMD transcript levels in Scn1A control and heterozygous mutant mice injected with either AAV-mu1a1-Scramble-HU1 or AAV-mu1a1-Candidate 1-HU1.

FIG. 14 shows total SCN1A mRNA levels in Scn1A control and heterozygous mutant mice injected with either AAV-mu1a1-Scramble-HU1 or AAV-mu1a1-Candidate 1-HU1.

FIG. 15 shows relative NaV1.1 levels in Scn1A control and heterozygous mutant mice injected with either AAV-mu1a1-Scramble-HU1 or AAV-mu1a1-Candidate 1-HU1.

FIG. 16 shows spontaneous seizure frequency (seizures/24 hr) measured by EEG in Scn1A control and heterozygous mutant mice injected with either saline or U7 AAV (Candidate 3; AAV9) at P2 via ICV. EEG recordings were made from P24-P45 and total seizure counts were averaged over the entire 21-day recording period.

FIG. 17 shows mean seizure duration in heterozygous Scn1A^{+/-} animals exhibiting spontaneous seizures after injection with either saline or U7 AAV (Candidate 3; AAV9) injected via ICV at P2. EEG recording was performed from P24-P45.

FIG. 18 shows survival curves of Scn1A control and heterozygous mutant mice injected with either saline or U7 AAV (Candidate 3; AAV9) at P2 via ICV.

FIG. 19 shows survival curves of Scn1A control and heterozygous mutant mice injected with either saline or U7 AAV (Candidate 3; AAV9) at P2 via ICV.

FIG. 20 shows relative NaV1.1 protein levels at P90 in Scn1A control and heterozygous mutant mice injected with either saline or U7 AAV (Candidate 3; AAV9) at P2 via ICV.

FIG. 21 shows relative NaV1.1 protein levels at P90 in Scn1A control and heterozygous mutant mice injected with either saline or U7 AAV (Candidate 3; PHP.eB) at P14 via retroorbital injection.

FIG. 22 shows quantification of SCN1A NMD isoform levels by end-point RT-PCR from differentiated human neuronal cultures (ReNcell CX) treated with formulation buffer (vehicle) or three different doses of AAV encoding Candidate 3.

FIG. 23 shows quantification of SCN1A productive isoform levels by end-point RT-PCR from differentiated human neuronal cultures (ReNcell CX) treated with formulation buffer (vehicle) or three different doses of AAV encoding Candidate 3.

FIG. 24 includes a schematic of a luciferase reporter containing a ubiquitous promoter (Ubc-SV40 pA), a canonical Kozak sequence, a fragment of mouse Scn2a exon 13, a fragment of mouse Scn2a intron 13, mouse Scn2a exon 13N, another fragment of mouse Scn2a intron 13, and a fragment of mouse Scn2a exon 14 fused in-frame to firefly luciferase.

FIG. 25 (top) includes results of end-point RT-PCR for NMD (upper band) and productive (lower band) and (bottom) an analysis of relative NMD and productive isoform transcript levels of Scn2a exon 13 in mouse Neuro-2a cells expressing a candidate ESS and U7 antisense sequence when driven by the combination of the mu1a1 promoter and HU1 terminator.

FIG. 26 shows relative NaV1.2 levels in differentiated ReNcell CX cultures transduced with scramble or three different U7 targeting sequences identified with this system. NaV1.2 levels in the plot within the figure were normalized to Actinin protein levels in each sample.

### DETAILED DESCRIPTION

Promoters and terminators play an indispensable role in regulating gene expression. These critical elements play a part in regulating both the strength of transcription and the longevity of the transcript. Together, promoters and terminators dictate the overall abundance of mRNA within the cell and ultimately play a significant role in determining protein contents within the cell. In the case of small nuclear RNAs, promoters and terminators may play a critical role in formation of secondary structures that may be required for snRNA function. Optimizing recombinant gene expression for gene therapy often requires the testing of multiple combinations of promoters and terminators to fine-tune the correct level and timing of expression. Initially, naturally occurring promoters and terminators were tested for their ability to drive their native gene expression in different cell types with some success but there was still a need to further control the level and timing of expression. To address this problem promoter and terminator swapping and mutagenesis was employed to optimize the level and timing of expression. Promoter and terminator swapping and mutagenesis has allowed researchers to specifically titrate the amount of transcription, the timing of transcription and the specific cell populations that express the gene in vivo. Nevertheless, this process of testing various promoter and terminator combinations requires the development of platforms for each gene therapy application. These platforms allow researchers to vary the amount of transcription, timing of transcription and the cell populations that express the gene therapy both in vitro and in vivo. Development of physiologically relevant platforms to test gene therapy variants may be necessary to develop the best gene therapies for a number of genetic disorders, including Dravet syndrome.

Precise pre-mRNA splicing may be essential for appropriate protein translation and may depend on the presence of consensus ‘cis’ sequences that define exon-intron boundaries and regulatory sequences recognized by splicing machinery. Point mutations at these consensus sequences can cause improper exon and intron recognition and may result in the formation of an aberrant transcript of the mutated gene. The splicing mutation may occur in both introns and exons and disrupt existing splice sites or splicing regulatory sequences (intronic and exonic silencers and enhancers), create new ones, or activate crypticones. Usually, such mutations result in errors during the splicing process and may lead to improper intron removal and thus cause alterations of an open reading frame (ORF). Alterations in the open reading frame may lead to truncated or extended mRNA products resulting in the translated protein unable to perform its normal function due to misfolding or degradation. Loss or reduced expression of proteins due to mRNA spicing errors lead to a number of genetic disorders, including Dravet syndrome.

Described herein are recombinant regulatory elements for expression systems. Also described are nucleic acid expression systems with recombinant regulatory elements. The regulatory elements and systems described herein address needs for improved expression systems, such as for gene editing or for transgenic expression.

Mutation of SCN1A, which encodes sodium channel NaV1.1, is a leading genetic cause of neurodevelopmental disorders such as Dravet syndrome. Analysis of ribonucleic acid (RNA) sequence data has identified an alternate SCN1A mRNA isoform that includes an extra exon, referred to as exon 20N. This alternate isoform is out of frame and results in nonsense-mediated decay (NMD).

Dravet syndrome is a devastating epileptic encephalopathy that may arise in otherwise normal babies in the first year of life, later accompanied by developmental delay, intellectual disability and mood disorders. The majority of cases (80%) may be caused by haploinsufficiency of SCN1A, a gene that encodes alpha subunit of the voltage-gated sodium channel protein, NaV1.1. NaV1.1 may also be referred to as sodium channel protein type 1 subunit alpha (SCN1A) protein. Current pharmacological treatments for Dravet syndrome are unable to completely control the epileptic seizures associated with the syndrome. Additionally, the pharmacological treatments are ineffective at inhibiting the subsequent neurological symptoms associated with Dravet syndrome. As a result, there are a number of gene-based therapies being developed for Dravet syndrome. One major obstacle for gene therapies for Dravet syndrome is that the SCN1A-coding sequence is too large to package in adeno-associated vectors (AAV) that are commonly employed for therapeutic gene delivery in the central nervous system. In recent years, alternative genetic approaches aiming to restore physiological levels of NaV1.1 to treat Dravet syndrome have been developed. These strategies rely on boosting the expression of the healthy copy of an SCN1A gene at the transcriptional or post-transcriptional level. One strategy to restore physiological levels of NaV1.1 is through targeted augmentation of nuclear gene output (TANGO). TANGO utilizes antisense oligonucleotides that are specifically designed to target the non-productive splicing site associated with Dravet syndrome and results in expression of the functional NaV1.1 protein. The current application includes a method of treating Dravet syndrome through the delivery of a modified U7 small nuclear RNA (snRNA) targeting exon 20N in humans (21N in mice) of neural cells via AAV so that a functional NaV1.1 protein is expressed and results in increased function of the NaV1.1 protein.

Described herein are compositions and methods for treating a neurodevelopmental disorder in a subject in need thereof. The compositions and methods described herein address an unmet need for safe and effective treatment of neurodevelopmental disorders. The neurodevelopmental disorder may be genetically caused, such as by a mutation in SCN1A (e.g. resulting in SCN1A haploinsufficiency). Described herein are compositions and methods for treating epileptic disorders such as Dravet syndrome in a subject in need thereof. The compositions and methods described herein address an unmet need for safe and effective treatment of epileptic disorders such as Dravet syndrome. The mutation may lead to aberrant SCN1A splicing, or result in inclusion of exon 20N in a mature SCN1A mRNA transcript. Examples of neurodevelopmental disorders may include an intellectual disability or epilepsy (e.g. including seizures).

The compositions and methods provided herein may improve upon pervious methods and systems. Some previous systems include are described at WO2017106377, WO2019040923, Han et al., Antisense oligonucleotides increase Scn1a expression and reduce seizures and SUDEP incidence in a mouse model of Dravet syndrome (Sci Transl Med. 2020 Aug. 26; 12(558):eaaz6100.), and Tanenhaus et al., Cell-Selective Adeno-Associated Virus-Mediated SCN1A Gene Regulation Therapy Rescues Mortality and Seizure Phenotypes in a Dravet Syndrome Mouse Model and Is Well Tolerated in Nonhuman Primates (Hum Gene Ther. 2022 June; 33(11-12):579-597), which references are incorporated by reference in their entirety.

In some embodiments, the system reduces a target (e.g. protein or RNA, such as a SCN1A protein or RNA) measurement in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80%, relative to a baseline target measurement. In some embodiments, the system reduces a target (e.g. protein or RNA, such as a SCN1A protein or RNA) measurement in a cell or population of cells by less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, or less than 80%, relative to a baseline target measurement.

Some embodiments include a system for modifying nucleic acid splicing. The system may include an exonic splicing silencer (ESS) nucleic acid sequence that enhances splicing suppression. The system may include an antisense nucleic acid sequence that targets an alternatively spliced region of an RNA. The antisense nucleic acid sequence may encode an SCN1A protein. Described herein, in some embodiments, is a system for modifying nucleic acid splicing comprising ESS nucleic acid sequence and an antisense nucleic acid sequence that targets an alternatively spliced region of an RNA encoding an SCN1A protein.

Some embodiments relate to a system for modifying nucleic acid splicing, comprising: an engineered U7 snRNA comprising an antisense nucleic acid sequence that targets an alternatively spliced region of an RNA encoding an SCN1A protein. In some embodiments, the engineered U7 snRNA further comprises an ESS nucleic acid sequence. In some embodiments, the engineered U7 snRNA does not comprise an ESS nucleic acid sequence.

Disclosed herein, in some embodiments, are systems for modifying nucleic acid splicing. In some embodiments, the system may include an ESS nucleic acid sequence. In some embodiments, the system may include an antisense nucleic acid sequence that targets an alternatively spliced region of an RNA. In some embodiments, the targeted RNA encodes an SCN1A protein.

Described herein, in some embodiments, is a nucleic acid system that may include deoxyribonucleic acid (DNA). The DNA may include an expression cassette. In some embodiments, the nucleic acid system that may include ribonucleic acid (RNA). The RNA may be encoded by the expression cassette.

### Targeted RNAs and Alternatively Spliced Regions of Targeted RNAs

Described herein, in some embodiments, are methods or systems that affect splicing of a target RNA. SCN1A RNA is an example of a target RNA. A target RNA may be or include a target transcript. A target RNA may be or include a target mRNA. A target RNA may be or include a target pre-mRNA. A target RNA may include a targeted exon. A target RNA may include a targeted intron. A target RNA may include a targeted intron/exon junction.

A target RNA may include a targeted region. A targeted region may bind with or be bound by an antisense nucleic acid sequence. A targeted region may include an alternatively spliced region such as an alternative exon (e.g. exon 20N). A targeted region may include a splice junction of an alternative exon (e.g. an intron/exon junction comprising at least part of exon 20N). A targeted region may include a region near an alternative exon such as an intron sequence. A targeted region may include an intron sequence. A targeted region may include an alternative exon. A targeted region may exclude an intron. A targeted region may exclude an alternative exon. A targeted region may include part of an intron sequence. A targeted region may include part of an alternative exon. A targeted region may exclude part of an intron sequence. A targeted region may exclude part of an alternative exon. A targeted region may encompass both a region near an alternative exon and at least part of the alternative exon.

Some embodiments refer to or include an alternatively spliced region of a target RNA, such as an alternatively spliced region of an SCN1A RNA. For example, some embodiments of a system or method include or refer to an antisense nucleic acid sequence that targets an alternatively spliced region of a target RNA such as an SCN1A RNA. An example of an alternatively spliced region is exon 20N of an SCN1A RNA. An example of an SCN1A mRNA transcript including exon 20N may be found at www.ncbi.nlm.nih.gov under reference sequence NR_148667.2, as last updated as of the effective filing date.

The alternatively spliced region may be or include an alternatively spliced exon. The alternatively spliced region may be out of frame with a productive transcript. This may lead to production of a downstream stop codon. In some embodiments, inclusion of the alternatively spliced region results in an mRNA (e.g. a mature SCN1A mRNA) having a premature stop codon. Inclusion of a premature stop codon may result in a non-productive mRNA. Inclusion of a premature stop codon may result in the mRNA being non-productive for functional or full-length SCN1A protein. Inclusion of a premature stop codon may result in nonsense mediated decay. In some embodiments, the alternatively spliced regions out of frame with an exon of a productive transcript. In some embodiments, the alternatively spliced region may include a stop codon. In some embodiments, the alternatively spliced region does not include a stop codon.

In some embodiments, the alternatively spliced region may be an alternatively spliced exon. In some embodiments, the alternatively spliced region may not be an alternatively spliced exon. In some embodiments, the alternatively spliced region is an alternatively spliced intron. In some embodiments, the alternatively spliced exon comprises exon 20N (e.g. of a human SCN1A mRNA). In some embodiments, the alternatively spliced exon comprises exon 21N (e.g. of a mouse SCN1A mRNA).

A targeted region may include SEQ ID NO: 153. A targeted region may include part of SEQ ID NO: 153. A targeted region may include a sequence at least 99% identical to SEQ ID NO: 153 or at least 99% identical to part of SEQ ID NO: 153. A targeted region may include a sequence at least 98% identical to SEQ ID NO: 153 or at least 98% identical to part of SEQ ID NO: 153. A targeted region may include a sequence at least 97% identical to SEQ ID NO: 153 or at least 97% identical to part of SEQ ID NO: 153. A targeted region may include a sequence at least 96% identical to SEQ ID NO: 153 or at least 96% identical to part of SEQ ID NO: 153. A targeted region may include a sequence at least 95% identical to SEQ ID NO: 153 or at least 95% identical to part of SEQ ID NO: 153. A targeted region may include a sequence at least 94% identical to SEQ ID NO: 153 or at least 94% identical to part of SEQ ID NO: 153. A targeted region may include a sequence at least 93% identical to SEQ ID NO: 153 or at least 93% identical to part of SEQ ID NO: 153. A targeted region may include a sequence at least 92% identical to SEQ ID NO: 153 or at least 92% identical to part of SEQ ID NO: 153. A targeted region may include a sequence at least 91% identical to SEQ ID NO: 153 or at least 91% identical to part of SEQ ID NO: 153. A targeted region may include a sequence at least 9′% identical to SEQ ID NO: 153 or at least 9′% identical to part of SEQ ID NO: 153.

A targeted region may include SEQ ID NO: 154. A targeted region may include part of SEQ ID NO: 154. A targeted region may include a sequence at least 99% identical to SEQ ID NO: 154 or at least 99% identical to part of SEQ ID NO: 154. A targeted region may include a sequence at least 98% identical to SEQ ID NO: 154 or at least 98% identical to part of SEQ ID NO: 154. A targeted region may include a sequence at least 97% identical to SEQ ID NO: 154 or at least 97% identical to part of SEQ ID NO: 154. A targeted region may include a sequence at least 96% identical to SEQ ID NO: 154 or at least 96% identical to part of SEQ ID NO: 154. A targeted region may include a sequence at least 95% identical to SEQ ID NO: 154 or at least 95% identical to part of SEQ ID NO: 154. A targeted region may include a sequence at least 94% identical to SEQ ID NO: 154 or at least 94% identical to part of SEQ ID NO: 154. A targeted region may include a sequence at least 93% identical to SEQ ID NO: 154 or at least 93% identical to part of SEQ ID NO: 154. A targeted region may include a sequence at least 92% identical to SEQ ID NO: 154 or at least 92% identical to part of SEQ ID NO: 154. A targeted region may include a sequence at least 91% identical to SEQ ID NO: 154 or at least 91% identical to part of SEQ ID NO: 154. A targeted region may include a sequence at least 9′% identical to SEQ ID NO: 154 or at least 9′% identical to part of SEQ ID NO: 154.

In some embodiments, the exon may include a nucleic acid sequence identical to SEQ ID NO: 1. In some embodiments, the exon may include a nucleic acid sequence at least 99% identical to SEQ ID NO: 1. In some embodiments, the exon may include a nucleic acid sequence at least 98% identical to SEQ ID NO: 1. In some embodiments, the exon may include a nucleic acid sequence at least 97% identical to SEQ ID NO: 1. In some embodiments, the exon may include a nucleic acid sequence at least 96% identical to SEQ ID NO: 1. In some embodiments, the exon may include a nucleic acid sequence at least 95% identical to SEQ ID NO: 1. In some embodiments, the exon may include a nucleic acid sequence at least 94% identical to SEQ ID NO: 1. In some embodiments, the exon may include a nucleic acid sequence at least 93% identical to SEQ ID NO: 1. In some embodiments, the exon may include a nucleic acid sequence at least 92% identical to SEQ ID NO: 1. In some embodiments, the exon may include a nucleic acid sequence at least 91% identical to SEQ ID NO: 1. In some embodiments, the exon may include a nucleic acid sequence at least 90% identical to SEQ ID NO: 1. In some embodiments, the exon may include a nucleic acid sequence at least 85% identical to SEQ ID NO: 1. In some embodiments, the exon may include a nucleic acid sequence at least 80% identical to SEQ ID NO: 1. In some embodiments, the exon may include a nucleic acid sequence at least 75% identical to SEQ ID NO: 1. In some embodiments, the exon may include a nucleic acid sequence of at least 70% identical to SEQ ID NO: 1.

In some embodiments, the exon may include a nucleic acid sequence identical to SEQ ID NO: 2. In some embodiments, the exon may include a nucleic acid sequence at least 99% identical to SEQ ID NO: 2. In some embodiments, the exon may include a nucleic acid sequence at least 98% identical to SEQ ID NO: 2. In some embodiments, the exon may include a nucleic acid sequence at least 97% identical to SEQ ID NO: 2. In some embodiments, the exon may include a nucleic acid sequence at least 96% identical to SEQ ID NO: 2. In some embodiments, the exon may include a nucleic acid sequence at least 95% identical to SEQ ID NO: 2. In some embodiments, the exon may include a nucleic acid sequence at least 94% identical to SEQ ID NO: 2. In some embodiments, the exon may include a nucleic acid sequence at least 93% identical to SEQ ID NO: 2. In some embodiments, the exon may include a nucleic acid sequence at least 92% identical to SEQ ID NO: 2. In some embodiments, the exon may include a nucleic acid sequence at least 91% identical to SEQ ID NO: 2. In some embodiments, the exon may include a nucleic acid sequence at least 90% identical to SEQ ID NO: 2. In some embodiments, the exon may include a nucleic acid sequence at least 85% identical to SEQ ID NO: 2. In some embodiments, the exon may include a nucleic acid sequence at least 80% identical to SEQ ID NO: 2. In some embodiments, the exon may include a nucleic acid sequence at least 75% identical to SEQ ID NO: 2. In some embodiments, the exon may include a nucleic acid sequence of at least 70% identical to SEQ ID NO: 2. A target nucleic acid sequence may target any of the aforementioned exon sequences. A target nucleic acid sequence may target a region of any of the aforementioned exon sequences.

A targeted region may be or comprise a length of nucleotides. For example, a targeted region may be about 10, about 15, about 20, about 25, about 30, about 35, about 40, about 45, about 50, about 60, about 70, about 80, about 90, about 100, about 110, about 120, about 130, about 140, or about 150 nucleotides in length, or a range of lengths defined by any 2 of the aforementioned lengths. In some embodiments, the length is at least 10 nucleotides. In some embodiments, the length is at least 15 nucleotides. In some embodiments, the length is at least 20 nucleotides. In some embodiments, the length is at least 25 nucleotides. In some embodiments, the length is at least 30 nucleotides. In some embodiments, the length is at least 35 nucleotides. In some embodiments, the length is at least 40 nucleotides. In some embodiments, the length is at least 45 nucleotides. In some embodiments, the length is at least 50 nucleotides. In some embodiments, the length is at least 60 nucleotides. In some embodiments, the length is at least 70 nucleotides. In some embodiments, the length is at least 80 nucleotides. In some embodiments, the length is at least 90 nucleotides. In some embodiments, the length is at least 100 nucleotides. In some embodiments, the length is at least 110 nucleotides. In some embodiments, the length is at least 120 nucleotides. In some embodiments, the length is at least 130 nucleotides. In some embodiments, the length is at least 140 nucleotides. In some embodiments, the length is at least 150 nucleotides. In some embodiments, the length is less than 15 nucleotides. In some embodiments, the length is less than 20 nucleotides. In some embodiments, the length is less than 25 nucleotides. In some embodiments, the length is less than 30 nucleotides. In some embodiments, the length is less than 35 nucleotides. In some embodiments, the length is less than 40 nucleotides. In some embodiments, the length is less than 45 nucleotides. In some embodiments, the length is less than 50 nucleotides. In some embodiments, the length is less than 60 nucleotides. In some embodiments, the length is less than 70 nucleotides. In some embodiments, the length is less than 80 nucleotides. In some embodiments, the length is less than 90 nucleotides. In some embodiments, the length is less than 100 nucleotides. In some embodiments, the length is less than 110 nucleotides. In some embodiments, the length is less than 120 nucleotides. In some embodiments, the length is less than 130 nucleotides. In some embodiments, the length is less than 140 nucleotides. In some embodiments, the length is less than 150 nucleotides.

In some embodiments, an antisense nucleic acid sequence binds a specific region of a target RNA such as an SCN1A RNA. In some embodiments, the targeted region is within an exon of an endogenous target RNA such as endogenous SCN1A RNA. In some embodiments, the targeted region is within an exon of an endogenous target RNA such as an SCN1A mRNA. In some embodiments, an antisense nucleic acid sequence targets an alternatively spliced exon of the endogenous target RNA such as SCN1A RNA. In some embodiments, the alternatively spliced exon includes an exon 20N of an SCN1A RNA.

In some embodiments, the targeted region is within a 5′ half or 5′ end of an intron or exon of the endogenous target RNA such as SCN1A RNA. For example, in some embodiments, the targeted region may be closer to the 5′ end of an intron of the endogenous target RNA. In some embodiments, the targeted region includes the 5′ end of an intron of the endogenous target RNA. In some embodiments, the targeted region may be closer to the 5′ end of an exon of the endogenous target RNA. In some embodiments, the targeted region includes the 5′ end of an exon of the endogenous target RNA.

In some embodiments, the targeted region is within a 3′ half or 3′ end of an intron or exon of the endogenous target RNA such as an endogenous SCN1A RNA. For example, in some embodiments, the targeted region may be closer to the 3′ end of an intron of the endogenous target RNA. In some embodiments, the targeted region includes the 3′ end of an intron of the endogenous target RNA. In some embodiments, the targeted region may be closer to the 3′ end of an exon of the endogenous target RNA. In some embodiments, the targeted region includes the 3′ end of an exon of the endogenous target RNA.

In some embodiments, the targeted region is within 100 nucleotides of an intron/exon junction. In some embodiments, the targeted region is within 10, 20, 30, 40, 50, 60, 70, 80, 90, or 100 nucleotides of an intron/exon junction. In some embodiments, the targeted region is not within 10, 20, 30, 40, 50, 60, 70, 80, 90, or 100 nucleotides of an intron/exon junction.

In some embodiments, the targeted region of the endogenous target RNA such as the endogenous SCN1A RNA (e.g. SCN1A mRNA or pre-mRNA) includes an intron-exon junction of the endogenous RNA. The intron-exon junction refers to the boundary between an intron and exon and includes the splice site that separates the intron and the exon upon pre-mRNA splicing. In some embodiments, the targeted region of the endogenous target RNA is within 0 nt and 150 nt of an intron-exon junction. In some embodiments, the targeted region of the endogenous target RNA is within 10 nt and 150 nt of an intron-exon junction. In some embodiments, the targeted region of the endogenous target RNA is within 20 nt and 150 nt of an intron-exon junction. In some embodiments, the targeted region of the endogenous target RNA is within 30 nt and 150 nt of an intron-exon junction. In some embodiments, the targeted region of the endogenous target RNA is within 40 nt and 150 nt of an intron-exon junction. In some embodiments, the targeted region of the endogenous target RNA is within 40 nt and 150 nt of an intron-exon junction. In some embodiments, the targeted region of the endogenous target RNA is within 60 nt and 150 nt of an intron-exon junction. In some embodiments, the targeted region of the endogenous target RNA is within 70 nt and 150 nt of an intron-exon junction. In some embodiments, the targeted region of the endogenous target RNA is within 80 nt and 150 nt of an intron-exon junction. In some embodiments, the targeted region of the endogenous target RNA is within 90 nt and 150 nt of an intron-exon junction. In some embodiments, the targeted region of the endogenous target RNA is within 100 nt and 150 nt of an intron-exon junction. In some embodiments, the targeted region of the endogenous target RNA is within 110 nt and 150 nt of an intron-exon junction. In some embodiments, the targeted region of the endogenous target RNA is within 120 nt and 150 nt of an intron-exon junction. In some embodiments, the targeted region of the endogenous target RNA is within 130 nt and 150 nt of an intron-exon junction. In some embodiments, the targeted region of the endogenous target RNA is within 140 nt and 150 nt of an intron-exon junction.

In some embodiments, the targeted region of the endogenous target RNA such as the endogenous SCN1A RNA is within 0 nt and 140 nt of an intron-exon junction. In some embodiments, the targeted region of the endogenous target RNA is within 0 nt and 130 nt of an intron-exon junction. In some embodiments, the targeted region of the endogenous target RNA is within 0 nt and 120 nt of an intron-exon junction. In some embodiments, the targeted region of the endogenous target RNA is within 0 nt and 110 nt of an intron-exon junction. In some embodiments, the targeted region of the endogenous target RNA is within 0 nt and 100 nt of an intron-exon junction. In some embodiments, the targeted region of the endogenous target RNA is within 0 nt and 90 nt of an intron-exon junction. In some embodiments, the targeted region of the endogenous target RNA is within 0 nt and 80 nt of an intron-exon junction. In some embodiments, the targeted region of the endogenous target RNA is within 0 nt and 70 nt of an intron-exon junction. In some embodiments, the targeted region of the endogenous target RNA is within 0 nt and 60 nt of an intron-exon junction. In some embodiments, the targeted region of the endogenous target RNA is within 0 nt and 50 nt of an intron-exon junction. In some embodiments, the targeted region of the endogenous target RNA is within 0 nt and 40 nt of an intron-exon junction. In some embodiments, the targeted region of the endogenous target RNA is within 0 nt and 30 nt of an intron-exon junction. In some embodiments, the targeted region of the endogenous target RNA is within 0 nt and 20 nt of an intron-exon junction.

In some embodiments, the targeted region of the endogenous target RNA is within 0 nt, 10 nt, 20 nt, 30 nt, 40 nt, 50 nt, 60 nt, 70 nt, 80 nt, 90 nt, 100 nt, 110 nt, 120 nt, 130 nt, 140 nt, or 150 of an intron-exon junction. In some embodiments, the targeted region of the endogenous target RNA includes an intro-exon junction.

A target RNA may be or include a target mRNA. A target mRNA may be or include an target pre-mRNA. For example, a target RNA may include a target pre-mRNA. A SCN1A RNA may be or include an SCN1A mRNA. An SCN1A mRNA may be or include an SCN1A pre-mRNA. For example, a target SCN1A RNA may include a SCN1A pre-mRNA. A pre-mRNA may include an mRNA before splicing, or before splicing is completed. Whan an mRNA has fully undergone splicing, it may be referred to as a mature mRNA.

Described herein, in some embodiments, are methods or systems that affect splicing of a target RNA such as an SCN1A RNA. In some embodiments, the target RNA comprises a mammalian target RNA. In some embodiments, the target RNA comprises a primate target RNA. In some embodiments, the target RNA comprises a human target RNA. In some embodiments, the target RNA comprises a rodent or mouse target RNA.

Some embodiments relate to a sodium channel protein type 1 subunit alpha (SCN1A) protein. For example, a method may be directed at reducing or preventing inclusion of a non-productive exon in a mature SCN1A mRNA, where inclusion of the non-productive exon results in nonsense-mediated decay (NMD). Some examples of SCN1A protein sequences are included at UniProt.org under accession numbers P35498 (human) and A2APX8 (mouse), as last updated as of the effective filing date. The SCN1A protein may include the amino acid sequence of SEQ ID NO: 155, which includes the sequence found at UniProt P35498-1 (human SCN1A protein). An SCN1A protein may include an amino acid sequence at least 85%, at least 90%, at least 91%, at least 92%, at least 93%, at least 94%, at least 95%, at least 96%, at least 97%, at least 98%, or at least 99% identical to SEQ ID NO: 155.

Modified U7 snRNAs

Disclosed herein, in some embodiments, are recombinant small nuclear RNAs (snRNAs) or snRNA sequences. Disclosed herein, in some embodiments, are modified or recombinant U7 small nuclear RNAs (snRNAs) or modified or recombinant U7 snRNA sequences. A modified or recombinant U7 snRNA, or a modified or recombinant U7 snRNA sequence may be included as part of a system, or may be used in a method herein. Disclosed herein, in some embodiments, are systems that include a modified or recombinant U7 snRNA or that include a modified or recombinant U7 snRNA sequence. The modified or recombinant U7 snRNA may be or include an engineered U7 snRNA. Terms such as modified, recombinant, and engineered may be used interchangeably herein. The U7 snRNA sequence may be useful for modifying nucleic acid splicing. In some embodiments, the U7 snRNA sequence may include an exonic splicing silencer (ESS) nucleic acid sequence that enhances splicing suppression. Some embodiments do not include an ESS nucleic acid sequence. In some embodiments, the U7 snRNA sequence may include an antisense nucleic acid sequence that targets an alternatively spliced region of a ribonucleic acid (RNA). In some embodiments, the targeted RNA may encode a sodium channel protein type 1 subunit alpha (SCN1A) protein. In some embodiments, the targeted RNA may encode a sodium channel protein type 2 subunit alpha (SCN2A) protein. The U7 snRNA sequence may include a smOPT sequence. The U7 snRNA sequence may include a hairpin.

Described herein, in some embodiments, is a nucleic acid system that may include one or more RNA molecules. In some embodiments, the system may include at least one RNA molecule. In some embodiments, the system may include at least two RNA molecules. In some embodiments, the system may include at least three RNA molecules. In some embodiments, the system may include at least four RNA molecules. In some embodiments, the system may include at least five RNA molecules. In some embodiments, the system may include at most one RNA molecule. In some embodiments, the system may include at most two RNA molecules. In some embodiments, the system may include at most three RNA molecules. In some embodiments, the system may include at most four RNA molecules. In some embodiments, the system may include at most five RNA molecules.

In some embodiments, the RNA may be a modified U7 snRNA. Some U rich small nuclear ribonucleoproteins (snRNPs) include complexes that mediate the splicing of pre-mRNAs. U7 snRNP may be an exception in not being involved in splicing but may be a key factor in 3′ end processing of replication-dependent histone mRNAs. However, by introducing controlled changes in the U7 snRNA histone binding sequence and in the Sm motif, U7 can be used as an effective tool for gene therapy. The modified U7 snRNP may thus be not involved in processing of replication-dependent histone pre-mRNA but instead target splicing by inducing efficient skipping or inclusion of selected exons. Modification of the sequence motif of U7 snRNA can make the snRNP particle hybridize to almost any RNA sequence within the nucleoplasm.

A modified U7 snRNA may include, in the following order from 5′ to 3′: (1) an optional exonic splicing silencer, (2) a targeting sequence (e.g. an antisense sequence) complementary to a region of a target mRNA, (4) a smOPT sequence, and (5) a hairpin sequence.

In some embodiments, a modified U7 snRNA includes a U7 core sequence, or an aspect of a U7 core sequence. In some embodiments, a recombinant U7 snRNA includes a U7 core sequence, or an aspect of a U7 core sequence. An example of a U7 core sequence is included in Table 1. For the U7 core sequence shown in Table 1, an antisense sequence is in parentheses, a smOPT is in in upper case, and a hairpin is in brackets. The U7 core sequence may also include an ESS sequence at its 5′ terminus. The antisense sequence may be or include a targeting sequence herein.

TABLE 1

| | | SEQ |
|---|---|---|
| | | ID |
| Component | Sequence (5′ to 3′) | NO: |
| Mouse Exon | GATAATCTTGCTCCAACTTGGATGGGGTGGAG | 1 |
| 21N | CGGTGGTTCCTCCCCTCAGCCCTTTATTATGG | |
| Human Exon | GATAATCTTGCTCCAACTTGGATGGGGTGGAG | 2 |
| 20N | CGCTGGTTCCTCCCCTGAGCCCTTTATTATGG | |
| U7 Core | a(N . . . N)AATTTTTGGAG[caggtttt | 3 |
| Sequence | ctgacttcggtcggaaaacccct] | |
| U7 Hairpin | CAGGUUUUCUGACUUCGGUCGGAAAACCCCU | 4 |
| Sequence | | |

Some embodiments include a modified U7 small nuclear RNA (snRNA). In some embodiments, the modified U7 snRNA includes a U7 core sequence at least 80% identical to a U7 core sequence set forth in Table 1. In some embodiments, the modified U7 snRNA includes a U7 core sequence at least 85% identical to a U7 core sequence set forth in Table 1. In some embodiments, the modified U7 snRNA includes a U7 core sequence at least 90% identical to a U7 core sequence set forth in Table 1. In some embodiments, the modified U7 snRNA includes a U7 core sequence at least 91% identical to a U7 core sequence set forth in Table 1. In some embodiments, the modified U7 snRNA includes a U7 core sequence at least 92% identical to a U7 core sequence set forth in Table 1. In some embodiments, the modified U7 snRNA includes a U7 core sequence at least 93% identical to a U7 core sequence set forth in Table 1. In some embodiments, the modified U7 snRNA includes a U7 core sequence at least 94% identical to a U7 core sequence set forth in Table 1. In some embodiments, the modified U7 snRNA includes a U7 core sequence at least 95% identical to a U7 core sequence set forth in Table 1. In some embodiments, the modified U7 snRNA includes a U7 core sequence at least 96% identical to a U7 core sequence set forth in Table 1. In some embodiments, the modified U7 snRNA includes a U7 core sequence at least 97% identical to a U7 core sequence set forth in Table 1. In some embodiments, the modified U7 snRNA includes a U7 core sequence at least 98% identical to a U7 core sequence set forth in Table 1. In some embodiments, the modified U7 snRNA includes a U7 core sequence at least 99% identical to a U7 core sequence set forth in Table 1. In some embodiments, the modified U7 snRNA includes a U7 core sequence set forth in Table 1. In some embodiments, the modified U7 snRNA is a U7 core sequence set forth in Table 1.

Some embodiments include a modified U7 small nuclear RNA (snRNA). Some examples of sequences of modified U7 snRNAs are in Table 14A. In some embodiments, the modified U7 snRNA is or includes an RNA sequence at least 80% identical to an RNA sequence set forth in Table 14A. In some embodiments, the modified U7 snRNA is or includes an RNA sequence at least 85% identical to an RNA sequence set forth in Table 14A. In some embodiments, the modified U7 snRNA is or includes an RNA sequence at least 90% identical to an RNA sequence set forth in Table 14A. In some embodiments, the modified U7 snRNA is or includes an RNA sequence at least 91% identical to an RNA sequence set forth in Table 14A. In some embodiments, the modified U7 snRNA is or includes an RNA sequence at least 92% identical to an RNA sequence set forth in Table 14A. In some embodiments, the modified U7 snRNA is or includes an RNA sequence at least 93% identical to an RNA sequence set forth in Table 14A. In some embodiments, the modified U7 snRNA is or includes an RNA sequence at least 94% identical to an RNA sequence set forth in Table 14A. In some embodiments, the modified U7 snRNA is or includes an RNA sequence at least 95% identical to an RNA sequence set forth in Table 14A. In some embodiments, the modified U7 snRNA is or includes an RNA sequence at least 96% identical to an RNA sequence set forth in Table 14A. In some embodiments, the modified U7 snRNA is or includes an RNA sequence at least 97% identical to an RNA sequence set forth in Table 14A. In some embodiments, the modified U7 snRNA is or includes an RNA sequence at least 98% identical to an RNA sequence set forth in Table 14A. In some embodiments, the modified U7 snRNA is or includes an RNA sequence at least 99% identical to an RNA sequence set forth in Table 14A. In some embodiments, the modified U7 snRNA is or includes an RNA sequence set forth in Table 14A. In some embodiments, the modified U7 snRNA is an RNA sequence set forth in Table 14A.

Some examples of sequences of modified U7 snRNAs are in Table 14B. In some embodiments, the modified U7 snRNA is or includes an RNA sequence at least 80% identical to an RNA sequence set forth in Table 14B. In some embodiments, the modified U7 snRNA is or includes an RNA sequence at least 85% identical to an RNA sequence set forth in Table 14B. In some embodiments, the modified U7 snRNA is or includes an RNA sequence at least 90% identical to an RNA sequence set forth in Table 14B. In some embodiments, the modified U7 snRNA is or includes an RNA sequence at least 91% identical to an RNA sequence set forth in Table 14B. In some embodiments, the modified U7 snRNA is or includes an RNA sequence at least 92% identical to an RNA sequence set forth in Table 14B. In some embodiments, the modified U7 snRNA is or includes an RNA sequence at least 93% identical to an RNA sequence set forth in Table 14B. In some embodiments, the modified U7 snRNA is or includes an RNA sequence at least 94% identical to an RNA sequence set forth in Table 14B. In some embodiments, the modified U7 snRNA is or includes an RNA sequence at least 95% identical to an RNA sequence set forth in Table 14B. In some embodiments, the modified U7 snRNA is or includes an RNA sequence at least 96% identical to an RNA sequence set forth in Table 14B. In some embodiments, the modified U7 snRNA is or includes an RNA sequence at least 97% identical to an RNA sequence set forth in Table 14B. In some embodiments, the modified U7 snRNA is or includes an RNA sequence at least 98% identical to an RNA sequence set forth in Table 14B. In some embodiments, the modified U7 snRNA is or includes an RNA sequence at least 99% identical to an RNA sequence set forth in Table 14B. In some embodiments, the modified U7 snRNA is or includes an RNA sequence set forth in Table 14B. In some embodiments, the modified U7 snRNA is an RNA sequence set forth in Table 14B.

Some examples of sequences of modified U7 snRNAs are in Table 14C. In some embodiments, the modified U7 snRNA is or includes an RNA sequence at least 80% identical to an RNA sequence set forth in Table 14C. In some embodiments, the modified U7 snRNA is or includes an RNA sequence at least 85% identical to an RNA sequence set forth in Table 14C. In some embodiments, the modified U7 snRNA is or includes an RNA sequence at least 90% identical to an RNA sequence set forth in Table 14C. In some embodiments, the modified U7 snRNA is or includes an RNA sequence at least 91% identical to an RNA sequence set forth in Table 14C. In some embodiments, the modified U7 snRNA is or includes an RNA sequence at least 92% identical to an RNA sequence set forth in Table 14C. In some embodiments, the modified U7 snRNA is or includes an RNA sequence at least 93% identical to an RNA sequence set forth in Table 14C. In some embodiments, the modified U7 snRNA is or includes an RNA sequence at least 94% identical to an RNA sequence set forth in Table 14C. In some embodiments, the modified U7 snRNA is or includes an RNA sequence at least 95% identical to an RNA sequence set forth in Table 14C. In some embodiments, the modified U7 snRNA is or includes an RNA sequence at least 96% identical to an RNA sequence set forth in Table 14C. In some embodiments, the modified U7 snRNA is or includes an RNA sequence at least 97% identical to an RNA sequence set forth in Table 14C. In some embodiments, the modified U7 snRNA is or includes an RNA sequence at least 98% identical to an RNA sequence set forth in Table 14C. In some embodiments, the modified U7 snRNA is or includes an RNA sequence at least 99% identical to an RNA sequence set forth in Table 14C. In some embodiments, the modified U7 snRNA is or includes an RNA sequence set forth in Table 14C. In some embodiments, the modified U7 snRNA is an RNA sequence set forth in Table 14C.

In some embodiments, the system reduces a nonsense-mediated decay (NMD) exon measurement (e.g. an exon 20N measurement) in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, relative to a baseline target measurement. In some embodiments, expression of the system in a cell or a population of cells reduces an exon 20N measurement in a cell or population of cells by at least 10%, at least 20%, at least 30%, at least 40%, at least 50%, at least 60%, at least 70%, at least 80%, or at least 90%, relative to a baseline exon 20N measurement. In some embodiments, the system reduces a nonsense-mediated decay (NMD) exon measurement (e.g. an exon 20N measurement) in a cell or population of cells by less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, or less than 95%, relative to a baseline target measurement.

In some embodiments, the system reduces a nonsense-mediated decay (NMD) transcript measurement (e.g. an NMD SCN1A RNA measurement) in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, relative to a baseline target measurement. In some embodiments, expression of the system in a cell or a population of cells increases a productive form of an SCN1A transcript measurement in a cell or population of cells by at least 10%, at least 20%, at least 30%, at least 40%, at least 50%, at least 60%, at least 70%, at least 80%, or at least 90%, relative to a baseline SCN1A transcript measurement. In some embodiments, expression of the system in a cell or a population of cells increases a productive form of an SCN1A transcript measurement in a cell or population of cells by more than 70%, relative to a baseline SCN1A transcript measurement. In some embodiments, the system reduces a nonsense-mediated decay (NMD) transcript measurement (e.g. an NMD SCN1A RNA measurement) in a cell or population of cells by less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, or less than 95%, relative to a baseline target measurement.

In some embodiments, expression of the system in a cell or a population of cells an TARGET protein measurement in a cell or population of cells by at least 10%, at least 20%, at least 30%, at least 40%, at least 50%, at least 60%, at least 70%, at least 80%, or at least 90%, relative to a baseline TARGET protein measurement. In some embodiments, expression of the system in a cell or a population of cells an TARGET protein measurement in a cell or population of cells by at least 70%, relative to a baseline TARGET protein measurement. In some embodiments, expression of the system in a cell or a population of cells an SCN1A protein measurement in a cell or population of cells by at least 10%, at least 20%, at least 30%, at least 40%, at least 50%, at least 60%, at least 70%, at least 80%, or at least 90%, relative to a baseline SCN1A protein measurement. In some embodiments, expression of the system in a cell or a population of cells an SCN1A protein measurement in a cell or population of cells by at least 70%, relative to a baseline SCN1A protein measurement. In some embodiments, expression of the system in a cell or a population of cells an target protein measurement in a cell or population of cells by less than 10%, less than 20%, less than 30%, less than 40%, less than 50%, less than 60%, less than 70%, less than 80%, or less than 90%, relative to a baseline target protein measurement. In some embodiments, expression of the system in a cell or a population of cells an target protein measurement in a cell or population of cells by less than 70%, relative to a baseline target protein measurement.

Disclosed herein, in some embodiments, are polynucleotides, comprising: an exonic splicing silencer (ESS) nucleic acid sequence; and an antisense nucleic acid sequence that binds to an alternatively spliced region of a ribonucleic acid (RNA) encoding sodium channel protein type 1 subunit alpha (SCN1A). In some embodiments, the ESS nucleic acid sequence comprises a sequence having at least 80%, at least 85%, at least 90%, at least 95%, at least 98%, at least 99%, or 100% identity to any one of SEQ ID NOs: 11-13, optionally wherein the ESS nucleic acid sequence comprises the nucleic acid sequence of any one of SEQ ID NOs: 11-13. In some embodiments, the antisense nucleic acid sequence comprises a sequence having at least 80%, at least 85%, at least 90%, at least 95%, at least 98%, at least 99%, or 100% identity to any one of SEQ ID NOs: 14 and 18-104, optionally wherein the antisense nucleic acid sequence comprises the nucleic acid sequence of any one of SEQ ID NOs: 14 and 18-104. In some embodiments, the polynucleotide further comprises a Sm binding site. In some embodiments, the Sm binding site comprises a nucleic acid sequence having at least 80%, at least 85%, at least 90%, at least 95%, at least 98%, at least 99%, or 100% identity to the nucleic acid sequence of any one of SEQ ID NOs: 150-151, optionally wherein the Sm binding site comprises the nucleic acid sequence of any one of SEQ ID NOs: 150-151. In some embodiments, the polynucleotide further comprises a hairpin sequence. In some embodiments, the hairpin sequence comprises a nucleic acid sequence having at least 80%, at least 85%, at least 90%, at least 95%, at least 98%, at least 99%, or 100% identity to the nucleic acid sequence of SEQ ID NO: 4, optionally wherein the hairpin sequence comprises the nucleic acid sequence of SEQ ID NO: 4. In some embodiments, the polynucleotide comprising a nucleic acid sequence having at least 80%, at least 85%, at least 90%, at least 95%, at least 98%, at least 99%, or 100% identity to the nucleic acid sequence of any one of SEQ ID NOs: 364-566.

Disclosed herein, in some embodiments, are polynucleotides, comprising: a first sequence comprising a nucleic acid sequence having at least 80%, at least 85%, at least 90%, at least 95%, at least 98%, at least 99%, or 100% identity to any one of SEQ ID NOs: 11-13; and a second sequence comprising a nucleic acid sequence having at least 80%, at least 85%, at least 90%, at least 95%, at least 98%, at least 99%, or 100% identity to any one of SEQ ID NOs: 14 and 18-104. In some embodiments, the first sequence comprises the nucleic acid sequence of any one of SEQ ID NOs: 11-13. In some embodiments, the second sequence comprises the nucleic acid sequence of any one of SEQ ID NOs: 14 and 18-104. In some embodiments, the polynucleotide further a third sequence, wherein the third sequence comprises a nucleic acid sequence having at least 80%, at least 85%, at least 90%, at least 95%, at least 98%, at least 99%, or 100% identity to the nucleic acid sequence of any one of SEQ ID NOs: 150-151, optionally wherein the third sequence comprises the nucleic acid sequence of any one of SEQ ID NOs: 150-151. In some embodiments, the polynucleotide further comprises a fourth sequence, wherein the fourth sequence comprises a nucleic acid sequence having at least 80%, at least 85%, at least 90%, at least 95%, at least 98%, at least 99%, or 100% identity to the nucleic acid sequence of SEQ ID NO: 4, optionally wherein the fourth sequence comprises the nucleic acid sequence of SEQ ID NO: 4. In some embodiments, the polynucleotide comprising a nucleic acid sequence having at least 90% identity to the nucleic acid sequence of any one of SEQ ID NOs: 364-566.

Disclosed herein, in some embodiments, are RNA polynucleotides, comprising a nucleic acid sequence having at least 80%, at least 85%, at least 90%, at least 95%, at least 98%, at least 99%, or 100% identity to the nucleic acid sequence of any one of SEQ ID NOs: 364-566.

### ESS Sequences

Described herein, in some embodiments, are exonic splicing silencer (ESS) sequences. An ESS may be or include a 10-20 nt sequence at a 5′ terminus of an snRNA (e.g. engineered snRNA) capable of enhancing splicing suppression. The ESS may be included in a modified or recombinant snRNA or U7 snRNA. The ESS may recruit a protein factor that reduces splicing of a target RNA. The ESS may recruit a protein factor that reduces splicing of the RNA encoding an SCN1A protein. An ESS sequence may refer to an ESS or to a sequence that encodes an ESS. An ESS may include a short region of an exon and be a cis-regulatory element (CREs). CREs are regions of non-coding DNA which regulate transcription of neighboring genes. CREs may include components of genetic regulatory networks that control the timing and the amount that a specific gene is expressed. An ESS may be bound by a negatively acting factor such as a heterogeneous ribonucleoprotein (hnRNP).

ESSs may work by inhibiting the splicing of pre-mRNA transcripts or promoting exon skipping. In some embodiments, the ESS may recruit a protein factor that silences splicing of a target RNA such as an RNA encoding an SCN1A protein. In some embodiments, the ESS may recruit a group of factors that reduce splicing of the target RNA. In some embodiments, the ESS may recruit a group of factors that silence splicing of the RNA encoding an SCN1A protein.

In some embodiments, the ESS may be about 20 nucleotides long. In some embodiments, the ESS may be at least 4 nucleotides long. In some embodiments, the ESS may be at least 5 nucleotides long. In some embodiments, the ESS may be at least 6 nucleotides long. In some embodiments, the ESS may be at least 7 nucleotides long. In some embodiments, the ESS may be at least 8 nucleotides long. In some embodiments, the ESS may be at least 9 nucleotides long. In some embodiments, the ESS may be at least 10 nucleotides long. In some embodiments, the ESS may be at least 11 nucleotides long. In some embodiments, the ESS may be at least 12 nucleotides long. In some embodiments, the ESS may be at least 13 nucleotides long. In some embodiments, the ESS may be at least 14 nucleotides long. In some embodiments, the ESS may be at least 15 nucleotides long. In some embodiments, the ESS may be at least 16 nucleotides long. In some embodiments, the ESS may be at least 17 nucleotides long. In some embodiments, the ESS may be at least 18 nucleotides long. In some embodiments, the ESS may be at least 19 nucleotides long. In some embodiments, the ESS may be at least 20 nucleotides long.

In some embodiments, the ESS nucleic acid sequence may include a nucleic acid sequence at least 90% identical to an ESS nucleic acid sequence in Tables 2A-2B. In some embodiments, the ESS nucleic acid sequence may include a nucleic acid sequence at least 99% identical to an ESS nucleic acid sequence in Tables 2A-2B. In some embodiments, the ESS nucleic acid sequence may include a nucleic acid sequence at least 98% identical to an ESS nucleic acid sequence in Tables 2A-2B. In some embodiments, the ESS nucleic acid sequence may include a nucleic acid sequence at least 97% identical to an ESS nucleic acid sequence in Tables 2A-2B. In some embodiments, the ESS nucleic acid sequence may include a nucleic acid sequence at least 96% identical to an ESS nucleic acid sequence in Tables 2A-2B. In some embodiments, the ESS nucleic acid sequence may include a nucleic acid sequence at least 95% identical to an ESS nucleic acid sequence in Tables 2A-2B. In some embodiments, the ESS nucleic acid sequence may include a nucleic acid sequence at least 94% identical to an ESS nucleic acid sequence in Tables 2A-2B. In some embodiments, the ESS nucleic acid sequence may include a nucleic acid sequence at least 93% identical to an ESS nucleic acid sequence in Tables 2A-2B. In some embodiments, the ESS nucleic acid sequence may include a nucleic acid sequence at least 92% identical to an ESS nucleic acid sequence in Tables 2A-2B. In some embodiments, the ESS nucleic acid sequence may include a nucleic acid sequence at least 91% identical to an ESS nucleic acid sequence in Tables 2A-2B. In some embodiments, the ESS nucleic acid sequence may include a nucleic acid sequence at least 90% identical to an ESS nucleic acid sequence in Tables 2A-2B. In some embodiments, the ESS nucleic acid sequence may include a nucleic acid sequence at least 85% identical to an ESS nucleic acid sequence in Tables 2A-2B. In some embodiments, the ESS nucleic acid sequence may include a nucleic acid sequence at least 80% identical to an ESS nucleic acid sequence in Tables 2A-2B. In some embodiments, the ESS nucleic acid sequence may include a nucleic acid sequence at least 75% identical to an ESS nucleic acid sequence in Tables 2A-2B. In some embodiments, the ESS nucleic acid sequence may include a nucleic acid sequence at least 70% identical to an ESS nucleic acid sequence in Tables 2A-2B.

In some embodiments, the ESS nucleic acid sequence may be or include ATGATAGGGACTTAGGGTGA (SEQ ID NO: 11). In some embodiments, the ESS nucleic acid sequence may be or include TTTGTTCCGTGGGTGGTTTA (SEQ ID NO: 12). In some embodiments, the ESS nucleic acid sequence may be or include TGGGGGGAGGTAGGTAGGTA (SEQ ID NO: 13).

### ESE Sequences

Described here, in some embodiments, are exonic splicing enhancers (ESE). The ESE may be included in a modified or recombinant U7 snRNA. The ESE may recruit a protein factor that enhances splicing of a target RNA. Some embodiments include an ESE sequence that enhances splicing of an exon of an RNA encoding SCN1A. An ESE sequence may refer to an ESE or to a sequence that encodes an ESE. An ESE may include a short region of an exon and be a cis-regulatory element (CRE). ESEs may work by directing or promoting the splicing of pre-mRNA transcripts. In some embodiments, the ESE may recruit a protein factor that promotes splicing of a target RNA such as an RNA encoding SCN1A. In some embodiments, the ESE may recruit a group of factors that increase splicing of the target RNA. Exonic or intronic splicing enhancers may be or include cis-acting elements bound by positive transactive factors such as a nuclear phosphoprotein family that may include serine/arginine-rich proteins.

In some embodiments, the ESE may be about 20 nucleotides. In some embodiments, the ESE may be at least 4 nucleotides long. In some embodiments, the ESE may be at least 5 nucleotides long. In some embodiments, the ESE may be at least 6 nucleotides long. In some embodiments, the ESE may be at least 7 nucleotides long. In some embodiments, the ESE may be at least 8 nucleotides long. In some embodiments, the ESE may be at least 9 nucleotides long. In some embodiments, the ESE may be at least 10 nucleotides long. In some embodiments, the ESE may be at least 11 nucleotides long. In some embodiments, the ESE may be at least 12 nucleotides long. In some embodiments, the ESE may be at least 13 nucleotides long. In some embodiments, the ESE may be at least 14 nucleotides long. In some embodiments, the ESE may be at least 15 nucleotides long. In some embodiments, the ESE may be at least 16 nucleotides long. In some embodiments, the ESE may be at least 17 nucleotides long. In some embodiments, the ESE may be at least 18 nucleotides long. In some embodiments, the ESE may be at least 19 nucleotides long. In some embodiments, the ESE may be at least 20 nucleotides long.

### U7 Targeting Sequences

Described herein, in some embodiments, are targeting nucleic acid sequences such as snRNA targeting sequences or U7 targeting sequences. A targeting nucleic acid sequence may be or include an antisense nucleic acid sequence. A U7 targeting nucleic acid sequence may be or include a U7 antisense nucleic acid sequence. An snRNA targeting nucleic acid sequence may be or include a snRNA antisense nucleic acid sequence. Described herein, in some embodiments, are antisense nucleic acid sequences such as snRNA antisense sequences or U7 antisense sequences. An antisense sequence may be referred to as a targeting sequence. The antisense nucleic acid sequence may be included in a modified or recombinant snRNA or U7 snRNA. The antisense nucleic acid sequence may target (e.g. bind or be reverse complementary to) a target RNA such as an RNA encoding an SCN1A protein. The antisense nucleic acid sequence may bind to the target RNA. In some embodiments, an antisense nucleic acid sequence is encoded by a DNA sequence (e.g. a DNA expression construct). Targeting a target RNA may include binding or being reverse complementary to the target RNA.

Described herein, in some embodiments, is a system wherein the antisense nucleic acid sequence may be 10-60 nucleotides in length. Some U rich small nuclear ribonucleoproteins (snRNPs) are complexes that may mediate the splicing of pre-mRNAs. U7 snRNP may be an exception in being uninvolved in splicing but may be a factor in unique 3′ end processing of replication-dependent histone mRNAs. However, by introducing controlled changes in the U7 snRNA histone binding sequence and in the Sm motif, U7 can be used as an effective tool for gene therapy. in some embodiments, the modified U7 snRNP may not be uninvolved in processing of replication-dependent histone pre-mRNA, but target splicing by inducing efficient skipping or inclusion of selected exons. Modification of the sequence motif of U7 snRNA can make the snRNP particle hybridize to almost any RNA sequence within the nucleoplasm.

A targeting nucleic sequence is or include a length of nucleotides. In some embodiments, the targeting nucleic sequence is at least 10 nucleotides. In some embodiments, the targeting nucleic sequence is at least 11 nucleotides. In some embodiments, the targeting nucleic sequence is at least 12 nucleotides. In some embodiments, the targeting nucleic sequence is at least 13 nucleotides. In some embodiments, the targeting nucleic sequence is at least 14 nucleotides. In some embodiments, the targeting nucleic sequence is at least 15 nucleotides. In some embodiments, the targeting nucleic sequence is at least 16 nucleotides. In some embodiments, the targeting nucleic sequence is at least 17 nucleotides. In some embodiments, the targeting nucleic sequence is at least 18 nucleotides. In some embodiments, the targeting nucleic sequence is at least 19 nucleotides. In some embodiments, the targeting nucleic sequence is at least 20 nucleotides. In some embodiments, the targeting nucleic sequence is at least 22 nucleotides. In some embodiments, the targeting nucleic sequence is at least 24 nucleotides. In some embodiments, the targeting nucleic sequence is at least 26 nucleotides. In some embodiments, the targeting nucleic sequence is at least 28 nucleotides. In some embodiments, the targeting nucleic sequence is at least 30 nucleotides. In some embodiments, the targeting nucleic sequence is at least 32 nucleotides. In some embodiments, the targeting nucleic sequence is at least 34 nucleotides. In some embodiments, the targeting nucleic sequence is at least 36 nucleotides. In some embodiments, the targeting nucleic sequence is at least 38 nucleotides. In some embodiments, the targeting nucleic sequence is at least 40 nucleotides. In some embodiments, the targeting nucleic sequence is at least 45 nucleotides. In some embodiments, the targeting nucleic sequence is at least 50 nucleotides. In some embodiments, the targeting nucleic sequence is at least 55 nucleotides. In some embodiments, the targeting nucleic sequence is at least 60 nucleotides. In some embodiments, the targeting nucleic sequence is at least 65 nucleotides. In some embodiments, the targeting nucleic sequence is at least 70 nucleotides. In some embodiments, the targeting nucleic sequence is at least 75 nucleotides. In some embodiments, the targeting nucleic sequence is at least 80 nucleotides. In some embodiments, the targeting nucleic sequence is at least 85 nucleotides. In some embodiments, the targeting nucleic sequence is at least 90 nucleotides. In some embodiments, the targeting nucleic sequence is at least 95 nucleotides. In some embodiments, the targeting nucleic sequence is at least 100 nucleotides. In some embodiments, the targeting nucleic sequence is at least 125 nucleotides. In some embodiments, the targeting nucleic sequence is at least 150 nucleotides. In some embodiments, the targeting nucleic sequence is at least 175 nucleotides. In some embodiments, the targeting nucleic sequence is at least 200 nucleotides. In some embodiments, the targeting nucleic sequence is at least 225 nucleotides. In some embodiments, the targeting nucleic sequence is at least 250 nucleotides. In some embodiments, the targeting nucleic sequence is at most 10 nucleotides. In some embodiments, the targeting nucleic sequence is at most 11 nucleotides. In some embodiments, the targeting nucleic sequence is at most 12 nucleotides. In some embodiments, the targeting nucleic sequence is at most 13 nucleotides. In some embodiments, the targeting nucleic sequence is at most 14 nucleotides. In some embodiments, the targeting nucleic sequence is at most 15 nucleotides. In some embodiments, the targeting nucleic sequence is at most 16 nucleotides. In some embodiments, the targeting nucleic sequence is at most 17 nucleotides. In some embodiments, the targeting nucleic sequence is at most 18 nucleotides. In some embodiments, the targeting nucleic sequence is at most 19 nucleotides. In some embodiments, the targeting nucleic sequence is at most 20 nucleotides. In some embodiments, the targeting nucleic sequence is at most 22 nucleotides. In some embodiments, the targeting nucleic sequence is at most 24 nucleotides. In some embodiments, the targeting nucleic sequence is at most 26 nucleotides. In some embodiments, the targeting nucleic sequence is at most 28 nucleotides. In some embodiments, the targeting nucleic sequence is at most 30 nucleotides. In some embodiments, the targeting nucleic sequence is at most 32 nucleotides. In some embodiments, the targeting nucleic sequence is at most 34 nucleotides. In some embodiments, the targeting nucleic sequence is at most 36 nucleotides. In some embodiments, the targeting nucleic sequence is at most 38 nucleotides. In some embodiments, the targeting nucleic sequence is at most 40 nucleotides. In some embodiments, the targeting nucleic sequence is at most 45 nucleotides. In some embodiments, the targeting nucleic sequence is at most 50 nucleotides. In some embodiments, the targeting nucleic sequence is at most 55 nucleotides. In some embodiments, the targeting nucleic sequence is at most 60 nucleotides. In some embodiments, the targeting nucleic sequence is at most 65 nucleotides. In some embodiments, the targeting nucleic sequence is at most 70 nucleotides. In some embodiments, the targeting nucleic sequence is at most 75 nucleotides. In some embodiments, the targeting nucleic sequence is at most 80 nucleotides. In some embodiments, the targeting nucleic sequence is at most 85 nucleotides. In some embodiments, the targeting nucleic sequence is at most 90 nucleotides. In some embodiments, the targeting nucleic sequence is at most 95 nucleotides. In some embodiments, the targeting nucleic sequence is at most 100 nucleotides. In some embodiments, the targeting nucleic sequence is at most 125 nucleotides. In some embodiments, the targeting nucleic sequence is at most 150 nucleotides. In some embodiments, the targeting nucleic sequence is at most 175 nucleotides. In some embodiments, the targeting nucleic sequence is at most 200 nucleotides. In some embodiments, the targeting nucleic sequence is at most 225 nucleotides. In some embodiments, the targeting nucleic sequence is at most 250 nucleotides. The antisense nucleic acid sequence may, in some embodiments, include a length defined by a range of any two of the aforementioned numbers of nucleotides.

In some embodiments, the antisense nucleic acid sequence binds an alternatively spliced region. The alternatively spliced region may be or include exon 20N, which is an example of a target RNA sequence. In some embodiments, the antisense nucleic acid sequence may bind to the alternatively spliced region at the 5′ end of the nucleic acid. In some embodiments, the antisense nucleic acid sequence binds to the alternatively spliced region at the 3′ end of the nucleic acid. In some embodiments, the antisense nucleic acid sequence bind to the alternatively spliced region in between the 5′ and 3′ ends of the target RNA.

An antisense nucleic acid sequence may hybridize or bind to a target RNA. In some embodiments, the antisense nucleic acid sequence is fully reverse complementary to a portion of the target RNA. In some embodiments, the antisense nucleic acid sequence is partially reverse complementary (e.g. at least 80% reverse complementary or at least 90% reverse complementary) to a portion of the target RNA. In some embodiments, the targeting nucleic acid is at least 99% reverse complementary to a portion of the target RNA. In some embodiments, the targeting nucleic acid is at least 98% reverse complementary to a portion of the target RNA. In some embodiments, the targeting nucleic acid is at least 97% reverse complementary to a portion of the target RNA. In some embodiments, the targeting nucleic acid is at least 96% reverse complementary to a portion of the target RNA. In some embodiments, the targeting nucleic acid is at least 95% reverse complementary to a portion of the target RNA. In some embodiments, the targeting nucleic acid is at least 94% reverse complementary to a portion of the target RNA. In some embodiments, the targeting nucleic acid is at least 93% reverse complementary to a portion of the target RNA. In some embodiments, the targeting nucleic acid is at least 92% reverse complementary to a portion of the target RNA. In some embodiments, the targeting nucleic acid is at least 91% reverse complementary to a portion of the target RNA. In some embodiments, the targeting nucleic acid is at least 90% reverse complementary to a portion of the target RNA. In some embodiments, the targeting nucleic acid is at least 85% reverse complementary to a portion of the target RNA. In some embodiments, the targeting nucleic acid is at least 80% reverse complementary to a portion of the target RNA. In some embodiments, the targeting nucleic acid is at least 75% reverse complementary to a portion of the target RNA. In some embodiments, the targeting nucleic acid is at least 70% reverse complementary to a portion of the target RNA. In some embodiments, the antisense nucleic acid sequence is fully reverse complementary or partially reverse complementary (e.g. at least 90% reverse complementary) to a portion of the target RNA. An example of a portion of a target RNA (e.g. targeted or bound by the antisense nucleic acid sequence) may be or include an alternatively spliced exon such as exon 20N. The portion of the target RNA (e.g. targeted or bound by the antisense nucleic acid sequence) may be or include a region of a target RNA near or adjacent to an alternatively spliced exon such as exon 20N.

An antisense nucleic acid sequence may hybridize or bind to an alternatively spliced region (e.g. of a target RNA). In some embodiments, the antisense nucleic acid sequence is fully reverse complementary to a portion of the alternatively spliced region. In some embodiments, the antisense nucleic acid sequence is partially reverse complementary (e.g. at least 80% reverse complementary or at least 90% reverse complementary) to a portion of the alternatively spliced region. In some embodiments, the targeting nucleic acid is at least 99% reverse complementary to a portion of the alternatively spliced region. In some embodiments, the targeting nucleic acid is at least 98% reverse complementary to a portion of the alternatively spliced region. In some embodiments, the targeting nucleic acid is at least 97% reverse complementary to a portion of the alternatively spliced region. In some embodiments, the targeting nucleic acid is at least 96% reverse complementary to a portion of the alternatively spliced region. In some embodiments, the targeting nucleic acid is at least 95% reverse complementary to a portion of the alternatively spliced region. In some embodiments, the targeting nucleic acid is at least 94% reverse complementary to a portion of the alternatively spliced region. In some embodiments, the targeting nucleic acid is at least 93% reverse complementary to a portion of the alternatively spliced region. In some embodiments, the targeting nucleic acid is at least 92% reverse complementary to a portion of the alternatively spliced region. In some embodiments, the targeting nucleic acid is at least 91% reverse complementary to a portion of the alternatively spliced region. In some embodiments, the targeting nucleic acid is at least 90% reverse complementary to a portion of the alternatively spliced region. In some embodiments, the targeting nucleic acid is at least 85% reverse complementary to a portion of the alternatively spliced region. In some embodiments, the targeting nucleic acid is at least 80% reverse complementary to a portion of the alternatively spliced region. In some embodiments, the targeting nucleic acid is at least 75% reverse complementary to a portion of the alternatively spliced region. In some embodiments, the targeting nucleic acid is at least 70% reverse complementary to a portion of the alternatively spliced region. In some embodiments, the antisense nucleic acid sequence is fully reverse complementary or partially reverse complementary (e.g. at least 90% reverse complementary) to a portion of the alternatively spliced region.

In some embodiments, the portion of the alternatively spliced region comprises nucleotide positions within 50 base pairs (bp) of a 5′ or 3′ end of the alternatively spliced region. In some embodiments, the portion of the alternatively spliced region comprises nucleotide positions within 45 bp of a 5′ or 3′ end of the alternatively spliced region. In some embodiments, the portion of the alternatively spliced region comprises nucleotide positions within 40 bp of a 5′ or 3′ end of the alternatively spliced region. In some embodiments, the portion of the alternatively spliced region comprises nucleotide positions within 35 bp of a 5′ or 3′ end of the alternatively spliced region. In some embodiments, the portion of the alternatively spliced region comprises nucleotide positions within 30 bp of a 5′ or 3′ end of the alternatively spliced region. In some embodiments, the portion of the alternatively spliced region comprises nucleotide positions within 25 bp of a 5′ or 3′ end of the alternatively spliced region. In some embodiments, the portion of the alternatively spliced region comprises nucleotide positions within 20 bp of a 5′ or 3′ end of the alternatively spliced region. In some embodiments, the portion of the alternatively spliced region comprises nucleotide positions within 15 bp of a 5′ or 3′ end of the alternatively spliced region. In some embodiments, the portion of the alternatively spliced region comprises nucleotide positions within 10 bp of a 5′ or 3′ end of the alternatively spliced region. In some embodiments, the portion of the alternatively spliced region excludes nucleotide positions within 45 bp of a 5′ or 3′ end of the alternatively spliced region. In some embodiments, the portion of the alternatively spliced region excludes nucleotide positions within 40 bp of a 5′ or 3′ end of the alternatively spliced region. In some embodiments, the portion of the alternatively spliced region excludes nucleotide positions within 35 bp of a 5′ or 3′ end of the alternatively spliced region. In some embodiments, the portion of the alternatively spliced region excludes nucleotide positions within 30 bp of a 5′ or 3′ end of the alternatively spliced region. In some embodiments, the portion of the alternatively spliced region excludes nucleotide positions within 25 bp of a 5′ or 3′ end of the alternatively spliced region. In some embodiments, the portion of the alternatively spliced region excludes nucleotide positions within 20 bp of a 5′ or 3′ end of the alternatively spliced region. In some embodiments, the portion of the alternatively spliced region excludes nucleotide positions within 15 bp of a 5′ or 3′ end of the alternatively spliced region. In some embodiments, the portion of the alternatively spliced region excludes nucleotide positions within 10 bp of a 5′ or 3′ end of the alternatively spliced region. In some embodiments, the 5′ or 3′ end is a 5′ end. In some embodiments, the 5′ or 3′ end is a 3′ end.

In some embodiments, the portion of the alternatively spliced region is within a 5′ half of the alternatively spliced region. In some embodiments, the portion of the alternatively spliced region is within a 5′ end of the alternatively spliced region. In some embodiments, the portion of the alternatively spliced region is within a 5′ half or 5′ end of the alternatively spliced region. In some embodiments, the portion of the alternatively spliced region is within 25 nucleotides, within 50 nucleotides, within 75 nucleotides, within 100 nucleotides, within 125 nucleotides, within 150 nucleotides, within 175 nucleotides, within 200 nucleotides, within 225 nucleotides, or within 250 nucleotides of a 5′ end of an alternatively spliced region.

In some embodiments, the portion of the alternatively spliced region is within a 3′ half of the alternatively spliced region. In some embodiments, the portion of the alternatively spliced region is within a 3′ end of the alternatively spliced region. In some embodiments, the portion of the alternatively spliced region is within a 3′ half or 3′ end of the alternatively spliced region. In some embodiments, the portion of the alternatively spliced region is within 25 nucleotides, within 50 nucleotides, within 75 nucleotides, within 100 nucleotides, within 125 nucleotides, within 150 nucleotides, within 175 nucleotides, within 200 nucleotides, within 225 nucleotides, or within 250 nucleotides of a 3′ end of an alternatively spliced region.

In some embodiments, the antisense nucleic acid sequence may include a nucleic acid sequence identical to an antisense nucleic acid sequence in Tables 2A-2B. In some embodiments, the antisense nucleic acid sequence may include a nucleic acid sequence at least 99% identical to an antisense nucleic acid sequence in Tables 2A-2B. In some embodiments, the antisense nucleic acid sequence may include a nucleic acid sequence at least 98% identical to an antisense nucleic acid sequence in Tables 2A-2B. In some embodiments, the antisense nucleic acid sequence may include a nucleic acid sequence at least 97% identical to an antisense nucleic acid sequence in Tables 2A-2B. In some embodiments, the antisense nucleic acid sequence may include a nucleic acid sequence at least 96% identical to an antisense nucleic acid sequence in Tables 2A-2B. In some embodiments, the antisense nucleic acid sequence may include a nucleic acid sequence at least 95% identical to an antisense nucleic acid sequence in Tables 2A-2B. In some embodiments, the antisense nucleic acid sequence may include a nucleic acid sequence at least 94% identical to an antisense nucleic acid sequence in Tables 2A-2B. In some embodiments, the antisense nucleic acid sequence may include a nucleic acid sequence at least 93% identical to an antisense nucleic acid sequence in Tables 2A-2B. In some embodiments, the antisense nucleic acid sequence may include a nucleic acid sequence at least 92% identical to an antisense nucleic acid sequence in Tables 2A-2B. In some embodiments, the antisense nucleic acid sequence may include a nucleic acid sequence at least 91% identical to an antisense nucleic acid sequence in Tables 2A-2B. In some embodiments, the antisense nucleic acid sequence may include a nucleic acid sequence at least 90% identical to an antisense nucleic acid sequence in Tables 2A-2B. In some embodiments, the antisense nucleic acid sequence may include a nucleic acid sequence at least 85% identical to an antisense nucleic acid sequence in Tables 2A-2B. In some embodiments, the antisense nucleic acid sequence may include a nucleic acid sequence at least 80% identical to an antisense nucleic acid sequence in Tables 2A-2B. In some embodiments, the antisense nucleic acid sequence may include a nucleic acid sequence at least 75% identical to an antisense nucleic acid sequence in Tables 2A-2B. In some embodiments, the antisense nucleic acid sequence may include a nucleic acid sequence at least 70% identical to an antisense nucleic acid sequence in Tables 2A-2B. In some embodiments, the targeting nucleic acid targets or binds with an RNA that includes reverse complement of any of the aforementioned sequences. In some embodiments, the antisense nucleic acid sequence is encoded by a reverse complement of any of the aforementioned sequences (such as when the antisense nucleic acid sequence is encoded in an expression construct described herein. A reverse complement may include Us in place of Ts, or vice versa.

In some embodiments, use of the target nucleic acid sequence results in a percentage of productive isoform of SCN1A, for example as determined in Table 2C. An example of a productive isoform may include an isoform excluding exon 21N of a mouse SCN1A transcript, or an isoform excluding exon 20N of a human SCN1A transcript. In some embodiments, use of the targeting nucleic acid results in at least 50% of the productive isoform. In some embodiments, use of an engineered snRNA comprising the targeting nucleic acid results in at least 55% of the productive isoform. In some embodiments, use of an engineered snRNA comprising the targeting nucleic acid results in at least 60% of the productive isoform. In some embodiments, use of an engineered snRNA comprising the targeting nucleic acid results in at least 65% of the productive isoform. In some embodiments, use of an engineered snRNA comprising the targeting nucleic acid results in at least 70% of the productive isoform. In some embodiments, use of an engineered snRNA comprising the targeting nucleic acid results in at least 75% of the productive isoform. In some embodiments, use of an engineered snRNA comprising the targeting nucleic acid results in at least 80% of the productive isoform. In some embodiments, use of an engineered snRNA comprising the targeting nucleic acid results in at least 85% of the productive isoform. In some embodiments, use of an engineered snRNA comprising the targeting nucleic acid results in at least 90% of the productive isoform. In some embodiments, use of an engineered snRNA comprising the targeting nucleic acid results in at least 91% of the productive isoform. In some embodiments, use of an engineered snRNA comprising the targeting nucleic acid results in at least 92% of the productive isoform. In some embodiments, use of an engineered snRNA comprising the targeting nucleic acid results in no more than 50% of the productive isoform. In some embodiments, use of an engineered snRNA comprising the targeting nucleic acid results in no more than 55% of the productive isoform. In some embodiments, use of an engineered snRNA comprising the targeting nucleic acid results in no more than 60% of the productive isoform. In some embodiments, use of an engineered snRNA comprising the targeting nucleic acid results in no more than 65% of the productive isoform. In some embodiments, use of an engineered snRNA comprising the targeting nucleic acid results in no more than 70% of the productive isoform. In some embodiments, use of an engineered snRNA comprising the targeting nucleic acid results in no more than 75% of the productive isoform. In some embodiments, use of an engineered snRNA comprising the targeting nucleic acid results in no more than 80% of the productive isoform. In some embodiments, use of an engineered snRNA comprising the targeting nucleic acid results in no more than 85% of the productive isoform. In some embodiments, use of an engineered snRNA comprising the targeting nucleic acid results in no more than 90% of the productive isoform. In some embodiments, use of an engineered snRNA comprising the targeting nucleic acid results in no more than 91% of the productive isoform. In some embodiments, use of an engineered snRNA comprising the targeting nucleic acid results in no more than 92% of the productive isoform.

In some embodiments, the antisense nucleic acid sequence may include the nucleic acid sequence of SEQ ID NO: 23, or a reverse complement thereof. In some embodiments, the antisense nucleic acid sequence is 85%, 86%, 87%, 88%, 89%, 90%, 91%, 92%, 93%, 94%, 95%, 96%, 97%, 98%, 99%, or 100% identical to SEQ ID NO: 23 or a reverse complement thereof, or a range of % identities defined by any two of the aforementioned percentages.

In some embodiments, the antisense nucleic acid sequence may include the nucleic acid sequence of SEQ ID NO: 53, or a reverse complement thereof. In some embodiments, the antisense nucleic acid sequence is 85%, 86%, 87%, 88%, 89%, 90%, 91%, 92%, 93%, 94%, 95%, 96%, 97%, 98%, 99%, or 100% identical to SEQ ID NO: 53 or a reverse complement thereof, or a range of % identities defined by any two of the aforementioned percentages.

In some embodiments, the antisense nucleic acid sequence may include the nucleic acid sequence of SEQ ID NO: 98, or a reverse complement thereof. In some embodiments, the antisense nucleic acid sequence is 85%, 86%, 87%, 88%, 89%, 90%, 91%, 92%, 93%, 94%, 95%, 96%, 97%, 98%, 99%, or 100% identical to SEQ ID NO: 98 or a reverse complement thereof, or a range of % identities defined by any two of the aforementioned percentages.

The antisense nucleic acid sequence may bind to or be reverse complementary to a targeted region. In some embodiments, the antisense nucleic acid sequence binds to a target mRNA at a region 5′ or 3′ relative to an alternatively spliced exon. In some embodiments, the antisense nucleic acid sequence binds to an SCN1A mRNA at a region 5′ or 3′ relative to exon 20N. The antisense nucleic acid sequence may bind to an SCN1A mRNA comprising the sequence of SEQ ID NO: 153. The antisense nucleic acid sequence may bind to an SCN1A mRNA comprising the sequence of SEQ ID NO: 154.

In some embodiments, the antisense nucleic acid sequence binds to a target pre-mRNA at a region 5′ relative to an alternatively spliced exon (e.g. an NMD exon). The region may be up to 25 nucleotides, up to 50 nucleotides, up to 75 nucleotides, up to 100 nucleotides, or more, upstream from or 5′ to the alternatively spliced exon in the target pre-mRNA.

In some embodiments, the antisense nucleic acid sequence binds to an SCN1A pre-mRNA at a region 5′ relative to exon 20N. The region may be up to 25 nucleotides, up to 50 nucleotides, up to 75 nucleotides, up to 100 nucleotides, or more, upstream from or 5′ to exon 20N in an SCN1A pre-mRNA.

In some embodiments, the antisense nucleic acid sequence binds to a target pre-mRNA at a region 3′ relative to an alternatively spliced exon (e.g. an NMD exon). The region may be up to 25 nucleotides, up to 50 nucleotides, up to 75 nucleotides, up to 100 nucleotides, or more, downstream from or 3′ to the alternatively spliced exon in the target pre-mRNA.

In some embodiments, the antisense nucleic acid sequence binds to an SCN1A pre-mRNA at a region 3′ relative to exon 20N. The region may be up to 25 nucleotides, up to 50 nucleotides, up to 75 nucleotides, up to 100 nucleotides, or more, downstream from or 3′ to exon 20N in an SCN1A pre-mRNA.

In some embodiments, the engineered U7 snRNA includes an antisense nucleic acid sequence 3′ to the ESS nucleic acid sequence. In some embodiments, the engineered U7 snRNA includes an antisense nucleic acid sequence downstream relative to the ESS nucleic acid sequence. In some embodiments, the engineered U7 snRNA includes an antisense nucleic acid sequence 3′ or downstream relative to the ESS nucleic acid sequence.

### Sm Binding Sites

Described herein, in some embodiments, are systems that include a Sm binding site. The Sm binding site may be included in a modified or recombinant snRNA or U7 snRNA. Sm proteins may bind to a U7 snRNA via the Sm binding site. Once the Sm proteins bind the U7 snRNA in the cytoplasm they may bind to a pre-mRNA and regulate splicing. In some embodiments, the system contains a Sm-like binding site. In some embodiments, the Sm binding site comprises AAUUUGUCUAG (SEQ ID NO: 150). In some embodiments, the Sm binding site comprises AAUUUUUGGAG (SEQ ID NO: 151; smOPT). A modified or recombinant U7 snRNA sequence may include a Sm binding site. A system may encode a modified or recombinant U7 snRNA sequence that includes a Sm binding site.

In some embodiments, the Sm binding site may be 3′ relative to the ESS nucleic acid sequence. In some embodiments, the Sm binding site may be downstream relative to the ESS nucleic acid sequence. In some embodiments, the Sm binding site may be 3′ relative to the antisense nucleic acid sequence. In some embodiments, the Sm binding site may be downstream relative to the antisense nucleic acid sequence. In some embodiments, the Sm binding site is 3′ or downstream relative to the ESS nucleic acid sequence or antisense nucleic acid sequence.

### U7 3′ Hairpins

Described herein, in some embodiments, are systems that include a hairpin sequence. The hairpin sequence may be included in a modified or recombinant snRNA or U7 snRNA. The hairpin sequence may include a U7 hairpin sequence. The U7 hairpin sequence may be a 3′ U7 hairpin sequence. A modified or recombinant U7 snRNA sequence may include the hairpin sequence. A system may encode a modified or recombinant U7 snRNA sequence that includes a hairpin sequence.

Described herein, in some embodiments, is a nucleic acid system that may contain a hairpin sequence comprising a U7 small nuclear RNA (snRNA) hairpin sequence. A hairpin may include an unpaired loop of RNA that is created when a RNA strand folds and forms complementary base pairs with another section of the same strand. The resulting structure may look like a loop or a U-shape. Hairpins may include a common type of secondary structure in RNA molecules and can be formed when two complementary sequences in a single RNA molecule meet and bind each other.

In some embodiments, the U7 snRNA hairpin sequence comprises the nucleic acid sequence of SEQ ID NO: 4. In some embodiments, the U7 snRNA hairpin sequence comprises a nucleic acid sequence at least 90%, at least 91%, at least 92%, at least 93%, at least 94%, at least 95%, at least 96%, at least 97%, at least 98%, or at least 99% identical to SEQ ID NO: 4.

In some embodiments, the hairpin sequence may include a 3′ hairpin sequence. In some embodiments, the hairpin sequence may be 3′ or downstream relative to the ESS nucleic acid sequence. In some embodiments, the hairpin sequence may be 3′ or downstream relative to the antisense nucleic acid sequence. In some embodiments, the hairpin sequence may be 3′ or downstream relative to or the Sm binding site. In some embodiments, the hairpin sequence is 3′ or downstream relative to the ESS nucleic acid sequence, the antisense nucleic acid sequence, or the Sm binding site.

### Expression Constructs

Described herein, in some embodiments, are expression constructs. An expression construct may include an expression cassette. The expression construct may be DNA. The expression construct may encode an RNA system described herein. The expression construct may encode an RNA such as a modified U7 snRNA. The expression construct may encode an ESS sequence, a U7 antisense sequence, a Sm binding site, or a U7 3′ hairpin, or a combination thereof. For example, an expression construct may encode an ESS sequence, a U7 antisense sequence, a Sm binding site, and a U7 3′ hairpin, which may be operably connected to a promoter within the expression construct. The expression construct may be or include a viral vector. The expression construct may be included in a composition herein. The expression construct may be included in a virus or viral delivery agent.

In some embodiments, the system may include an expression cassette. In some embodiments, the expression cassette may include a promoter. In some embodiments, the expression cassette may encode an exonic splicing sequence. In some embodiments, the expression cassette may encode a U7 targeting sequence. In some embodiments, the expression cassette may encode a smOPT sequence. In some embodiments, the expression cassette may encode a U7 3′ hairpin structure. In some embodiments, the expression cassette may include a 3′ terminator sequence. In some embodiments, the expression cassette may include or encode a combination of two or more of the following: a promoter, an exonic splicing sequence, a U7 targeting sequence, a smOPT, a U7 3′ hairpin structure, and a 3′ terminator sequence.

Some embodiments include an arrayed series of modified U7 snRNAs. In some embodiments, the array includes multiple U7 modules. Each module may encode a targeting sequence. The targeting sequences of multiple modules may be the same as each other. The targeting sequences of some modules may be different from each other.

An expression construct may encode multiple engineered or modified snRNAs. For example, an expression construct may encode 1, 2, 3, 4, 5, 6, 7, 8, 9, or 10 copies of a recombinant U7 snRNA. In some embodiments, the expression construct encodes 1 copy of a recombinant U7 snRNA. In some embodiments, the expression construct encodes 2 copies of the recombinant U7 snRNA. In some embodiments, the expression construct encodes 3 copies of the recombinant U7 snRNA. In some embodiments, the expression construct encodes 4 copies of the recombinant U7 snRNA. In some embodiments, the expression construct encodes 5 copies of the recombinant U7 snRNA. In some embodiments, the expression construct encodes 6 copies of the recombinant U7 snRNA. In some embodiments, the expression construct encodes 7 copies of the recombinant U7 snRNA. In some embodiments, the expression construct encodes 8 copies of the recombinant U7 snRNA. In some embodiments, the expression construct encodes 9 copies of the recombinant U7 snRNA. In some embodiments, the expression construct encodes 10 copies of the recombinant U7 snRNA. In some embodiments, all 1, 2, 3, 4, 5, 6, 7, 8, 9, or 10 copies of a recombinant U7 snRNA sequence are operably linked to one or more promoters. For example, multiple copies of the recombinant U7 snRNA sequence may be operably linked to a single promoter. In some embodiments, all 1, 2, 3, 4, 5, 6, 7, 8, 9, or 10 copies of a recombinant U7 snRNA sequence are operably linked to one or more 3′ terminator sequences.

In some embodiments, the expression system produces a greater amount of RNA from the transcribable region than an expression system having a wild-type promoter and wild-type terminator operably coupled to the transcribable region. In some embodiments, the amount of RNA produced is at least 10% greater. Described herein, in some embodiments, are methods of producing a ribonucleic acid (RNA), comprising contacting a cell with the expression system.

A modified U7 snRNA may be expressed from a U7 cassette. A functional U7 cassette may include, in the following order from 5′ to 3′: (1) a promoter, (2) an optional exonic splicing silencer, (3) a targeting sequence complementary to a region of a target mRNA, (4) an smOPT sequence, (5) a hairpin sequence, and (6) a 3′ termination signal. The expressed functional U7 snRNA consists of, in the following order from 5′ to 3′: (1) an optional exonic splicing silencer, (2) a targeting sequence complementary to a region of target mRNA, (3) an smOPT sequence, and (4) a hairpin sequence.

Some examples of expression constructs include a DNA sequence encoding an snRNA of Tables 14A-14C, or a sequence at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical thereto.

Disclosed herein, in some embodiments, are polynucleotides, comprising a promoter, a coding sequence, and a terminator sequence, wherein the coding sequence comprises a nucleic acid sequence having at least 80%, at least 85%, at least 90%, at least 95%, at least 98%, at least 99%, or 100% identity to the nucleic acid sequence of any one of SEQ ID NOs: 364-566.

### Promoters

Described herein, in some embodiments, is a nucleic acid system that may contain a promoter sequence. The promoter may be a part of an expression construct. A promoter may include a sequence of DNA to which proteins bind to initiate transcription of a single RNA transcript from the DNA downstream of the promoter. The RNA transcript may encode a protein (mRNA), or can have a function in and of itself, such as tRNA or snRNA. Promoters may be located near the transcription start sites of genes. The promoter may be operably connected with an ESS sequence, a U7 targeting sequence, a Sm binding site, or a U7 3′ hairpin, or a combination thereof. For example, the promoter may be operably connected with an ESS sequence, a U7 targeting sequence, a Sm binding site, and a U7 3′ hairpin within an expression construct.

In some embodiments, the promoter is directly upstream of a transcriptional start site for the engineered snRNA. In some embodiments, the promoter is 5 bps upstream of the transcriptional start site. In some embodiments, the promoter is 10 bps upstream of the transcriptional start site. In some embodiments, the promoter is 15 bps upstream of the transcriptional start site. In some embodiments, the promoter is 20 bps upstream of the transcriptional start site. In some embodiments, the promoter is 25 bps upstream of the transcriptional start site. In some embodiments, the promoter is 30 bps upstream of the transcriptional start site. In some embodiments, the promoter is 35 bps upstream of the transcriptional start site. In some embodiments, the promoter is 40 bps upstream of the transcriptional start site. In some embodiments, the promoter is 45 bps upstream of the transcriptional start site. In some embodiments, the promoter is 50 bps upstream of the transcriptional start site. In some embodiments, the promoter is 55 bps upstream of the transcriptional start site. In some embodiments, the promoter is 60 bps upstream of the transcriptional start site. In some embodiments, the promoter is 65 bps upstream of the transcriptional start site. In some embodiments, the promoter is 70 bps upstream of the transcriptional start site. In some embodiments, the promoter is 75 bps upstream of the transcriptional start site. In some embodiments, the promoter is 80 bps upstream of the transcriptional start site. In some embodiments, the promoter is 85 bps upstream of the transcriptional start site. In some embodiments, the promoter is 90 bps upstream of the transcriptional start site. In some embodiments, the promoter is 95 bps upstream of the transcriptional start site. In some embodiments, the promoter is 100 bps upstream of the transcriptional start site. In some embodiments, the promoter is 200 bps upstream of the transcriptional start site. In some embodiments, the promoter is 300 bps upstream of the transcriptional start site. In some embodiments, the promoter is 400 bps upstream of the transcriptional start site. In some embodiments, the promoter is 500 bps upstream of the transcriptional start site. In some embodiments, the promoter is 600 bps upstream of the transcriptional start site. In some embodiments, the promoter is 700 bps upstream of the transcriptional start site. In some embodiments, the promoter is 800 bps upstream of the transcriptional start site. In some embodiments, the promoter is 900 bps upstream of the transcriptional start site. In some embodiments, the promoter is 1000 bps upstream of the transcriptional start site. Promoters can be about 100-1000 base pairs long, the sequence of which is highly dependent on the gene and product of transcription, type or class of RNA polymerase recruited to the site, and species of organism. In some embodiments, the promoter is 100 base pairs long. In some embodiments, the promoter is 200 base pairs long. In some embodiments, the promoter is 300 base pairs long. In some embodiments, the promoter is 400 base pairs long. In some embodiments, the promoter is 500 base pairs long. In some embodiments, the promoter is 600 base pairs long. In some embodiments, the promoter is 700 base pairs long. In some embodiments, the promoter is 800 base pairs long. In some embodiments, the promoter is 900 base pairs long. In some embodiments, the promoter is 1000 base pairs long.

In some embodiments, the promoter sequence may include a mouse or human promoter sequence. In some embodiments, the promoter sequence may include a mouse promoter sequence. In some embodiments, the promoter sequence may include a human promoter sequence.

The promoter may include an snRNA promoter. In some embodiments, the snRNA promoter includes a mouse U1 snRNA (“MmU1”) promoter, a mouse U2 snRNA (“MmU2”) promoter, a mouse U3 snRNA (“MmU3”) promoter, a mouse U4 snRNA (“MmU4”) promoter, a mouse U5 snRNA (“MmU5”) promoter, a mouse U6 snRNA (“MmU6”) promoter, a mouse U7 snRNA (“MmU7”) promoter, a mouse U11 snRNA (“MmU11”) promoter, a mouse U12 snRNA (“MmU12”) promoter, a mouse U7SK snRNA (“MmU7SK”) promoter, a human U1 snRNA (“HsU1”) promoter, a human U2 snRNA (“HsU2”) promoter, a human U3 snRNA (“HsU3”) promoter, a human U4 snRNA (“HsU4”) promoter, a human U5 snRNA (“HsU5”) promoter, a human U6 snRNA (“HsU6”) promoter, a human U7 snRNA (“HsU7”) promoter, a human U11 snRNA (“HsU11”) promoter, a human U12 snRNA (“HsU12”) promoter, a human U7SK snRNA (“HsU7SK”) promoter, or a functional combination of fragments thereof.

The snRNA promoter may include a MmU1 promoter. The snRNA promoter may include a MmU2 promoter. The snRNA promoter may include a MmU3 promoter. The snRNA promoter may include a MmU4 promoter. The snRNA promoter may include a MmU5 promoter. The snRNA promoter may include a MmU6 promoter. The snRNA promoter may include a MmU7 promoter. The snRNA promoter may include a MmU11 promoter. The snRNA promoter may include a MmU12 promoter. The snRNA promoter may include a MmU7SK promoter. The snRNA promoter may include a HsU1 promoter. The snRNA promoter may include a HsU2 promoter. The snRNA promoter may include a HsU3 promoter. The snRNA promoter may include a HsU4 promoter. The snRNA promoter may include a HsU5 promoter. The snRNA promoter may include a HsU6 promoter. The snRNA promoter may include a HsU7 promoter. The snRNA promoter may include a HsU11 promoter. The snRNA promoter may include a HsU12 promoter. The snRNA promoter may include a HsU7SK promoter.

The snRNA promoter may include a promoter fragment or a combination of promoter fragments. The snRNA promoter may include a MmU1 promoter fragment. The snRNA promoter fragment may include a MmU2 promoter fragment. The snRNA promoter fragment may include a MmU3 promoter fragment. The snRNA promoter fragment may include a MmU4 promoter fragment. The snRNA promoter fragment may include a MmU5 promoter fragment. The snRNA promoter fragment may include a MmU6 promoter fragment. The snRNA promoter fragment may include a MmU7 promoter fragment. The snRNA promoter fragment may include a MmU11 promoter fragment. The snRNA promoter fragment may include a MmU12 promoter fragment. The snRNA promoter fragment may include a MmU7SK promoter fragment. The snRNA promoter fragment may include a HsU1 promoter fragment. The snRNA promoter fragment may include a HsU2 promoter fragment. The snRNA promoter fragment may include a HsU3 promoter fragment. The snRNA promoter fragment may include a HsU4 promoter fragment. The snRNA promoter fragment may include a HsU5 promoter fragment. The snRNA promoter fragment may include a HsU6 promoter fragment. The snRNA promoter fragment may include a HsU7 promoter fragment. The snRNA promoter fragment may include a HsU11 promoter fragment. The snRNA promoter fragment may include a HsU12 promoter fragment. The snRNA promoter fragment may include a HsU7SK promoter fragment.

The snRNA promoter may include a promoter proximal end. The snRNA promoter proximal end may include a MmU1 promoter proximal end fragment. The snRNA promoter proximal end fragment may include a MmU2 promoter proximal end fragment. The snRNA promoter proximal end fragment may include a MmU3 promoter proximal end fragment. The snRNA promoter proximal end fragment may include a MmU4 promoter proximal end fragment. The snRNA promoter proximal end fragment may include a MmU5 promoter proximal end fragment. The snRNA promoter proximal end fragment may include a MmU6 promoter proximal end fragment. The snRNA promoter proximal end fragment may include a MmU7 promoter proximal end fragment. The snRNA promoter proximal end fragment may include a MmU11 promoter proximal end fragment. The snRNA promoter proximal end fragment may include a MmU12 promoter proximal end fragment. The snRNA promoter proximal end fragment may include a MmU7SK promoter proximal end fragment. The snRNA promoter proximal end fragment may include a HsU1 promoter proximal end fragment. The snRNA promoter proximal end fragment may include a HsU2 promoter proximal end fragment. The snRNA promoter proximal end fragment may include a HsU3 promoter proximal end fragment. The snRNA promoter proximal end fragment may include a HsU4 promoter proximal end fragment. The snRNA promoter proximal end fragment may include a HsU5 promoter proximal end fragment. The snRNA promoter proximal end fragment may include a HsU6 promoter proximal end fragment. The snRNA promoter proximal end fragment may include a HsU7 promoter proximal end fragment. The snRNA promoter proximal end fragment may include a HsU11 promoter proximal end fragment. The snRNA promoter proximal end fragment may include a HsU12 promoter proximal end fragment. The snRNA promoter proximal end fragment may include a HsU7SK promoter proximal end fragment.

The snRNA promoter may include a promoter distal end. The snRNA promoter distal end may include a MmU1 promoter distal end fragment. The snRNA promoter distal end fragment may include a MmU2 promoter distal end fragment. The snRNA promoter distal end fragment may include a MmU3 promoter distal end fragment. The snRNA promoter distal end fragment may include a MmU4 promoter distal end fragment. The snRNA promoter distal end fragment may include a MmU5 promoter distal end fragment. The snRNA promoter distal end fragment may include a MmU6 promoter distal end fragment. The snRNA promoter distal end fragment may include a MmU7 promoter distal end fragment. The snRNA promoter distal end fragment may include a MmU11 promoter distal end fragment. The snRNA promoter distal end fragment may include a MmU12 promoter distal end fragment. The snRNA promoter distal end fragment may include a MmU7SK promoter distal end fragment. The snRNA promoter distal end fragment may include a HsU1 promoter distal end fragment. The snRNA promoter distal end fragment may include a HsU2 promoter distal end fragment. The snRNA promoter distal end fragment may include a HsU3 promoter distal end fragment. The snRNA promoter distal end fragment may include a HsU4 promoter distal end fragment. The snRNA promoter distal end fragment may include a HsU5 promoter distal end fragment. The snRNA promoter distal end fragment may include a HsU6 promoter distal end fragment. The snRNA promoter distal end fragment may include a HsU7 promoter distal end fragment. The snRNA promoter distal end fragment may include a HsU11 promoter distal end fragment. The snRNA promoter distal end fragment may include a HsU12 promoter distal end fragment. The snRNA promoter distal end fragment may include a HsU7SK promoter distal end fragment.

In some embodiments, the promoter sequence may include an snRNA promoter sequence. In some embodiments, the promoter sequence may include a U7 snRNA promoter sequence. In some embodiments, the promoter sequence may include a U1 promoter sequence. In some embodiments, the promoter sequence may include a mouse U7 snRNA (“Mm U7”) promoter sequence. In some embodiments, the promoter sequence may include a human U7 snRNA (“Hs U7”) promoter sequence. In some embodiments, the promoter sequence may include a mouse U1a1 (“mu1a1” or “Mm U1a1”) promoter sequence. In some embodiments, the promoter sequence may include a human U1-1 (“HU1” or “Hs U1-1”) promoter sequence. In some embodiments, the promoter sequence may include a fragment or combination of fragments of the promotors listed above. In some embodiments, the promoter sequence comprises a mouse U7 snRNA (“Mm U7”) promoter sequence, a human U7 snRNA (“Hs U7”) promoter sequence, a mouse U1a1 (“mu1a1” or “Mm U1a1”) promoter sequence, or a human U1-1 (“HU1” or “Hs U1-1”) promoter sequence, or a fragment or combination of fragments thereof.

In some embodiments, the promoter sequence may include a U7 snRNA promoter sequence having a distal sequence element (DSE) replaced with a DSE of a U1-1 promoter sequence. In some embodiments, the promoter sequence may include a U7 snRNA promoter sequence having a DSE replaced with a U1a1 promoter sequence. In some embodiments, the promoter sequence comprises a U7 snRNA promoter sequence having a distal sequence element (DSE) replaced with a DSE of a U1-1 or U1a1 promoter sequence. The promoter DSE is a region of the DNA that is normally found upstream of a transcriptional start site. The DSE is usually found upstream, from −250 to −170 bp. In some embodiments, the DSE is at least 10 base pairs long. In some embodiments, the DSE is at least 20 base pairs long. In some embodiments, the DSE is at least 30 base pairs long. In some embodiments, the DSE is at least 40 base pairs long. In some embodiments, the DSE is at least 50 base pairs long. In some embodiments, the DSE is at least 60 base pairs long. In some embodiments, the DSE is at least 70 base pairs long. In some embodiments, the DSE is at least 80 base pairs long. In some embodiments, the DSE is at least 90 base pairs long. In some embodiments, the DSE is at least 100 base pairs long. In some embodiments, the DSE is at least 110 base pairs long. In some embodiments, the DSE is at least 120 base pairs long. In some embodiments, the DSE is at least 130 base pairs long. In some embodiments, the DSE is at least 140 base pairs long. In some embodiments, the DSE is at least 150 base pairs long. In some embodiments, the DSE is at least 160 base pairs long.

In some embodiments, the promoter sequence may include a mouse U7 promoter sequence having a proximal sequence element (PSE) replaced with a PSE of a U1-1 promoter sequence. In some embodiments, the promoter sequence may include a mouse U7 promoter sequence having a PSE replaced with a U1a1 promoter sequence. In some embodiments, the promoter sequence comprises a mouse U7 promoter sequence having a proximal sequence element (PSE) replaced with a PSE of a U1-1 or U1a1 promoter sequence. The promoter PSE may be an essential promoter element located 40-70 bp upstream of the ORF. The promoter PSE may be recognized by a specific transcription factor, snRNA activating protein complex (SNAPc). In some embodiments, the promoter PSE is 5 base pairs long. In some embodiments, the promoter PSE is 10 base pairs long. In some embodiments, the promoter PSE is 15 base pairs long. In some embodiments, the promoter PSE is 20 base pairs long. In some embodiments, the promoter PSE is 25 base pairs long. In some embodiments, the promoter PSE is 30 base pairs long. In some embodiments, the promoter PSE is 35 base pairs long. In some embodiments, the promoter PSE is 40 base pairs long. In some embodiments, the promoter PSE is 45 base pairs long. In some embodiments, the promoter PSE is 50 base pairs long. In some embodiments, the promoter PSE is 55 base pairs long. In some embodiments, the promoter PSE is 60 base pairs long.

In some embodiments, the promoter sequence may include a nucleic acid sequence identical to a promoter sequence in Table 5. In some embodiments, the promoter sequence may include a nucleic acid sequence at least 99% identical to a promoter sequence in Table 5. In some embodiments, the promoter sequence may include a nucleic acid sequence at least 98% identical to a promoter sequence in Table 5. In some embodiments, the promoter sequence may include a nucleic acid sequence at least 97% identical to a promoter sequence in Table 5. In some embodiments, the promoter sequence may include a nucleic acid sequence at least 96% identical to a promoter sequence in Table 5. In some embodiments, the promoter sequence may include a nucleic acid sequence at least 95% identical to a promoter sequence in Table 5. In some embodiments, the promoter sequence may include a nucleic acid sequence at least 94% identical to a promoter sequence in Table 5. In some embodiments, the promoter sequence may include a nucleic acid sequence at least 93% identical to a promoter sequence in Table 5. In some embodiments, the promoter sequence may include a nucleic acid sequence at least 92% identical to a promoter sequence in Table 5. In some embodiments, the promoter sequence may include a nucleic acid sequence at least 91% identical to a promoter sequence in Table 5. In some embodiments, the promoter sequence may include a nucleic acid sequence at least 90% identical to a promoter sequence in Table 5. In some embodiments, the promoter sequence may include a nucleic acid sequence at least 85% identical to a promoter sequence in Table 5. In some embodiments, the promoter sequence may include a nucleic acid sequence at least 80% identical to a promoter sequence in Table 5. In some embodiments, the promoter sequence may include a nucleic acid sequence at least 75% identical to a promoter sequence in Table 5. In some embodiments, the promoter sequence may include a nucleic acid sequence at least 70% identical to a promoter sequence in Table 5. For the modified Mm U7 promoter in Table 5 (SEQ ID NO: 8), a U7 distal sequence element has been replaced with that of human U1-1 and a U7 proximal sequence element has been replaced with that of mouse U1a1.

In some embodiments, the promoter sequence may be 5′ or upstream relative to the ESS nucleic acid sequence. In some embodiments, the promoter sequence may be 5′ or upstream relative to the antisense nucleic acid sequence. In some embodiments, the promoter sequence may be 5′ or upstream relative to the Sm binding site. In some embodiments, the promoter sequence may be 5′ or upstream relative to the hairpin sequence. In some embodiments, the promoter sequence is 5′ or upstream relative to the ESS nucleic acid sequence, the antisense nucleic acid sequence, the Sm binding site, or the hairpin sequence.

### 3′ Terminator Sequences

Described herein, in some embodiments, is a nucleic acid system that may contain a terminator sequence. The terminator sequence may be a part of an expression construct. Described herein, in some embodiments, the nucleic acid system may include a terminator sequence. The terminator sequence may include a region of a nucleic acid sequence that marks the end of a gene or operon during transcription. The terminator sequence may mediate transcriptional termination by providing signals in the newly synthesized transcript RNA that triggers processes which release the transcript RNA from the transcriptional complex. The terminator sequence may be operably connected with an ESS sequence, a U7 targeting sequence, a Sm binding site, or a U7 3′ hairpin, or a combination thereof. For example, the terminator sequence may be operably connected with an ESS sequence, a U7 targeting sequence, a Sm binding site, and a U7 3′ hairpin within an expression construct.

The terminator may include an snRNA terminator. In some embodiments, the snRNA terminator includes a mouse U1 snRNA (“MmU1”) terminator, a mouse U2 snRNA (“MmU2”) terminator, a mouse U3 snRNA (“MmU3”) terminator, a mouse U4 snRNA (“MmU4”) terminator, a mouse U5 snRNA (“MmU5”) terminator, a mouse U6 snRNA (“MmU6”) terminator, a mouse U7 snRNA (“MmU7”) terminator, a mouse U11 snRNA (“MmU11”) terminator, a mouse U12 snRNA (“MmU12”) terminator, a mouse U7SK snRNA (“MmU7SK”) terminator, a human U1 snRNA (“HsU1”) terminator, a human U2 snRNA (“HsU2”) terminator, a human U3 snRNA (“HsU3”) terminator, a human U4 snRNA (“HsU4”) terminator, a human U5 snRNA (“HsU5”) terminator, a human U6 snRNA (“HsU6”) terminator, a human U7 snRNA (“HsU7”) terminator, a human U11 snRNA (“HsU11”) terminator, a human U12 snRNA (“HsU12”) terminator, a human U7SK snRNA (“HsU7SK”) terminator, or a functional combination of fragments thereof.

The snRNA terminator may include a MmU1 terminator. The snRNA terminator may include a MmU2 terminator. The snRNA terminator may include a MmU3 terminator. The snRNA terminator may include a MmU4 terminator. The snRNA terminator may include a MmU5 terminator. The snRNA terminator may include a MmU6 terminator. The snRNA terminator may include a MmU7 terminator. The snRNA terminator may include a MmU11 terminator. The snRNA terminator may include a MmU12 terminator. The snRNA terminator may include a MmU7SK terminator. The snRNA terminator may include a HsU1 terminator. The snRNA terminator may include a HsU2 terminator. The snRNA terminator may include a HsU3 terminator. The snRNA terminator may include a HsU4 terminator. The snRNA terminator may include a HsU5 terminator. The snRNA terminator may include a HsU6 terminator. The snRNA terminator may include a HsU7 terminator. The snRNA terminator may include a HsU11 terminator. The snRNA terminator may include a HsU12 terminator. The snRNA terminator may include a HsU7SK terminator.

The snRNA terminator may include a terminator fragment or a combination of terminator fragments. The snRNA terminator may include a MmU1 terminator fragment. The snRNA terminator fragment may include a MmU2 terminator fragment. The snRNA terminator fragment may include a MmU3 terminator fragment. The snRNA terminator fragment may include a MmU4 terminator fragment. The snRNA terminator fragment may include a MmU5 terminator fragment. The snRNA terminator fragment may include a MmU6 terminator fragment. The snRNA terminator fragment may include a MmU7 terminator fragment. The snRNA terminator fragment may include a MmU11 terminator fragment. The snRNA terminator fragment may include a MmU12 terminator fragment. The snRNA terminator fragment may include a MmU7SK terminator fragment. The snRNA terminator fragment may include a HsU1 terminator fragment. The snRNA terminator fragment may include a HsU2 terminator fragment. The snRNA terminator fragment may include a HsU3 terminator fragment. The snRNA terminator fragment may include a HsU4 terminator fragment. The snRNA terminator fragment may include a HsU5 terminator fragment. The snRNA terminator fragment may include a HsU6 terminator fragment. The snRNA terminator fragment may include a HsU7 terminator fragment. The snRNA terminator fragment may include a HsU11 terminator fragment. The snRNA terminator fragment may include a HsU12 terminator fragment. The snRNA terminator fragment may include a HsU7SK terminator fragment.

The snRNA terminator may include a terminator proximal end. The snRNA terminator proximal end may include a MmU1 terminator proximal end fragment. The snRNA terminator proximal end fragment may include a MmU2 terminator proximal end fragment. The snRNA terminator proximal end fragment may include a MmU3 terminator proximal end fragment. The snRNA terminator proximal end fragment may include a MmU4 terminator proximal end fragment. The snRNA terminator proximal end fragment may include a MmU5 terminator proximal end fragment. The snRNA terminator proximal end fragment may include a MmU6 terminator proximal end fragment. The snRNA terminator proximal end fragment may include a MmU7 terminator proximal end fragment. The snRNA terminator proximal end fragment may include a MmU11 terminator proximal end fragment. The snRNA terminator proximal end fragment may include a MmU12 terminator proximal end fragment. The snRNA terminator proximal end fragment may include a MmU7SK terminator proximal end fragment. The snRNA terminator proximal end fragment may include a HsU1 terminator proximal end fragment. The snRNA terminator proximal end fragment may include a HsU2 terminator proximal end fragment. The snRNA terminator proximal end fragment may include a HsU3 terminator proximal end fragment. The snRNA terminator proximal end fragment may include a HsU4 terminator proximal end fragment. The snRNA terminator proximal end fragment may include a HsU5 terminator proximal end fragment. The snRNA terminator proximal end fragment may include a HsU6 terminator proximal end fragment. The snRNA terminator proximal end fragment may include a HsU7 terminator proximal end fragment. The snRNA terminator proximal end fragment may include a HsU11 terminator proximal end fragment. The snRNA terminator proximal end fragment may include a HsU12 terminator proximal end fragment. The snRNA terminator proximal end fragment may include a HsU7SK terminator proximal end fragment.

The snRNA terminator may include a terminator distal end. The snRNA terminator distal end may include a MmU1 terminator distal end fragment. The snRNA terminator distal end fragment may include a MmU2 terminator distal end fragment. The snRNA terminator distal end fragment may include a MmU3 terminator distal end fragment. The snRNA terminator distal end fragment may include a MmU4 terminator distal end fragment. The snRNA terminator distal end fragment may include a MmU5 terminator distal end fragment. The snRNA terminator distal end fragment may include a MmU6 terminator distal end fragment. The snRNA terminator distal end fragment may include a MmU7 terminator distal end fragment. The snRNA terminator distal end fragment may include a MmU11 terminator distal end fragment. The snRNA terminator distal end fragment may include a MmU12 terminator distal end fragment. The snRNA terminator distal end fragment may include a MmU7SK terminator distal end fragment. The snRNA terminator distal end fragment may include a HsU1 terminator distal end fragment. The snRNA terminator distal end fragment may include a HsU2 terminator distal end fragment. The snRNA terminator distal end fragment may include a HsU3 terminator distal end fragment. The snRNA terminator distal end fragment may include a HsU4 terminator distal end fragment. The snRNA terminator distal end fragment may include a HsU5 terminator distal end fragment. The snRNA terminator distal end fragment may include a HsU6 terminator distal end fragment. The snRNA terminator distal end fragment may include a HsU7 terminator distal end fragment. The snRNA terminator distal end fragment may include a HsU11 terminator distal end fragment. The snRNA terminator distal end fragment may include a HsU12 terminator distal end fragment. The snRNA terminator distal end fragment may include a HsU7SK terminator distal end fragment.

In some embodiments, the terminator sequence may include a mouse or human terminator sequence. In some embodiments, the terminator sequence may include an snRNA terminator sequence. In some embodiments, the terminator sequence may include a U7 snRNA terminator sequence. In some embodiments, the terminator sequence may include a U1 terminator sequence. In some embodiments, the terminator sequence may include a Mm U7 terminator sequence. In some embodiments, the terminator sequence may include a Hs U7 terminator sequence. In some embodiments, the terminator sequence may include a mu1a1 terminator sequence. In some embodiments, the terminator sequence may include a HU1 terminator sequence. In some embodiments, the terminator sequence may include a fragment or a combination of fragments of the terminators listed above. In some embodiments, the terminator sequence comprises a Mm U7 terminator sequence, a Hs U7 terminator sequence, a mu1a1 terminator sequence, or a HU1 terminator sequence, or a fragment or combination of fragments thereof.

In some embodiments, the terminator sequence comprises a U7 snRNA terminator sequence having a distal sequence element (DSE) replaced with a DSE of a U1-1 terminator sequence. In some embodiments, the terminator sequence comprises a U7 snRNA terminator sequence having a DSE replaced with a DSE of a U1a1 terminator sequence. In some embodiments, the terminator sequence comprises a U7 snRNA terminator sequence having a distal sequence element (DSE) replaced with a DSE of a U1-1 or U1a1 terminator sequence. The terminator DSE is a region of the DNA that is normally found downstream of the snRNA at the position from +250 to +170 bp. In some embodiments, the terminator DSE is at least 10 base pairs long. In some embodiments, the terminator DSE is at least 20 base pairs long. In some embodiments, the terminator DSE is at least 30 base pairs long. In some embodiments, the terminator DSE is at least 40 base pairs long. In some embodiments, the terminator DSE is at least 50 base pairs long. In some embodiments, the terminator DSE is at least 60 base pairs long. In some embodiments, the terminator DSE is at least 70 base pairs long. In some embodiments, the terminator DSE is at least 80 base pairs long. In some embodiments, the terminator DSE is at least 90 base pairs long. In some embodiments, the terminator DSE is at least 100 base pairs long. In some embodiments, the terminator DSE is at least 110 base pairs long. In some embodiments, the terminator DSE is at least 120 base pairs long. In some embodiments, the terminator DSE is at least 130 base pairs long. In some embodiments, the terminator DSE is at least 140 base pairs long. In some embodiments, the terminator DSE is at least 150 base pairs long. In some embodiments, the terminator DSE is at least 160 base pairs long.

In some embodiments, the terminator sequence may include a mouse U7 snRNA terminator sequence having a proximal sequence element (PSE) replaced with a PSE of a U1-1 terminator sequence. In some embodiments, the terminator sequence may include a mouse U7 snRNA terminator sequence having a PSE replaced with a PSE of a U1a1 terminator sequence. In some embodiments, the terminator sequence comprises a mouse U7 snRNA terminator sequence having a proximal sequence element (PSE) replaced with a PSE of a U1-1 or U1a1 terminator sequence. The terminator PSE may be an essential terminator element located 40-70 bp downstream of the terminator start site. In some embodiments, the terminator PSE is 5 base pairs long. In some embodiments, the terminator PSE is 10 base pairs long. In some embodiments, the terminator PSE is 15 base pairs long. In some embodiments, the terminator PSE is 20 base pairs long. In some embodiments, the terminator PSE is 25 base pairs long. In some embodiments, the terminator PSE is 30 base pairs long. In some embodiments, the terminator PSE is 35 base pairs long. In some embodiments, the terminator PSE is 40 base pairs long. In some embodiments, the terminator PSE is 45 base pairs long. In some embodiments, the terminator PSE is 50 base pairs long. In some embodiments, the terminator PSE is 55 base pairs long. In some embodiments, the terminator PSE is 60 base pairs long.

In some embodiments, the terminator sequence may include a nucleic acid sequence. In some embodiments, the terminator sequence may include a nucleic acid sequence identical to a terminator sequence in Table 5. In some embodiments, the terminator sequence may include a nucleic acid sequence at least 99% identical to a terminator sequence in Table 5. In some embodiments, the terminator sequence may include a nucleic acid sequence at least 98% identical to a terminator sequence in Table 5. In some embodiments, the terminator sequence may include a nucleic acid sequence at least 97% identical to a terminator sequence in Table 5. In some embodiments, the terminator sequence may include a nucleic acid sequence at least 96% identical to a terminator sequence in Table 5. In some embodiments, the terminator sequence may include a nucleic acid sequence at least 95% identical to a terminator sequence in Table 5. In some embodiments, the terminator sequence may include a nucleic acid sequence at least 94% identical to a terminator sequence in Table 5. In some embodiments, the terminator sequence may include a nucleic acid sequence at least 93% identical to a terminator sequence in Table 5. In some embodiments, the terminator sequence may include a nucleic acid sequence at least 92% identical to a terminator sequence in Table 5. In some embodiments, the terminator sequence may include a nucleic acid sequence at least 910% identical to a terminator sequence in Table 5. In some embodiments, the terminator sequence may include a nucleic acid sequence at least 90% identical to a terminator sequence in Table 5. In some embodiments, the terminator sequence may include a nucleic acid sequence at least 85% identical to a terminator sequence in Table 5. In some embodiments, the terminator sequence may include a nucleic acid sequence at least 80% identical to a terminator sequence in Table 5. In some embodiments, the terminator sequence may include a nucleic acid sequence at least 75% identical to a terminator sequence in Table 5. In some embodiments, the terminator sequence may include a nucleic acid sequence at least 70% identical to a terminator sequence in Table 5.

In some embodiments, the terminator sequence may include a 3′ terminator sequence. In some embodiments, the terminator sequence is 3′ or downstream relative to the promoter sequence. In some embodiments, the terminator sequence is 3′ or downstream relative to the ESS nucleic acid sequence. In some embodiments, the terminator sequence is 3′ or downstream relative to the antisense nucleic acid sequence. In some embodiments, the terminator sequence is 3′ or downstream relative to the Sm binding site. In some embodiments, the terminator sequence is 3′ or downstream relative to the hairpin sequence. In some embodiments, the terminator sequence is 3′ or downstream relative to the promoter sequence, the ESS nucleic acid sequence, the antisense nucleic acid sequence, the Sm binding site, or the hairpin sequence.

### Recombinant Regulatory Elements

Described herein, in some embodiments, is a nucleic acid system that may contain one or more regulatory elements such as a promoter sequence or a terminator sequence. The promoter or terminator sequence may be a part of an expression construct. The expression construct may mix and match regulatory sequences such as promoter sequences or terminator sequences from various organism species, or from various genes of a species. A regulatory sequence may mix and match elements such as a distal or proximal end sequence from various organism species, or from various genes of a species.

Described herein, in some embodiments, is a nucleic acid expression system. In some embodiments, the nucleic acid expression system may contain a regulatory sequence operably coupled to a transcribable region of a nucleic acid. In some embodiments, the regulatory sequence may comprise a proximal regulatory sequence element (PSE). In some embodiments, the regulatory sequence may comprise a distal regulatory sequence element (DSE). In some embodiments, the regulatory sequence may comprise a PSE and a DSE. In some embodiments, the PSE may be comprised of a PSE sequence of a first small nuclear RNA (snRNA) of a first organism species. In some embodiments, the DSE may include a DSE sequence of a second small nuclear RNA (snRNA) of a second organism species. In some embodiments, the first and second snRNAs may be different. In some embodiments, the first and second snRNAs may be the same. In some embodiments, the first and second organism species may be different. In some embodiments, the first and second organism species may be the same. Described herein, in some embodiments, is a nucleic acid system comprised of a regulatory sequence operably coupled to a transcribable region of a nucleic acid, with the regulatory sequence comprised of a proximal regulatory sequence element (PSE) and a distal regulatory sequence element (DSE), wherein the PSE is comprised of a PSE sequence of a first small nuclear RNA (snRNA) of a first organism species, wherein the DSE is comprised of a DSE sequence of a second small nuclear RNA (snRNA) of a second organism species, and wherein the first and second snRNAs are different or wherein the first and second organism species are different.

In some embodiments, the first snRNA may be a snRNA U1. In some embodiments, the first snRNA may be a snRNA U2. In some embodiments, the first snRNA may be a snRNA U3. In some embodiments, the first snRNA may be a snRNA U4. In some embodiments, the first snRNA may be a snRNA U5. In some embodiments, the first snRNA may be a snRNA U6. In some embodiments, the first snRNA may be a snRNA U7. In some embodiments, the first snRNA may be a snRNA U11. In some embodiments, the first snRNA may be a snRNA U12. In some embodiments, the first snRNA may be a snRNA 7SK. In some embodiments, the first snRNA is selected from the group consisting of snRNA U1, snRNA U2, snRNA U3, snRNA U4, snRNA U5, snRNA U6 snRNA U7, snRNA U11 and snRNA 7SK. In some embodiments, the first snRNA is selected from the group consisting of snRNA U1, snRNA U2, snRNA U3, snRNA U4, snRNA U5, snRNA U6 snRNA U7, snRNA U11, snRNA U12 and snRNA 7SK.

In some embodiments, the second snRNA may be a snRNA U1. In some embodiments, the second snRNA may be a snRNA U2. In some embodiments, the second snRNA may be a snRNA U3. In some embodiments, the second snRNA may be a snRNA U4. In some embodiments, the second snRNA may be a snRNA U5. In some embodiments, the second snRNA may be a snRNA U6. In some embodiments, the second snRNA may be a snRNA U7. In some embodiments, the second snRNA may be a snRNA U11. In some embodiments, the second snRNA may be a snRNA U12. In some embodiments, the second snRNA may be a snRNA 7SK. In some embodiments, the second snRNA is selected from the group consisting of snRNA U1, snRNA U2, snRNA U3, snRNA U4, snRNA U5, snRNA U6 snRNA U7, snRNA U11 and snRNA 7SK. In some embodiments, the second snRNA is selected from the group consisting of snRNA U1, snRNA U2, snRNA U3, snRNA U4, snRNA U5, snRNA U6 snRNA U7, snRNA U11, snRNA U12 and snRNA 7SK.

In some embodiments, the first organism species may be a human. In some embodiments, the first organism species may be a mouse. In some embodiments, the second organism species may be a human. In some embodiments, the second organism species may be a mouse.

Described herein, in some embodiments, are nucleic acid expression systems, comprising: a regulatory sequence operably coupled to a transcribable region of a nucleic acid, the regulatory sequence comprising a proximal regulatory sequence element (PSE) and a distal regulatory sequence element (DSE); wherein the PSE comprises a mouse U1 snRNA (“MmU1”) PSE, a mouse U2 snRNA (“MmU2”) PSE, a mouse U3 snRNA (“MmU3”) PSE, a mouse U4 snRNA (“MmU4”) PSE, a mouse U5 snRNA (“MmU5”) PSE, a mouse U6 snRNA (“MmU6”) PSE, a mouse U7 snRNA (“MmU7”) PSE, a mouse U11 snRNA (“MmU11”) PSE, a mouse U12 snRNA (“MmU12”) PSE, a mouse U7SK snRNA (“MmU7SK”) PSE, a human U1 snRNA (“HsU1”) PSE, a human U2 snRNA (“HsU2”) PSE, a human U3 snRNA (“HsU3”) PSE, a human U4 snRNA (“HsU4”) PSE, a human U5 snRNA (“HsU5”) PSE, a human U6 snRNA (“HsU6”) PSE, a human U7 snRNA (“HsU7”) PSE, a human U11 snRNA (“HsU11”) PSE, a human U12 snRNA (“HsU12”) PSE, or a human U7SK snRNA (“HsU7SK”) PSE; wherein the DSE comprises a mouse U1 snRNA (“MmU1”) DSE, a mouse U2 snRNA (“MmU2”) DSE, a mouse U3 snRNA (“MmU3”) DSE, a mouse U4 snRNA (“MmU4”) DSE, a mouse U5 snRNA (“MmU5”) DSE, a mouse U6 snRNA (“MmU6”) DSE, a mouse U7 snRNA (“MmU7”) DSE, a mouse U11 snRNA (“MmU11”) DSE, a mouse U12 snRNA (“MmU12”) DSE, a mouse U7SK snRNA (“MmU7SK”) DSE, a human U1 snRNA (“HsU1”) DSE, a human U2 snRNA (“HsU2”) DSE, a human U3 snRNA (“HsU3”) DSE, a human U4 snRNA (“HsU4”) DSE, a human U5 snRNA (“HsU5”) DSE, a human U6 snRNA (“HsU6”) DSE, a human U7 snRNA (“HsU7”) DSE, a human U11 snRNA (“HsU11”) DSE, a human U12 snRNA (“HsU12”) DSE, or a human U7SK snRNA (“HsU7SK”) DSE; and wherein the PSE and the DSE are from a different species as each other, or from a different snRNA as each other.

An example of a species includes a human. An example of a species includes a mouse. An example of an snRNA is an snRNA U1. An example of an snRNA is an snRNA U2. In some embodiments, the first snRNA may be an snRNA U3. An example of an snRNA is an snRNA U4. An example of an snRNA is an snRNA U5. In some embodiments, the first snRNA may be an snRNA U6. An example of an snRNA is an snRNA U7. In some embodiments, the first snRNA may be an snRNA U11. In some embodiments, the first snRNA may be an snRNA U12. In some embodiments, the first snRNA may be an snRNA 7SK.

In some embodiments, the PSE comprises a MmU1 PSE. In some embodiments, the PSE comprises a MmU2 PSE. In some embodiments, the PSE comprises a MmU3 PSE. In some embodiments, the PSE comprises a MmU4 PSE. In some embodiments, the PSE comprises a MmU5 PSE. In some embodiments, the PSE comprises a MmU6 PSE. In some embodiments, the PSE comprises a MmU7 PSE. In some embodiments, the PSE comprises a MmU11 PSE. In some embodiments, the PSE comprises a MmU12 PSE. In some embodiments, the PSE comprises a MmU7SK PSE. In some embodiments, the PSE comprises a HsU1 PSE. In some embodiments, the PSE comprises a HsU2 PSE. In some embodiments, the PSE comprises a HsU3 PSE. In some embodiments, the PSE comprises a HsU4 PSE. In some embodiments, the PSE comprises a HsU5 PSE. In some embodiments, the PSE comprises a HsU6 PSE. In some embodiments, the PSE comprises a HsU7 PSE. In some embodiments, the PSE comprises a HsU11 PSE. In some embodiments, the PSE comprises a HsU12 PSE. In some embodiments, the PSE comprises a HsU7SK PSE. In some embodiments, the PSE comprises a PSE of a regulatory element in Table 5, or a variant thereof.

In some embodiments, the DSE comprises a MmU1 DSE. In some embodiments, the DSE comprises a MmU2 DSE. In some embodiments, the DSE comprises a MmU3 DSE. In some embodiments, the DSE comprises a MmU4 DSE. In some embodiments, the DSE comprises a MmU5 DSE. In some embodiments, the DSE comprises a MmU6 DSE. In some embodiments, the DSE comprises a MmU7 DSE. In some embodiments, the DSE comprises a MmU11 DSE. In some embodiments, the DSE comprises a MmU12 DSE. In some embodiments, the DSE comprises a MmU7SK DSE. In some embodiments, the DSE comprises a HsU1 DSE. In some embodiments, the DSE comprises a HsU2 DSE. In some embodiments, the DSE comprises a HsU3 DSE. In some embodiments, the DSE comprises a HsU4 DSE. In some embodiments, the DSE comprises a HsU5 DSE. In some embodiments, the DSE comprises a HsU6 DSE. In some embodiments, the DSE comprises a HsU7 DSE. In some embodiments, the DSE comprises a HsU11 DSE. In some embodiments, the DSE comprises a HsU12 DSE. In some embodiments, the DSE comprises a HsU7SK DSE. In some embodiments, the DSE comprises a PSE of a regulatory element in Table 5, or a variant thereof.

Described herein, in some embodiments, is a nucleic acid expression system. In some embodiments, the nucleic acid expression system may contain a regulatory sequence operably coupled to a transcribable region of a nucleic acid. In some embodiments, the regulatory sequence may include a proximal regulatory sequence element (PSE). In some embodiments, the regulatory sequence may include a distal regulatory sequence element (DSE). In some embodiments, the regulatory sequence may include a PSE and a DSE. In some embodiments, the PSE may include a mouse U7 snRNA (“Mm U7”) PSE. In some embodiments, the PSE may include a human U7 snRNA (“Hs U7”) PSE. In some embodiments, the PSE may include a mouse U1a1 (“mu1a1”) PSE. In some embodiments, the PSE may include a human U1-1 (“HU1” or “Hs U1-1”) PSE.

In some embodiments, the DSE may include a Mm U7 DSE. In some embodiments, the DSE may include a Hs U7 DSE. In some embodiments, the DSE may include a mu1a1 DSE. In some embodiments, the DSE may include a HU1 DSE. In some embodiments, when the PSE comprises the Mm U7 PSE, the DSE does not comprise the Mm U7 DSE. In some embodiments, when the PSE comprises the Hs U7 PSE, the DSE does not comprise the Hs U7 DSE. In some embodiments, when the PSE comprises the mu1a1 PSE, the DSE does not comprise the mu1a1 DSE. In some embodiments, when the PSE comprises the HU1 PSE, the DSE does not comprise the HU1 DSE.

Described herein, in some embodiments, is a nucleic acid system comprised of a regulatory sequence operably coupled to a transcribable region of a nucleic acid, with the regulatory sequence comprised of a proximal regulatory sequence element (PSE) and a distal regulatory sequence element (DSE), wherein the PSE is comprised of a mouse U7 snRNA (“Mm U7”) PSE, a human U7 snRNA (“Hs U7”) PSE, a mouse U1a1 (“mu1a1”) PSE, or a human U1-1 (“HU1” or “Hs U1-1”) PSE; and wherein the DSE is comprised of a Mm U7 DSE, a Hs U7 DSE, a mu1a1 DSE, or a HU1 DSE, wherein when the PSE comprises the Mm U7 PSE, the DSE does not comprise the Mm U7 DSE, when the PSE comprises the Hs U7 PSE, the DSE does not comprise the Hs U7 DSE, when the PSE comprises the mu1a1 PSE, the DSE does not comprise the mu1a1 DSE, and when the PSE comprises the HU1 PSE, the DSE does not comprise the HU1 DSE.

In some embodiments, the regulatory sequence may include a promoter sequence. In some embodiments, the regulatory sequence may not include a promoter sequence. In some embodiments, the regulatory sequence may include a terminator sequence. In some embodiments, the regulatory sequence may not include a terminator sequence.

Described herein, in some embodiments, are nucleic acid expression systems, comprising: a promoter sequence comprising a promoter proximal sequence element (PSE) and a promoter distal sequence element (DSE); wherein the PSE comprises a mouse U1 snRNA (“MmU1”) promoter PSE, a mouse U2 snRNA (“MmU2”) promoter PSE, a mouse U3 snRNA (“MmU3”) promoter PSE, a mouse U4 snRNA (“MmU4”) promoter PSE, a mouse U5 snRNA (“MmU5”) promoter PSE, a mouse U6 snRNA (“MmU6”) promoter PSE, a mouse U7 snRNA (“MmU7”) promoter PSE, a mouse U11 snRNA (“MmU11”) promoter PSE, a mouse U12 snRNA (“MmU12”) promoter PSE, a mouse U7SK snRNA (“MmU7SK”) promoter PSE, a human U1 snRNA (“HsU1”) promoter PSE, a human U2 snRNA (“HsU2”) promoter PSE, a human U3 snRNA (“HsU3”) promoter PSE, a human U4 snRNA (“HsU4”) promoter PSE, a human U5 snRNA (“HsU5”) promoter PSE, a human U6 snRNA (“HsU6”) promoter PSE, a human U7 snRNA (“HsU7”) promoter PSE, a human U11 snRNA (“HsU11”) promoter PSE, a human U12 snRNA (“HsU12”) promoter PSE, or a human U7SK snRNA (“HsU7SK”) promoter PSE; wherein the DSE comprises a mouse U1 snRNA (“MmU1”) promoter DSE, a mouse U2 snRNA (“MmU2”) promoter DSE, a mouse U3 snRNA (“MmU3”) promoter DSE, a mouse U4 snRNA (“MmU4”) promoter DSE, a mouse U5 snRNA (“MmU5”) promoter DSE, a mouse U6 snRNA (“MmU6”) promoter DSE, a mouse U7 snRNA (“MmU7”) promoter DSE, a mouse U11 snRNA (“MmU11”) promoter DSE, a mouse U12 snRNA (“MmU12”) promoter DSE, a mouse U7SK snRNA (“MmU7SK”) promoter DSE, a human U1 snRNA (“HsU1”) promoter DSE, a human U2 snRNA (“HsU2”) promoter DSE, a human U3 snRNA (“HsU3”) promoter DSE, a human U4 snRNA (“HsU4”) promoter DSE, a human U5 snRNA (“HsU5”) promoter DSE, a human U6 snRNA (“HsU6”) promoter DSE, a human U7 snRNA (“HsU7”) promoter DSE, a human U11 snRNA (“HsU11”) promoter DSE, a human U12 snRNA (“HsU12”) promoter DSE, or a human U7SK snRNA (“HsU7SK”) promoter DSE; and wherein the promoter PSE and the promoter DSE are from a different species as each other, or from a different snRNA as each other. In some embodiments, the promoter PSE and the promoter DSE are from a different species as each other, and from a different snRNA as each other. Some embodiments include a transcribable region operably coupled to the promoter sequence. Some embodiments include a terminator sequence 3′ to the transcribable region.

In some embodiments, the promoter PSE comprises a MmU1 promoter PSE. In some embodiments, the promoter PSE comprises a MmU2 promoter PSE. In some embodiments, the promoter PSE comprises a MmU3 promoter PSE. In some embodiments, the promoter PSE comprises a MmU4 promoter PSE. In some embodiments, the promoter PSE comprises a MmU5 promoter PSE. In some embodiments, the promoter PSE comprises a MmU6 promoter PSE. In some embodiments, the promoter PSE comprises a MmU7 promoter PSE. In some embodiments, the promoter PSE comprises a MmU11 promoter PSE. In some embodiments, the promoter PSE comprises a MmU12 promoter PSE. In some embodiments, the promoter PSE comprises a MmU7SK promoter PSE. In some embodiments, the promoter PSE comprises a HsU1 promoter PSE. In some embodiments, the promoter PSE comprises a HsU2 promoter PSE. In some embodiments, the promoter PSE comprises a HsU3 promoter PSE. In some embodiments, the promoter PSE comprises a HsU4 promoter PSE. In some embodiments, the promoter PSE comprises a HsU5 promoter PSE. In some embodiments, the promoter PSE comprises a HsU6 promoter PSE. In some embodiments, the promoter PSE comprises a HsU7 promoter PSE. In some embodiments, the promoter PSE comprises a HsU11 promoter PSE. In some embodiments, the promoter PSE comprises a HsU12 promoter PSE. In some embodiments, the promoter PSE comprises a HsU7SK promoter PSE. In some embodiments, the promoter PSE comprises a promoter PSE of a regulatory element in Table 5, or a variant thereof.

In some embodiments, the promoter DSE comprises a MmU1 promoter DSE. In some embodiments, the promoter DSE comprises a MmU2 promoter DSE. In some embodiments, the promoter DSE comprises a MmU3 promoter DSE. In some embodiments, the promoter DSE comprises a MmU4 promoter DSE. In some embodiments, the promoter DSE comprises a MmU5 promoter DSE. In some embodiments, the promoter DSE comprises a MmU6 promoter DSE. In some embodiments, the promoter DSE comprises a MmU7 promoter DSE. In some embodiments, the promoter DSE comprises a MmU11 promoter DSE. In some embodiments, the promoter DSE comprises a MmU12 promoter DSE. In some embodiments, the promoter DSE comprises a MmU7SK promoter DSE. In some embodiments, the promoter DSE comprises a HsU1 promoter DSE. In some embodiments, the promoter DSE comprises a HsU2 promoter DSE. In some embodiments, the promoter DSE comprises a HsU3 promoter DSE. In some embodiments, the promoter DSE comprises a HsU4 promoter DSE. In some embodiments, the promoter DSE comprises a HsU5 promoter DSE. In some embodiments, the promoter DSE comprises a HsU6 promoter DSE. In some embodiments, the promoter DSE comprises a HsU7 promoter DSE. In some embodiments, the promoter DSE comprises a HsU11 promoter DSE. In some embodiments, the promoter DSE comprises a HsU12 promoter DSE. In some embodiments, the promoter DSE comprises a HsU7SK promoter DSE. In some embodiments, the promoter DSE comprises a promoter DSE of a regulatory element in Table 5, or a variant thereof.

Described herein, in some embodiments, is a nucleic acid expression system. In some embodiments, the nucleic acid expression system may include a promoter sequence. In some embodiments, the promoter sequence may include a promoter proximal sequence element (PSE). In some embodiments, the promoter sequence may include a promoter distal sequence element (DSE). In some embodiments, the promoter sequence may include a PSE and DSE. In some embodiments, the promoter PSE may include a mouse U7 snRNA (“Mm U7”) promoter PSE. In some embodiments, the promoter PSE may include a human U7 snRNA (“Hs U7”) promoter PSE. In some embodiments, the promoter PSE may include a mouse U1a1 (“mu1a1”) promoter PSE. In some embodiments, the promoter PSE may include a human U1-1 (“HU1” or “Hs U1-1”) promoter PSE. In some embodiments, the promoter DSE may include a Mm U7 promoter DSE. In some embodiments, the promoter DSE may include a Hs U7 promoter DSE. In some embodiments, the promoter DSE may include a mu1a1 promoter DSE. In some embodiments, the promoter DSE may include a HU1 promoter DSE. In some embodiments, when the promoter PSE comprises the Mm U7 promoter PSE, the promoter DSE does not comprise the Mm U7 promoter DSE. In some embodiments, when the promoter PSE comprises the Hs U7 promoter PSE, the promoter DSE does not comprise the Hs U7 promoter DSE. In some embodiments, when the promoter PSE comprises the mu1a1 promoter PSE, the promoter DSE does not comprise the mu1a1 promoter DSE. In some embodiments, when the promoter PSE comprises the HU1 promoter PSE, the promoter DSE does not comprise the HU1 promoter DSE.

Described herein, in some embodiments, is a nucleic acid system comprised of a promoter sequence comprised of a promoter proximal sequence element (PSE) and a promoter distal sequence element (DSE), wherein the promoter PSE is comprised of a mouse U7 snRNA (“Mm U7”) promoter PSE, a human U7 snRNA (“Hs U7”) promoter PSE, a mouse U1a1 (“mu1a1”) promoter PSE, or a human U1-1 (“HU1” or “Hs U1-1”) promoter PSE; and wherein the promoter DSE is comprised of a Mm U7 promoter DSE, a Hs U7 promoter DSE, a mu1a1 promoter DSE, or a HU1 promoter DSE, wherein, when the promoter PSE comprises the Mm U7 promoter PSE, the promoter DSE does not comprise the Mm U7 promoter DSE, when the promoter PSE comprises the Hs U7 promoter PSE, the promoter DSE does not comprise the Hs U7 promoter DSE, when the promoter PSE comprises the mu1a1 promoter PSE, the promoter DSE does not comprise the mu1a1 promoter DSE, and when the promoter PSE comprises the HU1 promoter PSE, the promoter DSE does not comprise the HU1 promoter DSE.

In some embodiments, the system may further comprise a transcribable region operably coupled to the promoter sequence. In some embodiments, the system may further comprise a terminator sequence 3′ to the transcribable region. In some embodiments, the system may further comprise a transcribable region operably coupled to the promoter sequence and a terminator sequence 3′ to the transcribable region.

Described herein, in some embodiments, are nucleic acid expression systems, comprising, comprising: a terminator sequence 3′ to a transcribable region, the terminator sequence comprising a terminator proximal sequence element (PSE) and a terminator distal sequence element (DSE); wherein the PSE comprises a mouse U1 snRNA (“MmU1”) terminator PSE, a mouse U2 snRNA (“MmU2”) terminator PSE, a mouse U3 snRNA (“MmU3”) terminator PSE, a mouse U4 snRNA (“MmU4”) terminator PSE, a mouse U5 snRNA (“MmU5”) terminator PSE, a mouse U6 snRNA (“MmU6”) terminator PSE, a mouse U7 snRNA (“MmU7”) terminator PSE, a mouse U11 snRNA (“MmU11”) terminator PSE, a mouse U12 snRNA (“MmU12”) terminator PSE, a mouse U7SK snRNA (“MmU7SK”) terminator PSE, a human U1 snRNA (“HsU1”) terminator PSE, a human U2 snRNA (“HsU2”) terminator PSE, a human U3 snRNA (“HsU3”) terminator PSE, a human U4 snRNA (“HsU4”) terminator PSE, a human U5 snRNA (“HsU5”) terminator PSE, a human U6 snRNA (“HsU6”) terminator PSE, a human U7 snRNA (“HsU7”) terminator PSE, a human U11 snRNA (“HsU11”) terminator PSE, a human U12 snRNA (“HsU12”) terminator PSE, or a human U7SK snRNA (“HsU7SK”) terminator PSE; wherein the DSE comprises a mouse U1 snRNA (“MmU1”) terminator DSE, a mouse U2 snRNA (“MmU2”) terminator DSE, a mouse U3 snRNA (“MmU3”) terminator DSE, a mouse U4 snRNA (“MmU4”) terminator DSE, a mouse U5 snRNA (“MmU5”) terminator DSE, a mouse U6 snRNA (“MmU6”) terminator DSE, a mouse U7 snRNA (“MmU7”) terminator DSE, a mouse U11 snRNA (“MmU11”) terminator DSE, a mouse U12 snRNA (“MmU12”) terminator DSE, a mouse U7SK snRNA (“MmU7SK”) terminator DSE, a human U1 snRNA (“HsU1”) terminator DSE, a human U2 snRNA (“HsU2”) terminator DSE, a human U3 snRNA (“HsU3”) terminator DSE, a human U4 snRNA (“HsU4”) terminator DSE, a human U5 snRNA (“HsU5”) terminator DSE, a human U6 snRNA (“HsU6”) terminator DSE, a human U7 snRNA (“HsU7”) terminator DSE, a human U11 snRNA (“HsU11”) terminator DSE, a human U12 snRNA (“HsU12”) terminator DSE, or a human U7SK snRNA (“HsU7SK”) terminator DSE; and wherein the terminator PSE and the terminator DSE are from a different species as each other, or from a different snRNA as each other. In some embodiments, the terminator PSE and the terminator DSE are from a different species as each other, and from a different snRNA as each other. Some embodiments include a promoter sequence, wherein the transcribable region is operably coupled to the promoter sequence.

In some embodiments, the terminator PSE comprises a MmU1 terminator PSE. In some embodiments, the terminator PSE comprises a MmU2 terminator PSE. In some embodiments, the terminator PSE comprises a MmU3 terminator PSE. In some embodiments, the terminator PSE comprises a MmU4 terminator PSE. In some embodiments, the terminator PSE comprises a MmU5 terminator PSE. In some embodiments, the terminator PSE comprises a MmU6 terminator PSE. In some embodiments, the terminator PSE comprises a MmU7 terminator PSE. In some embodiments, the terminator PSE comprises a MmU11 terminator PSE. In some embodiments, the terminator PSE comprises a MmU12 terminator PSE. In some embodiments, the terminator PSE comprises a MmU7SK terminator PSE. In some embodiments, the terminator PSE comprises a HsU1 terminator PSE. In some embodiments, the terminator PSE comprises a HsU2 terminator PSE. In some embodiments, the terminator PSE comprises a HsU3 terminator PSE. In some embodiments, the terminator PSE comprises a HsU4 terminator PSE. In some embodiments, the terminator PSE comprises a HsU5 terminator PSE. In some embodiments, the terminator PSE comprises a HsU6 terminator PSE. In some embodiments, the terminator PSE comprises a HsU7 terminator PSE. In some embodiments, the terminator PSE comprises a HsU11 terminator PSE. In some embodiments, the terminator PSE comprises a HsU12 terminator PSE. In some embodiments, the terminator PSE comprises a HsU7SK terminator PSE. In some embodiments, the terminator PSE comprises a terminator PSE of a regulatory element in Table 5, or a variant thereof.

In some embodiments, the terminator DSE comprises a MmU1 terminator DSE. In some embodiments, the terminator DSE comprises a MmU2 terminator DSE. In some embodiments, the terminator DSE comprises a MmU3 terminator DSE. In some embodiments, the terminator DSE comprises a MmU4 terminator DSE. In some embodiments, the terminator DSE comprises a MmU5 terminator DSE. In some embodiments, the terminator DSE comprises a MmU6 terminator DSE. In some embodiments, the terminator DSE comprises a MmU7 terminator DSE. In some embodiments, the terminator DSE comprises a MmU11 terminator DSE. In some embodiments, the terminator DSE comprises a MmU12 terminator DSE. In some embodiments, the terminator DSE comprises a MmU7SK terminator DSE. In some embodiments, the terminator DSE comprises a HsU1 terminator DSE. In some embodiments, the terminator DSE comprises a HsU2 terminator DSE. In some embodiments, the terminator DSE comprises a HsU3 terminator DSE. In some embodiments, the terminator DSE comprises a HsU4 terminator DSE. In some embodiments, the terminator DSE comprises a HsU5 terminator DSE. In some embodiments, the terminator DSE comprises a HsU6 terminator DSE. In some embodiments, the terminator DSE comprises a HsU7 terminator DSE. In some embodiments, the terminator DSE comprises a HsU11 terminator DSE. In some embodiments, the terminator DSE comprises a HsU12 terminator DSE. In some embodiments, the terminator DSE comprises a HsU7SK terminator DSE. In some embodiments, the terminator DSE comprises a terminator DSE of a regulatory element in Table 5, or a variant thereof.

Described herein, in some embodiments, is a nucleic acid expression system. In some embodiments, the nucleic acid expression system may include a terminator sequence 3′ to a transcribable region. In some embodiments, the terminator sequence may include a terminator proximal sequence element (PSE). In some embodiments, the terminator sequence may include a terminator distal sequence element (DSE). In some embodiments, the terminator sequence may include a terminator PSE and a terminator DSE. In some embodiments, the terminator PSE may include a mouse U7 snRNA (“Mm U7”) terminator PSE. In some embodiments, the terminator PSE may include a human U7 snRNA (“Hs U7”) terminator PSE. In some embodiments, the terminator PSE may include a mouse U1a1 (“mu1a1”) terminator PSE. In some embodiments, the terminator PSE may include a human U1-1 (“HU1” or “Hs U1-1”) terminator PSE. In some embodiments, the terminator DSE may include a Mm U7 terminator DSE. In some embodiments, the terminator DSE may include a Hs U7 terminator DSE. In some embodiments, the terminator DSE may include a mu1a1 terminator DSE. In some embodiments, the terminator DSE may include a HU1 terminator DSE. In some embodiments, when the terminator PSE comprises the Mm U7 terminator PSE, the terminator DSE does not comprise the Mm U7 terminator DSE. In some embodiments, when the terminator PSE comprises the Hs U7 terminator PSE, the terminator DSE does not comprise the Hs U7 terminator DSE. In some embodiments, when the terminator PSE comprises the mu1a1 terminator PSE, the terminator DSE does not comprise the mu1a1 terminator DSE. In some embodiments, when the terminator PSE comprises the HU1 terminator PSE, the terminator DSE does not comprise the HU1 terminator DSE. In some embodiments, the nucleic acid expression system may include a promoter sequence. In some embodiments, the promoter sequence may include the transcribable region operably coupled to the promoter sequence.

Described herein, in some embodiments, is a nucleic acid system comprised of a terminator sequence 3′ to a transcribable region, the terminator sequence comprised of a terminator proximal sequence element (PSE) and a terminator distal sequence element (DSE), wherein, the terminator PSE is comprised of a mouse U7 snRNA (“Mm U7”) terminator PSE, a human U7 snRNA (“Hs U7”) terminator PSE, a mouse U1a1 (“mu1a1”) terminator PSE, or a human U1-1 (“HU1” or “Hs U1-1”) terminator PSE; and wherein, the terminator DSE is comprised of a Mm U7 terminator DSE, a Hs U7 terminator DSE, a mu1a1 terminator DSE, or a HU1 terminator DSE, wherein, when the terminator PSE comprises the Mm U7 terminator PSE, the terminator DSE does not comprise the Mm U7 terminator DSE, when the terminator PSE comprises the Hs U7 terminator PSE, the terminator DSE does not comprise the Hs U7 terminator DSE, when the terminator PSE comprises the mu1a1 terminator PSE, the terminator DSE does not comprise the mu1a1 terminator DSE, and when the terminator PSE comprises the HU1 terminator PSE, the terminator DSE does not comprise the HU1 terminator DSE.

Described herein, in some embodiments, is a nucleic acid expression system. In some embodiments, the nucleic acid expression system may include a promoter sequence operably coupled to a transcribable region of a nucleic acid. In some embodiments, the nucleic acid expression system may include a terminator sequence coupled to the transcribable region. In some embodiments, the nucleic acid expression system may include a promoter sequence operably coupled to a transcribable region of a nucleic acid and a terminator sequence coupled to the transcribable region. In some embodiments, the promoter may include a first small nuclear RNA (snRNA) of a first organism species. In some embodiments, the terminator sequence may include a terminator sequence of a second small nuclear RNA (snRNA) of a second organism species. In some embodiments, the first and second snRNAs are different. In some embodiments, the first and second snRNAs are the same. In some embodiments, the first and second organism species are different. In some embodiments, the first and second organism species are the same.

Described herein, in some embodiments, is a nucleic acid system comprised of a promoter sequence operably coupled to a transcribable region of a nucleic acid, and a terminator sequence coupled to the transcribable region; wherein the promoter sequence is comprised of a promoter sequence of a first small nuclear RNA (snRNA) of a first organism species, wherein the terminator sequence is comprised of a terminator sequence of a second small nuclear RNA (snRNA) of a second organism species, and wherein the first and second snRNAs are different or wherein the first and second organism species are different.

In some embodiments, the first snRNA may be a snRNA U1. In some embodiments, the first snRNA may be a snRNA U2. In some embodiments, the first snRNA may be a snRNA U3. In some embodiments, the first snRNA may be a snRNA U4. In some embodiments, the first snRNA may be a snRNA U5. In some embodiments, the first snRNA may be a snRNA U6. In some embodiments, the first snRNA may be a snRNA U7. In some embodiments, the first snRNA may be a snRNA U11. In some embodiments, the first snRNA may be a snRNA U12. In some embodiments, the first snRNA may be a snRNA 7SK. In some embodiments, the first snRNA is selected from the group consisting of snRNA U1, snRNA U2, snRNA U3, snRNA U4, snRNA U5, snRNA U6 snRNA U7, snRNA U11 and snRNA 7SK. In some embodiments, the first snRNA is selected from the group consisting of snRNA U1, snRNA U2, snRNA U3, snRNA U4, snRNA U5, snRNA U6 snRNA U7, snRNA U11, snRNA U12 and snRNA 7SK.

In some embodiments, the second snRNA may be a snRNA U1. In some embodiments, the second snRNA may be a snRNA U2. In some embodiments, the second snRNA may be a snRNA U3. In some embodiments, the second snRNA may be a snRNA U4. In some embodiments, the second snRNA may be a snRNA U5. In some embodiments, the second snRNA may be a snRNA U6. In some embodiments, the second snRNA may be a snRNA U7. In some embodiments, the second snRNA may be a snRNA U11. In some embodiments, the second snRNA may be a snRNA U12. In some embodiments, the second snRNA may be a snRNA 7SK. In some embodiments, the second snRNA is selected from the group consisting of snRNA U1, snRNA U2, snRNA U3, snRNA U4, snRNA U5, snRNA U6 snRNA U7, snRNA U11 and snRNA 7SK. In some embodiments, the second snRNA is selected from the group consisting of snRNA U1, snRNA U2, snRNA U3, snRNA U4, snRNA U5, snRNA U6 snRNA U7, snRNA U11, snRNA U12 and snRNA 7SK.

In some embodiments, the first organism species may be a human. In some embodiments, the first organism species may be a mouse. In some embodiments, the second organism species may be a human. In some embodiments, the second organism species may be a mouse.

Described herein, in some embodiments, are nucleic acid expression systems, comprising, comprising: a promoter sequence operably coupled to a transcribable region of a nucleic acid, and a terminator sequence coupled to the transcribable region; wherein the promoter sequence comprises a promoter sequence of a first small nuclear RNA (snRNA) of a first organism species, wherein the terminator sequence comprises a terminator sequence of a second small nuclear RNA (snRNA) of a second organism species, and wherein the first and second snRNAs are different or wherein the first and second organism species are different. In some embodiments, the first snRNA is selected from the group consisting of snRNA U1, snRNA U2, snRNA U3, snRNA U4, snRNA U5, snRNA U6, snRNA U7, snRNA U11, snRNA U12 and snRNA 7SK. In some embodiments, the second snRNA is selected from the group consisting of snRNA U7, snRNA U2, snRNA U3, snRNA U4, snRNA U5, snRNA U6, snRNA U1, snRNA U11, snRNA U12 and snRNA 7SK. In some embodiments, the first snRNA is selected from the group consisting of snRNA U1, snRNA U2, snRNA U3, snRNA U4, snRNA U5, snRNA U6, snRNA U7, snRNA U11, snRNA U12 and snRNA 7SK; the second snRNA is selected from the group consisting of snRNA U7, snRNA U2, snRNA U3, snRNA U4, snRNA U5, snRNA U6, snRNA U1, snRNA U11, snRNA U12 and snRNA 7SK; the first organism species is selected from the group consisting of human and mouse; and the second organism species is selected from the group consisting of mouse and human. In some embodiments, the promoter comprises a promoter PSE sequence and a promoter DSE sequence, and wherein the either the promoter PSE or the promoter DSE is of a different organism species than the first organism species. In some embodiments, the promoter comprises a promoter PSE sequence and a promoter DSE sequence, and wherein the either the promoter PSE or the promoter DSE is of a different snRNA than the first snRNA. In some embodiments, the promoter comprises a promoter PSE sequence and a promoter DSE sequence, and wherein the either the promoter PSE or the promoter DSE is of a different organism species than the first organism species; and wherein the promoter comprises a promoter PSE sequence and a promoter DSE sequence, and wherein the either the promoter PSE or the promoter DSE is of a different snRNA than the first snRNA. In some embodiments, the terminator comprises a terminator PSE sequence and a terminator DSE sequence, and wherein the either the terminator PSE or the terminator DSE is of a different organism species than the second organism species. In some embodiments, the terminator comprises a terminator PSE sequence and a terminator DSE sequence, and wherein the either the terminator PSE or the terminator DSE is of a different snRNA than the second snRNA. In some embodiments, the terminator comprises a terminator PSE sequence and a terminator DSE sequence, and wherein the either the terminator PSE or the terminator DSE is of a different organism species than the second organism species; and wherein the terminator comprises a terminator PSE sequence and a terminator DSE sequence, and wherein the either the terminator PSE or the terminator DSE is of a different snRNA than the second snRNA. In some embodiments, the promoter comprises a promoter PSE sequence and a promoter DSE sequence, and wherein the either the promoter PSE or the promoter DSE is of a different organism species than the first organism species; and wherein the promoter comprises a promoter PSE sequence and a promoter DSE sequence, and wherein the either the promoter PSE or the promoter DSE is of a different snRNA than the first snRNA; wherein the terminator comprises a terminator PSE sequence and a terminator DSE sequence, and wherein the either the terminator PSE or the terminator DSE is of a different organism species than the second organism species; and wherein the terminator comprises a terminator PSE sequence and a terminator DSE sequence, and wherein the either the terminator PSE or the terminator DSE is of a different snRNA than the second snRNA. In some embodiments, the promoter sequence comprises a promoter sequence in Table 5, or a variant thereof. In some embodiments, the terminator sequence comprises a terminator sequence in Table 5, or a variant thereof. In some embodiments, the promoter DSE or promoter PSE comprises a promoter DSE or promoter PSE of a promoter in Table 5, or a variant thereof. In some embodiments, the terminator DSE or terminator PSE comprises a terminator DSE or terminator PSE of a terminator in Table 5, or a variant thereof.

Described herein, in some embodiments, are nucleic acid expression systems, comprising: a promoter sequence comprising a mouse U1 snRNA (“MmU1”) promoter sequence, a mouse U2 snRNA (“MmU2”) promoter sequence, a mouse U3 snRNA (“MmU3”) promoter sequence, a mouse U4 snRNA (“MmU4”) promoter sequence, a mouse U5 snRNA (“MmU5”) promoter sequence, a mouse U6 snRNA (“MmU6”) promoter sequence, a mouse U7 snRNA (“MmU7”) promoter sequence, a mouse U11 snRNA (“MmU11”) promoter sequence, a mouse U12 snRNA (“MmU12”) promoter sequence, a mouse U7SK snRNA (“MmU7SK”) promoter sequence, a human U1 snRNA (“HsU1”) promoter sequence, a human U2 snRNA (“HsU2”) promoter sequence, a human U3 snRNA (“HsU3”) promoter sequence, a human U4 snRNA (“HsU4”) promoter sequence, a human U5 snRNA (“HsU5”) promoter sequence, a human U6 snRNA (“HsU6”) promoter sequence, a human U7 snRNA (“HsU7”) promoter sequence, a human U11 snRNA (“HsU11”) promoter sequence, a human U12 snRNA (“HsU12”) promoter sequence, or a human U7SK snRNA (“HsU7SK”) promoter sequence, or a fragment or combination of fragments thereof; and a transcribable region operably coupled to the promoter sequence and to a terminator sequence comprising a mouse U1 snRNA (“MmU1”) terminator sequence, a mouse U2 snRNA (“MmU2”) terminator sequence, a mouse U3 snRNA (“MmU3”) terminator sequence, a mouse U4 snRNA (“MmU4”) terminator sequence, a mouse U5 snRNA (“MmU5”) terminator sequence, a mouse U6 snRNA (“MmU6”) terminator sequence, a mouse U7 snRNA (“MmU7”) terminator sequence, a mouse U11 snRNA (“MmU11”) terminator sequence, a mouse U12 snRNA (“MmU12”) terminator sequence, a mouse U7SK snRNA (“MmU7SK”) terminator sequence, a human U1 snRNA (“HsU1”) terminator sequence, a human U2 snRNA (“HsU2”) terminator sequence, a human U3 snRNA (“HsU3”) terminator sequence, a human U4 snRNA (“HsU4”) terminator sequence, a human U5 snRNA (“HsU5”) terminator sequence, a human U6 snRNA (“HsU6”) terminator sequence, a human U7 snRNA (“HsU7”) terminator sequence, a human U11 snRNA (“HsU11”) terminator sequence, a human U12 snRNA (“HsU12”) terminator sequence, or a human U7SK snRNA (“HsU7SK”) terminator sequence, or a fragment or combination of fragments thereof; wherein the promoter sequence and the terminator sequence are at least partially of a different organism species as each other, or are at least partially of a different snRNA as each other. In some embodiments, the promoter sequence and the terminator sequence are at least partially of a different organism species as each other, and are at least partially of a different snRNA as each other. In some embodiments, the promoter comprises a promoter PSE sequence and a promoter DSE sequence, and wherein the either the promoter PSE or the promoter DSE is of a different organism species than the first organism species. In some embodiments, the promoter comprises a promoter PSE sequence and a promoter DSE sequence, and wherein the either the promoter PSE or the promoter DSE is of a different snRNA than the first snRNA. In some embodiments, the promoter comprises a promoter PSE sequence and a promoter DSE sequence, and wherein the either the promoter PSE or the promoter DSE is of a different organism species than the first organism species; and wherein the promoter comprises a promoter PSE sequence and a promoter DSE sequence, and wherein the either the promoter PSE or the promoter DSE is of a different snRNA than the first snRNA. In some embodiments, the terminator comprises a terminator PSE sequence and a terminator DSE sequence, and wherein the either the terminator PSE or the terminator DSE is of a different organism species than the second organism species. In some embodiments, the terminator comprises a terminator PSE sequence and a terminator DSE sequence, and wherein the either the terminator PSE or the terminator DSE is of a different snRNA than the second snRNA. In some embodiments, the terminator comprises a terminator PSE sequence and a terminator DSE sequence, and wherein the either the terminator PSE or the terminator DSE is of a different organism species than the second organism species; and wherein the terminator comprises a terminator PSE sequence and a terminator DSE sequence, and wherein the either the terminator PSE or the terminator DSE is of a different snRNA than the second snRNA. In some embodiments, the promoter comprises a promoter PSE sequence and a promoter DSE sequence, and wherein the either the promoter PSE or the promoter DSE is of a different organism species than the first organism species; and wherein the promoter comprises a promoter PSE sequence and a promoter DSE sequence, and wherein the either the promoter PSE or the promoter DSE is of a different snRNA than the first snRNA; wherein the terminator comprises a terminator PSE sequence and a terminator DSE sequence, and wherein the either the terminator PSE or the terminator DSE is of a different organism species than the second organism species; and wherein the terminator comprises a terminator PSE sequence and a terminator DSE sequence, and wherein the either the terminator PSE or the terminator DSE is of a different snRNA than the second snRNA. In some embodiments, the promoter sequence comprises a promoter sequence in Table 5, or a variant thereof. In some embodiments, the terminator sequence comprises a terminator sequence in Table 5, or a variant thereof. In some embodiments, the promoter DSE or promoter PSE comprises a promoter DSE or promoter PSE of a promoter in Table 5, or a variant thereof. In some embodiments, the terminator DSE or terminator PSE comprises a terminator DSE or terminator PSE of a terminator in Table 5, or a variant thereof.

Described herein, in some embodiments, is a nucleic acid expression system. In some embodiments, the nucleic acid expression system may include a promoter sequence. In some embodiments, the promoter sequence may include a mouse U7 snRNA (“Mm U7”) promoter sequence. In some embodiments, the promoter sequence may include a human U7 snRNA (“Hs U7”) promoter sequence. In some embodiments, the promoter sequence may include a mouse U1a1 (“mu1a1”) promoter sequence. In some embodiments, the promoter sequence may include a human U1-1 (“HU1” or “Hs U1-1”) promoter sequence. In some embodiments, the promoter sequence may include a fragment of the promoters listed above. In some embodiments, the promoter sequence may include a combination of fragments listed above. In some embodiments, the nucleic acid expression system may include a transcribable region operably coupled to the promoter sequence. In some embodiments, the nucleic acid expression system may include a transcribable region operably coupled to a terminator sequence. In some embodiments, the nucleic acid expression system may include a transcribable region operably coupled to the promoter sequence and a terminator sequence. In some embodiments, the terminator sequence may include a Mm U7 terminator sequence. In some embodiments, the terminator sequence may include a Hs U7 terminator sequence. In some embodiments, the terminator sequence may include a mu1a1 terminator sequence. In some embodiments, the terminator sequence may include a HU1 terminator sequence. In some embodiments, the terminator sequence may include a fragment of the terminators listed above. In some embodiments, the terminator sequence may include a combination of fragments of the terminators listed above. In some embodiments, when the promoter sequence comprises the Mm U7 promoter sequence or a fragment thereof, the terminator sequence does not comprise the Mm U7 terminator sequence or a fragment thereof. In some embodiments, when the promoter sequence comprises the Hs U7 promoter sequence or a fragment thereof, the terminator sequence does not comprise the Hs U7 terminator sequence or a fragment thereof. In some embodiments, when the promoter sequence comprises the mu1a1 promoter sequence or a fragment thereof, the terminator sequence does not comprise the mu1a1 terminator sequence or a fragment thereof. In some embodiments, when the promoter sequence comprises the HU1 promoter sequence or a fragment thereof, the terminator sequence does not comprise the HU1 terminator sequence or a fragment thereof.

Described herein, in some embodiments, is a nucleic acid system comprised of a promoter sequence comprised of a mouse U7 snRNA (“Mm U7”) promoter sequence, a human U7 snRNA (“Hs U7”) promoter sequence, a mouse U1a1 (“mu1a1”) promoter sequence, or a human U1-1 (“HU1” or “Hs U1-1”) promoter sequence, or a fragment or combination of fragments thereof; and a transcribable region operably coupled to the promoter sequence and to a terminator sequence comprised of a Mm U7 terminator sequence, a Hs U7 terminator sequence, a mu1a1 terminator sequence, or a HU1 terminator sequence, or a fragment or combination of fragments thereof, wherein: when the promoter sequence comprises the Mm U7 promoter sequence or a fragment thereof, the terminator sequence does not comprise the Mm U7 terminator sequence or a fragment thereof, when the promoter sequence comprises the Hs U7 promoter sequence or a fragment thereof, the terminator sequence does not comprise the Hs U7 terminator sequence or a fragment thereof, when the promoter sequence comprises the mu1a1 promoter sequence or a fragment thereof, the terminator sequence does not comprise the mu1a1 terminator sequence or a fragment thereof, and when the promoter sequence comprises the HU1 promoter sequence or a fragment thereof, the terminator sequence does not comprise the HU1 terminator sequence or a fragment thereof.

Described herein, in some embodiments, is a method of producing a ribonucleic acid (RNA). In some embodiments, the method comprises contacting a cell with an expression system. In some embodiments, the expression system is a virus. In some embodiments, the virus is an adeno-associated virus (AAV). In some embodiments, the expression system may include a regulatory sequence operably coupled to a transcribable region of a nucleic acid. In some embodiments, the regulatory sequence may include a proximal regulatory sequence element (PSE). In some embodiments, the regulatory sequence may include a distal regulatory sequence element (DSE). In some embodiments, the regulatory sequence may include a PSE and a DSE. In some embodiments, the PSE may include a mouse U7 snRNA (“Mm U7”) PSE. In some embodiments, the PSE may include a human U7 snRNA (“Hs U7”) PSE. In some embodiments, the PSE may include a mouse U1a1 (“mu1a1”) PSE. In some embodiments, the PSE may include a human U1-1 (“HU1” or “Hs U1-1”) PSE. In some embodiments, the DSE may include a Mm U7 DSE. In some embodiments, the DSE may include a Hs U7 DSE. In some embodiments, the DSE may include a mu1a1 DSE. In some embodiments, the DSE may include a HU1 DSE. In some embodiments, when the PSE comprises the Mm U7 PSE, the DSE does not comprise the Mm U7 DSE. In some embodiments, when the PSE comprises the Hs U7 PSE, the DSE does not comprise the Hs U7 DSE. In some embodiments, when the PSE comprises the mu1a1 PSE, the DSE does not comprise the mu1a1 DSE. In some embodiments, when the PSE comprises the HU1 PSE, the DSE does not comprise the HU1 DSE. In some embodiments, the regulatory sequence may include a promoter sequence. In some embodiments, the regulatory sequence may include a terminator sequence. In some embodiments, the regulatory sequence may include a promoter sequence and a terminator sequence.

Described herein, in some embodiments, is a method for producing a ribonucleic acid (RNA), comprised of contacting a cell with an expression system comprised of a regulatory sequence operably coupled to a transcribable region of a nucleic acid, the regulatory sequence comprised of a proximal regulatory sequence element (PSE) and a distal regulatory sequence element (DSE); wherein the PSE is comprised of a mouse U7 snRNA (“Mm U7”) PSE, a human U7 snRNA (“Hs U7”) PSE, a mouse U1a1 (“mu1a1”) PSE, or a human U1-1 (“HU1” or “Hs U1-1”) PSE; and wherein the PSE is comprised of a Mm U7 DSE, a Hs U7 DSE, a mu1a1 DSE, or a HU1 DSE, wherein: when the PSE comprises the Mm U7 PSE, the DSE does not comprise the Mm U7 DSE, when the PSE comprises the Hs U7 PSE, the DSE does not comprise the Hs U7 DSE, when the PSE comprises the mu1a1 PSE, the DSE does not comprise the mu1a1 DSE, and when the PSE comprises the HU1 PSE, the DSE does not comprise the HU1 DSE.

Described herein, in some embodiments, is a nucleic system. In some embodiments, the nucleic acid system may include a transcribable region. In some embodiments, the transcribable region may include an exonic splicing silencer (ESS) nucleic acid sequence. In some embodiments, the transcribable region may include an exonic splicing enhancer (ESE) nucleic acid sequence. In some embodiments, the transcribable region may include a target nucleic acid sequence. In some embodiments, the target nucleic acid sequence may be targeting a target ribonucleic acid (RNA). In some embodiments, the transcribable region may include a nucleic acid coding sequence (CDS).

In some embodiments, the nucleic acid expression system may produce a greater amount of RNA from the transcribable region than an expression system having a wild-type promoter sequence operably coupled to the transcribable region. In some embodiments, the nucleic acid expression system may produce a greater amount of RNA from the transcribable region than an expression system having a wild-type terminator sequence 3′ to the transcribable region. In some embodiments, the amount of RNA produced is at least 1% greater. In some embodiments, the amount of RNA produced is at least 2% greater. In some embodiments, the amount of RNA produced is at least 3% greater. In some embodiments, the amount of RNA produced is at least 4% greater. In some embodiments, the amount of RNA produced is at least 5% greater. In some embodiments, the amount of RNA produced is at least 10% greater. In some embodiments, the amount of RNA produced is at least 20% greater. In some embodiments, the amount of RNA produced is at least 30% greater. In some embodiments, the amount of RNA produced is at least 40% greater. In some embodiments, the amount of RNA produced is at least 50% greater. In some embodiments, the amount of RNA produced is at least 60% greater. In some embodiments, the amount of RNA produced is at least 70% greater. In some embodiments, the amount of RNA produced is at least 80% greater. In some embodiments, the amount of RNA produced is at least 90% greater. In some embodiments, the amount of RNA produced is at least 100% greater. In some embodiments, the amount of RNA produced is at least 150% greater. In some embodiments, the amount of RNA produced is at least 200% greater. In some embodiments, the amount of RNA produced is at least 250% greater. In some embodiments, the amount of RNA produced is at least 300% greater. In some embodiments, the amount of RNA produced is at least 350% greater. In some embodiments, the amount of RNA produced is at least 400% greater. In some embodiments, the amount of RNA produced is at least 450% greater. In some embodiments, the amount of RNA produced is at least 500% greater.

In some embodiments, the expression system may produce RNA from the transcribable region at a greater rate than an expression system having a wild-type promoter sequence operably coupled to the transcribable region. In some embodiments, the expression system may produce RNA from the transcribable region at a greater rate than an expression system having a wild-type terminator sequence 3′ to the transcribable region. In some embodiments, the greater rate of RNA produced is at least 1% greater. In some embodiments, the greater rate of RNA produced is at least 2% greater. In some embodiments, the greater rate of RNA produced is at least 3% greater. In some embodiments, the greater rate of RNA produced is at least 4% greater. In some embodiments, the greater rate of RNA produced is at least 5% greater. In some embodiments, the greater rate of RNA produced is at least 10% greater. In some embodiments, the greater rate of RNA produced is at least 20% greater. In some embodiments, the greater rate of RNA produced is at least 30% greater. In some embodiments, the greater rate of RNA produced is at least 40% greater. In some embodiments, the greater rate of RNA produced is at least 50% greater. In some embodiments, the greater rate of RNA produced is at least 60% greater. In some embodiments, the greater rate of RNA produced is at least 70% greater. In some embodiments, the greater rate of RNA produced is at least 80% greater. In some embodiments, the greater rate of RNA produced is at least 90% greater. In some embodiments, the greater rate of RNA produced is at least 100% greater. In some embodiments, the greater rate of RNA produced is at least 150% greater. In some embodiments, the greater rate of RNA produced is at least 200% greater. In some embodiments, the greater rate of RNA produced is at least 250% greater. In some embodiments, the greater rate of RNA produced is at least 300% greater. In some embodiments, the greater rate of RNA produced is at least 350% greater. In some embodiments, the greater rate of RNA produced is at least 400% greater. In some embodiments, the greater rate of RNA produced is at least 450% greater. In some embodiments, the greater rate of RNA produced is at least 500% greater.

### Compositions

Disclosed herein, in some embodiments, are compositions. The composition may be a pharmaceutical composition. The composition may include RNAs such as recombinant or modified U7 snRNA sequences described herein, or an expression construct. The composition may include an expression construct encoding a recombinant or modified U7 snRNA. The composition may include a viral vector.

Described herein, in some embodiments, the components of the system may be combined together within a single nucleic acid. In some embodiments, the components are separated among multiple nucleic acids.

In some embodiments, a pharmaceutical composition comprising the system and a pharmaceutically acceptable carrier is described. A composition may include a carrier such as a pharmaceutically acceptable carrier. Examples of carriers may include a solution such as water, a buffer, or saline, or a lipid composition.

The composition may include a delivery agent such as a virus, liposome, or nanoparticle. In some embodiments, the composition includes a delivery agent. In some embodiments, the delivery agent includes a virus. In some embodiments, the delivery agent includes a liposome. In some embodiments, the delivery agent includes a nanoparticle.

A virus may include the nucleic acid system. In some embodiments, the virus is a parvovirus. An example of a parvovirus may include a dependoparvovirus. An example of a dependoparvovirus may include an adeno-associated virus (AAV). In some embodiments, the virus may be an adeno-associated virus (AAV). In some embodiments, the AAV is a self-complementary AAV. In some embodiments, the AAV is a single-strand AAV. In some embodiments, the AAV may be serotype AAV1. In some embodiments, the AAV may be serotype AAV2. In some embodiments, the AAV may be serotype AAV4. In some embodiments, the AAV may be serotype AAV5. In some embodiments, the AAV may be serotype AAV6. In some embodiments, the AAV may be serotype AAV7. In some embodiments, the AAV may be serotype AAV8. In some embodiments, the AAV may be serotype AAV9.

In some embodiments, the, AAV may express one U7 cassette. Gene cassettes may include small mobile elements, consisting of a single gene and a recombination site, which may be integrated into larger elements called integrons. Several gene cassettes can be inserted into the same integrin forming a tandem array where cassettes can be expressed together. In some embodiments, the AAV may express an array of two U7 cassettes. In some embodiments, the AAV may express an array of three U7 cassettes. In some embodiments, the AAV may express an array of four U7 cassettes. In some embodiments, the AAV may express an array of five U7 cassettes. In some embodiments, the AAV may express at least one U7 cassette. In some embodiments, the AAV may express an array of at least two U7 cassettes. In some embodiments, the AAV may express an array of at least three U7 cassettes. In some embodiments, the AAV may express an array of at least four U7 cassettes. In some embodiments, the AAV may express an array of at least five U7 cassettes. In some embodiments, the AAV may express at most one U7 cassette. In some embodiments, the AAV may express an array of at most two U7 cassettes. In some embodiments, the AAV may express an array of at most three U7 cassettes. In some embodiments, the AAV may express an array of at most four U7 cassettes. In some embodiments, the AAV may express an array of at most five U7 cassettes.

Disclosed herein, in some embodiments, are viral particles, comprising a polynucleotide comprising one or more copies of a cassette comprising a promoter, a coding sequence, and a terminator sequence, wherein the coding sequence comprises a nucleic acid sequence having at least 80%, at least 85%, at least 90%, at least 95%, at least 98%, at least 99%, or 100% identity to the nucleic acid sequence of any one of SEQ ID NOs: 364-566. In some embodiments, the viral particle is an AAV viral particle. In some embodiments, the polynucleotide comprises 2, 3, or 4 copies of the cassette.

In some embodiments, a cell comprises the system (e.g. modified U7 snRNA). For example, the system may be delivered to a cell. The system may be delivered to the cell in vivo. The system may be delivered to the cell in vitro. The system may be administered to a subject, and thereby enter a cell of the subject. In some embodiments, the cell is a brain cell. In some embodiments, the cell is a neural cell. In some embodiments, the cell is a neuron.

In some embodiments, a tissue or biofluid of a subject comprises the system (e.g. modified U7 snRNA). For example, the system may be delivered to a tissue or biofluid. The system may be delivered to the tissue or biofluid in vivo. The system may be delivered to the tissue or biofluid in vitro. The system may be administered to a subject, and thereby enter a tissue or biofluid of the subject. In some embodiments, the tissue includes neural tissue. In some embodiments, the tissue includes brain tissue. In some embodiments, the tissue includes nerve tissue. In some embodiments, the biofluid includes blood. In some embodiments, the biofluid includes serum. In some embodiments, the biofluid includes serum.

### Methods of Use

Described herein, in some embodiments, are methods. The method may include administering a composition or system described herein. The method may include delivering a composition or system described herein in a cell. The method may include expressing a composition or system described herein in a cell. The cell may be in vivo (e.g. in a living body). The cell may be in vitro. The method may include a method of treatment. The method may include modifying splicing of a target nucleic acid such as an SCN1A RNA. The method may be performed on a subject, or on a cell such as a cell of a subject.

Described herein, in some embodiments, is a method comprising administering a pharmaceutical composition. In some embodiments, the pharmaceutical composition comprises a virus. The composition or virus may be modified. The composition or virus may be recombinant. In some embodiments, the pharmaceutical composition comprises a virus that is an adeno-associated virus (AAV). In some embodiments, the virus comprises a promoter, an exonic splicing silencer sequence, a U7 targeting sequence, a smOPT, a U7 3′ hairpin structure, and a 3′ terminal sequence. Described herein, in some embodiments, is a method comprised of administering a pharmaceutical composition or virus comprised of the nucleic acid system.

The method may be used to treat or modify splicing in a subject. The method may include administering a composition to a subject. The method may be used to treat a cell. The method may include administering a composition to a cell. The cell may be a brain cell. The cell may be a neural cell. The cell may be a neuron.

In some embodiments, the cell is in a subject. In some embodiments, the subject is an animal. In some embodiments, the subject is a vertebrate. In some embodiments, the subject is a mammal. In some embodiments, the subject is a human.

In some embodiments, the subject may have one mutated copy of a gene that encodes a target RNA per cell. In some embodiments, the subject may have two mutated copies of a gene that encodes a target RNA per cell. In some embodiments, the subject may have no mutated copies of a gene that encodes a target RNA per cell. A mutated copy of a gene that encodes a target RNA may lead to splicing that includes an alternatively spliced exon of the gene that encodes a target RNA. A mutated copy of a gene that encodes a target RNA may result in nonsense mediated decay (NMD) of a target mRNA.

In some embodiments, the subject may have one mutated copy of SCN1A DNA per cell. In some embodiments, the subject may have two mutated copies of SCN1A DNA per cell. In some embodiments, the subject may have no mutated copies of SCN1A DNA per cell. A mutated copy of SCN1A DNA may lead to splicing that includes an alternatively spliced exon of the SCN1A. A mutated copy of SCN1A DNA may result in nonsense mediated decay (NMD) of an SCN1A mRNA.

### Modifying Splicing

Described herein, in some embodiments, is a method for modifying splicing. The method may include modifying splicing in a subject. The method may include modifying splicing in a cell. In some embodiments, the method may include contacting a pre-mRNA with a system or composition herein. In some embodiments, the method may include contacting a pre-mRNA. The pre-mRNA may be a target RNA. In some embodiments, the pre-mRNA is or includes a target pre-mRNA. The method may include contacting a target pre-mRNA with a recombinant nucleic acid sequence that induces exclusion of an exon (e.g. an alternative exon) from a mature mRNA generated by the target pre-mRNA. Described herein, in some embodiments, is a method of modifying splicing comprised of contacting a target pre-mRNA with a recombinant nucleic acid sequence that induces exclusion of an exon such as an alternative exon from a mature mRNA generated by the target pre-mRNA.

In some embodiments, the pre-mRNA may encode an SCN1A protein. In some embodiments, the method may include contacting a pre-mRNA encoding an SCN1A protein with a recombinant nucleic acid sequence that induces exclusion of exon 20N from a mature mRNA generated by the pre-mRNA. Described herein, in some embodiments, is a method of modifying splicing comprised of contacting a pre-mRNA encoding an SCN1A protein with a recombinant nucleic acid sequence that induces exclusion of exon 20N from a mature mRNA generated by the pre-mRNA.

In some embodiments, the pre-mRNA is in a cell. In some embodiments, the pre-mRNA is in a neural cell. The cell may be a brain cell. The cell may be a neuron.

In some embodiments, the subject has epilepsy. In some embodiments, the subject is at risk of having epilepsy. Epilepsy is a chronic noncommunicable disease of the brain that affects around 50 million people worldwide. Epilepsy can be characterized by recurrent seizures, which are brief episodes of involuntary movement that may involve a part of the body (partial) or the entire body (generalized) and are sometimes accompanied by loss of consciousness and control of bowel or bladder function. In some embodiments, the subject may suffer from tonic-clonic seizures. In some embodiments, the subject may suffer from myoclonic seizures. In some embodiments, the subject may suffer from atypical absence seizures. In some embodiments, the subject may suffer from atonic seizures. In some embodiments, the subject may suffer from focal aware or impaired awareness seizures. In some embodiments, the subject may suffer from tonic seizures. In some embodiments, the subject may suffer from non-convulsive status epilepticus. In some embodiments, the subject may be at risk of having tonic-clonic seizures. In some embodiments, the subject is at risk of having myoclonic seizures. In some embodiments, the subject may be at risk of having atypical absence seizures. In some embodiments, the subject may be at risk of having atonic seizures. In some embodiments, the subject may be at risk of having focal aware or impaired awareness seizures. In some embodiments, the subject may be at risk of having tonic seizures. In some embodiments, the subject may be at risk of having non-convulsive status epilepticus seizures.

In some embodiments, the subject has a genetic disorder such as Dravet syndrome. In some embodiments, the subject is at risk of having a genetic disorder such as Dravet syndrome. In some embodiments, the subject has some level of developmental disability. In some embodiments, the subject has a crouched gait while walking. In some embodiments, the subject is at risk of having some level of developmental disability. In some embodiments, the subject is at risk of having a crouched gait while walking. In some embodiments, the subject has or is at risk of having a genetic disorder such as Dravet syndrome.

Described herein, in some embodiments, is a method of preventing, reducing or inhibiting nonsense-mediated decay (NMD). The NMD may be prevented. The NMD may be reduced. The NMD may be inhibited. The NMD may be prevented, reduced, or inhibited in a subject. The NMD may be prevented, reduced, or inhibited in a cell of a subject. The NMD may be prevented, reduced, or inhibited with regard to a target transcript (e.g. a SCN1A transcript). Some embodiments include preventing, reducing or inhibiting nonsense-mediated decay (NMD) by contacting a target pre-mRNA (e.g. an SCN1A pre-mRNA) with a composition or system described herein, such as a modified U7 snRNA. Some embodiments include preventing, reducing or inhibiting nonsense-mediated decay (NMD) by administering composition or system described herein to a subject. The prevention, reduction, or inhibition may be relative to a baseline or control.

Some embodiments include reducing or inhibiting NMD in cells (e.g. cells of a subject) by at least 10%. Some embodiments include reducing or inhibiting NMD in cells by at least 20%. Some embodiments include reducing or inhibiting NMD in cells by at least 30%. Some embodiments include reducing or inhibiting NMD in cells by at least 40%. Some embodiments include reducing or inhibiting NMD in cells by at least 50%. Some embodiments include reducing or inhibiting NMD in cells by at least 60%. Some embodiments include reducing or inhibiting NMD in cells by at least 70%. Some embodiments include reducing or inhibiting NMD in cells by at least 80%. Some embodiments include reducing or inhibiting NMD in cells by at least 90%. Some embodiments include reducing or inhibiting NMD in cells by less than 10%. Some embodiments include reducing or inhibiting NMD in cells by less than 20%. Some embodiments include reducing or inhibiting NMD in cells by less than 30%. Some embodiments include reducing or inhibiting NMD in cells by less than 40%. Some embodiments include reducing or inhibiting NMD in cells by less than 50%. Some embodiments include reducing or inhibiting NMD in cells by less than 60%. Some embodiments include reducing or inhibiting NMD in cells by less than 70%. Some embodiments include reducing or inhibiting NMD in cells by less than 80%. Some embodiments include reducing or inhibiting NMD in cells by less than 90%. Some embodiments include reducing or inhibiting NMD in cells by less than 95%. Some embodiments include reducing or inhibiting NMD in cells by less than 100%. Some embodiments include a range of any of the above percentages.

Described herein, in some embodiments, is a method comprised of administering to the subject a composition that silences or reduces said splicing, the composition comprising the system, virus, or composition described above. In some embodiments, the method comprises administering to the subject a composition that silences the splicing. In some embodiments, the method comprises administering to the subject a composition that reduces the splicing.

In some embodiments, the method may increase an amount of a productive isoform of a target RNA, or a productive isoform of a protein encoded by the target RNA. In some embodiments, the increase in the amount of a productive isoform of a target RNA or protein encoded by the target RNA is relative to a control. In some embodiments, the increase in the amount of a productive isoform of a target RNA or protein encoded by the target RNA is relative to a baseline amount of said productive isoform. In some embodiments, the method increases an amount of a productive isoform of a target RNA or protein encoded by the target RNA, relative to a control or baseline amount of said productive isoform.

In some embodiments, the method may increase an amount of a productive isoform of SCN1A mRNA. In some embodiments, the increase in the amount of a productive isoform of SCN1A mRNA is relative to a control. In some embodiments, the increase in the amount of a productive isoform of SCN1A mRNA is relative to a baseline amount of said productive isoform. In some embodiments, the method increases an amount of a productive isoform of SCN1A mRNA, relative to a control or baseline amount of said productive isoform.

In some embodiments, the method may increase an amount of a protein encoded by a target RNA. In some embodiments, the method may increase an amount of a protein encoded by a target RNA, relative to a control. In some embodiments, the method may increase an amount of a protein encoded by a target RNA, relative to a baseline amount of the protein encoded by a target RNA. In some embodiments, the method increases an amount of a protein encoded by a target RNA, relative to a control or baseline amount of the protein encoded by the target RNA. The improvement may be by at least 10%.

In some embodiments, the method may increase an amount of a NaV1.1 channel or protein (e.g. SCN1A protein). In some embodiments, the method may increase an amount of a NaV1.1 channel or protein (e.g. SCN1A protein), relative to a control. In some embodiments, the method may increase an amount of a NaV1.1 channel or protein (e.g. SCN1A protein), relative to a baseline amount of the NaV1.1 channel or protein. In some embodiments, the method increases an amount of a NaV1.1 channel or protein (e.g. SCN1A protein), relative to a control or baseline amount of the NaV1.1 channel or protein. The improvement may be by at least 10%.

In some embodiments, the method improves sodium transport. In some embodiments, the method improves sodium transport relative to a control. In some embodiments, the method improves sodium transport relative to a baseline amount. In some embodiments, the method improves sodium transport, relative to a control or baseline amount. The improvement may be by at least 10%.

Some embodiments relate to or include a method of reducing an amount of a non-productive target transcript (e.g. a SCN1A transcript). The non-productive target transcript may include a mature target mRNA that includes an exon such as an alternative exon (e.g. alternative exon 20N of an SCN1A mRNA). The amount of non-productive target transcript may be reduced in a cell or subject. The amount of non-productive target transcript may be reduced relative to a baseline measurement. The amount of non-productive target transcript may be reduced relative to a control measurement. The amount of non-productive target transcript may be reduced by at least 10%, by at least 20%, by at least 30%, by at least 40%, by at least 50%, by at least 60%, by at least 70%, by at least 80%, or by at least 90%. In some embodiments, the amount of non-productive target transcript is reduced by less than 10%, by less than 20%, by less than 30%, by less than 40%, by less than 50%, by less than 60%, by less than 70%, by less than 80%, by less than 90%, or by less than 100%. The amount of non-productive target transcript may be reduced by a range of percentages herein.

The amount of non-productive target transcript (e.g. a SCN1A transcript) may be measured in a sample of a subject. The sample may include a biofluid such as blood, serum, plasma, or cerebrospinal fluid, or may include a tissue sample such as neural or brain tissue. The baseline amount of non-productive target transcript may be measured in a baseline sample obtained before treatment of the subject (e.g. before administration of a composition herein to the subject). The amount of productive target transcript may be measured with an assay method such as a PCR assay. Examples of PCR assays may include quantitative PCR (qPCR) or reverse transcription quantitative PCR (RT-qPCR). The amount of non-productive target transcript may be normalized to a control, such as a measurement of a housekeeping mRNA.

Some embodiments relate to or include a method of increasing an amount of a productive target transcript (e.g. a SCN1A transcript). The productive target transcript may include a mature target mRNA that does not include an exon such as an alternative exon (e.g. alternative exon 20N of an SCN1A mRNA). The amount of productive target transcript may be increased in a cell or subject. The amount of productive target transcript may be increased relative to a baseline measurement. The amount of productive target transcript may be increased relative to a control measurement. The amount of productive target transcript may be increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 225%, or by at least 250%. In some embodiments, the amount of productive target transcript is increased by less than 10%, by less than 20%, by less than 30%, by less than 40%, by less than 50%, by less than 60%, by less than 70%, by less than 80%, by less than 90%, by less than 100%, by less than 125%, by less than 150%, by less than 175%, by less than 200%, by less than 225%, by less than 250%, by less than 275%, by less than 300%, by less than 350%, or by less than 400%. The amount of productive target transcript may be increased by a range of percentages herein.

The amount of productive target transcript (e.g. a SCN1A transcript) may be measured in a sample of a subject. The sample may include a biofluid such as blood, serum, plasma, or cerebrospinal fluid, or may include a tissue sample such as neural or brain tissue. The baseline amount of productive target transcript may be measured in a baseline sample obtained before treatment of the subject. The amount of productive target transcript may be measured with an assay method such as a PCR assay. The amount of productive target transcript may be normalized to a control, such as a measurement of a housekeeping mRNA.

Some embodiments relate to or include a method of increasing an amount of a protein encoded by a target transcript (e.g. a NaV1.1 protein encoded by a SCN1A transcript). The amount of the protein encoded by the target transcript may be increased in a cell or subject. The amount of the protein encoded by the target transcript may be increased relative to a baseline measurement. The amount of the protein encoded by the target transcript may be increased relative to a control measurement. The amount of the protein encoded by the target transcript may be increased by at least 10%, by at least 20%, by at least 30%, by at least 40%, by at least 50%, by at least 60%, by at least 70%, by at least 80%, by at least 90%, by at least 100%, by at least 125%, by at least 150%, or by at least 160%. In some embodiments, the amount of the protein encoded by the target transcript is increased by less than 10%, by less than 20%, by less than 30%, by less than 40%, by less than 50%, by less than 60%, by less than 70%, by less than 80%, by less than 90%, by less than 100%, by less than 125%, by less than 150%, by less than 160%, by less than 175%, or by less than 200%. The amount of the protein encoded by the target transcript may be increased by a range of percentages herein.

The amount of a protein encoded by a target transcript (e.g. a NaV1.1 protein encoded by a SCN1A transcript) may be measured in a sample of a subject. The sample may include a biofluid such as blood, serum, plasma, or cerebrospinal fluid, or may include a tissue sample such as neural or brain tissue. The baseline amount of the protein encoded by the target transcript may be measured in a baseline sample obtained before treatment of the subject. The amount of the protein encoded by the target transcript may be measured with an assay method such as an immunoblot. The amount of the protein encoded by the target transcript may be normalized to a control, such as a measurement of total protein or of a housekeeping protein.

### Treating a Disorder

Described herein, in some embodiments, is a method for treating a disorder in a subject in need thereof. In some embodiments, the subject has the disorder. In some embodiments, the subject is identified as having the disorder. In some embodiments, the subject is at risk of having the disorder. In some embodiments, the subject is identified as at risk of having the disorder. The treatment may have a prophylactic effect. The method may include any aspect of another method described herein, such as modifying splicing, reducing an amount of a non-productive target transcript (e.g. a SCN1A transcript) relative to a baseline or control measurement, increasing an amount of a non-productive target transcript relative to a baseline or control measurement, or increasing an amount of protein encoded by the target transcript relative to a baseline or control measurement.

The disorder may be or include a genetic disorder. In some embodiments, the subject has or is at risk of having a genetic disorder. The disorder may be or include a neurodevelopmental disorder. In some embodiments, the subject has or is at risk of having a neurodevelopmental disorder. In some embodiments, the neurodevelopmental disorder comprises an intellectual disability or epilepsy. The disorder may include an intellectual disability. The disorder may include epilepsy. The disorder may include Dravet syndrome.

Described herein, in some embodiments, is a method for preventing a disorder in a subject in need thereof. Described herein, in some embodiments, is a method for treating a disorder in a subject in need thereof. In some embodiments, the method comprises administering a therapeutically effective amount of a synthetic composition that may silence splicing of an alternatively spliced region of a ribonucleic acid (RNA). In some embodiments, the method comprises administering a therapeutically effective amount of a synthetic composition that may reduce splicing of an alternatively spliced region of RNA. In some embodiments, the method comprises administering a therapeutically effective amount of a synthetic composition that may silence splicing of an alternatively spliced region of a target RNA (e.g. an RNA encoding SCN1A). In some embodiments, the method comprises administering a therapeutically effective amount of a synthetic composition that may reduce splicing of an alternatively spliced region of a target RNA (e.g. an RNA encoding SCN1A). In some embodiments, the alternatively spliced region may include an exon. In some embodiments, the alternatively spliced region may include a NMD exon (e.g. 20N of an SCN1A RNA) of the RNA.

Described herein, in some embodiments, is a method of treating or preventing a neurodevelopmental disorder in a subject in need thereof, comprising administering a therapeutically effective amount of a recombinant U7 small nuclear RNA (snRNA) composition that silences or reduces splicing of an alternatively spliced region of a target RNA (e.g. a SCN1A SCN1A). In some embodiments, the neurodevelopmental disorder comprises an intellectual disability or epilepsy.

In some embodiments, the administration may increase the amount of a productive isoform of a target RNA (e.g. an SCN1A RNA) or a protein encoded by the target RNA in the subject. In some embodiments, the administration may increase the amount of a productive isoform of a target RNA in the subject. In some embodiments, the administration may increase the amount of a productive isoform of a target RNA in the subject relative to a control. In some embodiments, the administration may increase the amount of a productive isoform of target RNA in the subject relative to a baseline amount of said productive isoform. In some embodiments, the method increases an amount of a productive isoform of target RNA in the subject, relative to a control or baseline amount of said productive isoform.

In some embodiments, the administration may increase the amount of a protein encoded by a target RNA (e.g. a NaV1.1 channel or protein encoded by an SCN1A RNA) in the subject. In some embodiments, the administration may increase the amount of a protein encoded by a target RNA in the subject relative to a control. In some embodiments, the administration may increase the amount of a protein encoded by a target RNA in the subject relative to a baseline amount of the protein encoded by the target RNA. In some embodiments, the administration increases an amount of a protein encoded by a target RNA in the subject, relative to a control or baseline amount of the protein encoded by the target RNA.

In some embodiments, the administration improves an aspect in the subject. In some embodiments, the administration improves a symptom of the disorder. In some embodiments, the administration improves the symptom relative to a control. In some embodiments, the administration improves the symptom relative to a baseline amount of the symptom. In some embodiments, the administration improves the symptom, relative to a control or baseline amount.

In some embodiments, the administration improves sodium transport in the subject. In some embodiments, the administration may improve sodium transport in the subject relative to a control. In some embodiments, the administration may improve sodium transport in the subject relative to a baseline amount of sodium transport. In some embodiments, the administration improves sodium transport in the subject, relative to a control or baseline amount.

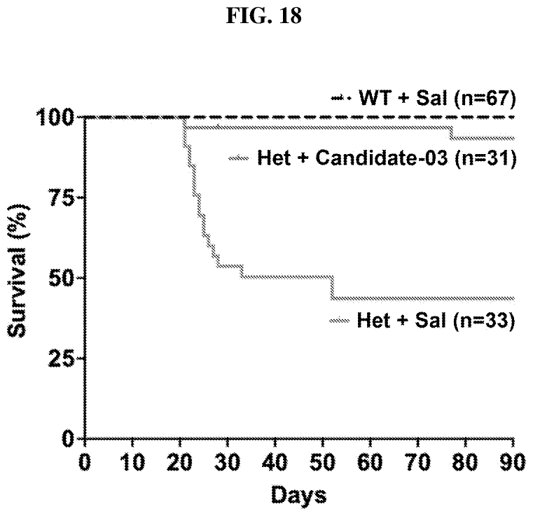

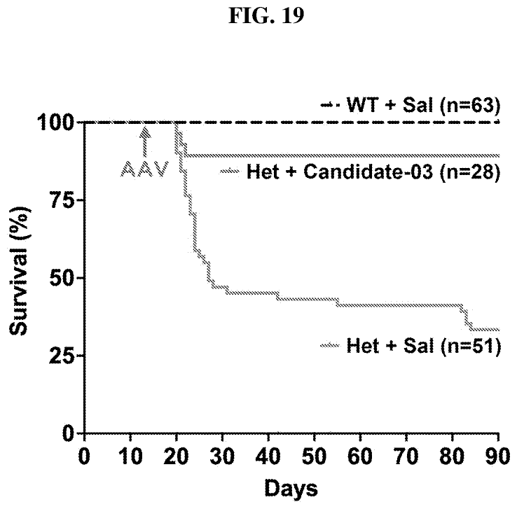

The method or administration may improve a survival chance of a subject, for example as shown in FIG. 18 or FIG. 19. The method may reduce a number of seizures, seizure frequency, or seizure duration as provided herein.

### Treating Epilepsy

Described herein, in some embodiments, is a method for treating epilepsy in a subject in need thereof. Described herein, in some embodiments, is a method for preventing epilepsy in a subject in need thereof. In some embodiments, the method comprises administering a therapeutically effective amount of a synthetic composition that may silence splicing of an alternatively spliced region of a ribonucleic acid (RNA). In some embodiments, the method comprises administering a therapeutically effective amount of a synthetic composition that may reduce splicing of an alternatively spliced region of RNA. In some embodiments, the method comprises administering a therapeutically effective amount of a synthetic composition that may silence splicing of an alternatively spliced region of RNA encoding SCN1A. In some embodiments, the method comprises administering a therapeutically effective amount of a synthetic composition that may reduce splicing of an alternatively spliced region of RNA encoding sodium channel protein type 1 subunit alpha (SCN1A). Described herein, in some embodiments, is a method of treating or preventing epilepsy in a subject in need thereof, comprised of administering a therapeutically effective amount of a synthetic composition that silences or reduces splicing of an alternatively spliced region of a ribonucleic acid (RNA) encoding sodium channel protein type 1 subunit alpha (SCN1A). In some embodiments, the alternatively spliced region may include an exon. In some embodiments, the alternatively spliced region may include exon 20N of the RNA.

In some embodiments, the subject is in need of treatment for an epileptic disorder. In some embodiments, the subject is in need of treatment for a genetic disorder such as Dravet syndrome.

In some embodiments, the administration may increase the amount of a productive isoform of SCN1A in the subject. In some embodiments, the administration may increase the amount of a productive isoform of SCN1A in the subject relative to a control. In some embodiments, the administration may increase the amount of a productive isoform of SCN1A in the subject relative to a baseline amount of said productive isoform. In some embodiments, the method increases an amount of a productive isoform of SCN1A in the subject, relative to a control or baseline amount of said productive isoform.

In some embodiments, the administration may increase the amount of a NaV1.1 channel or protein (e.g. SCN1A protein) in the subject. In some embodiments, the administration may increase the amount of a NaV1.1 channel or protein (e.g. SCN1A protein) in the subject relative to a control. In some embodiments, the administration may increase the amount of a NaV1.1 channel or protein (e.g. SCN1A protein) in the subject relative to a baseline amount of the NaV1.1 channel or protein. In some embodiments, the administration increases an amount of a NaV1.1 channel or protein (e.g. SCN1A protein) in the subject, relative to a control or baseline amount of the NaV1.1 channel or protein.

In some embodiments, the administration improves an aspect in the subject. In some embodiments, the administration improves a symptom of the disorder. In some embodiments, the administration improves the symptom relative to a control. In some embodiments, the administration improves the symptom relative to a baseline amount of the symptom. In some embodiments, the administration improves the symptom, relative to a control or baseline amount.

In some embodiments, the administration may improve sodium transport in the subject. In some embodiments, the administration may improve sodium transport in the subject relative to a control. In some embodiments, the administration may improve sodium transport in the subject relative to a baseline amount of sodium transport. In some embodiments, the administration improves sodium transport in the subject, relative to a control or baseline amount.

In some embodiments, the administration prevents the subject from having seizures. In some embodiments, the administration reduces the amount of the seizures of the subject relative to a baseline amount. In some embodiments, the administration reduces the severity of the seizures of the subject relative to a baseline severity. In some embodiments, the administration prevents the subject from having seizures, or reduces an amount or severity of the seizures of the subject relative to a baseline amount or severity.

### Treating a Genetic Disorder

Described herein, in some embodiments, is a method for treating a genetic disorder such as Dravet syndrome in a subject in need thereof. In some embodiments, the method may prevent a manifestation of a genetic disorder such as Dravet syndrome in a subject in need thereof. In some embodiments, the method comprises administering a therapeutically effective amount of a synthetic composition that may silence splicing of an alternatively spliced region of a target RNA (e.g. an RNA encoding SCN1A). In some embodiments, the method comprises administering a therapeutically effective amount of a synthetic composition that may reduce splicing of an alternatively spliced region of a target RNA (e.g. an RNA encoding SCN1A). Described herein, in some embodiments, is a method of treating a genetic disorder such as Dravet syndrome in a subject in need thereof, comprised of administering a therapeutically effective amount of a synthetic composition that silences or reduces splicing of an alternatively spliced region of a target RNA (e.g. an RNA encoding SCN1A). In some embodiments, the alternatively spliced region comprises a nonsense-mediated decay (NMD) exon of the target RNA (e.g. exon 20N of an SCN1A RNA), or an equivalent region depending on the subject's species.

In some embodiments, the administration may increase an amount of a productive isoform of a target RNA (e.g. a SCN1A RNA) in the subject. In some embodiments, the administration may increase an amount of a productive isoform of a target RNA in the subject relative to a control. In some embodiments, the administration may increase an amount of a productive isoform of a target RNA in the subject relative to a baseline amount of said productive isoform. In some embodiments, the administration increases an amount of a productive isoform of a target RNA in the subject, relative to a control or baseline amount of said productive isoform.

In some embodiments, the administration may increase an amount of a protein encoded by the target RNA (e.g. a NaV1.1 protein encoded by an SCN1A RNA) in the subject. In some embodiments, the administration may increase an amount of a protein encoded by the target RNA in the subject relative to a control amount of the protein encoded by the target RNA. In some embodiments, the administration may increase an amount of a protein encoded by the target RNA in the subject relative to a baseline amount of the protein encoded by the target RNA. In some embodiments, the administration increases an amount of protein encoded by the target RNA in the subject, relative to a control or baseline amount of the protein encoded by the target RNA.

In some embodiments, the administration improves an aspect in the subject. In some embodiments, the administration improves a symptom of the disorder. In some embodiments, the administration improves the symptom relative to a control. In some embodiments, the administration improves the symptom relative to a baseline amount of the symptom. In some embodiments, the administration improves the symptom, relative to a control or baseline amount.

In some embodiments, the administration may improve sodium transport in the subject. In some embodiments, the administration may improve sodium transport in the subject relative to a control. In some embodiments, the administration may improve sodium transport in the subject relative to a baseline amount. In some embodiments, the administration improves sodium transport in the subject, relative to a control or baseline amount.

In some embodiments, the administration may prevent the subject from having seizures. Characteristics of seizures vary and depend on where in the brain the disturbance first starts, and how far it spreads. Temporary symptoms occur, such as loss of awareness or consciousness, and disturbances of movement, sensation (including vision, hearing and taste), mood, or other cognitive functions. Subjects with epilepsy tend to have more physical problems (such as fractures and bruising from injuries related to seizures) as well as higher rates of psychological conditions, including anxiety and depression. The risk of premature death in people with epilepsy is up to three times higher than in the general population. In some embodiments, the administration may reduce the amount of the seizures of the subject relative to a baseline amount. In some embodiments, the administration may reduce the severity of the seizures of the subject relative to a baseline severity. In some embodiments, the administration prevents the subject from having seizures, or reduces an amount or severity of the seizures of the subject relative to a baseline amount or severity.

A genetic disorder such as Dravet syndrome may include epilepsy. The epilepsy of a subject having the genetic disorder may be treated or improved. The genetic disorder may include seizures. The seizures of a subject having the genetic disorder may be treated or improved. For example, a seizure number, frequency, or duration may be reduced in a subject with the genetic disorder upon treatment.

### Administration

Disclosed herein, in some embodiments, are methods that include administering a composition. The administration may be to a subject. The administration may be to a human subject. The administered composition may include an engineered snRNA. The administered composition may include an expression vector. The administered composition may include an expression vector encoding an engineered snRNA. The administered composition may include a pharmaceutical composition. The administered composition may include a virus. The administered composition may include a virus comprising an expression vector. The administered composition may include a liposome. The administered composition may include a nanoparticle.

The administration may be by a route of administration. The administration be systemic. The administration be intravenous. The administration may include an injection.

The administration may be at a site of administration. The administration may be intracerebroventricular (ICV). The administration may be retroorbital.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 15% of that number. The term “about” a range refers to that range minus 15% of its lowest value and plus 15% of its greatest value.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

Some embodiments relate to a small nuclear RNA (snRNA). An snRNA may include a class of small RNA molecules found within splicing speckles or Cajal bodies of a eukaryotic cell nucleus. A length of an unmodified snRNA may average about 150 nucleotides. An snRNA may be transcribed by RNA polymerase II or RNA polymerase III. An snRNA may function in the processing of pre-messenger RNA (hnRNA) in the nucleus. Any of these aspects may be modified or missing in a modified or engineered snRNA. An snRNA may associate with a protein or set of proteins to form a complex. The complex may be referred to as a small nuclear ribonucleoprotein (snRNP). Some examples of human snRNA components of such complexes may include: U1 spliceosomal RNA, U2 spliceosomal RNA, U4 spliceosomal RNA, U5 spliceosomal RNA, or U6 spliceosomal RNA. An snRNA may have a high uridine content.

Some embodiments relate to a U7 snRNA. A U7 snRNA may include an RNA molecule and a component of a small nuclear ribonucleoprotein complex (U7 snRNP). The U7 snRNA may affect histone pre-mRNA processing. The U7 snRNA may be modified or engineered. In some embodiments, the modified or engineered U7 snRNA does not affect histone pre-mRNA processing, or has little effect on such. In some embodiments, a U7 snRNA has a 5′ end that binds an HDE (histone downstream element), a conserved purine-rich region, located 15 nucleotides downstream a histone mRNA cleavage site. Any of these aspects may be modified or missing in a modified or engineered U7 snRNA. Binding of an HDE region by a U7 snRNA, through complementary base-pairing, may affect recruitment of cleavage factors during histone pre-mRNA processing.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

A percent sequence identity may be determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the % sequence identity. A sequence identity may include a sequence identity to a reverse complement. In determining a sequence identity, thymine (T) and uracil (U) may be interchangeable. T and U may be interchangeable when describing an oligonucleotide. In some embodiments, Ts and Us are interchangeable depending on whether the oligonucleotide is an RNA or DNA, where RNA includes U and DNA includes T.

A discrepancy between the written description and a sequence listing submitted herein may be resolved in favor of the written description.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

The following examples are included for illustrative purposes only and are not intended to limit the scope of the invention. The data in these examples are indicative of improvements in the compositions and methods described herein over existing compositions and methods.

### Example 1: Screen to Identify U7-Based Splice Modulation Candidates of SCN1A

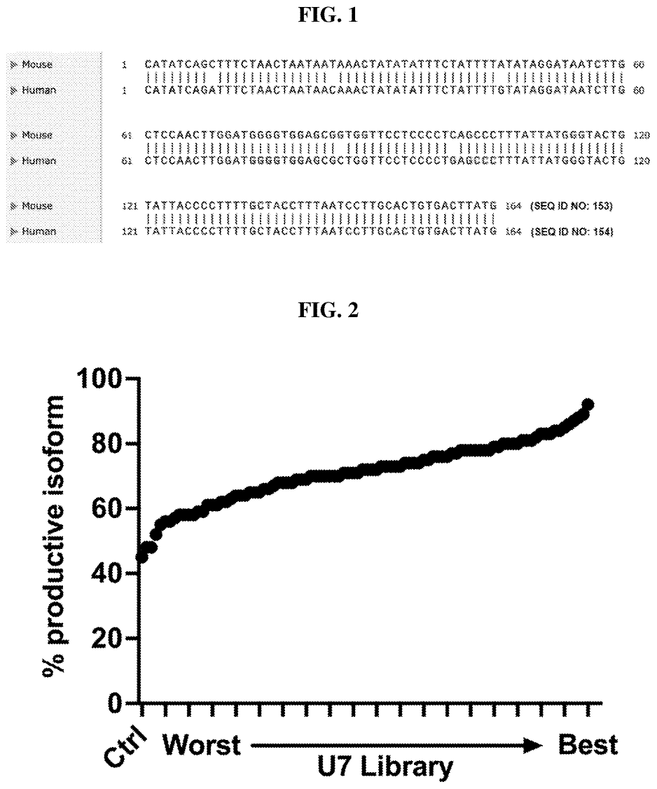

The alternatively spliced NMD exon of SEQ ID NO: 2 present in humans (exon 20N) is 96% conserved in mice (62/64 identity) (mouse exon 21N, SEQ ID NO: 1). The region encompassing the NMD exon, excluding 50 bp upstream and downstream, has similar identity (159/164 identity) (FIG. 1) A screen was performed in mouse neuroblast cells (Neuro-2a cell line) with a plasmid library encoding U7 expression cassettes (which included a promoter, ESS, antisense sequence, smOPT, hairpin, and 3′ terminator) designed to target the entire NMD exon and flanking regions. The library consisted of 32 unique U7 targeting sequences of 20, 24, 26, or 30 nucleotides in length. Each targeting sequence was paired with each of three unique exonic splicing silencer (ESS) sequences (Tables 2A-2B3). Neuro-2a cells were then transfected with plasmid DNA and suppression of NMD exon inclusion was analyzed via end point RT-PCR. This method identified 19 U7 targeting sequence/ESS combinations capable of increasing SCN1A isoform expression by 50% or greater (FIG. 2, Table 2C).

TABLE 2A

| Construct | | ESS SEQ | Mouse U7 Targeting | SEQ ID |
|---|---|---|---|---|
| Name | ESS Sequence (5′ to 3′) | ID NO: | Sequence (5′ to 3′) | NO: |
| Scramble- | ATGATAGGGACTTAGGGT | 11 | GCACCCCATCCCCCACTACA | 14 |
| 01 | GA | | | |
| N5-01 | ATGATAGGGACTTAGGGT | 11 | AACCACCGCTCCACCCCATC | 18 |
| | GA | | | |
| N5-02 | ATGATAGGGACTTAGGGT | 11 | CAAGTTGGAGCAAGATTATC | 19 |
| | GA | | | |
| N5-03 | ATGATAGGGACTTAGGGT | 11 | CTATATAAAATAGAAATATA | 20 |
| | GA | | | |
| N5-04 | ATGATAGGGACTTAGGGT | 11 | TAGTTTATTATTAGTTAGAA | 21 |
| | GA | | | |
| N5-05 | ATGATAGGGACTTAGGGT | 11 | GAGGAACCACCGCTCCACCCC | 22 |
| | GA | | ATC | |
| N5-06 | ATGATAGGGACTTAGGGT | 11 | CATCCAAGTTGGAGCAAGATT | 23 |
| | GA | | ATC | |
| N5-07 | ATGATAGGGACTTAGGGT | 11 | TATCCTATATAAAATAGAAAT | 24 |
| | GA | | ATA | |
| N5-08 | ATGATAGGGACTTAGGGT | 11 | TATATAGTTTATTATTAGTTAG | 25 |
| | GA | | AA | |
| N5-09 | ATGATAGGGACTTAGGGT | 11 | GGGAGGAACCACCGCTCCACC | 26 |
| | GA | | CCATC | |
| N5-10 | ATGATAGGGACTTAGGGT | 11 | CCCATCCAAGTTGGAGCAAGA | 27 |
| | GA | | TTATC | |
| N5-11 | ATGATAGGGACTTAGGGT | 11 | ATTATCCTATATAAAATAGAA | 28 |
| | GA | | ATATA | |
| N5-12 | ATGATAGGGACTTAGGGT | 11 | AATATATAGTTTATTATTAGTT | 29 |
| | GA | | AGAA | |
| N5-13 | ATGATAGGGACTTAGGGT | 11 | TGAGGGGAGGAACCACCGCTC | 30 |
| | GA | | CACCCCATC | |
| N5-14 | ATGATAGGGACTTAGGGT | 11 | CCACCCCATCCAAGTTGGAGC | 31 |
| | GA | | AAGATTATC | |
| N5-15 | ATGATAGGGACTTAGGGT | 11 | CAAGATTATCCTATATAAAAT | 32 |
| | GA | | AGAAATATA | |
| N5-16 | ATGATAGGGACTTAGGGT | 11 | TAGAAATATATAGTTTATTATT | 33 |
| | GA | | AGTTAGAA | |
| N3-01 | ATGATAGGGACTTAGGGT | 11 | AGTGCAAGGATTAAAGGTAG | 34 |
| | GA | | | |
| N3-02 | ATGATAGGGACTTAGGGT | 11 | CAAAAGGGGTAATACAGTAC | 35 |
| | GA | | | |
| N3-03 | ATGATAGGGACTTAGGGT | 11 | CCATAATAAAGGGCTGAGGG | 36 |
| | GA | | | |
| N3-04 | ATGATAGGGACTTAGGGT | 11 | GAGGAACCACCGCTCCACCC | 37 |
| | GA | | | |
| N3-05 | ATGATAGGGACTTAGGGT | 11 | TCACAGTGCAAGGATTAAAGG | 38 |
| | GA | | TAG | |
| N3-06 | ATGATAGGGACTTAGGGT | 11 | GTAGCAAAAGGGGTAATACA | 39 |
| | GA | | GTAC | |
| N3-07 | ATGATAGGGACTTAGGGT | 11 | GTACCCATAATAAAGGGCTGA | 40 |
| | GA | | GGG | |
| N3-08 | ATGATAGGGACTTAGGGT | 11 | AGGGGAGGAACCACCGCTCCA | 41 |
| | GA | | CCC | |
| N3-09 | ATGATAGGGACTTAGGGT | 11 | AGTCACAGTGCAAGGATTAAA | 42 |
| | GA | | GGTAG | |
| N3-10 | ATGATAGGGACTTAGGGT | 11 | AGGTAGCAAAAGGGGTAATA | 43 |
| | GA | | CAGTAC | |
| N3-11 | ATGATAGGGACTTAGGGT | 11 | CAGTACCCATAATAAAGGGCT | 44 |
| | GA | | GAGGG | |
| N3-12 | ATGATAGGGACTTAGGGT | 11 | TGAGGGGAGGAACCACCGCTC | 45 |
| | GA | | CACCC | |
| N3-13 | ATGATAGGGACTTAGGGT | 11 | CATAAGTCACAGTGCAAGGAT | 46 |
| | GA | | TAAAGGTAG | |
| N3-14 | ATGATAGGGACTTAGGGT | 11 | TTAAAGGTAGCAAAAGGGGTA | 47 |
| | GA | | ATACAGTAC | |
| N3-15 | ATGATAGGGACTTAGGGT | 11 | AATACAGTACCCATAATAAAG | 48 |
| | GA | | GGCTGAGGG | |
| N3-16 | ATGATAGGGACTTAGGGT | 11 | GGGCTGAGGGGAGGAACCAC | 49 |
| | GA | | CGCTCCACCC | |
| N5- | TTTGTTCCGTGGGTGGTTT | 12 | AACCACCGCTCCACCCCATC | 18 |
| ESSalt1-01 | A | | | |
| N5- | TTTGTTCCGTGGGTGGTTT | 12 | CAAGTTGGAGCAAGATTATC | 19 |
| ESSalt1-02 | A | | | |
| N5- | TTTGTTCCGTGGGTGGTTT | 12 | CTATATAAAATAGAAATATA | 20 |
| ESSalt1-03 | A | | | |
| N5- | TTTGTTCCGTGGGTGGTTT | 12 | TAGTTTATTATTAGTTAGAA | 21 |
| ESSalt1-04 | A | | | |
| N5- | TTTGTTCCGTGGGTGGTTT | 12 | GAGGAACCACCGCTCCACCCC | 22 |
| ESSalt1-05 | A | | ATC | |
| N5- | TTTGTTCCGTGGGTGGTTT | 12 | CATCCAAGTTGGAGCAAGATT | 23 |
| ESSalt1-06 | A | | ATC | |
| N5- | TTTGTTCCGTGGGTGGTTT | 12 | TATCCTATATAAAATAGAAAT | 24 |
| ESSalt1-07 | A | | ATA | |
| N5- | TTTGTTCCGTGGGTGGTTT | 12 | TATATAGTTTATTATTAGTTAG | 25 |
| ESSalt1-08 | A | | AA | |
| N5- | TTTGTTCCGTGGGTGGTTT | 12 | GGGAGGAACCACCGCTCCACC | 26 |
| ESSalt1-09 | A | | CCATC | |
| Scramble- | TTTGTTCCGTGGGTGGTTT | 12 | GGAGAATCACCTTATAGGAT | 15 |
| 02 | A | | | |
| N5- | TTTGTTCCGTGGGTGGTTT | 12 | CCCATCCAAGTTGGAGCAAGA | 27 |
| ESSalt1-10 | A | | TTATC | |
| N5- | TTTGTTCCGTGGGTGGTTT | 12 | ATTATCCTATATAAAATAGAA | 28 |
| ESSalt1-11 | A | | ATATA | |
| N5- | TTTGTTCCGTGGGTGGTTT | 12 | AATATATAGTTTATTATTAGTT | 29 |
| ESSalt1-12 | A | | AGAA | |
| N5- | TTTGTTCCGTGGGTGGTTT | 12 | TGAGGGGAGGAACCACCGCTC | 30 |
| ESSalt1-13 | A | | CACCCCATC | |
| N5- | TTTGTTCCGTGGGTGGTTT | 12 | CCACCCCATCCAAGTTGGAGC | 31 |
| ESSalt1-14 | A | | AAGATTATC | |
| N5- | TTTGTTCCGTGGGTGGTTT | 12 | CAAGATTATCCTATATAAAAT | 32 |
| ESSalt1-15 | A | | AGAAATATA | |
| N5 | TTTGTTCCGTGGGTGGTTT | 12 | TAGAAATATATAGTTTATTATT | 33 |
| ESSalt1-16 | A | | AGTTAGAA | |
| N3- | TTTGTTCCGTGGGTGGTTT | 12 | AGTGCAAGGATTAAAGGTAG | 34 |
| ESSalt1-01 | A | | | |
| N3- | TTTGTTCCGTGGGTGGTTT | 12 | CAAAAGGGGTAATACAGTAC | 35 |
| ESSalt1-02 | A | | | |
| N3- | TTTGTTCCGTGGGTGGTTT | 12 | CCATAATAAAGGGCTGAGGG | 36 |
| ESSalt1-03 | A | | | |
| N3- | TTTGTTCCGTGGGTGGTTT | 12 | GAGGAACCACCGCTCCACCC | 37 |
| ESSalt1-04 | A | | | |
| Scramble- | TTTGTTCCGTGGGTGGTTT | 12 | GAATAATAACATAATAATAT | 16 |
| 03 | A | | | |
| N3- | TTTGTTCCGTGGGTGGTTT | 12 | TCACAGTGCAAGGATTAAAGG | 38 |
| ESSalt1-05 | A | | TAG | |
| N3 | TTTGTTCCGTGGGTGGTTT | 12 | GTAGCAAAAGGGGTAATACA | 39 |
| ESSalt1-06 | A | | GTAC | |
| N3- | TTTGTTCCGTGGGTGGTTT | 12 | GTACCCATAATAAAGGGCTGA | 40 |
| ESSalt1-07 | A | | GGG | |
| N3 | TTTGTTCCGTGGGTGGTTT | 12 | AGGGGAGGAACCACCGCTCCA | 41 |
| ESSalt1-08 | A | | CCC | |
| N3- | TTTGTTCCGTGGGTGGTTT | 12 | AGTCACAGTGCAAGGATTAAA | 42 |
| ESSalt1-09 | A | | GGTAG | |
| N3- | TTTGTTCCGTGGGTGGTTT | 12 | AGGTAGCAAAAGGGGTAATA | 43 |
| ESSalt1-10 | A | | CAGTAC | |
| N3- | TTTGTTCCGTGGGTGGTTT | 12 | CAGTACCCATAATAAAGGGCT | 44 |
| ESSalt1-11 | A | | GAGGG | |
| N3- | TTTGTTCCGTGGGTGGTTT | 12 | TGAGGGGAGGAACCACCGCTC | 45 |
| ESSalt1-12 | A | | CACCC | |
| N3- | TTTGTTCCGTGGGTGGTTT | 12 | CATAAGTCACAGTGCAAGGAT | 46 |
| ESSalt1-13 | A | | TAAAGGTAG | |
| N3- | TTTGTTCCGTGGGTGGTTT | 12 | TTAAAGGTAGCAAAAGGGGTA | 47 |
| ESSalt1-14 | A | | ATACAGTAC | |
| N3- | TTTGTTCCGTGGGTGGTTT | 12 | AATACAGTACCCATAATAAAG | 48 |
| ESSalt1-15 | A | | GGCTGAGGG | |
| N3- | TTTGTTCCGTGGGTGGTTT | 12 | GGGCTGAGGGGAGGAACCAC | 49 |
| ESSalt1-16 | A | | CGCTCCACCC | |
| N5- | TGGGGGGAGGTAGGTAGG | 13 | AACCACCGCTCCACCCCATC | 18 |
| ESSalt2-01 | TA | | | |
| N5- | TGGGGGGAGGTAGGTAGG | 13 | CAAGTTGGAGCAAGATTATC | 19 |
| ESSalt2-02 | TA | | | |
| N5- | TGGGGGGAGGTAGGTAGG | 13 | CTATATAAAATAGAAATATA | 20 |
| ESSalt2-03 | TA | | | |
| N5- | TGGGGGGAGGTAGGTAGG | 13 | TAGTTTATTATTAGTTAGAA | 21 |
| ESSalt2-04 | TA | | | |
| N5- | TGGGGGGAGGTAGGTAGG | 13 | GAGGAACCACCGCTCCACCCC | 22 |
| ESSalt2-05 | TA | | ATC | |
| N5- | TGGGGGGAGGTAGGTAGG | 13 | CATCCAAGTTGGAGCAAGATT | 23 |
| ESSalt2-06 | TA | | ATC | |
| N5- | TGGGGGGAGGTAGGTAGG | 13 | TATCCTATATAAAATAGAAAT | 24 |
| ESSalt2-07 | TA | | ATA | |
| N5- | TGGGGGGAGGTAGGTAGG | 13 | TATATAGTTTATTATTAGTTAG | 25 |
| ESSalt2-08 | TA | | AA | |
| N5- | TGGGGGGAGGTAGGTAGG | 13 | GGGAGGAACCACCGCTCCACC | 26 |
| ESSalt2-09 | TA | | CCATC | |
| N5- | TGGGGGGAGGTAGGTAGG | 13 | CCCATCCAAGTTGGAGCAAGA | 27 |
| ESSalt2-10 | TA | | TTATC | |
| N5- | TGGGGGGAGGTAGGTAGG | 13 | ATTATCCTATATAAAATAGAA | 28 |
| ESSalt2-11 | TA | | ATATA | |
| N5- | TGGGGGGAGGTAGGTAGG | 13 | TGAGGGGAGGAACCACCGCTC | 30 |
| ESSalt2-13 | TA | | CACCCCATC | |
| N5- | TGGGGGGAGGTAGGTAGG | 13 | CCACCCCATCCAAGTTGGAGC | 31 |
| ESSalt2-14 | TA | | AAGATTATC | |
| N5- | TGGGGGGAGGTAGGTAGG | 13 | CAAGATTATCCTATATAAAAT | 32 |
| ESSalt2-15 | TA | | AGAAATATA | |
| N3- | TGGGGGGAGGTAGGTAGG | 13 | AGTGCAAGGATTAAAGGTAG | 34 |
| ESSalt2-01 | TA | | | |
| N3- | TGGGGGGAGGTAGGTAGG | 13 | CAAAAGGGGTAATACAGTAC | 35 |
| ESSalt2-02 | TA | | | |
| N3- | TGGGGGGAGGTAGGTAGG | 13 | CCATAATAAAGGGCTGAGGG | 36 |
| ESSalt2-03 | TA | | | |
| N3- | TGGGGGGAGGTAGGTAGG | 13 | GAGGAACCACCGCTCCACCC | 37 |
| ESSalt2-04 | TA | | | |
| N3- | TGGGGGGAGGTAGGTAGG | 13 | TCACAGTGCAAGGATTAAAGG | 38 |
| ESSalt2-05 | TA | | TAG | |
| N3- | TGGGGGGAGGTAGGTAGG | 13 | GTAGCAAAAGGGGTAATACA | 39 |
| ESSalt2-06 | TA | | GTAC | |
| N3- | TGGGGGGAGGTAGGTAGG | 13 | GTACCCATAATAAAGGGCTGA | 40 |
| ESSalt2-07 | TA | | GGG | |
| N3- | TGGGGGGAGGTAGGTAGG | 13 | AGGGGAGGAACCACCGCTCCA | 41 |
| ESSalt2-08 | TA | | CCC | |
| N3- | TGGGGGGAGGTAGGTAGG | 13 | AGTCACAGTGCAAGGATTAAA | 42 |
| ESSalt2-09 | TA | | GGTAG | |
| N3- | TGGGGGGAGGTAGGTAGG | 13 | AGGTAGCAAAAGGGGTAATA | 43 |
| ESSalt2-10 | TA | | CAGTAC | |
| N3- | TGGGGGGAGGTAGGTAGG | 13 | CAGTACCCATAATAAAGGGCT | 44 |
| ESSalt2-11 | TA | | GAGGG | |
| N3- | TGGGGGGAGGTAGGTAGG | 13 | CATAAGTCACAGTGCAAGGAT | 46 |
| ESSalt2-13 | TA | | TAAAGGTAG | |
| N3- | TGGGGGGAGGTAGGTAGG | 13 | TTAAAGGTAGCAAAAGGGGTA | 47 |
| ESSalt2-14 | TA | | ATACAGTAC | |
| N3- | TGGGGGGAGGTAGGTAGG | 13 | AATACAGTACCCATAATAAAG | 48 |
| ESSalt2-15 | TA | | GGCTGAGGG | |
| Scramble- | TGGGGGGAGGTAGGTAGG | 13 | GAAATTTATGTATTGATTAT | 17 |
| 04 | TA | | | |

TABLE 2B

| Construct | Humanized U7 Targeting | SEQ ID | % Identity to |
|---|---|---|---|
| Name | Sequence (5′ to 3′) | NO: | Mouse Sequence |
| N5-01 | AACCAGCGCTCCACCCCATC | 151 | 95.0 |
| N5-02 | CAAGTTGGAGCAAGATTATC | 19 | 100.0 |
| N5-03 | CTATACAAAATAGAAATATA | 50 | 95.0 |
| N5-04 | TAGTTTGTTATTAGTTAGAA | 51 | 95.0 |
| N5-05 | GAGGAACCAGCGCTCCACCCCATC | 52 | 95.8 |
| N5-06 | CATCCAAGTTGGAGCAAGATTATC | 23 | 100.0 |
| N5-07 | TATCCTATACAAAATAGAAATATA | 53 | 95.8 |
| N5-08 | TATATAGTTTGTTATTAGTTAGAA | 54 | 95.8 |
| N5-09 | GGGAGGAACCAGCGCTCCACCCCATC | 55 | 96.2 |
| N5-10 | CCCATCCAAGTTGGAGCAAGATTATC | 27 | 100.0 |
| N5-11 | ATTATCCTATACAAAATAGAAATATA | 56 | 96.2 |
| N5-12 | AATATATAGTTTGTTATTAGTTAGAA | 57 | 96.2 |
| N5-13 | TCAGGGGAGGAACCAGCGCTCCACCCCATC | 58 | 93.3 |
| N5-14 | CCACCCCATCCAAGTTGGAGCAAGATTATC | 31 | 100.0 |
| N5-15 | CAAGATTATCCTATACAAAATAGAAATATA | 59 | 96.7 |
| N5-16 | TAGAAATATATAGTTTGTTATTAGTTAGAA | 60 | 96.7 |
| N3-01 | AGTGCAAGGATTAAAGGTAG | 34 | 100.0 |
| N3-02 | CAAAAGGGGTAATACAGTAC | 35 | 100.0 |
| N3-03 | CCATAATAAAGGGCTCAGGG | 61 | 95.0 |
| N3-04 | GAGGAACCAGCGCTCCACCC | 62 | 95.0 |
| N3-05 | TCACAGTGCAAGGATTAAAGGTAG | 38 | 100.0 |
| N3-06 | GTAGCAAAAGGGGTAATACAGTAC | 39 | 100.0 |
| N3-07 | GTACCCATAATAAAGGGCTCAGGG | 63 | 95.8 |
| N3-08 | AGGGGAGGAACCAGCGCTCCACCC | 64 | 95.8 |
| N3-09 | AGTCACAGTGCAAGGATTAAAGGTAG | 42 | 100.0 |
| N3-10 | AGGTAGCAAAAGGGGTAATACAGTAC | 43 | 100.0 |
| N3-11 | CAGTACCCATAATAAAGGGCTCAGGG | 65 | 96.2 |
| N3-12 | TCAGGGGAGGAACCAGCGCTCCACCC | 66 | 92.3 |
| N3-13 | CATAAGTCACAGTGCAAGGATTAAAGGTAG | 46 | 100.0 |
| N3-14 | TTAAAGGTAGCAAAAGGGGTAATACAGTAC | 47 | 100.0 |
| N3-15 | AATACAGTACCCATAATAAAGGGCTCAGGG | 67 | 96.7 |
| N3-16 | GGGCTCAGGGGAGGAACCAGCGCTCCACCC | 68 | 93.3 |
| N5-ESSalt1-01 | AACCAGCGCTCCACCCCATC | 69 | 95.0 |
| N5-ESSalt1-02 | CAAGTTGGAGCAAGATTATC | 19 | 100.0 |
| N5-ESSalt1-03 | CTATACAAAATAGAAATATA | 70 | 95.0 |
| N5-ESSalt1-04 | TAGTTTGTTATTAGTTAGAA | 71 | 95.0 |
| N5-ESSalt1-05 | GAGGAACCAGCGCTCCACCCCATC | 72 | 95.8 |
| N5-ESSalt1-06 | CATCCAAGTTGGAGCAAGATTATC | 23 | 100.0 |
| N5-ESSalt1-07 | TATCCTATACAAAATAGAAATATA | 73 | 95.8 |
| N5-ESSalt1-08 | TATATAGTTTGTTATTAGTTAGAA | 74 | 95.8 |
| N5-ESSalt1-09 | GGGAGGAACCAGCGCTCCACCCCATC | 75 | 96.2 |
| N5-ESSalt1-10 | CCCATCCAAGTTGGAGCAAGATTATC | 27 | 100.0 |
| N5-ESSalt1-11 | ATTATCCTATACAAAATAGAAATATA | 76 | 96.2 |
| N5-ESSalt1-12 | AATATATAGTTTGTTATTAGTTAGAA | 77 | 96.2 |
| N5-ESSalt1-13 | TCAGGGGAGGAACCAGCGCTCCACCCCATC | 78 | 93.3 |
| N5-ESSalt1-14 | CCACCCCATCCAAGTTGGAGCAAGATTATC | 31 | 100.0 |
| N5-ESSalt1-15 | CAAGATTATCCTATACAAAATAGAAATATA | 79 | 96.7 |
| N5-ESSalt1-16 | TAGAAATATATAGTTTGTTATTAGTTAGAA | 80 | 96.7 |
| N3-ESSalt1-01 | AGTGCAAGGATTAAAGGTAG | 34 | 100.0 |
| N3-ESSalt1-02 | CAAAAGGGGTAATACAGTAC | 35 | 100.0 |
| N3-ESSalt1-03 | CCATAATAAAGGGCTCAGGG | 81 | 95.0 |
| N3-ESSalt1-04 | GAGGAACCAGCGCTCCACCC | 82 | 95.0 |
| N3-ESSalt1-05 | TCACAGTGCAAGGATTAAAGGTAG | 38 | 100.0 |
| N3-ESSalt1-06 | GTAGCAAAAGGGGTAATACAGTAC | 39 | 100.0 |
| N3-ESSalt1-07 | GTACCCATAATAAAGGGCTCAGGG | 83 | 95.8 |
| N3-ESSalt1-08 | AGGGGAGGAACCAGCGCTCCACCC | 84 | 95.8 |
| N3-ESSalt1-09 | AGTCACAGTGCAAGGATTAAAGGTAG | 42 | 100.0 |
| N3-ESSalt1-10 | AGGTAGCAAAAGGGGTAATACAGTAC | 43 | 100.0 |
| N3-ESSalt1-11 | CAGTACCCATAATAAAGGGCTCAGGG | 85 | 96.2 |
| N3-ESSalt1-12 | TCAGGGGAGGAACCAGCGCTCCACCC | 86 | 92.3 |
| N3-ESSalt1-13 | CATAAGTCACAGTGCAAGGATTAAAGGTAG | 46 | 100.0 |
| N3-ESSalt1-14 | TTAAAGGTAGCAAAAGGGGTAATACAGTAC | 47 | 100.0 |
| N3-ESSalt1-15 | AATACAGTACCCATAATAAAGGGCTCAGGG | 87 | 96.7 |
| N3-ESSalt1-16 | GGGCTCAGGGGAGGAACCAGCGCTCCACCC | 88 | 93.3 |
| N5-ESSalt2-01 | AACCAGCGCTCCACCCCATC | 89 | 95.0 |
| N5-ESSalt2-02 | CAAGTTGGAGCAAGATTATC | 19 | 100.0 |
| N5-ESSalt2-03 | CTATACAAAATAGAAATATA | 90 | 95.0 |
| N5-ESSalt2-04 | TAGTTTGTTATTAGTTAGAA | 91 | 95.0 |
| N5-ESSalt2-05 | GAGGAACCAGCGCTCCACCCCATC | 92 | 95.8 |
| N5-ESSalt2-06 | CATCCAAGTTGGAGCAAGATTATC | 23 | 100.0 |
| N5-ESSalt2-07 | TATCCTATACAAAATAGAAATATA | 93 | 95.8 |
| N5-ESSalt2-08 | TATATAGTTTGTTATTAGTTAGAA | 94 | 95.8 |
| N5-ESSalt2-09 | GGGAGGAACCAGCGCTCCACCCCATC | 95 | 96.2 |
| N5-ESSalt2-10 | CCCATCCAAGTTGGAGCAAGATTATC | 27 | 100.0 |
| N5-ESSalt2-11 | ATTATCCTATACAAAATAGAAATATA | 96 | 96.2 |
| N5-ESSalt2-13 | TCAGGGGAGGAACCAGCGCTCCACCCCATC | 97 | 93.3 |
| N5-ESSalt2-14 | CCACCCCATCCAAGTTGGAGCAAGATTATC | 31 | 100.0 |
| N5-ESSalt2-15 | CAAGATTATCCTATACAAAATAGAAATATA | 98 | 96.7 |
| N3-ESSalt2-01 | AGTGCAAGGATTAAAGGTAG | 34 | 100.0 |
| N3-ESSalt2-02 | CAAAAGGGGTAATACAGTAC | 35 | 100.0 |
| N3-ESSalt2-03 | CCATAATAAAGGGCTCAGGG | 99 | 95.0 |
| N3-ESSalt2-04 | GAGGAACCAGCGCTCCACCC | 100 | 95.0 |
| N3-ESSalt2-05 | TCACAGTGCAAGGATTAAAGGTAG | 38 | 100.0 |
| N3-ESSalt2-06 | GTAGCAAAAGGGGTAATACAGTAC | 39 | 100.0 |
| N3-ESSalt2-07 | GTACCCATAATAAAGGGCTCAGGG | 101 | 95.8 |
| N3-ESSalt2-08 | AGGGGAGGAACCAGCGCTCCACCC | 102 | 95.8 |
| N3-ESSalt2-09 | AGTCACAGTGCAAGGATTAAAGGTAG | 42 | 100.0 |
| N3-ESSalt2-10 | AGGTAGCAAAAGGGGTAATACAGTAC | 43 | 100.0 |
| N3-ESSalt2-11 | CAGTACCCATAATAAAGGGCTCAGGG | 103 | 96.2 |
| N3-ESSalt2-13 | CATAAGTCACAGTGCAAGGATTAAAGGTAG | 46 | 100.0 |
| N3-ESSalt2-14 | TTAAAGGTAGCAAAAGGGGTAATACAGTAC | 47 | 100.0 |
| N3-ESSalt2-15 | AATACAGTACCCATAATAAAGGGCTCAGGG | 104 | 96.7 |

TABLE 2C

| Construct Name | Relative Productive Transcript (Mouse, N2a cells) |
|---|---|
| N5-ESSalt2-15 | 1.73 |
| N5-ESSalt2-02 | 1.67 |
| N5-02 | 1.65 |
| N3-13 | 1.63 |
| N3-16 | 1.62 |
| N5-10 | 1.60 |
| N3-07 | 1.58 |
| N5-06 | 1.58 |
| N5-01 | 1.56 |
| N5-04 | 1.56 |
| N5-ESSalt2-14 | 1.56 |
| N3-09 | 1.54 |
| N3-ESSalt2-01 | 1.52 |
| N5-08 | 1.52 |
| N5-ESSalt2-04 | 1.52 |
| N3-10 | 1.50 |
| N5-09 | 1.50 |
| N5-11 | 1.50 |
| N5-15 | 1.50 |
| N5-07 | 1.48 |
| N5-ESSalt1-12 | 1.48 |
| N3-05 | 1.46 |
| N3-ESSalt1-13 | 1.46 |
| N3-ESSalt2-13 | 1.46 |
| N5-03 | 1.46 |
| N5-05 | 1.46 |
| N5-12 | 1.46 |
| N5-14 | 1.46 |
| N3-01 | 1.45 |
| N5-ESSalt1-15 | 1.45 |
| N3-14 | 1.43 |
| N3-ESSalt2-05 | 1.43 |
| N5-13 | 1.43 |
| N5-ESSalt1-13 | 1.43 |
| N3-02 | 1.41 |
| N3-ESSalt1-01 | 1.41 |
| N3-ESSalt1-06 | 1.39 |
| N5-16 | 1.39 |
| N5-ESSalt1-01 | 1.39 |
| N5-ESSalt1-05 | 1.39 |
| N3-04 | 1.37 |
| N3-12 | 1.37 |
| N3-ESSalt1-02 | 1.37 |
| N3-ESSalt1-09 | 1.37 |
| N3-ESSalt2-09 | 1.37 |
| N3-06 | 1.35 |
| N3-ESSalt1-10 | 1.35 |
| N5-ESSalt1-02 | 1.35 |
| N5-ESSalt2-11 | 1.35 |
| N3-08 | 1.33 |
| N3-11 | 1.33 |
| N3-ESSalt2-14 | 1.33 |
| N5-ESSalt1-04 | 1.33 |
| N3-03 | 1.31 |
| N3-ESSalt1-05 | 1.31 |
| N3-ESSalt1-14 | 1.31 |
| N3-ESSalt2-02 | 1.31 |
| N5-ESSalt1-14 | 1.31 |
| N5-ESSalt2-06 | 1.31 |
| N5-ESSalt2-10 | 1.31 |
| N5-ESSalt1-08 | 1.30 |
| N5-ESSalt1-09 | 1.30 |
| N5-ESSalt2-07 | 1.30 |
| N3-ESSalt1-16 | 1.28 |
| N3-ESSalt2-06 | 1.28 |
| N3-ESSalt2-15 | 1.28 |
| N5-ESSalt1-11 | 1.28 |
| N3-15 | 1.26 |
| N3-ESSalt1-11 | 1.24 |
| N5-ESSalt2-03 | 1.24 |
| N3-ESSalt1-15 | 1.22 |
| N3-ESSalt2-04 | 1.22 |
| N5-ESSalt1-10 | 1.22 |
| N5-ESSalt1-07 | 1.20 |
| N5-ESSalt1-16 | 1.20 |
| N5-ESSalt2-13 | 1.20 |
| N3-ESSalt2-03 | 1.18 |
| N3-ESSalt2-10 | 1.16 |
| N5-ESSalt1-06 | 1.16 |
| N3-ESSalt1-08 | 1.15 |
| N3-ESSalt2-11 | 1.15 |
| Scramble-01 | 1.15 |
| N3-ESSalt1-07 | 1.11 |
| N5-ESSalt2-08 | 1.11 |
| N3-ESSalt1-03 | 1.09 |
| N3-ESSalt1-04 | 1.09 |
| N3-ESSalt1-12 | 1.09 |
| N5-ESSalt2-05 | 1.09 |
| N5-ESSalt2-01 | 1.07 |
| N3-ESSalt2-07 | 1.05 |
| N5-ESSalt1-03 | 1.05 |
| Scramble-04 | 1.03 |
| Scramble-02 | 0.98 |
| N3-ESSalt2-08 | 0.90 |
| N5-ESSalt2-09 | 0.90 |
| Scramble-03 | 0.85 |

### Example 2: U7 Constructs with 5′ Targeting Sequence Directly Fused to 3′ Targeting Sequence

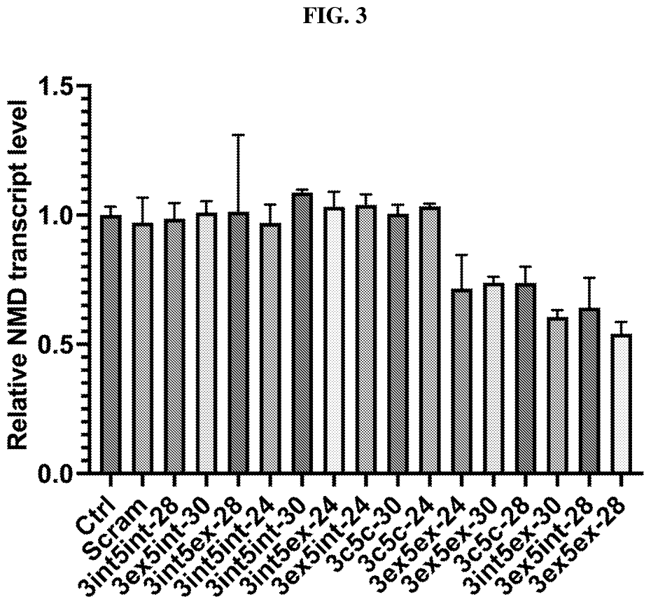

A second U7-based method for suppression of Scn1A NMD transcript was tested. A previous study reported decreased inclusion of an alternative exon using U7 vectors with targeting sequences composed of a 5′-targeting sequence directly fused to a 3′ targeting sequence. 15 different U7 constructs were engineered with different combinations of fused 5′/3′ targeting sequences of varied lengths where targeted sequences were either exonic, intronic, or centered upon the intron/exon junction (Tables 3A-3B). The constructs were tested for their abilities to suppress NMD transcript inclusion in Neuro-2a cells as in Example 1. NMD transcript suppression was observed in 5 out of 15 constructs tested, but none of the sequences suppressed NMD transcript levels by 50% (FIG. 3, Table 3C). Thus although, the different combinations of fused 5′/3′ targeting sequences did suppress NMD transcript levels, they all showed inferior efficacy to the hits identified in the original screen described in Example 1.

TABLE 3A

| Construct | 5′ Targeting Sequence | SEQ ID | 3′ Targeting Sequence | SEQ ID |
|---|---|---|---|---|
| Name | (5′ to 3′) | NO: | (5′ to 3′) | NO: |
| Control | N/A | | N/A | |
| Scramble | GCACCCCATCCCCCACTACA | 14 | N/A | |
| 3c5c-24 | GTAATACAGTACCCATAATA | 105 | AGCAAGATTATCCTATATAAAAT | 120 |
| | AAGG | | A | |
| 3c5c-28 | GGGTAATACAGTACCCATAA | 106 | GGAGCAAGATTATCCTATATAAA | 121 |
| | TAAAGGGC | | ATAGA | |
| 3c5c-30 | GGGGTAATACAGTACCCATA | 107 | TGGAGCAAGATTATCCTATATAA | 122 |
| | ATAAAGGGCT | | AATAGAA | |
| 3ex5ex-24 | AGGGGTAATACAGTACCCAT | 108 | AGATTATCCTATATAAAATAGAA | 123 |
| | AATA | | A | |
| 3ex5ex-28 | AAAAGGGGTAATACAGTACC | 109 | AAGATTATCCTATATAAAATAGA | 124 |
| | CATAATAA | | AATAT | |
| 3ex5ex-30 | CAAAAGGGGTAATACAGTAC | 110 | CAAGATTATCCTATATAAAATAG | 125 |
| | CCATAATAAA | | AAATATA | |
| 3ex5int-24 | TACAGTACCCATAATAAAGG | 111 | TTGGAGCAAGATTATCCTATATA | 126 |
| | GCTG | | A | |
| 3ex5int-28 | ATACAGTACCCATAATAAAG | 112 | AAGTTGGAGCAAGATTATCCTAT | 127 |
| | GGCTGAGG | | ATAAA | |
| 3ex5int30 | AATACAGTACCCATAATAAA | 113 | CAAGTTGGAGCAAGATTATCCTA | 128 |
| | GGGCTGAGGG | | TATAAAA | |
| 3int5ex-24 | AGGGGTAATACAGTACCCAT | 114 | TTGGAGCAAGATTATCCTATATA | 129 |
| | AATA | | A | |
| 3int5ex-28 | AAAAGGGGTAATACAGTACC | 115 | AAGTTGGAGCAAGATTATCCTAT | 130 |
| | CATAATAA | | ATAAA | |
| 3int5ex-30 | CAAAAGGGGTAATACAGTAC | 116 | CAAGTTGGAGCAAGATTATCCTA | 131 |
| | CCATAATAAA | | TATAAAA | |
| 3int5int-24 | TACAGTACCCATAATAAAGG | 117 | AGATTATCCTATATAAAATAGAA | 132 |
| | GCTG | | A | |
| 3int5int-28 | ATACAGTACCCATAATAAAG | 118 | AAGATTATCCTATATAAAATAGA | 133 |
| | GGCTGAGG | | AATAT | |
| 3int5int30 | AATACAGTACCCATAATAAA | 119 | CAAGATTATCCTATATAAAATAG | 134 |
| | GGGCTGAGGG | | AAATATA | |

TABLE 3B

| Construct | | SEQ ID |
|---|---|---|
| Name | Full Targeting Sequence (5′ to 3′) | NO: |
| Control | N/A | |
| Scramble | GCACCCCATCCCCCACTACA | 14 |
| 3c5c-24 | GTAATACAGTACCCATAATAAAGGAGCAAGATTATCCTATATAAAATA | 135 |
| 3c5c-28 | GGGTAATACAGTACCCATAATAAAGGGCGGAGCAAGATTATCCTATATA | 136 |
| | AAATAGA | |
| 3c5c-30 | GGGGTAATACAGTACCCATAATAAAGGGCTTGGAGCAAGATTATCCTAT | 137 |
| | ATAAAATAGAA | |
| 3ex5ex-24 | AGGGGTAATACAGTACCCATAATAAGATTATCCTATATAAAATAGAAA | 138 |
| 3ex5ex-28 | AAAAGGGGTAATACAGTACCCATAATAAAAGATTATCCTATATAAAATA | 139 |
| | GAAATAT | |
| 3ex5ex-30 | CAAAAGGGGTAATACAGTACCCATAATAAACAAGATTATCCTATATAAA | 140 |
| | ATAGAAATATA | |
| 3ex5int-24 | TACAGTACCCATAATAAAGGGCTGTTGGAGCAAGATTATCCTATATAA | 141 |
| 3ex5int-28 | ATACAGTACCCATAATAAAGGGCTGAGGAAGTTGGAGCAAGATTATCCT | 142 |
| | ATATAAA | |
| 3ex5int30 | AATACAGTACCCATAATAAAGGGCTGAGGGCAAGTTGGAGCAAGATTAT | 143 |
| | CCTATATAAAA | |
| 3int5ex-24 | AGGGGTAATACAGTACCCATAATATTGGAGCAAGATTATCCTATATAA | 144 |
| 3int5ex-28 | AAAAGGGGTAATACAGTACCCATAATAAAAGTTGGAGCAAGATTATCCT | 145 |
| | ATATAAA | |
| 3int5ex-30 | CAAAAGGGGTAATACAGTACCCATAATAAACAAGTTGGAGCAAGATTAT | 146 |
| | CCTATATAAAA | |
| 3int5int-24 | TACAGTACCCATAATAAAGGGCTGAGATTATCCTATATAAAATAGAAA | 147 |
| 3int5int-28 | ATACAGTACCCATAATAAAGGGCTGAGGAAGATTATCCTATATAAAATA | 148 |
| | GAAATAT | |
| 3int5int30 | AATACAGTACCCATAATAAAGGGCTGAGGGCAAGATTATCCTATATAAA | 149 |
| | ATAGAAATATA | |

TABLE 3C

| Construct Name | Relative NMD Transcript Level |
|---|---|
| Control | 1 |
| Scramble | 0.9708 |
| 3c5c-24 | 1.0047 |
| 3c5c-28 | 0.7382 |
| 3c5c-30 | 1.0385 |
| 3ex5ex-24 | 1.0328 |
| 3ex5ex-28 | 0.5406 |
| 3ex5ex-30 | 0.751 |
| 3ex5int-24 | 1.0319 |
| 3ex5int-28 | 0.6416 |
| 3ex5int30 | 1.0099 |
| 3int5ex-24 | 1.0866 |
| 3int5ex-28 | 0.967 |
| 3int5ex-30 | 0.6057 |
| 3int5int-24 | 1.0128 |
| 3int5int-28 | 0.967 |
| 3int5int30 | 0.9856 |

### Example 3 Optimization of 5′ and 3′ U7 Pairs

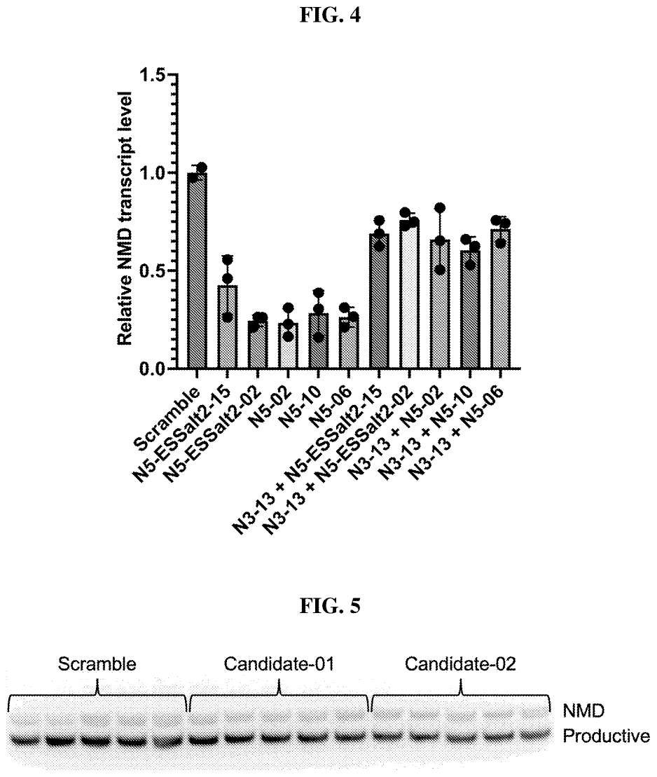

Previous work has also shown improved modulation of splicing using pairs of U7 constructs targeting the 5′ and 3′ end of a target exon. The U7 screen described in example 1 identified a cluster of high-efficacy target sequences at the 5′ end of mouse exon 21N. The top 5 5′ target sequences were paired with the top 3′ target sequence (Tables 4A-41B). The paired constructs were then compared against single-U7 constructs for each targeting sequence. Evaluation of NMD transcript levels in Neuro-2a cells, as performed in Example 1, showed that the combination of two U7 constructs targeting the 5′ and 3′ ends of the target exon partially inhibited NMD suppression when compared to the 5′- and 3′-targeting sequences alone (FIG. 4, Table 4C). This example shows that the pairing of constructs targeting the 5′ and 3′ end of a target exon is worse at NMD transcript suppression than a single construct.

TABLE 4A

| | 5′ Targeted Sequence | | | |
|---|---|---|---|---|
| Construct | | SEQ ID | Targeting Sequence | SEQ ID |
| Name | ESS (5′ to 3′) | NO: | (5′ to 3′) | NO: |
| N5- | TGGGGGGAGGTAGGTAGGT | 13 | CAAGATTATCCTATATAAA | 32 |
| ESSalt2- | A | | ATAGAAATATA | |
| 15 | | | | |
| N5- | TGGGGGGAGGTAGGTAGGT | 13 | CAAGTTGGAGCAAGATTAT | 19 |
| ESSalt2- | A | | C | |
| 02 | | | | |
| N5-02 | ATGATAGGGACTTAGGGTG | 11 | CAAGTTGGAGCAAGATTAT | 19 |
| | A | | C | |
| N5-10 | ATGATAGGGACTTAGGGTG | 11 | CCCATCCAAGTTGGAGCAA | 27 |
| | A | | GATTATC | |
| N5-06 | ATGATAGGGACTTAGGGTG | 11 | CATCCAAGTTGGAGCAAGA | 23 |
| | A | | TTATC | |
| N3-13 + | TGGGGGGAGGTAGGTAGGT | 13 | CAAGATTATCCTATATAAA | 32 |
| N5- | A | | ATAGAAATATA | |
| ESSal2-15 | | | | |
| N3-13 + | TGGGGGGAGGTAGGTAGGT | 13 | CAAGTTGGAGCAAGATTAT | 19 |
| N5- | A | | C | |
| ESSalt2- | | | | |
| 02 | | | | |
| N3-13 + | ATGATAGGGACTTAGGGTG | 11 | CAAGTTGGAGCAAGATTAT | 19 |
| N5-02 | A | | C | |
| N3-13 + | ATGATAGGGACTTAGGGTG | 11 | CCCATCCAAGTTGGAGCAA | 27 |
| N5-10 | A | | GATTATC | |
| N3-13 + | ATGATAGGGACTTAGGGTG | 11 | CATCCAAGTTGGAGCAAGA | 23 |
| N5-06 | A | | TTATC | |

TABLE 4B

| | 3′ Targeted Sequence | | | |
|---|---|---|---|---|
| Construct | | SEQ ID | Targeting Sequence | SEQ ID |
| Name | ESS (5′ to 3′) | NO: | (5′ to 3′) | NO: |
| N5-ESSalt2-15 | N/A | N/A | N/A | N/A |
| N5-ESSalt2-02 | N/A | N/A | N/A | N/A |
| N5-02 | N/A | N/A | N/A | N/A |
| N5-10 | N/A | N/A | N/A | N/A |
| N5-06 | N/A | N/A | N/A | N/A |
| N3-13 + N5- | ATGATAGGGACTTAGGGTGA | 11 | CATAAGTCACAGTGCAAGG | 46 |
| ESSal2-15 | | | ATTAAAGGTAG | |
| N3-13 + N5- | ATGATAGGGACTTAGGGTGA | 11 | CATAAGTCACAGTGCAAGG | 46 |
| ESSalt2-02 | | | ATTAAAGGTAG | |
| N3-13 + N5-02 | ATGATAGGGACTTAGGGTGA | 11 | CATAAGTCACAGTGCAAGG | 46 |
| | | | ATTAAAGGTAG | |
| N3-13 + N5-10 | ATGATAGGGACTTAGGGTGA | 11 | CATAAGTCACAGTGCAAGG | 46 |
| | | | ATTAAAGGTAG | |
| N3-13 + N5-06 | ATGATAGGGACTTAGGGTGA | 11 | CATAAGTCACAGTGCAAGG | 46 |
| | | | ATTAAAGGTAG | |

TABLE 4C

| Construct Name | Relative NMD Transcript Level |
|---|---|
| N5-ESSalt2-15 | 0.4261 |
| N5-ESSalt2-02 | 0.2451 |
| N5-02 | 0.2341 |
| N5-10 | 0.2836 |
| N5-06 | 0.2631 |
| N3-13 + N5-ESSal2-15 | 0.6895 |
| N3-13 + N5-ESSalt2-02 | 0.7586 |
| N3-13 + N5-02 | 0.6593 |
| N3-13 + N5-10 | 0.6043 |
| N3-13 + N5-06 | 0.7126 |

### Example 4: Validation of U7 Candidates Identified in Screen in Post-Mitotic Cells

Two candidates identified in Example 1 (Candidate 1: N5-ESSalt2-15 and Candidate 2: N5-05) were further tested for their ability to increase production of the productive transcript. First, AAVs encoding these two constructs were produced and systemic injection of wildtype mice at P28 was performed. Brain tissue was collected at 4-weeks post-injection and levels of productive and NMD transcripts were assessed. Although widespread AAV transduction was observed in the brain, there was no significant reduction of NMD transcript levels or increased productive Scn1a transcript levels (FIG. 5). Endogenous U7 expression is regulated by the cell cycle, suggesting that expression may be weak in post-mitotic cells of the central nervous system. Thus, additional approaches were explored to assess whether increasing U7 expression could overcome this barrier, resulting in efficient splice modulation in the central nervous system.

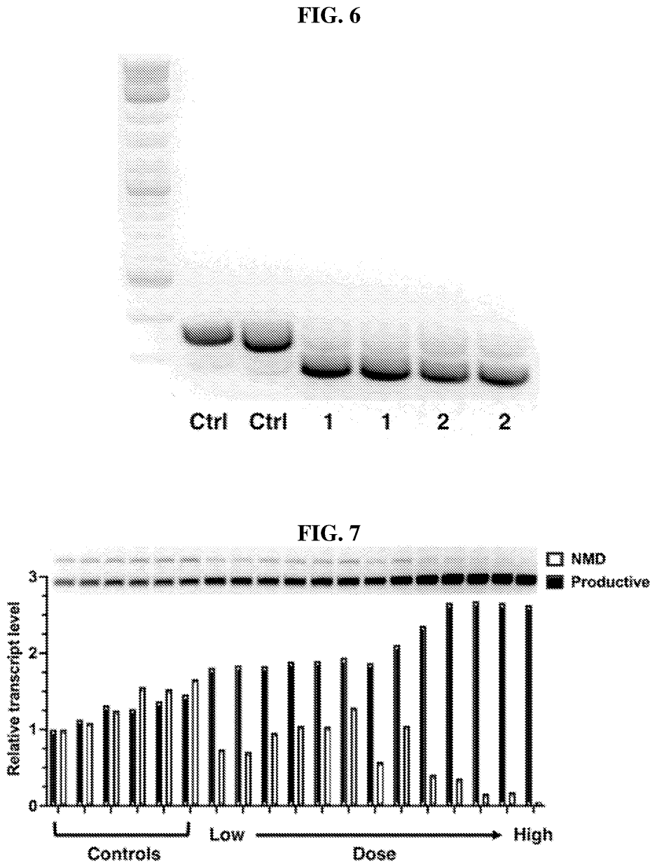

To increase U7 expression, AAV constructs encoding an array of four U7 expression cassettes of either candidates 1 or 2 were generated. Each construct contained 4 U7 cassettes, each consisting of the U7 promoter, ESS, antisense sequence, smOPT, hairpin, and 3′ terminator sequence separated by a spacer consisting of random nucleotides. Primary mouse hippocampal neurons were treated with the AAVs expressing the U7 cassettes of candidates 1 or 2 and then cultured for 1 week. After 1 week of culture, RNA was collected and analyzed for the amount of NMD transcript and the productive transcript via end-point RT-PCR. FIG. 6 shows control cells treated with the scrambled U7 sequence express low amounts of the productive transcript but high levels of NMD transcript, as evidenced by the weak low molecular weight band (productive transcript) and the strong high molecular weight transcript (NMD transcript). All samples treated with either U7 candidate 1 or 2 show near-complete suppression of the NMD transcript and high levels of the productive transcript. This example demonstrates that increasing the expression levels of the candidate U7 constructs through increasing U7 cassette copy number can suppress inclusion of the NMD transcript and increase expression of the productive transcript in post-mitotic neuronal cells.

### Example 5: In Vivo Validation of U7 Candidates

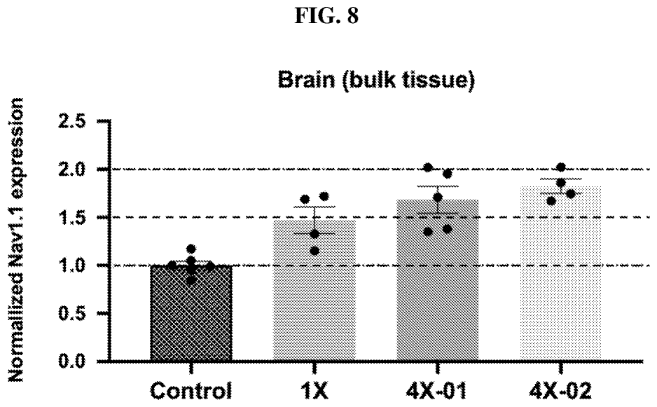

Wild type C57Bl/6J mice were injected at P28 with varying doses of AAVs encoding 4 copies of either scramble, candidate 1 or candidate 2 U7 cassettes (described in Example 4). At 3-weeks post injection, RNA and protein were isolated from brain tissue. Analysis of NMD transcript levels showed that the 4×U7 constructs can suppress NMD transcript formation in a dose-dependent manner. High-dose delivery of AAV can almost completely suppress NMD transcript production and nearly triple productive transcript levels (FIG. 7). Western blot analysis of protein confirmed NaV1.1 levels were double those of control samples in animals injected with 4×AAVs (FIG. 8). This example shows that both U7 candidates expressed via AAV are able to suppress NMD transcripts and increase productive transcripts at both the mRNA and protein level in vivo when expressed at sufficient levels.

### Example 6: Additional Vector Modifications

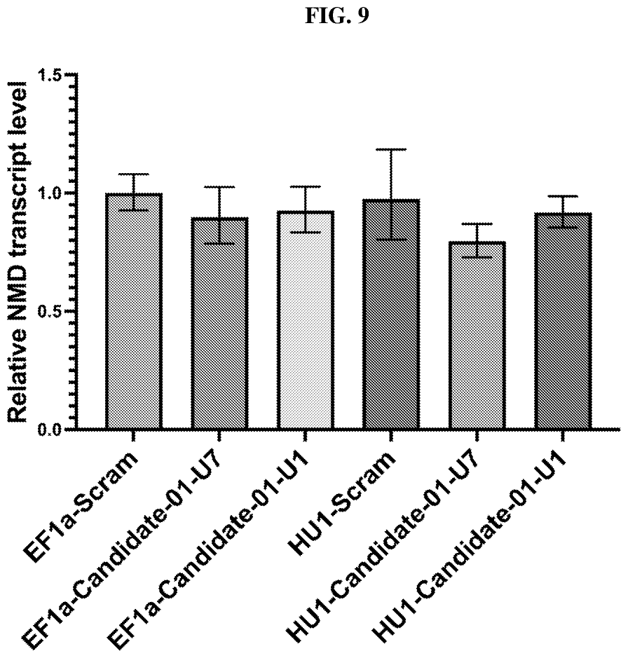

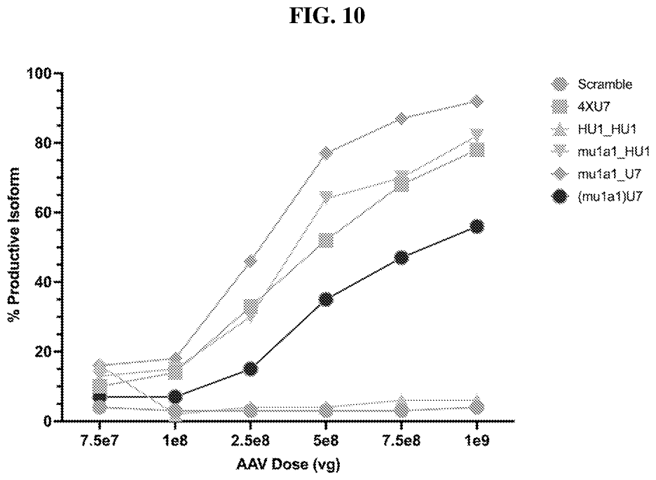

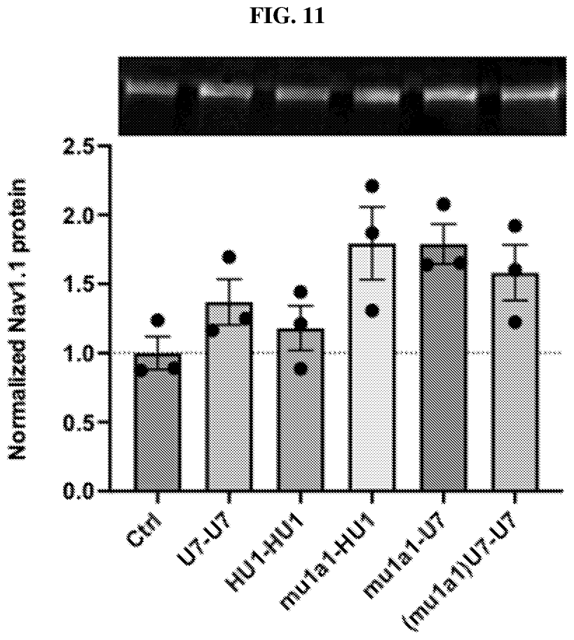

Although administration of high doses of AAVs expressing the U7 candidates resulted in near-complete elimination of NMD transcripts in vivo, delivery of AAV-based gene therapies to the human brain suffer from low efficiency, both in terms of cell numbers and genome copies transduced per cell. Based on this observation, designing gene therapy vectors with high molecular efficacy even at low genome copies per cell is imperative for clinical success. To increase U7 expression from each construct the U7 promoter and the 3′ termination signal sequence were modified. Like U7, expression of the U1 family of small RNAs is regulated by RNA polymerase II. However, U1 RNAs are ubiquitously expressed at high levels compared to U7. Based on this observation, the 5′ and 3′ regulatory elements of U1 were tested for their ability to drive stronger expression of the U7 backbone compared to the endogenous U7 sequences. Additionally, the RNA polymerase II dependent element was switched out with the standard PolII-dependent promoter (ex. EF1alpha) to test the ability of the standard PolII-dependent promoter to increase U7 expression. These two concepts were tested by engineering constructs comprising the human HU1-1 promoter or the EF1alpha core promoter. The HU1-1 and U7 3′ regulatory elements may be important for proper folding and functional U7 expression, so the different promoters were paired with either the standard U7 3′ signal or with the U7 3′ signal immediately followed by the HU1-1 3′ signal (Table 1). Initial testing of the ability of these constructs to suppress Scn1A NMD transcript inclusion in Neuro-2a cells was done with a set of U7 expression plasmids with either the scramble targeting sequence or the Candidate 1 targeting sequence. However, analysis of NMD transcript levels showed that these vectors lacked functional U7 activity (FIG. 9). Functional expression of snRNAs relies upon proper RNA folding after transcription, which is orchestrated by protein factors recruited by the distal and proximal sequence elements (DSE and PSE) encoded by snRNA promoters, as well as regulatory sequences located within the transcriptional terminator sequences. Thus, two potential reasons why this initial set of vectors with modified regulatory elements failed are 1) improper recruitment of accessory factors by the EF1a promoter and 2) incorrect secondary structure formation due to combined effects from the promoter and 3′ signals. To test this, U7 constructs with ESS/antisense candidate 1 were engineered by combining the core U7 structure (ESS-antisense sequence-smOPT-hairpin) with varying combinations of the mouse U7 promoter, mouse U1a1 promoter, human U1-1 promoter, mouse U7 promoter with its distal sequence element replaced with that of the human U1-1 and its proximal sequence element replaced with that of mouse U1a1, and either the mouse U7 3′ terminator or the human U1-1 3′ terminator. The different combinations were then tested for their ability to suppress SCN1A NMD transcript inclusion in cultured primary cortical neurons. The mu1a1 promoter paired with either the HU1 terminator or U7 terminator was as efficient or greater than the 4× standard U7 array, but the U7 promoter with modified PSE and DSE elements worked less efficiently (FIG. 10). Pairing the HU1 promoter and terminator did not result in any functional U7 expression (FIG. 10). AAVs encoding these U7 constructs were then injected into wildtype mice at a low dose and their ability to increase NaV1.1 levels through their suppression of SCN1A NMD transcript formation was assessed. Based on NaV1.1 protein levels, pairing the mu1a1 promoter with the HU1 terminator or U7 terminator once again outperformed the standard 4×U7 array. In contrast to experiments in human neurons, replacing the U7 promoter DSE and PSE with that of mu1a1 was also capable of increasing functional U7 expression (FIG. 11). Once again, the combination of the HU1 promoter and terminator did not result in any functional U7 expression. This example shows that multiple combinations of promoters and 3′ terminal sequences are sufficient to increase functional U7 expression and ultimately the production of NaV1.1 in vivo when compared to wild-type U7. However, not all promoter/terminator combinations are functional and there can be species-specific differences in the efficiency of those that are function, as demonstrated by the (mu1a1)U7 promoter.

### Example 7: Therapeutic Validation of U7 Candidates

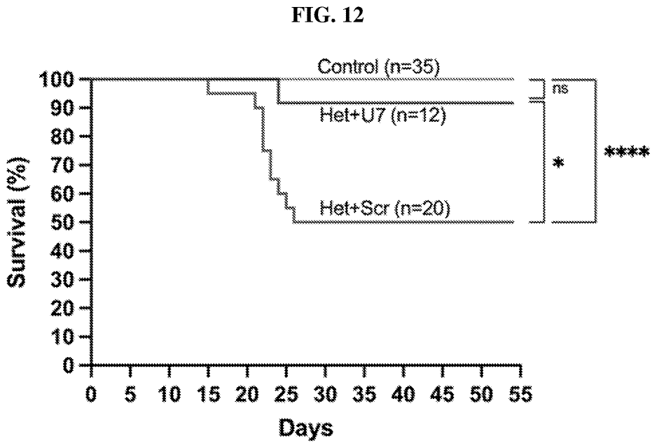

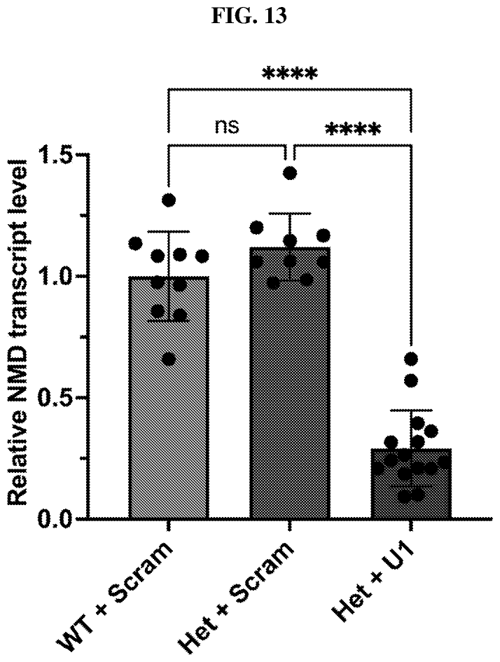

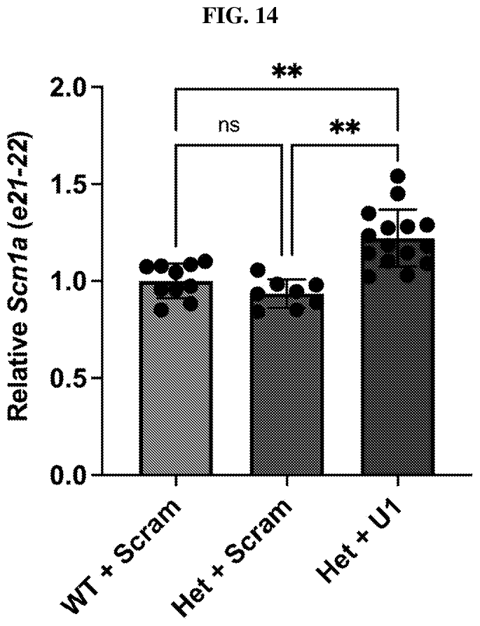

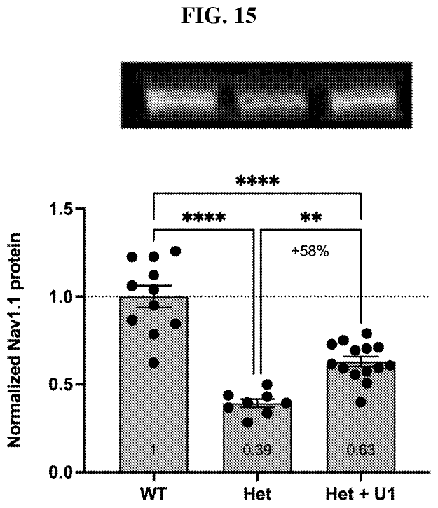

To test the therapeutic potential of a modified U7 construct identified in Example 6 (mu1a1-Candidate 1-HU1), AAV encoding a 3× array of this candidate was produced. The AAV was then systemically injected into Scna1 heterozygous mice via retroorbital injection at postnatal day 14. Beginning at approximately postnatal day 17, untreated Scn1A heterozygous mutants begin to spontaneously die from sudden epileptic death. To test the efficacy of the U7 construct, the survival in a cohort of animals injected with either the modified U7 vector or a scramble control was evaluated, along with Scn1a transcript and NaV1.1 levels. Significant rescue of the spontaneous death phenotype was observed, with 11/12 U7-injected animals surviving until P55 compared to 10/20 scramble-injected animals (FIG. 12). At ~P55, whole brain tissue of surviving animals was harvested and the levels of Scn1A NMD transcript, the total Scn1A mRNA, and the NaV1.1 protein were assessed. A significant reduction of NMD transcript levels (FIG. 13), an increase in total Scn1A transcript levels (FIG. 14), and a 58% increase in NaV1.1 levels compared to scramble-injected heterozygous mutants (FIG. 15) was observed. This example demonstrated a strong molecular and functional rescue of Dravet-associated phenotypes in a disease-relevant Scn1A mutant mouse model.

### Example 8: Therapeutic Validation of U7 Candidates

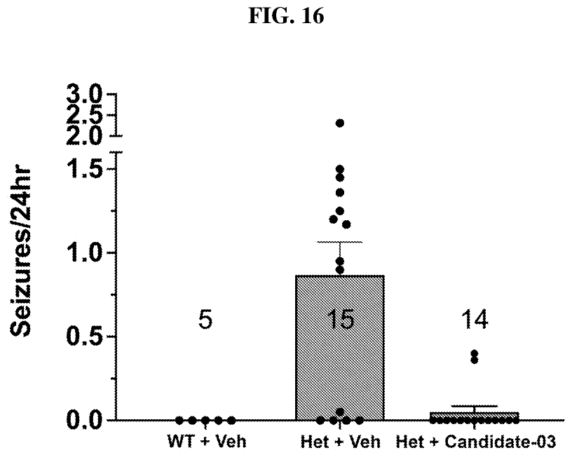

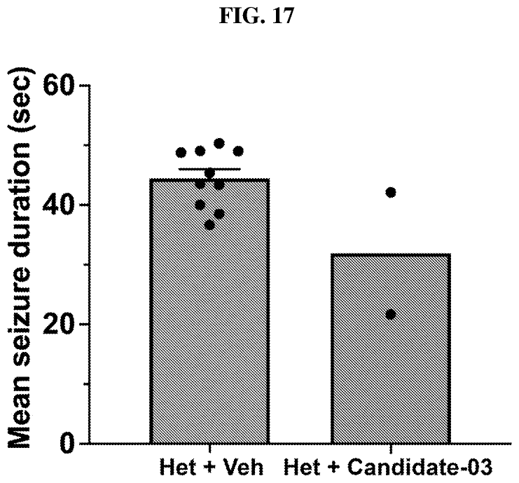

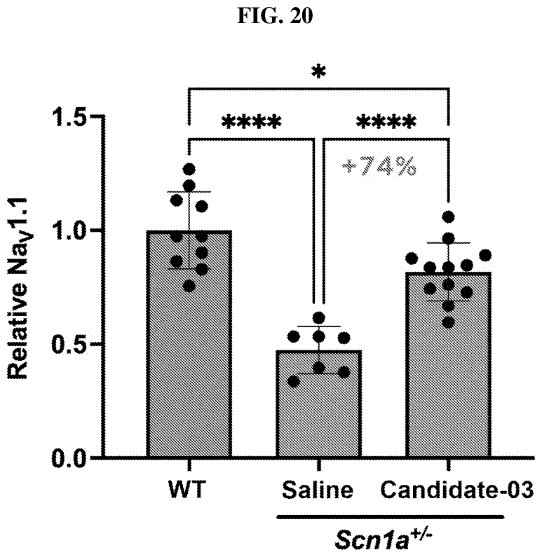

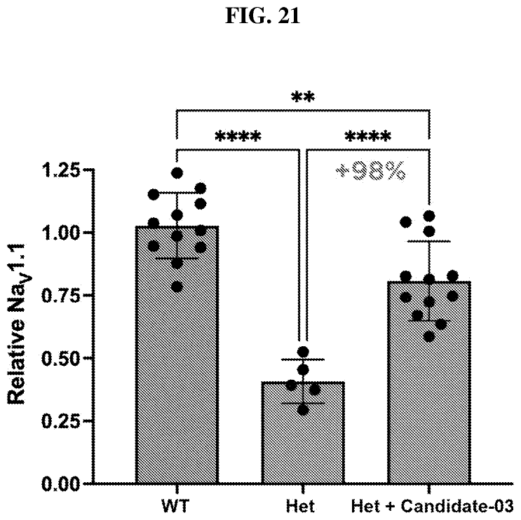

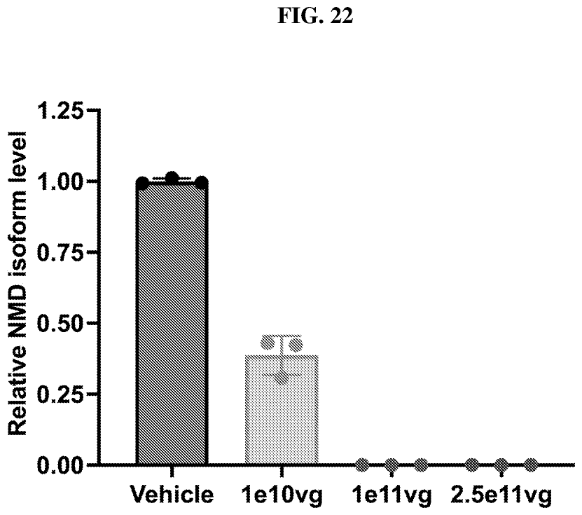

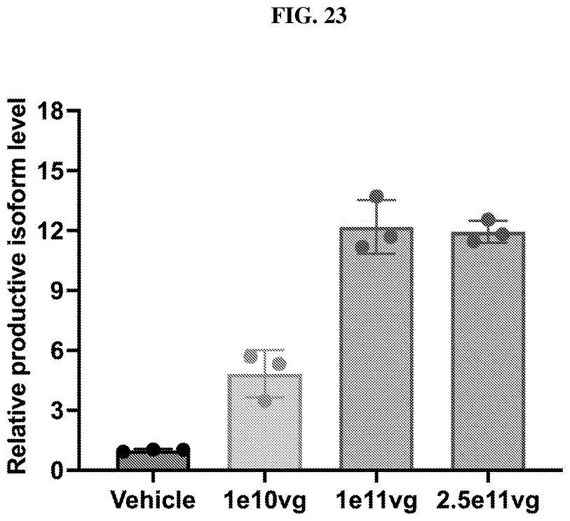

This example continues the experiments of Example 7. The therapeutic potential of an optimized U7 vector, referred to here as Candidate 3 (scAAV-(3×)mu1a1-N5-06-HU1), was tested via EEG-based analysis of seizure suppression. Candidate 3 is a different configuration of Candidate 2, and includes a self-complementary AAV (scAAV) driving a 3× array of mu1a1-Candidate 2-HU1. Scn1a^{+/-} mice were administered 5e10 vector genomes (vg) of Candidate 3 packaged with AAV9 capsid via ICV injection at postnatal day 2. EEG electrodes were implanted at ~P20 and EEG recording was subsequently performed between P24 and P45. Analysis of saline-injected controls showed significant seizure activity, with 10/15 animals experiencing spontaneous seizures at an average rate of >1 seizure per 24-hour period (FIG. 16). In contrast, only 2/14 animals injected with AAV encoding Candidate 3 exhibited spontaneous seizure activity at an average rate of <0.5 seizures per 24-hour period (FIG. 16). AAV-injected animals exhibiting spontaneous seizures also showed a trend toward shorter seizure durations (FIG. 17). To assess potential differences in efficacy related to delivery method and timing, additional survival studies were performed with AAV9-packaged virus injected via ICV at P2 (5e10 vg) and PHP.eB-packaged virus injected at P14 (1e14 vg/kg). Injection at both timepoints showed near-complete suppression of SUDEP (FIGS. 18-19) and analysis of brain tissue collected from surviving animals at P90 revealed robust rescue of NaV1.1 protein levels (FIG. 20-21). Last, to assess target engagement in human neurons, differentiated ReNcell CX cultures were treated with either AAV formulation buffer or 1e10 vg, 1e11 vg, or 2.5e11 vg of AAV encoding Candidate 3 and cultured for 7 days. Purified RNA was used for end-point RT-PCR to assess suppression of NMD isoform production, as well as production of productive isoform. RT-PCR showed dose-dependent suppression of NMD isoform splicing (FIG. 22) with concomitant up-regulation of productive SCN1A isoform production (FIG. 23), validating the target engagement with Candidate 3 across species. These data support the use of Candidate 3 for therapeutic restoration of NaV1.1 levels in the treatment of SCN1A haploinsufficiency disorders.

### Example 9: Platform Expansion and Validation of Promoter/Terminator Combinations Driving U7 Expression and U7-Mediated Splice Modulation

Given the success in improving programmed U7-mediated splice modulation with promoters and terminators from the U1 family of snRNAs, the platform was extended by testing additional combinations of promoter and terminator regions from mouse and human snRNAs. Because some core promoter and terminator elements are not well-defined for all small RNAs across species, regions of ~500 bp upstream and downstream of the coding regions of human and mouse small RNAs were selected for testing. A library of constructs, each encoding an engineered U7 snRNA targeting exon 13N of mouse Scn2a driven by one of 529 combinations of the chosen promoters and terminators was generated (Table 7A). Construct fragments in Table 7A were named sequentially in the order of a three-digit fragment ID number, a recombinant promoter sequence (SEQ ID NOs: 6 and 318-340 from Table 5), a U7 core sequence (Table 7B), and finally a recombinant 3′ signal/terminator sequence (SEQ ID NOs: 10 and 341-363 from Table 5), and were constructed 5′ to 3′ in the same order.

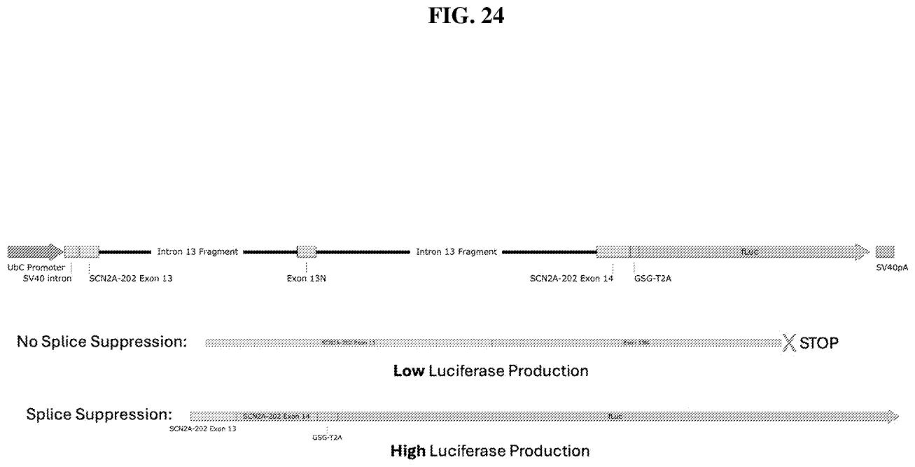

To screen for the ability of these promoters and terminators to drive high levels of functional U7 expression, a luciferase reporter with the following features was generated: a relatively weak ubiquitous promoter (Ubc-SV40 pA), a canonical Kozak sequence, a fragment of mouse Scn2a exon 13, a fragment of mouse Scn2a intron 13, mouse Scn2a exon 13N, another fragment of mouse Scn2a intron 13, and a fragment of mouse Scn2a exon 14 fused in-frame to firefly luciferase (Table 6, FIG. 24). Under normal conditions a significant fraction of transcripts include exon 13N, which is out-of-frame and leads to nonsense-mediated decay, thus reducing luciferase levels. Successful suppression of exon 13N inclusion by U7 results in proper in-frame expression of luciferase, which can be read out via luciferase enzymatic activity.

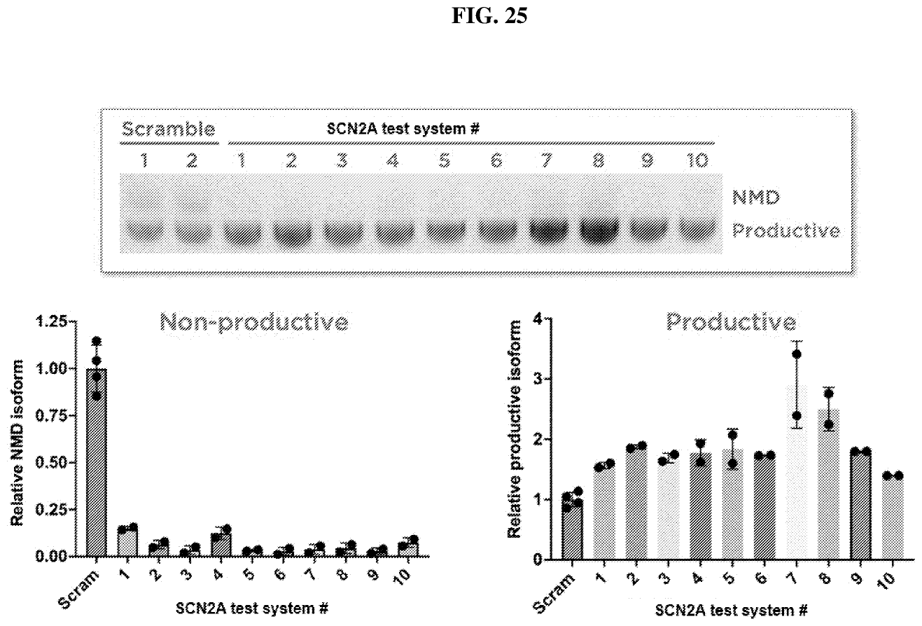

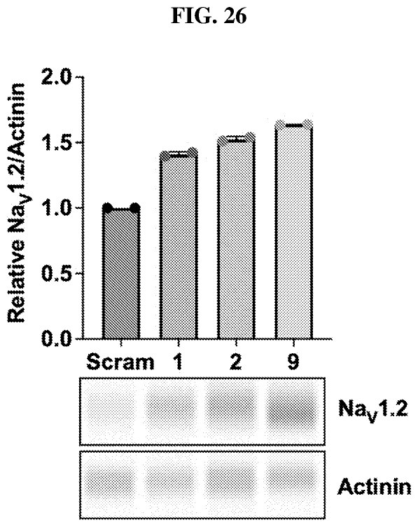

Using this system a candidate ESS and U7 antisense sequence were identified that can efficiently suppress exon 13N inclusion and increase luciferase activity when driven by the combination of the mu1a1 promoter and HU1 terminator. The screen was then validated in mouse Neuro-2a cells, where it was demonstrated that treatment with multiple candidate mu1a1-ESS-Scn2a-U7-HU1 plasmids identified in the screen could efficiently suppress Scn2a exon 13N splicing and increase productive Scn2a transcript levels (FIG. 25). Virus was also produced using three different U7 targeting sequences identified with this system and transduction of differentiated ReNcell CX cultures revealed significant up-regulation of Nav1.2 levels (FIG. 26), verifying that the luciferase reporter activity faithfully predicts modulation of splicing of endogenous transcripts.

Using this validated system, Neuro-2a cells were co-transfected with the luciferase reporter, a control plasmid expressing Renilla luciferase, and individual plasmids from the 529 candidates in this example. The library was split into 6 sub-libraries and each plate was screened in triplicate. Multiple control plasmids were included on each plate for reference. Following 24 hours of culture, average firefly luciferase levels, normalized to renilla luciferase, were calculated for each construct and an ANOVA was run for each sub-library to identify promoter/terminator combinations capable of significantly suppressing exon 13N inclusion, a direct proxy for functional U7 expression. Across libraries, 324/529 promoter/terminator combinations resulted in a statistically significant increase in luciferase activity (Tables 8-13). In line with previous findings that functional combinations are not obvious, even when combining elements from the same gene and species, there were many instances where a given promoter was only functional with the terminator of a different gene or species, suggesting there are unknown sequence determinants required for functional U7 expression. Together, these studies identify a suite of promoter/terminator combinations capable of driving high levels of functional U7 expression and expand the toolkit of regulatory elements for use of U7-mediated splice modulation.

TABLE 5
Example Regulatory Elements

| Sequence | | SEQ ID |
|---|---|---|
| Name | Sequence | NO: |
| Mm U7 | Aacaacataggagctgtgattggctgttttcagccaatcagcactgactcatttgcatagccttta | 5 |
| Promoter | caagcggtcacaaactcaagaaacgagcggttttaatagtcttttagaatattgtttatcgaaccg | |
| | aataaggaactgtgctttgtgattcacatatcagtggaggggtgtggaaatggcaccttgatctca | |
| | ccctcatcgaaagtggagttgatgtccttccctggctcgctacagacgcacttccgc | |
| Mm U1a1 | TAAAGACTGTGCATCCGACTCCTACATTTATGAAAGTAAATGCCTATTGTTAG | 6 |
| (a.k.a. mu1a1) | AACAAAAAAGGCTACAGAACAAAAAACAAAGCGAAATACCATCTGCTTTAGG | |
| Promoter | TTCAGTGGTATTTTCCCGCTGACAGGGAGGCGGGTTTTTGGGTACAGGAAACG | |
| | AGTCACTATGGAGGCGGTACTATGTAGATGAGAATTCAGGAGCAAACTGGGA | |
| | AAAGCAACTGCTTCCAAATATTTGTGATTTTTACAGTGTAGTTTTGGAAAAAC | |
| | TCTTAGCCTACCAATTCTTCTAAGTGTTTTAAAATGTGGGAGCCAGTACACAT | |
| | GAAGTTATAGAGTGTTTTAATGAGGCTTAAATATTTACCGTAACTATGAAATG | |
| | CTACGCATATCATGCTGTTCAGGCTCCGTGGCCACGCAACTC | |
| Hs U1-1 | aacaacataggagctgtgattggctgttttcagccaatcagcactgactcAGGGCGACTTCTATGT | 7 |
| Promoter | AGATGAGGCAGCGCAGAGGCTGCTgcctttacaagcggtcacaaactcaagaaacgagcggtttta | |
| | atagtcttttagaatattgtttatcgaaccgaataaggaactgtgctttgtgattcacatatcagt | |
| | ggaggggtgtggaaatggcaccttgatGGGAGTGCGCGAGGCAAGTGACCGTGTGTGgagttgatg | |
| | tccttccctggctcgctacagacgcacttccgc | |
| Modified Mm | aacaacataggagctgtgattggctgttttcagccaatcagcactgactcAGGGCGACTTCTATGT | 8 |
| U7 Promoter | AGATGAGGCAGCGCAGAGGCTGCTgcctttacaagcggtcacaaactcaagaaacgagcggtttta | |
| | atagtcttttagaatattgtttatcgaaccgaataaggaactgtgctttgtgattcacatatcagt | |
| | ggaggggtgtggaaatggcaccttgatTTACCGTAACTATGAAATGgagttgatgtccttccctgg | |
| | ctcgctacagacgcacttccgc | |
| hRNU1-1- | TTTCTTTTGTAATCCGAAACATTCTAGTCTGCGAATTAAAAGCCATTATTTGAA | 318 |
| Promoter | GAAGGATGCCCCGGCTCCATCTGGCCACCGAAAGATTGCTCCTTAACACAGG | |
| | CTAAGGACCAGCTTCTTTGGGAGAGAACAGACGCAGGGGGGGGAGGGAAAA | |
| | AGGGAGAGGCAGACGTCACTTCCTCTTGGCGACTCTGGCAGCAGATTGGTCG | |
| | GTTGAGTGGCAGAAAGGCAGACGGGGACTGGGCAAGGCACTGTCGGTGACAT | |
| | CACGGACAGGGCGACTTCTATGTAGATGAGGCAGCGCAGAGGCTGCTGCTTC | |
| | GCCACTTGCTGCTTCGCCACGAAGGGAGTTCCCGTGCCCTGGGAGCGGGTTCA | |
| | GGACCGCTGATCGGAAGTGAGAATCCCAGCTGTGTGTCAGGGCTGGAAAGGG | |
| | CTCGGGAGTGCGCGGGGCAAGTGACCGTGTGTGTAAAGAGTGAGGCGTATGA | |
| | GGCTGTGTCGGGGCAGAGGCACAACGTTTC | |
| hRNU1-2- | GTTTCTTCTGTAATCCGAAACATTCTAGTCTGCGAATTAAAAGCCATTATTTGA | 319 |
| Promoter | AGAAGGATGCCCCGGCTCCATCTGGCCACCGAAAGGTTGCTCCTTAACACAG | |
| | GCTAAGGACCAGCTTTTTTGGGAGAGAACAGACGCAGGGGGGGGAGGGAAA | |
| | AAGGGAGAGGCAGACGTCACTTCCTCTTGGCGGCTCTGGCAGCAGATTGGTC | |
| | AGTTGAGTGGCAGAAAAGCAGACGGGGACTGGGCAAGGCACTGTCGGTGAC | |
| | ATCACGGACAGGGCGACTTCTATGTAGATGAGGCAGCGCAGAGGCTGCTGCT | |
| | TCGCCACTTGCTGCTTCGCCACGAAGGAGTTCCCCTGCCCTGGGAGCGGGTTC | |
| | AGGACCGCGGATCGGAAGAGAGAATCCCAGCTGTGTGTCAGGGCTGGAAAGG | |
| | GCTCGGGAGTGCGCGGGGCAAGTGACCGTGTGTGTAAAGAGTGAGGCGTATG | |
| | AGGCTGTGTCGGGGCAGAGCCCGAAGATCTC | |
| hRNU1-3- | GTTTCTTTTGTAATCCGAAACATTCTAGTCTGCGAATTAAAAGCCATTATTTGA | 320 |
| Promoter | AGAAGGATGCCCCGGCTCCATCTGGCCACCGAAAGGTTGCTCCTTAACACAG | |
| | GCTAAGGACCAGCTTCTTTGGGAGAGAACAGACGCAGGGGGGGGAGGGAAA | |
| | AAGGGAGAGGCAGACGTCACTTCCCCTTGGCGGCTCTGGCAGCAGATTGGTC | |
| | GGTTGAGTGGCAGAAAGGCAGACGGGGACTGGGCAAGGCACTGTCGGTGAC | |
| | ATCACGGACAGGGCGACTTCTATGTAGATGAGGCAGCGCAGAGGCTGCTGCT | |
| | TCGCCACTTGCTGCTTCGCCACGAAGGAGTTCCCGTGCCCTGGGAGCGGGTTC | |
| | AGGACCGCGGATCGGAAGTGAGAATCCCAGCTGTGTGTCAGGGCTGGAAAGG | |
| | GCTCGGGAGTGCGCGGGGCAAGTGACCGTGTGTGTAAAGAGTGAGGCGTATG | |
| | AGGCTGTGTCGGGGCAGAGCCCGAAGATCTC | |
| hRNU2-1- | TCTCCCCGCCCGCGGGCGGCGAAGTAAAGGCCCAGCGCAGCCCGCGCTCCTG | 321 |
| Promoter | CCCTGGAGCCTCGTCTTTCTCCAGGAAAACGTGGACCGCTCTCCGCCGACAGG | |
| | TCTCTTCCACAGACCCCTGTCGCCTTCGCCCCCGGTCTCTTCCGGTTCTGTCTT | |
| | TTCGCTGGCTCGATACGAACAAGGAAGTCGCCCCCAGCGGAGCCCCGGCTCC | |
| | CCCAGGCAGAGGCGGCCCCGGGGGCGGAGTCAACGGCGGAGGCCACGCCCTC | |
| | TGTGAAAGGGCGGGGCATGCAAATTCGAAATGAAAGCCCGGGAACGCCGGA | |
| | AGAAGCACGGGTGTAAGATTTCCCTTTTCAAAGGCAGAGAATAAGAAATCAG | |
| | CCCGAGAGTGTAAGGGCGTCAATAGCGCTGTGGACGAGACAGAGGGAATGG | |
| | GGCAAGGAGCGAGGCTGGGGCTCTCACCGCGACTTGAATGTGGATGAGAGTG | |
| | GGACGGTGACGGCGGGCGCGAAGGCGAGCGC | |
| hU3-Promoter | CCAACAAGAGAGACCCTCATCTCTACAAAATATTTTTTAAAATAACTGGGCCT | 322 |
| | CATGGTGCATTCCTGTAATCTGAGCTAGTTGGGAGGATAGCTAGAGCCCAGG | |
| | AGATCGAGGCTGCAGAGAACCGTGATCACACGACTGCACTCCAGCCTGGGTG | |
| | ACAGAGCAAGACCCTGTCTCAATAGATAGATAGATAGATAGATAGATAGATA | |
| | GATAGATAGATAGATAGATAAAAACAGAGAGGTGAAAAATATGGTTTTAAGG | |
| | GTTCTGTTTAAGAATACTCTGTTCCAACATGGAGTTTGTCAACTCTAAAGTGG | |
| | TAAAAAACAAGACAGATTGTAGACATTTTCATAAAAGGCAGGATTATTTGGT | |
| | GTCACCAAATTAAATATGTTAATGGTCACAGCGGAGGCCGACTCAATCAGCCT | |
| | TTTATTCTGCCAGGCTATTTGATACTGCTGAGGGCTTACCTATTTCCTGAGGAG | |
| | AAAAATAGTCATTAAAATGACCTTATA | |
| hRNU4-1- | GGGCATGAAAGATAAAGTTCTGGAGGTAAGTAGTGATGATGGTTACACAACA | 323 |
| Promoter | ATGTGAACATATTCTATGCCAGTGAATTTACACTTAAGGTTAAAATGGTAAAT | |
| | TTTATGTATACTTTGGCATAATTTAAAAAAAAGTTTGAAAAAGAAAAAAGAC | |
| | AAAACAATAATCGTCTTTGGAGAAGTCTGTTACACTGAATCTGGAGCCAAGTG | |
| | AACAGAACCCTGATTTTGATCCCTTTTCTCTCAAAAGCCCTTCGCAGTCTCTGA | |
| | ATTAAGTCTATTAGCATGTTCCTCCCATAGTGCTTTGCTTCATATCAACAAAAA | |
| | CCTAGCTAAGTGAAATCAGCAACGATATGCAGAAACCACCTACGCAGGTCAC | |
| | AAACATCTTTCTATGATTGTATAATTTTCAAGCAAGCAATAAGTGAAGATTTT | |
| | TCCATAGGCCCTAAACTCACCTTTGCGAAATAGGAAGCTGGTTTATTGGGAGT | |
| | GATGAGCAGGGGGCGTAACAAATT | |
| hRNU5A-1- | GAGACAGGGCTTAAAAGTCCTGGGCAATTAGCTGGGCACAGTGTCGTGCGCC | 324 |
| Promoter | TGTAATCCCACCTAGTTGGGAGGCTGAGGCAGAAGAATCTCTTGAACCCGAG | |
| | GTAGAGGTTGCAGTGAGCCGAGATCGCGCCACTGCACTCCAGCCTGGATGAC | |
| | AGAGTGAGACCCTGTCTCAAAAAAAAAAAAAGTCCTGGGCAGCAAGGCCTCC | |
| | ACTTCACCCCCTAAAGGTTGCCCCAAGAGCACCGTGTGACTGCTAAGGTATTT | |
| | CCGGAGTCTAAAGACGATTATTCAGGTCTCATTTGCATACCCATAATACACTG | |
| | CAAACAGTATTTTTTTCGGAAAAACATTTATATATTGCTTGACATTTTTAAGTA | |
| | TGAGAATTTTGCATGCAGAATTTTTTTGTATAAACTTTCTCAGGTAGTAACCCT | |
| | TGGGATTAGTAGACACCATCAGTGTACTAGGAATTGCAGTTACCCGAAAATTG | |
| | AGTTACAGAAGTAACTGGT | |
| hRNU6-1- | CGACTCGCAACCTTTTCGGGGTCCCGAGTCCAACACCCGTGGGAATCCCATGG | 325 |
| Promoter | GCACCATGGCCCCTCGCTCCAAAAATGCTTTCGCGTCGCGCAGACACTGCTCG | |
| | GTAGTTTCGGGGATCAGCGTTTGAGTAAGAGCCCGCGTCTGAACCCTCCGCGC | |
| | CGCCCCGGCCCCAGTGGAAAGACGCGCAGGCAAAACGCACCACGTGACGGA | |
| | GCGTGACCGCGCGCCGAGCGCGCGCCAAGGTCGGGCAGGAAGAGGGCCTATT | |
| | TCCCATGATTCCTTCATATTTGCATATACGATACAAGGCTGTTAGAGAGATAA | |
| | TTAGAATTAATTTGACTGTAAACACAAAGATATTAGTACAAAATACGTGACGT | |
| | AGAAAGTAATAATTTCTTGGGTAGTTTGCAGTTTTAAAATTATGTTTTAAAAT | |
| | GGACTATCATATGCTTACCGTAACTTGAAAGTATTTCGATTTCTTGGCTTTATA | |
| | TATCTTGTGGAAAGGACGAAACACC | |
| RNU7-1- | CAAAGCCGGTTATGAAGCGGGGGTGGGGTGGGCTAGTTTTAATAGGTCCAGG | 326 |
| Promoter | CGATTAGTACCTGGCATCCTTAACCACCTACAGTTTGAGAAGGGAGTGGGTGA | |
| | TAAAAGCCTGGAAGGGAAGGGAAATCGGGCCGGGCATTAGGCTCCATCGCTC | |
| | ATCAATAGACAAGGCCTTTAGGAAACTGCGACAACGGCTTTTGCTCTGGGCCT | |
| | TTACTGCCGAATCCAGGTCTCCGGGCTTAACAACAACGAAGGGGCTGTGACT | |
| | GGCTGCTTTCTCAACCAATCAGCACCGAACTCATTTGCATGGGCTGAGAACAA | |
| | ATGTTCGCGAACTCTAGAAATGAATGACTTAAGTAAGTTCCTTAGAATATTAT | |
| | TTTTCCTACTGAAAGTTACCACATGCGTCGTTGTTTATACAGTAATAGGAACA | |
| | AGAAAAAAGTCACCTAAGCTCACCCTCATCAATTGTGGAGTTCCTTTATATCC | |
| | CATCTTCTCTCCAAACACATACGCAG | |
| hRNU11- | CACGCCTGCCTGGGCTGCTCCAAGGTGGTGGCTAGGGGTGGCACAAGTTACA | 327 |
| Promoter | CATTTTCAATTTGGATAGGTTGTGATTCTTGTCTTCCCCAGTCAGATACCTACA | |
| | GCCGCTGAGAGCCGGTATTCCAACGCCATGAGACGCCTTCTGTCGTGAGCAGT | |
| | TGACAAGAAAGACCCAGCACTTACTGCAACCGGAAGGAGCGGTTCCTCCCTC | |
| | GCTCGCGCAGCCGCTCTTCCCCGCCACTCCCTCGGTGCCCGCCAGCACATTCC | |
| | CAGCAAGCCCTGAGTATATTTGCATATCAACTCACTACATTTTTTTCTTCTAAC | |
| | TAAAAAATCGAAAGGACAAATTCCAGATTCTCCTTGTGAAGTCTTCCTTTCAG | |
| | TTCAGAAGAAATGGAATTCGCTCTTCAACTTCAGGAAGTTGAAATAAAGAGTT | |
| | GCTTGGATTTTGTGTTCACCTTTACCAAAAAATGGATTTGGTAACACTGCCAC | |
| | CCTGCTTTGGTGACAGAGAAAGC | |
| hRNU12- | TCGAGCTCCACTAGGCCGAGGACCCTAAACCCCGAAGAAAGGAAAGGCCTCT | 328 |
| Promoter | CACCGTCCTTTCGCCGCCGCCCCGCGCTTCCTGATGAGTTCGTCCAGGGAGAT | |
| | GTCCGCCATCTTGCTCCGCCGAGCAAGCCGAAAGCAGTCGAGACCCCGCGAG | |
| | CCCGCCCCTTCGGCCGCTAGGGAGCGACGAACTACTTCCGGCGTCCGCGGACC | |
| | AACTCTCGCGACAGCCAGCTCAAAGCAGGCAAGAACCGGAAGGGGGGGGA | |
| | CGTTCCCCGTGAGCCTTCGCGGTGCTGGCTGCTCATCTGCATACGGAAGTTCG | |
| | GCACATTATGAATTATTTATTTTCCTCGAGGGAAAAAATTAAATGAAAAGCAA | |
| | CAAAATACATTATTAACAAGTGAGACAAACTTCAATGGAACTGGATCATGAC | |
| | CTCAACAGTCAACTACGATAGTCATCATACGCCTAATGAGAATAGAATTCATT | |
| | ACCTAGGAAATAAACTAAAAACGTCCTT | |
| hRN7SK- | GACAAGAGAAGGACTGAACTGGGGGAGTGAGGGAAGAATGCGAGAGTGAGA | 329 |
| Promoter | CTCAGAGAGTAAAGGGGAGGGAAAAAAAAGAAGAGAGAAAGGGCGAGGAA | |
| | GGGGGCGACCGCAGGAGGGAGACAGACCTGGAGTCCACTCGGAGGCCTGGA | |
| | GCCGCACAAAGCGCCAGGTCAGCGGTCCCGGCTGGGTGAGACCAGCAGGCGG | |
| | CTCTAGCGCGCGGGAGCTGGGCGAGGCTCCGGGACGACCTCACCAATGGAGA | |
| | CTGCAGTATTTAGCATGCCCCACCCATCTGCAAGGCATTCTGGATAGTGTCAA | |
| | AACAGCCGGAAATCAAGTCCGTTTATCTCAAACTTTAGCATTTTGGGAATAAA | |
| | TGATATTTGCTATGCTGGTTAAATTAGATTTTAGTTAAATTTCCTGCTGAAGCT | |
| | CTAGTACGATAAGCAACTTGACCTAAGTGTAAAGTTGAGACTTCCTTCAGGTT | |
| | TATATAGCTTGTGCGCCGCTTGGGTACCTCG | |
| mRnu1a1- | CACAAGTAATGTACTTTTAAAATTAACCTTTCTATACCGGTTTTCACAGTGCG | 330 |
| Promoter | GCTGTTTTTGTTGTTCCTCTTAGTGTTAATTCACACTAAAGACTGTGCATCCGA | |
| | CTCCTACATTTATGAAAGTAAATGCCTATTGTTAGAACAAAAAAGGCTACAGA | |
| | ACAAAAAACAAAGCGAAATACCATCTGCTTTAGGTTCAGTGGTATTTTCCCGC | |
| | TGACAGGGAGGCGGGTTTTTGGGTACAGGAAACGAGTCACTATGGAGGCGGT | |
| | ACTATGTAGATGAGAATTCAGGAGCAAACTGGGAAAAGCAACTGCTTCCAAA | |
| | TATTTGTGATTTTTACAGTGTAGTTTTGGAAAAACTCTTAGCCTACCAATTCTT | |
| | CTAAGTGTTTTAAAATGTGGGAGCCAGTACACATGAAGTTATAGAGTGTTTTA | |
| | ATGAGGCTTAAATATTTACCGTAACTATGAAATGCTACGCATATCATGCTGTT | |
| | CAGGCTCCGTGGCCACGCAACTC | |
| mRnu1b1- | AGTCCTTGCTCTTAACTACTGAACCAACTCTCCCGTCCTATAAGTAAATTTTCA | 331 |
| Promoter | GAGCACTAATCTATTCTACTTCAAGTTTCTCCTGCATCAGATCCAGGCCAGCT | |
| | GGAGCGAGCTACACGGTTACCCCCCCTCCTCCCAAAGGGGAAAATAAGGATC | |
| | AGGTGAAAATGTTTGGAAATTACAATAGTAGAATATAAAGAAAGAATATGAA | |
| | GGCGGCCATGAAGGGGTAGAAAGGTAGATGACCAGGCGTCATCCCTGATGTC | |
| | ACGGGTATGGGCGTGGTGTATGCAGATAAGCGCGCAGGGTCTGCCGGGTCCG | |
| | CGCTCCGTGCAGAAATGTGAGGGGTTTCTCCCGCGGCGAGGGAGGGAGCGCG | |
| | GATACGCGGGTGTCAGCAGAGCGCAAGAATGTCGTGTGCGACGAGGGAGACC | |
| | CGAGATCGGCCAGGGCTAAGTGACCGTGTGTTAAGAGTGTAGAGGCGACGGG | |
| | TGTGAGCCGGGGCCGGGCAGTGGGAAAGC | |
| mRnu1b6- | CGGTGGGGCTGGCTCCTGTGCTGCCCGACTCCTGTGCTCCGACTTGGAGAAGA | 332 |
| Promoter | CGGGCGTCCGTTCGTCCATACCCAAAAGAGTCGGCCACAAGGATGTTGGCAC | |
| | CTGGGGGTGGGCCACGAGCGGCCAGTTAGGGGTAAGAGACGGTGCAAGGTG | |
| | ACTTGTCCCTGGTCCTTCACCGCTCCAGCACTGCGCCAGTGAGAAAGTCCAAC | |
| | AGGCCCCGGGATCGGGCGGCGAAAGGAAGGGGCGTGATGTCACTCCTGGGTG | |
| | GCTGACGGGGGGGGGTGTATGCAGATGAGGGTCGCGCAGGGGCTGCGGCGTC | |
| | CGCAGCGGCCGGGGAAACGCGAGAGATTTCTCGCGCGGCGTGGGAGAGAAC | |
| | GCGGGTCGCCAGATGTCAGCGGAGCGCCGAGACGGCGTGTGCGACGAGGGA | |
| | GACCCGAGATCGGGCGGGGCTAAGTGACCGTGTGTTAAGAGTGCAGGGGCGG | |
| | CGGGTGTGAGCCGGGGCCGGGCGCTGGGAGAGC | |
| mRnu2-10- | GCTCCTCCTAGGAGCTAACACTGGAGCCAGGGGGATCAAAGCTGTGTAGTGA | 333 |
| Promoter | GTGTCACAAAACATCCTAGCACTAAGGAGACTGAGACGGGAACATTGTAGTA | |
| | AGTTCTAGCCTGGGCTACAGTAAGAGACGTAGTCTCAAACAAGGAAATGCGC | |
| | TATAAATAAATAAGATGGAGCTTCAATGGTCTGTGCATAACTGGGGGGTTGA | |
| | AGGAGCCTTCCAAAGGCTGGGTTCCGGCTCCGTTCAGTCGTTGACTTGAGCTG | |
| | GGCGGGGCATGCAAATAAGGACGCCGCCTGGTCTCCGCGGAACTCTGGGAAC | |
| | GAAAAGCGCAACAAAATCCTCTCGGCCCCCTTGGATTCCTCGGGCTTTCCCTG | |
| | GCGTAAAGGTGGTGGACAGGATAGAAGAGGAGTTTCGGCGTCGGGCGCGTGT | |
| | TTGCGGTCGCGCGAGTGTTGGCGACCGTGAGTTGAAGGTGTGTGTGGTGTTGA | |
| | GTGTATGGGGCGTGTGGGCGCAGTTCGGT | |
| mRnu3b1- | ACAGAATTTACTAAATAGAGAGTGCAGTCATGGAATTGCCATTTTCCCCACGT | 334 |
| Promoter | GGTTTTTCGTTTGTTCCTCACCGGAGGCCTCCGGTTTGAGCCCGAAGTCTAAA | |
| | CCGGTGCGCGTCTGCGAGCTCGCGTGCGCCTCCGCGGCCGGAGCACAGTCGC | |
| | GGGCGGGCATCAGGGCGGTGCGTGGCCGTCAGTGACGCATGACCCTTCGCAG | |
| | GCAGAAGTGGGACGGGTCAAGAGAGGACTGTGATCCGATTGGCTGTTCGGCG | |
| | ACCGTATGCTAATCGCGGTGACAGTCTGCGTGGTGTAGGGACACGGTAGCGA | |
| | CTCAAAATGCCCCGTGGACGGGGTCTGGAGAGGTGTCGGTCTGTCAGAGAAG | |
| | GTGTCCCCGCGAGGGCAGACGCGGTGTGAGATAGAGGCGCGGACAGGGATGT | |
| | CACGGGTCGTCTGTGCGTCAGTCGCCGTGACTAAGATGTGGGGGCGGCCGGT | |
| | GCAGTGCGGCGCGGTGTGGAGCGTGAGGTTT | |
| mRnu5g- | TTATCTACGTTGGTTTTACTACAGAAACTACTGTTCTCAGTAAAAGCAGAGAG | 335 |
| Promoter | AGTACAATGCCAACAGCTTTGAAATCCTCTGTGCTCTTGTGCCAATCCCACCC | |
| | TCAGAACAGGGAGTCTGTCGGTTATGTTTTCGTCCTTTACAGTTCTTCGCCGTC | |
| | TAATCCATTGGAAACCTCCTTTATTTAAAGGAAACCGTGTCCAAATTCAGTGT | |
| | ACAGAGGTGCAAACCTCCGCCCCGCTCCTTGTAGGAAGCCCACGGGAACTGG | |
| | GCCAATCGGACGGCGCTTACTCGGCTCATTTACATACCCATAACACACCGCGG | |
| | CTAATGCAAATATTTTCGTGAAAAGATCTTCCATTACTCGAAGTTTTAAAACT | |
| | AAAAACAAAATATTATTTCAAAAGATCATGAAGAAATTTCTGTCGGTAACAG | |
| | CAGTTTCAATTGATAAATCACCATCACTTATCTAGGAGGTTCTGTTACTCTAG | |
| | AAGTGAATTAAGCAGGACAGCTGT | |
| mRnu6- | GGCATGTGATTTTGTTTTGTTTAGGTTTTTTTTTAACTTTATTTGATCAACATGA | 336 |
| Promoter | AATAATACAATAAGCTATGTCTTCTAACCAGCAGGCAGCCAGCCAGGAGCCA | |
| | TGTGTAGCAGTGTTAGAAGCTGCAGCAGGCATGTGGGTAATTGTGGGGAGCT | |
| | TTTAGGAACAGCTAGGGAGCTGTGTGGAGCTGCAGAGATGCAGAGGAGCCTG | |
| | GAGGAACAGACCAGTGCTGTGGGCTGGGAGGAGCCCCCCTCACAGGTACTTG | |
| | CTCAACGGCTCCACGACCATAAGCCTTGAATAAGTTACATGGACATTTTACAA | |
| | ATGGCACAGGATTGATTATGTCTGCTGCCAGAAGCCTCATGTCGATAAGGAG | |
| | AGAGCTGGCCAAAAGGTAAAGTATGCTAGCTCCATACAGTGCAATAGCCACG | |
| | TAGTCTCACAGAGAAAGAGGCAGGCCTGCATCATATCCAAATATGGCCAAGA | |
| | ATTTTATTAAATTAAAATGCAAACAGCA | |
| mRnu7- | TATTTTTAATGAAGCAGGGCCATGGAGCCCAAGCTGGCCTTGAACTTCTCAGC | 337 |
| Promoter | TTTTGTGCTGGTATCACAGAAATGTGGCGCTATGCCCGGTCGGGCCTGATGTT | |
| | CCAGAGTCTTTGCAGTGTGGGGAACTTGTAAGGATGAGACTACAAGACATCG | |
| | GGCCACATCGCCTGCCACTACTTAAGTCCGATTCACTTCGGCTTTAGCTCCAA | |
| | GCCTTTAATCTCGCGAAGCTCTTTTTTTTTTTTTAACAACATAGGAGCTGTGAT | |
| | TGGCTGTTTTCAGCCAATCAGCACTGACTCATTTGCATAGCCTTTACAAGCGG | |
| | TCACAAACTCAAGAAACGAGCGGTTTTAATAGTCTTTTAGAATATTGTTTATC | |
| | GAACCGAATAAGGAACTGTGCTTTGTGATTCACATATCAGTGGAGGGGTGTG | |
| | GAAATGGCACCTTGATCTCACCCTCATCGAAAGTGGAGTTGATGTCCTTCCCT | |
| | GGCTCGCTACAGACGCACTTCCGC | |
| mRnu11- | GTTCTTTGCTTTTACAGACCGTCTCCTGTGTGTCCCTGTCAGAGACCTGAATTC | 338 |
| Promoter | GGTTCCTAGCACCCATATGTTAGAACATAACGTCCAAGTTGGAGGCTACAAGT | |
| | GGCACTAAATTACACATTATCAGTTTTGACAGGTTGGGGCACCGTGTAACCCT | |
| | AGTAACTTAGCGAAAGGCTCCTCGAACCCAACATCCCACCTCTAGCTGGGAG | |
| | CAGCTGTTTTGCACCAGCGCCGCCTACCTCTCCCTCCGCGTCGGCCAGAGCAT | |
| | TCCCAGCAAGCACTGCTCTGATTTGCATATTGACTCACGGACTTTCCTTTACTG | |
| | CTATACAAAAAACGACACAACAGTTCTCTTTGTGAAGAGCTTTCCCTCAGCCC | |
| | GAGAAGAATCGATAGTTTGCCTTTTAGCTTCGACAATATACAGTTGAAAGTTG | |
| | GTTAGACTTTATTTTCACCTTTACCGTAAGATGGGTGGACCGACAATGCCACT | |
| | GTGCTTCCGAACGCGCTCGAGC | |
| mRnu12- | GAAGCAGCGAGGCCCCTACGAGCCTCGAGGAAAAGGGAAAGGGCCGCTCAC | 339 |
| Promoter | CGTCCCTTGGCCGCCGTCCCGCGCTTCCGGATGAGCTCGTCCAGCGAGAGATC | |
| | GGCCATCTTGCGGCGCGCAGCTAGCGGAGAACGGAGGAGAAGCCGACTGCGG | |
| | CGAGCCCGCCCCGCGGCCGGAGTCAAGAGCGGCTTCCGGCAACCCGGACGGC | |
| | CAACTCTCGCGACAGCTCAAACGAAAAGCTCGAGACGGAAGGGGTGGAGCCT | |
| | CTCTCCCCGTGAGCCTTTGCGGTGCTCGCTTCTAATTTGCATGGAGGAGTCCG | |
| | GAGCCTTATGGGCTCTCTCTCCCCCCTCCTCCTTTTGCTAATGAGGAATTAGAA | |
| | TTATATCGATCATAAACCATTAAAAAGCATTATCAAGAGGACAAGTCTTTTAG | |
| | TCATACAGCCGATCGGACACTCACCATACTTGTAATGAGAATATAATTTCTTA | |
| | CCCCTGAGGAACGTAGACACGTTCCTC | |
| mRn7sk- | CGCTTCGGTGTGTCACTTCGGCCACAGAGGGAGACAGACGATTACGGCCCAC | 340 |
| Promoter | GGAAGGCCGCGTGAGTCTCGTGAAGCTGGAGATGCGCTGGGTCTACCTTTCGC | |
| | CGTCCGCTCCGCCTTGGCAGAGGCGCCGACACGCACCGAAGGCAGAGAACCC | |
| | ATCTCGTATGGGACCGCACCGGACACGGGCGCCTGGGCCAGGAGCAGAGCCG | |
| | GGCCGTAGAATAGACATGGCCGTCGGGGGCGGGGCTTCGGAAGGTTTAACCA | |
| | ATCCAAACTGTTGTATTTTGCATAGCCCCAAAGCATTTTGGTTAACAGTAAAA | |
| | ACATCCTAAATTTAAGTATTTTAATTTAAACTTAGAACGAAGCGAGTATAAAA | |
| | AGGATTATTTAACCCTAAAACGGATTCAGGATTTGTTATAATATCAAGTACAG | |
| | TCGGCTACATAAGGTCACCACATGTGTAAAGTTACAAAATTCTATGGCCTTAT | |
| | ATACCTACCAAGAGCCTGCTTACTCTC | |
| Mm U7 3′ | Cccaatttcactggtctacaatgaaagcaaaacagttctcttccccgctccccggtgtgtgagagg | 9 |
| Region | ggctttgatccttctctggtttcctaggaaacgcgtat | |
| Hs U1-1 3′ | ACTTTCTGGAGTTTCAAAAGTAGACTGTACGCTAA | 10 |
| Region (U1 | | |
| terminator) | | |
| hRNU1-1- | ACTTTCTGGAGTTTCAAAAACAGACTGTACGCCAAGGGTCATATCTTTTTTTGT | 341 |
| Terminator | ATTGGTTTGTGTCTTGGTTGGTGTCTTAGGTGTTAATCCTACAGTGGAGGGTTT | |
| | ATGGGAATAGGAAGTAACATGTCGCCTGCATGTCATAGGAGAAAAAGCGAGC | |
| | ATCAGCCGTATCGGCTTTGTAACACAAATTAGCTATCGTGAAGTCCGCTCAGC | |
| | TCTTCCCTTTCTACCCTGGCTGCTTTTTGCAGGGATTGGTCCGTGGTCTCCAGT | |
| | CTCTTGGGTTCTCACCCTGTGTGAAAATCTTCGTGTTTTTCCCTACCCCCCAGT | |
| | CACCTCTTACACAGCCTCTGCTTCCAAGCGCAGCCCCCACAGGAGTTTGTAGG | |
| | ATTTCTGTGCTAGCGGGGAATGTGTTCTCACCTCATAGAGCCAGGTAGAAATT | |
| | ATGCAGATGGGCAGTTCTCTGGGAAGAAAGCAGGGCCTTTGGGGCTCTCAGT | |
| | GTCCCCGTTGGGTTGTAGACA | |
| hRNU1-2- | ACTTTCTGGAGTTTCTAAAAGTAGACTGTACGCTAAGGGTCATATCTTTTTTTG | 342 |
| Terminator | TTTTGGTTTGTGTCTTGGTTGGCGTCTTAAATGTTAATCCTACAGTGGAGGGCT | |
| | GGCGAATAGGAAGTAACATGTCGCCTGCACGCCATAGGAGAAAAAGCGAGC | |
| | ATCAGCCGTATCGGCTTTGTAACACAAATTAGCTATCGTGAAGTCCGCTCAGC | |
| | TCTTCCCTTTCTACCCTGGCTGCTTTTTGCAGGGATTGGTCCGTGGTCTCCAGT | |
| | CTCTTGGGTTCTCACCCTGTGTGAAAATCTTCCTGTTTTTCCCTACCCCCCAAG | |
| | TCACCTCTTACACAGCCTCTGCTTCCAAGCGCAGCCCCCACAGGAGTTTGTAG | |
| | GATTTCTGTGCTAGCGGGGAGTGTGTTCTCACCTCATAGAGCCAGGTAGAAAC | |
| | TACGCAGATGGGTGCTGTTCTCCAGGAAGAAAGCAGGGCCTTTGGGGCTCTC | |
| | AGTGTCCCCGTTGGGTTGTAGA | |
| hRNU1-3- | ACTTTCTGGAGTTTCAAAAACAGACCGTACGCCAAGGGTCATGTCTTTTTTCG | 343 |
| Terminator | TATTGGTTTGTGTCTTAGTTGTTAATCCTACAGTGGAGGCCTGGGGAATAAGA | |
| | AGTAACATGTGGCCTGCACGCCATAGGAGAAAAAGCGAGCATCAGCCGTATC | |
| | GGCTTTGTAACACAAATTAGCTATCGTGAAGTCCGCTCAGCTCTTCCATTTCTA | |
| | CCCTGGCTGCTTTTTGCAGGGATTGGTCCGTGGTCTCCAGTCTCTTGGGTTCTC | |
| | ACCCTGTGTGAAAATCTTCGTGTTTTTCCCTACCCCCCAAGTCACCTCTTACAC | |
| | AGCCTCTGCTTCCAAGCGCAGCCCCCACAGGAGTTTGTAGGATTTCTGTGCTA | |
| | GCGGGGAGTGTGTTCTCACCTCATAGAGCCAGGTAGAAATTATGCAGATGGG | |
| | CGCTGTTCTCTGGGAAGAAAGCAGGGCCTTTGGGGCTCTCAGTGTCCCCGTTG | |
| | GGTTGTAGACATAACACTCTTA | |
| hRNU2-1- | CCTCCGGGGATACAACGTGTTTCCTAAAAGTAGAGGGAGGTAAGAGACGGTA | 344 |
| Terminator | GCACCTGCGGGGCGGCTTGCACGCCGAGTGCCTGTGACGCGCCGGCTTAACTT | |
| | AACTGCTTCCCTGAAGTACCTTGAGGTTCCTGATGTGCGGGCGGTAGACGGTA | |
| | GGCTTATGCGGCACGCTGTCGTTTCCACCGTGGCTACTGCGCTTTGGGAAGGC | |
| | CACGACCTCCTCCTTTGGGGAGGTCCTTAGGATCTCAGCTTGGCAGTCGAGTG | |
| | GGTGGCGACCTTTTAAAGGAATGGGACCCACCCGGAGTTC | |
| hU3- | TTCCCTTCTTGTAATACAAAATAATAACAACCACAGAGGACATCGCAAGTAA | 345 |
| Terminator | AATGTGTATAACAACCCCATCTAAAGAACCCTATCATCTTTCTACTATCTGTCT | |
| | ACCTAAATATGTATAGTATAAATAACCCTATATAGCACTCTAAGCAACTTAAG | |
| | TTAGGTAACTTAGAAAACTCTTTTTTTTTTTTGAGACTGAGTCTCACTCTGTCG | |
| | CACCCAGGCTGGAGTGCAGTGGCGTGATCTCGGCTCACTGCAACCTCCACCTT | |
| | CTGGGTTCAAGTGATTCTCCCATCTCAGTCTCCCAAGTAGCTGGGATTACAGG | |
| | CACGCCACCATGCCCTGCTACATTTTTTTTTTTTTAGTAGAGATGAGGTTTCAC | |
| | CACATTGGCCAGGCTGTTCTCGAACTCCTGACCTCAGGTGATCCGCCCACCTT | |
| | GGCCTCATGAAGTGCTGGGATTACAGGTGTGAGCCACTGTACCCAGCCAGAA | |
| | AACTCTTAAAAAAC | |
| hRNU4-1- | CTGAATTTTCTTGCAGTTGAACAACAGAGGCTTTTTTTGTGTGTGTGGGGGTG | 346 |
| Terminator | CTTGGTTTTGGGAGGTTGAAGAGTACTTGTTCGCAAACTCTCTAAATGAGAGA | |
| | TGGGGAAGTGTCTTACCAGCTGATTCGCGAGAGCACCTGCAAGTTTACCTGTT | |
| | TTAGTGATTTGCGTTTATGAAAGTTGGAGTTGCTGATTTTGTGATCTCTACTTT | |
| | TAAGGTAAAAGCAGGGCGGTGTTGACTTTTCTTGCCAGTTGGCTGTCCTGTTT | |
| | TCTTTCTCGCACTGTTTTCTTTTTGAGACGAAGTCTGTCGCTCAGGCTGGAGTG | |
| | CACTGGCGTGATCTCGGCTCGCTGCAAACTTTGCCGAACCCCTGTTTAGCGAT | |
| | TTCCGTTTG | |
| hRNU5A-1- | ATATGTGGTAATCCAACAATAGAAATTATTTTTAAGTTTGTGTGTTCCTTTTTC | 347 |
| Terminator | TGTTCAATGGTGCTTTTGATATTGTTGTAAAGCAGTGACTAGCAGATTCTGTGT | |
| | GGTAAAAGCACTGAACCCTGTTTAGTTCTTTTCTCCCTACCTTATTGGTAATTA | |
| | TTTCTGTGTATATATGTTCCAAAGAAATGCGGCTTGTATATTCATGCTTTTCAA | |
| | CTTTTTGTGATAGACAGTTGATACACAAGTAGTACCTCCTCCGGGATCTTCTTA | |
| | GTCAAGACTGTTTGGTAGATGCATGTTTTTTGTTACTTGTAATGTACAGTATTC | |
| | AGTTCATTTTCTTTTTGCAGCACTGTCCGGTTGAAACACAACGCGAGGATCGG | |
| | GCGTGGTGGCTCACGCCTGTAATCCCAGCACTTTGGGAGGCCGAGGCAGGAC | |
| | AATCAAGACCAATCCTGGTCATCAAGGCAGGAGAATCAAGACCATCGAGGTC | |
| | AGGAGATCAAGACCATCCT | |
| hRNU6-1- | ACATCAGGTTGTTTTTCTGTTTTTACATCAGGTTGTTTTTCTGTTTGGTTTTTTT | 348 |
| Terminator | TTTACACCACGTTTATACGCCGGTGCACGGTTTACCACTGAAAACACCTTTCA | |
| | TCTACAGGTGATATCTTTTAACACAAATAAAATGTAGTAGTCCTAGGAGACGG | |
| | AATAGAAGGAGGTGGGGCCTAGGCAGATTCATCTCTGCGGTGCATTTTGCCTC | |
| | TGGCCCTCGGGCCCTAGTTTGCGATCTTTCCCATCTTGCCTCTGCTTCCCTCTG | |
| | CACAATCCTGAGTATTGCAGTACCTTACACTTAAGGAAATTTCGTCTTGCCAA | |
| | AGTTGATCCATTTCAGCTCCGAAACATAACAGGAACTAGTGCAACGATAAAC | |
| | GTATGAGGAACTTGCAATAAGAGTGACAGAATCCTTTGAATCAAAAAAAAAA | |
| | AAAGCCACCACTTAATGAGTATCTACCATATGTCAAGGGCTTCACCAATATTT | |
| | CATTGAAATATTCCAGACCA | |
| RNU7-1- | CTTATGATGTTTGTTGCCAATGATAGATTGTTTTCACTGTGCAAAAATTATGGG | 349 |
| Terminator | TAGTTTTGGTGGTCTTGATGCAGTTGTAAGCTTGGGGTATGAAGGTTTGGGCC | |
| | ACGCCTGGGCGCTTCCGGCTGCGCCGGATGCTGTTTCCTTTCCGCTCCCAGGG | |
| | GCGTTGGGAACGGTTGTAGGACGTGGCTCTTTATTCGTGAGTTTTCCATTTACC | |
| | TCCGCTGAACCTAGAGCTTCAGACGCCCTATGGCGTCCGCCTCGACCCAACCG | |
| | GCGGCCTTGAGCGCTGAGCAAGCAAAGGGTGAGAATCGTCCTAGTCAAGGCA | |
| | TAGGCTGCTGGCCTGGGGTAGTCAAGGCATGGGCTGCTGGTCTGGGGAGGAT | |
| | GCGGGCGGAGGATGTGGGGCGACAAACCTGGCTACGTCCGCCGGGAAAATGG | |
| | GGTAGGGGACGCCGGGTACCGTCCTTCTAAGTGGGGCGCTTGCCCCCAAGAC | |
| | TTGGGATCTTATTGGGTTACCTACA | |
| hRNU11- | TCTTTACTGTTATATGTTAGGCGAAATATTACGCGTTTGGAGTAAGTGGTGCTT | 350 |
| Terminator | TTTGTAACTGAAAAGAGATTCTGTGTGTGTTTTTTTTTTTTTAGAGGCTGCATA | |
| | GTTATTTGTATGAACCGCAGGTGACCTAATTAGCAAGGTAATTCAGACCTGTA | |
| | TCCTAGCACTTAAGAAGTGGAAGTGGGATAACCCTGCGTGTTCTCGTGTGGGA | |
| | GGCTAGCTTAAGCAACAAAGCAGGAAGGCCCCACCAACAAAAGAATTTAAAA | |
| | ATTATACGTGCGTGGTGGCGCGCGTATGAGGAAATTATTTGGCAACTGTGTTA | |
| | CAGTATAGCAGCAAATGGAATAGACCTAGTCCCTGACTTTAGGAGACCTGCCT | |
| | CAGCCCCCCGAATGGCTGGGACTACAGGCGTGCGCCTCCGCGCCCAGCTAATT | |
| | TTTGCATTTTTGGTAGAGACGGGTTCACCATGTTGGCCAGGATGGTCTCCATC | |
| | TCTTCATCTGGTGATCCGCC | |
| hRNU12- | ACCTTATTCACGCCTAAAAAGTAGACTGACTGTGGGGTGGTCGTGTTTTTTGT | 351 |
| Terminator | TTCTTGTTGGTAGGTGGTGAATGCGTTTTTTTCGTTGTTTTCTCCGTTACTCAG | |
| | GCTGCCAGTTGCTTGGCAGTCTTGTCGCTGGCTGTGGACGCTCTGCACTTCATC | |
| | ACCGTCCCCAGCTGCGCTTTGAGCCGGTCTAAGGTTGCCCACAGTATTGACGT | |
| | TGGGACCCACGAAACTGGCAATTTCCATGTGTGCGGCCTTACCCAGTTCTCCC | |
| | AAACTTCTGCGTATAACCGCTTCTCTGTGCTTCAGAAAGATAAGTCCGGCCCC | |
| | GGCCTCACACAGCAGCTCAGTTCGTGACAGCGTTTAATATGTAAAATGGAGC | |
| | AAATAATTTATAGGGCGTTGGGAGAGTTAAAGAGACTAATTCATTTACGTGTT | |
| | AAATACAGGGAATGGCTTTTGCATTGATATAACCCCAGCCTCCAGCATGCGCC | |
| | AGACCTGACGTAGGTGTTCGGC | |
| hRN7SK- | TTTGACCCATTACCCATCTAAGTTAGATGCTTTTTTAAATGTTTTTTAATTTTTA | 352 |
| Terminator | AATTTTTAATTTTTTTCATTATTTATTTTTTATTTTTGAGACGGATCTCGGCTCA | |
| | CTGTAACCTCCACCTCCCGGGTTCAAGCGATTCTCCTGACTCAGCCTCCCGAG | |
| | TAGCTGGGATTACAGGCGCGCGCCACCATGCCCGGCTAATTTTTCTATTTTTA | |
| | GTAAAGACGGGGTTTCGCCATGTTGACCAGGGTGGTCTTGAACTCCTGACGTC | |
| | AGGTGATCCTCCTGCCTCGTGCTGGGATTGCAGGCGTGAGCCACCGCTCCCGG | |
| | CCCTTAGATGCTTTTTAATCAGCAAGTACGCAATGCTTTCCTGACAGGAATGT | |
| | CAAGTCCGTCAGGAAATCCACATAAGGGTGTTAAGCAGGCAAAGTACCACAT | |
| | ACAGATTAAAATCAAAAGCACTGTCAACGCTTATTTAATTTATACAGTAGACC | |
| | CTAACTTTTCTAATTTTGTG | |
| mRnu1a1- | GTTTACTTGGTTTTAAAAATAGCTTGCACTAGCGATACCGCGAATATGGTTAT | 353 |
| Terminator | TAGGTTTGTTAGGCACAGTCGTGTCTTACTATAGAAAAATAACGTAGTGTTCA | |
| | TTTTGCTGCCTGTATGTGTTAATTTGTCCTTATTGCGCCATTGTTCTTGTTAAGT | |
| | CTTCTGTAAGGAGTTGCGGGTTTCAAACTGTCAGTCTGAGAGCATAAGCTCAC | |
| | TTTTCAGTCAGTTGAATTGAATGTTACATTCGATTGGTTCTGGATATTGATATT | |
| | ACCCAAGGCTTGTCCTGTTTGTAAGTCTTTTCCTACACCTGACAATATGATTAT | |
| | GTTTACAATGTCCCTAGAACTCTGGGTGTATCGTTCCCACTGAATAAAATTTG | |
| | ATCTCTAAGTTGACCTATAAATTTAAGTACCAAAAAACTGGTTTGTAAGATTT | |
| | TGGAAGGCAGAGGCAGGGAAGAGACCAACGTGGTCTACATATAAAGTACCA | |
| | GGTACATATAAAGTACCAGGT | |
| mRnu1b1- | ATTTTTGTGGTGCTAAAAGTTAGATTGCATTCCGCCCTTCTCACGTCTCTTTAC | 354 |
| Terminator | GTGTTGTTTGCGAGGCGTGGCCTCGGAGGTAAATGGTCTCCTTTTACCTCAGA | |
| | TAAGCTTACCTTTGCTCTGTGAGTAAGAATGTAGCTACACTTCCAAATTTAGC | |
| | TATTGTCCTTTGCGGTCCAAATGAAAATATTACCTTCCGATCTAACAATGTTA | |
| | AACTAGTCCCAGGCCCTTCTATCGATTGCCAGAATAGACTAGACTACTACACA | |
| | GACACACCCTCTCAAATCAGGCCAGCTCCTGCAACCTGCACCTACAGTTTGTC | |
| | CCCTGGGACAAATGAAAGGACAGGCCCTCTTTCCCCCCCCCCCCCAAATCAGA | |
| | GCACACACAGATCTTGGGGTCTGGGGCAGAAATTTCGTTCATGGAAAAAACA | |
| | AACAAACAAACAAACACGATCAAAGGAAGCTCGTTCCCTTTGGCCTCAGGGT | |
| | TTCTTAGACGCCGTGTTCCC | |
| mRnu1b6- | ATAACTGGTAACCAAAAGCAGATTACTTTCCCCCTTGTGGTCTTCGCGTTTGC | 355 |
| Terminator | GGACGGGAGAGAACTTTCGGCCATTTTCCTTTTAGAAATTTAACTTCTTGAAG | |
| | GCCACCAGGTGACCCTTACTTCCCTCTCACCTTTCCTTCTACCTGTACTTTCTCT | |
| | ATTAAAAAGCCTGCAGAAGTACCCTTGACAAATTGCCAAAAATGACACCTAA | |
| | AAAAAAAAAAAGGAAAAACGACACCTTAGGTAATTATAGGTTGTAAGAAAA | |
| | AGATTTGACCTCTTGGAGTTTTTATTTCTTAATACCAAAGGACACCCAAGGAA | |
| | CGTATCTCTCAAAATTGGGTTTCAAACCTCAGCCGTTTCCTTCTTAGTTCCAGT | |
| | GCTTAACACGCAACAGACCTGCACACCACTACACTGCATACGAGTTTTAAAGT | |
| | TAATAAATCAGTCACCCCTCCATAGTCTTCTACCTACTTGAAGACAGAAACGG | |
| | CTGCTAGGAAGCAATTCTTC | |
| mRnu2-10- | CTGGGGAATAAGAGTGGTTTAAAGAAAGAACAATTTGAGAGATCTGCTTAGA | 356 |
| Terminator | TCTAATAAAACCCAAGTATAGTTTCTTTAGACGTCTTATATCTCACGTAGGAC | |
| | CTCTTATTTCAGACTCACTGCAAGAACAAGCTAAAGAACTATATACAGTCAGG | |
| | AACAAGGTGAGCCATCGCGGTTCTCAGTCCCATGATCTTAAGAGTTTTTCCTC | |
| | TTCCCCTCTTCCCTGGCTGTCCTGGAGCTCACTCTGTAGACCAGGCTGGTCTCA | |
| | AACTCAGAAATCCACTTACCTCTGCCTCCCAAGGGCTGGGATTAAAGGCATGC | |
| | ACCACCATTGCCCGGCAGGGTATGTGGACTATGCTGTCCTTCAACTCAGAGAT | |
| | CGTGGTACTTTAAATCTCATTTTCCGAAAGAAAAGAAAAGAAAGGAAGGGGG | |
| | GAAATAAAGCCAGTATATTTTGCACCCACAATCTGAAGTTCTACAAATATTGT | |
| | TAGAAAATGCAAAGGACCCAAGTC | |
| mRnu3b1- | CTCGGTTTATCTTAAAGGGTTTTAAATATAGAAAGCTTCCTTAGTCTTATGGGT | 357 |
| Terminator | GAGAGTGCTGTTTAAGCCTTACCTAGGTATGTCTTTGAACATTTATTACAGATT | |
| | GTTATTTGTGACTATGACCACTCAGGTGTTCTGTTCTTGTGAATTTAGACACAC | |
| | TCAGAAAGGCTACACACTCAAAGACCACACCAAAGTGAAGTACTATCTGGGC | |
| | CGTGAAACTGGTCACATATCAGTTATTTTGCAGTAAAACAAGGAAGTCAATCA | |
| | TACTGCTTCAGTGGGTCAGGGAGTCAAGAATGCACAGTACAAGAGCTGGTGA | |
| | GATTCCTCAGCAGGTAAGAGCACTGAATGCTCTTCCAAAGATTCTGAGTTCAA | |
| | ATCCCAGCAACCACGTGGTGGATTTGAACAAAGGATGAAAACAACCATCCAT | |
| | AACCAGATCTGATGCCCTCTTCTGGAGTGTCAAAAGACAGCTATGGTGTACTT | |
| | ACATAAATAAATAAGTAAAAACA | |
| mRnu5g- | CCACATTTTGTGTTTAAAAATAGAAATATTTAAGTGAGATCAGTTTAAATCTG | 358 |
| Terminator | CTTTATCTAGGGTGTCTAACTGCTTGCATCTTTTTAACTTTTCCTTATTTGTGAG | |
| | AAGGTCTGTAAAACTTATTATATGTTAGTACACTGTAGCTGTCTTCAGACACT | |
| | CCAGAAGAGGGATTCAGATCACCTTACAGATGGTTTTGAGCCACCATGTGGTT | |
| | GCTGGGATTTGAACTCAGGACCTTTAGAAAAGTAGTTGGGTGCTCTTACCCGC | |
| | TGAGCCATCTCACCAGCCCTTTTTTTTTCTTTTTTCCAGTTCGGGTTTCTCTGTG | |
| | TGTAGCCCTGGCTGTCCTGGAACTCATTCTGTAGACCGGGCTGGCCTCGAACT | |
| | CAGAAATCTGCCTGCCTCTGCCTCCCAAGTGCTGGGATTAAAGGAATGTGCCT | |
| | TCATTGCCCGGCTCTAGGGCTTTTTGTTGTTGTTTGTTTGTTTGTTTGTTTGTTT | |
| | GCTTTTGTGGATGGCCT | |
| mRnu6- | AAAACCAGTAAGGGAGAAAGGTCAAGTAACATATAAAGGAAGGCCTATCAG | 359 |
| Terminator | AATTACACCAGACTTTTCACCAGAGACTATGAAAGCCAGAAGAGCCTGGACA | |
| | GATGTTATACAGACACTAAGAGAACACAAATGCCAGCCCAGGCTACTATACC | |
| | TGGCCAAACTCTCAATTACCATAGATGGAGAAACCAAAGTATTCCACGACAA | |
| | AAACAAATTCACACAATATCTTTCCACGAATCCAGCCCTTCAAAGGATAATGA | |
| | CAGAAAAGAAGCAATACAAGGACGGAAATCACGCCCTAGAACAACCAAGAA | |
| | ACTAATCATTCAACAAACCAAAAAGAAGACAGCCACAAGAACAGAATGCCA | |
| | ACTCTAACAACAAAAATAAAAGGAAGCAACAATTACTTTTCCTTAATATCTCT | |
| | TAATATCAATGGACTCAATTCCCCAATAAAAAGACATAGACTAACAGACTGG | |
| | CTACACAAACAGGACCCAACATTCTGCTGCTTA | |
| mRnu7- | CCCAATTTCACTGGTCTACAATGAAAGCAAAACAGTTCTCTTCCCCGCTCCCC | 360 |
| Terminator | GGTGTGTGAGAGGGGCTTTGATCCTTCTCTGGTTTCCTAGGAAACGCGTATGT | |
| | GCTAGAGCCACGCTCTGAGACTTCCGCCTCGTGCGGTCCCGCTTCCTTTCTGCC | |
| | TCCTCTGGCCTGCATCCGTGGGGGAGGTGGCTGGCTGCAGGGTTCTCCGGATC | |
| | TCTTCTGTGTTCCCACTACTCAAGCACCGAGTGGCGTTCTATGGCGTCCGCCTC | |
| | GGCTCAGCCCGCGGCCCTGAGCGCGGAGCAGGCCAAGGGTGAGGAAAGAGC | |
| | CTAGTCTGGGTGTGGGCCGTCTTCTGGGGGGAGGGCGCGATACTATTCTTTGC | |
| | CTCTCCAAGTGGGGCCCTTGCCCATTAGGGCGAAGGGTTGGGAGGACAAGCT | |
| | TATCGGGTTGCTTGCAGGCCTCGGCCCACTAATTCCCTGCCTCTTCCCTCCTCT | |
| | GGGCTCGCCCGCCGTCCTCCGCG | |
| mRnu11- | GAATTTGTTAAATTACAGGAGGAAGCTTTTTTAGTTCATTTTACAAATACTTTC | 361 |
| Terminator | TTGTTAGGTAGATAATACCTTCTAAGGTTATTATCTGGATCTTCTAAGTCTTTT | |
| | TATCGCTAGCAAGTCTAAGTTTCTGGACTTTGGAATTAGAAGTTAATGTCGTA | |
| | GGCGGGCATGGTGGTCCACGCCTTTAATCCCAGAGTTTGGGAGGCAGACGCA | |
| | GGTGGTTTTCTGAGTTCGAGGCCATCCTGCTCTTCAATGTTAGTTCCAGGACA | |
| | GCCAGGGTTATACAAAGAAACCCTGTCTCGAGAAACAAAACAAACAAAAAA | |
| | AGAGTAGCTGAGTGCTCTGAGTTGTCTCTCCAGCCCCTTAGCTATTTATTTTCA | |
| | TTTAAATTATTTTTGTTTTATTCGCTTGTGTGCCTGAGTTTGAATTTCCCGGGAT | |
| | TAACAGTTGAAGACTAGGGCTGGTGAGATGGCTCAGCGGTTAAGATTGCCGA | |
| | CTGCTCTTCCAAAGGTCCTGAG | |
| mRnu12- | ACCCCCTAATTCTACAAGAATAGAAAGCTTAGCAGCTCCTCTGAATGGTTGCT | 362 |
| Terminator | CTCAGGTGCTAGTGGTTGCAAGTCGTATGTTGATGCCTGCCTTTGCCCTACCTG | |
| | GTGTGTGAGCGTGAGCGCGCCCCACCTCTCCAGCCTCTCTCTTACTGTTTTTCA | |
| | GACAAGATGGGACTGTTGTTCTCCTACTGCTATGTACCTCAATTTCAATTGATC | |
| | ATGCCTGGACATAACTGCCCATGTTGTAAGGTCGTCTACGTTGTAAGGTCGTG | |
| | CTCAGTTTCTGATGCCTAGACTAGTCACCTTTCCGTTTGCCTCCTGACTGTGTA | |
| | AATGAGGCAAATGAAGGTGGTCTCTTAACTGACATGTTAGTGTTTGACCTATG | |
| | GGATAGTTTCTCATTGTTGAGACAGAATTTCTAGAAGCTGGTCCGCTGAGCGG | |
| | CTAAGAGCACTTGCTGATATTGCAAAGGATCTACCTGGGTTTGGTTCTCAGCA | |
| | CTCACATGGAAGTTCACAT | |
| mRn7sk- | TGTAGTTCCTGCAATTTAATTTTCGTTTAAAACTTTATTTTATTCTTTAACTTAA | 363 |
| Terminator | AAAAAATATATA | |

TABLE 6

| Sequence | | SEQ |
|---|---|---|
| Name | Sequence | ID NO: |
| Scn2a | ggcctccgcgccgggttttggcgcctcccgcgggcgcccccctcctcacggcgagcgctgccacgtcaga | 364 |
| Luciferase | cgaagggcgcagcgagcgtcctgatccttccgcccggacgctcaggacagcggcccgctgctcataagac | |
| Reporter | tcggccttagaaccccagtatcagcagaaggacattttaggacgggacttgggtgactctagggcactgg | |
| | ttttctttccagagagcggaacaggcgaggaaaagtagtcccttctcggcgattctgcggagggatctcc | |
| | gtggggcggtgaacgccgatgattatataaggacgcgccgggtgtggcacagctagttccgtcgcagccg | |
| | ggatttgggtcgcggttcttgtttgtggatcgctgtgatcgtcacttggtCGGTAAGTTTAGTCTTTTTG | |
| | TCTTTTATTTCAGGTCCCGGATCCGGTGGTGGTGCAAATCAAAGAACTGCTCCTCAGTGGATGTTGCCTT | |
| | TACTTCTAGggtacGCCACCATGGGCACAACTACTGAAACAGAAATAAGAAAGAGACGG | |
| | TCCAGTTCTTATCATGTTTCCATGGATTTATTGGAAGATCCTACATCAAGG | |
| | CAAAGAGCAATGAGTATAGCCAGTATTTTGACCAACACCATGGAAGGTA | |
| | TGTTAAAAGTCCTGCGTCACAGTTACTTGGTGCTTTGGTAATGATGAAAA | |
| | AACACTTCATAAATTTCAATAAAATACTTCCTGACTTGATATTGTATCATT | |
| | ATTACACATTTTACTAAATAACAGTAAAATCCGTGCATAACTCATGGATT | |
| | CTATTATCTTCCACAGAttttttttttttATATTTAGCCTCCAGAAAGCTGCTGCAA | |
| | ATGTAAGGTATATTTTGAACACCACTTTCATACATTAAATTCTAAACATT | |
| | GAAACTTGTGTGCATGACGTTGAAAAGAGTGTAATGATAAATGCTTATAC | |
| | TTATGATGATGCTAAGCCATTTGGATTATATTAACTGCTTGAGACACAAG | |
| | TTATAAAATCCTATGACTTAACCAGAAATATAAATTAAAAATGTGAATTA | |
| | GGGTTTGATATTAACTTCCTTGAAGCAAAGTGTTTAAAATTTTGTAGTCCT | |
| | ACTTTTGCCTTTCTCTGACCAGATTCTTACAATATATCAGCTTTCTCTTTA | |
| | GTTGCAGATTTTATCTGAATAGTTAACATAATGTGTAGCAGTCTGGATCT | |
| | CAGAATGCCaaaataaagactttggggacagcttaatctgtgatcaatttctggctctgccatatgttaa | |
| | atgtgttaatttgtgactttgaatttcagtctcctcatcagtaaaatgtggatgatgatgtttaggcata | |
| | aggttgttgaatggattaaataagccttcttagataaaacactgatgtatttggcatgcagaagacagtt | |
| | aataaatattatcaatattaGTTGTTTTGTTGTTGTTATTTTTGTTAATTCACATGTTTTTGCCTTTCCA | |
| | TACTGTAAGTGAATTCAAACAACTGTCAACTTCAACTACTTGGAAAATATTTTCATGTAAAATGT | |
| | ATTCTATCCCCCTTCCTTGCCCTCCTATTCCCTCCTCTCCCTATCTCTTTAC | |
| | AAACCTTCTCCCTTGTACCCCTTCCCAGGTAtgtgtgtgagtgtgagtgtgtgtagatgtgtC | |
| | AAGGGAGAAGAGAAAAGGAGAATGAAAGCAAAAGAGAGCAAGCATAC | |
| | ACGTCCCTTTCTTATTGATAATTAGATTTTCTCTTGAGATTGGATAGATTC | |
| | CTGGAATAATTCTTTTCCTGTCTGTATGCAAAGATCCCATAATATTATTAA | |
| | TACCAATACGAAAAGCCTGAAAATCACAGCCAGAAAAAATTCACAGTGT | |
| | AGACGACTGTGTACATCACAGACAAGTCAGTATTACAAAACCCAATTTTC | |
| | ATAGTGTCCTATTTCAGTATCCTAATGCAATTCACTGATTTCAATTGAATA | |
| | TTAAACTCTAGTACGTTCTTCCCCAACCTCGCCTGCGTTAGCTTGCACTCC | |
| | CTCTTCCCCCCAGCTGCCAGTAGCTTGCTCCTCCCTGTCCCTCCAGGTAAA | |
| | TCTTTTGAAGATTGTCTGGCCTTCCGCTCCTTGCCATAGCAAAACCACTG | |
| | AGAGGAAGCTGCCAGTGGTTCTGCTACCGATGTCAGCAGCATGTCTGCTC | |
| | CCTAAAGCAGGAAGTAGAGAAGGAGACAGGGTAAGTCTAAATCAACAG | |
| | TCATGCTTTGCACTTCTGATAGCATTAAGTTTGAGCTAAATAAGACATTA | |
| | CTTAAAAAACCTCAAATATCCACAAGATTGGACTTGCCAACTAATTAAGA | |
| | TTTGGAGTTCAAAATAAATGCACCCACACCTTTCTCCATGGAACTATGTG | |
| | ACATGGGGTTGCTTAGGATGGAAAGGATGTTCTAGGAATAAGTGCAATC | |
| | TAGGAAGCTGAAGACTGAGAGTGTTTTCGTTTTATTATCTGCAGAGCTTT | |
| | TGACTTGTGTATTTGTGAGAAATAATGGCCAAGTTTTTATTCTGTTTTTAA | |
| | TCAGTTATCTAGAATGAAAACTGACTTTTCTTTATTCAATTGTATGTAGAC | |
| | ACATTGAGTGTGACATTTGTCAAGGTTGGTTGTTAGCAATATCACATACA | |
| | TGCATACTCAAGCAGACTTAAGATAGTCCtttttttttttttttttttttttGGTTTCTGATA | |
| | ATGGTGCAAAGTTTTCCTGGTTGACATAATCTCTTTTCTTGGGGATCCTTT | |
| | CTTCTATGTCTGATTATTGTTTATTTCACCTTTCCTTTTTATGAACCAGGCT | |
| | TGTTGATCCGGTTGGCAATTTTTGTTCTCCTTCTTTTTAACTACAGCCAAG | |
| | TCTCCGTTGTCCAGGGTAATGGATAGCCTCATGCTTAATGAAGCAGTAGT | |
| | GGATAAGCAAAAATGAACCATTTGCGTTTCAAATTTTTAAAAGTGCAAA | |
| | ATCACATAGAAATGTTTTCTGGTCACACCTTTGTGAAGGATGGTGGGAGG | |
| | GTGAGTTAGAAGCGCCTGAAGAATCAAGGTGAGCCAGCAAAAGACACA | |
| | GATTTACTGTAAGTGATTCTATGTGGATAACCCATGCAGGAATAATGGAG | |
| | ATGTGGCTGCAGTTCTCTCCTGAATGCTTTGCTTTGTTTTAAAGTGTGAGA | |
| | TTCCCCCCttttttttGGAATGAATAATTGAATGATTTTATTTTAGAACTTAAAC | |
| | AACTTTGCTCCGGTTAAGTTGATGAAGAGATTATCTTGCCAAAGGAAGAT | |
| | GGTAGACAGTTTTTCACTTCCTAATTCCCCAAATTTCTTTGCCAATATTCA | |
| | CCAAATTTCAGTATTTTGGGTGTGACCTTAAAGATCTGTGCATTTCTGTCT | |
| | TCTCTCCCAATGTTTGGTTGAAATTCTTCTTGACATCATTGTACATTTTCC | |
| | TTGAATAAATGCATTTTAATATAAAATTTTATGTCATGTTTGATATGAGA | |
| | GttatatatttaaatacatttaaataaatGTTTACCATGAAAATGTATGAATTATATGTATGT | |
| | TTCACCTAAAATCCTTTGTATTTTTCCAGTAATAAATGAGTTCCACTTTGT | |
| | GAAATGTTGATTTGTAACAACAGTGAGGACTCCAGTTCCTTAGGCTGGGG | |
| | TATTTTCTCTTCTTTTATGCCCTCTAGTTAAATGAGAAATGTAGAGAGATG | |
| | GAACTTTGTTGTGTCTAATATGCAAGCCTATAATCTAATAAAATTTAATTT | |
| | GAGACTTTTAAACTGAGATTGGTGACACTGACAAAATTATCTAATTAGAA | |
| | GATCACCAAAACATATCTAATCCAAGAAACTGACATTCAGTGTGACTGAT | |
| | TAAGGTTCTTAGGACATCTCCTGAGATATCTCTGATAACATATATACTTCT | |
| | TGCTCTACCTGGAACATGGATGAGCTTTAAGTGTATGCAATGCAAGTTCT | |
| | ACCCATTAGTTTCTAGCAGCCTTGAAGATAAGTATCAGACAGTTTAGTGT | |
| | TGCCAATAGAATCTTGGAAGCTATGTTTAGCCAGGATACATTTGGAAAGC | |
| | TTACTAGCCTTTCTGTACTGATCCTTTCTATGACAGCAAACCCATTGTAAA | |
| | ATTTTCCCTGTTCCTCCAGCAGATTAACCCATAATATCTTTTAACAACTTT | |
| | AGATTTTTTAAATTCCTTTTAATTTAAACCAAATCTGCTTAATAGAAAGTA | |
| | AGCAGTTTTCATGAGGATTCTAACTTTTTTTCTTCCAGAACTTGAAGAATC | |
| | CAGACAGAAATGCCCACCATGCTGGTATAAATTTGCTAATATGTGTTTGA | |
| | TTTGGGACTGTTGTAAACCATGGTTAAAGGTGAAACACCTTGTCAACCTG | |
| | GTTGTAATGGACCCATTTGTTGACCTGGCCATCACCATCTGCATTGTCTTA | |
| | AATACACTCTTCATGGCTATGGAGCACTATCCCATGACGGAGCAGTTCAG | |
| | CAGTGTACTGTCTGTTGGAAACCTGCTTGGCAGCGGCgagggcagaggcagtctgct | |
| | gacatgcggtgacgtggaagagaatcccggccctCTTAAGCAAAGGAAATCTGGCCGCTCTG | |
| | CTGGGAAGTATGATGTGTATTTGATCTCCGGGatggaagacgccaaaaacataaagaaag | |
| | gcccggcgccattctatccgctggaagatggaaccgctggagagcaactgcataaggctatgaagagata | |
| | cgccctggttcctggaacaattgcttttacagatgcacatatcgaggtggacatcacttacgctgagtac | |
| | ttcgaaatgtccgttcggttggcagaagctatgaaacgatatgggctgaatacaaatcacagaatcgtcg | |
| | tatgcagtgaaaactctcttcaattctttatgccggtgttgggcgcgttatttatcggagttgcagttgc | |
| | gcccgcgaacgacatttataatgaacgtgaattgctcaacagtatgggcatttcgcagcctaccgtggtg | |
| | ttcgtttccaaaaaggggttgcaaaaaattttgaacgtgcaaaaaaagctcccaatcatccaaaaaatta | |
| | ttatcatggattctaaaacggattaccagggatttcagtcgatgtacacgttcgtcacatctcatctacc | |
| | tcccggttttaatgaatacgattttgtgccagagtccttcgatagggacaagacaattgcactgatcatg | |
| | aactcctctggatctactggtctgcctaaaggtgtcgctctgcctcatagaactgcctgcgtgagattct | |
| | cgcatgccagagatcctatttttggcaatcaaatcattccggatactgcgattttaagtgttgttccatt | |
| | ccatcacggttttggaatgtttactacactcggatatttgatatgtggatttcgagtcgtcttaatgtat | |
| | agatttgaagaagagctgtttctgaggagccttcaggattacaagattcaaagtgcgctgctggtgccaa | |
| | ccctattctccttcttcgccaaaagcactctgattgacaaatacgatttatctaatttacacgaaattgc | |
| | ttctggtggcgctcccctctctaaggaagtcggggaagcggttgccaagaggttccatctgccaggtatc | |
| | aggcaaggatatgggctcactgagactacatcagctattctgattacacccgagggggatgataaaccgg | |
| | gcgcggtcggtaaagttgttccattttttgaagcgaaggttgtggatctggataccgggaaaacgctggg | |
| | cgttaatcaaagaggcgaactgtgtgtgagaggtcctatgattatgtccggttatgtaaacaatccggaa | |
| | gcgaccaacgccttgattgacaaggatggatggctacattctggagacatagcttactgggacgaagacg | |
| | aacacttcttcatcgttgaccgcctgaagtctctgattaagtacaaaggctatcaggtggctcccgctga | |
| | attggaatccatcttgctccaacaccccaacatcttcgacgcaggtgtcgcaggtcttcccgacgatgac | |
| | gccggtgaacttcccgccgccgttgttgttttggagcacggaaagacgatgacggaaaaagagatcgtgg | |
| | attacgtcgccagtcaagtaacaaccgcgaaaaagttgcgcggaggagttgtgtttgtggacgaagtacc | |
| | gaaaggtcttaccggaaaactcgacgcaagaaaaatcagagagatcctcataaaggccaagaagggcgga | |
| | aagatcgccgtgtaattctagagtcggggcggccggaattcagatctaccagacatgataagatacattg | |
| | atgagtttggacaaaccacaactagaatgcagtgaaaaaaatgctttatttgtgaaatttgtgatgctat | |
| | tgctttatttGTAACCATTATAAGCTGCAATAAACAAGTT | |

TABLE 7A
Example Fragment Sequences in a Library of Constructs

| Fragment Name | SEQ ID NO: |
|---|---|
| 001_mu1a1_Scn2aF1Scr2_U1 | 365 |
| 002_mu1a1_Scr2_U1 | 366 |
| 003_mu1a1_Scn2aD12_U1 | 367 |
| 004_mu1a1_Scn2aB10_U1 | 368 |
| 005_hRNU1-1_Scn2aB10_hRNU1-1 | 369 |
| 006_hRNU1-1_Scn2aB10_hRNU1-2 | 370 |
| 007_hRNU1-1_Scn2aB10_hRNU1-3 | 371 |
| 008_hRNU1-1_Scn2aB10_hRNU2-1 | 372 |
| 009_hRNU1-1_Scn2aB10_hU3 | 373 |
| 010_hRNU1-1_Scn2aB10_hRNU4-1 | 374 |
| 011_hRNU1-1_Scn2aB10_hRNU5A-1 | 375 |
| 012_hRNU1-1_Scn2aB10_hRNU6-1 | 376 |
| 013_hRNU1-1_Scn2aB10_RNU7-1 | 377 |
| 014_hRNU1-1_Scn2aB10_hRNU11 | 378 |
| 015_hRNU1-1_Scn2aB10_hRNU12 | 379 |
| 016_hRNU1-1_Scn2aB10_hRN7SK | 380 |
| 017_hRNU1-1_Scn2aB10_mRnu1a1 | 381 |
| 018_hRNU1-1_Scn2aB10_mRnu1b1 | 382 |
| 019_hRNU1-1_Scn2aB10_mRnu1b6 | 383 |
| 020_hRNU1-1_Scn2aB10_mRnu2-10 | 384 |
| 021_hRNU1-1_Scn2aB10_mRnu3b1 | 385 |
| 022_hRNU1-1_Scn2aB10_mRnu5g | 386 |
| 023_hRNU1-1_Scn2aB10_mRnu6 | 387 |
| 024_hRNU1-1_Scn2aB10_mRnu7 | 388 |
| 025_hRNU1-1_Scn2aB10_mRnu11 | 389 |
| 026_hRNU1-1_Scn2aB10_mRnu12 | 390 |
| 027_hRNU1-1_Scn2aB10_mRn7sk | 391 |
| 028_hRNU1-2_Scn2aB10_hRNU1-1 | 392 |
| 029_hRNU1-2_Scn2aB10_hRNU1-2 | 393 |
| 030_hRNU1-2_Scn2aB10_hRNU1-3 | 394 |
| 031_hRNU1-2_Scn2aB10_hRNU2-1 | 395 |
| 032_hRNU1-2_Scn2aB10_hU3 | 396 |
| 033_hRNU1-2_Scn2aB10_hRNU4-1 | 397 |
| 034_hRNU1-2_Scn2aB10_hRNU5A-1 | 398 |
| 035_hRNU1-2_Scn2aB10_hRNU6-1 | 399 |
| 036_hRNU1-2_Scn2aB10_RNU7-1 | 400 |
| 037_hRNU1-2_Scn2aB10_hRNU11 | 401 |
| 038_hRNU1-2_Scn2aB10_hRNU12 | 402 |
| 039_hRNU1-2_Scn2aB10_hRN7SK | 403 |
| 040_hRNU1-2_Scn2aB10_mRnu1a1 | 404 |
| 041_hRNU1-2_Scn2aB10_mRnu1b1 | 405 |
| 042_hRNU1-2_Scn2aB10_mRnu1b6 | 406 |
| 043_hRNU1-2_Scn2aB10_mRnu2-10 | 407 |
| 044_hRNU1-2_Scn2aB10_mRnu3b1 | 408 |
| 045_hRNU1-2_Scn2aB10_mRnu5g | 409 |
| 046_hRNU1-2_Scn2aB10_mRnu6 | 410 |
| 047_hRNU1-2_Scn2aB10_mRnu7 | 411 |
| 048_hRNU1-2_Scn2aB10_mRnu11 | 412 |
| 049_hRNU1-2_Scn2aB10_mRnu12 | 413 |
| 050_hRNU1-2_Scn2aB10_mRn7sk | 414 |
| 051_hRNU1-3_Scn2aB10_hRNU1-1 | 415 |
| 052_hRNU1-3_Scn2aB10_hRNU1-2 | 416 |
| 053_hRNU1-3_Scn2aB10_hRNU1-3 | 417 |
| 054_hRNU1-3_Scn2aB10_hRNU2-1 | 418 |
| 055_hRNU1-3_Scn2aB10_hU3 | 419 |
| 056_hRNU1-3_Scn2aB10_hRNU4-1 | 420 |
| 057_hRNU1-3_Scn2aB10_hRNU5A-1 | 421 |
| 058_hRNU1-3_Scn2aB10_hRNU6-1 | 422 |
| 059_hRNU1-3_Scn2aB10_RNU7-1 | 423 |
| 060_hRNU1-3_Scn2aB10_hRNU11 | 424 |
| 061_hRNU1-3_Scn2aB10_hRNU12 | 425 |
| 062_hRNU1-3_Scn2aB10_hRN7SK | 426 |
| 063_hRNU1-3_Scn2aB10_mRnu1a1 | 427 |
| 064_hRNU1-3_Scn2aB10_mRnu1b1 | 428 |
| 065_hRNU1-3_Scn2aB10_mRnu1b6 | 429 |
| 066_hRNU1-3_Scn2aB10_mRnu2-10 | 430 |
| 067_hRNU1-3_Scn2aB10_mRnu3b1 | 431 |
| 068_hRNU1-3_Scn2aB10_mRnu5g | 432 |
| 069_hRNU1-3_Scn2aB10_mRnu6 | 433 |
| 070_hRNU1-3_Scn2aB10_mRnu7 | 434 |
| 071_hRNU1-3_Scn2aB10_mRnu11 | 435 |
| 072_hRNU1-3_Scn2aB10_mRnu12 | 436 |
| 073_hRNU1-3_Scn2aB10_mRn7sk | 437 |
| 074_hRNU2-1_Scn2aB10_hRNU1-1 | 438 |
| 075_hRNU2-1_Scn2aB10_hRNU1-2 | 439 |
| 076_hRNU2-1_Scn2aB10_hRNU1-3 | 440 |
| 077_hRNU2-1_Scn2aB10_hRNU2-1 | 441 |
| 078_hRNU2-1_Scn2aB10_hU3 | 442 |
| 079_hRNU2-1_Scn2aB10_hRNU4-1 | 443 |
| 080_hRNU2-1_Scn2aB10_hRNU5A-1 | 444 |
| 081_hRNU2-1_Scn2aB10_hRNU6-1 | 445 |
| 082_hRNU2-1_Scn2aB10_RNU7-1 | 446 |
| 083_hRNU2-1_Scn2aB10_hRNU11 | 447 |
| 084_hRNU2-1_Scn2aB10_hRNU12 | 448 |
| 085_hRNU2-1_Scn2aB10_hRN7SK | 449 |
| 086_hRNU2-1_Scn2aB10_mRnu1a1 | 450 |
| 087_hRNU2-1_Scn2aB10_mRnu1b1 | 451 |
| 088_hRNU2-1_Scn2aB10_mRnu1b6 | 452 |
| 089_hRNU2-1_Scn2aB10_mRnu2-10 | 453 |
| 090_hRNU2-1_Scn2aB10_mRnu3b1 | 454 |
| 091_hRNU2-1_Scn2aB10_mRnu5g | 455 |
| 092_hRNU2-1_Scn2aB10_mRnu6 | 456 |
| 093_hRNU2-1_Scn2aB10_mRnu7 | 457 |
| 094_hRNU2-1_Scn2aB10_mRnu11 | 458 |
| 095_hRNU2-1_Scn2aB10_mRnu12 | 459 |
| 096_hRNU2-1_Scn2aB10_mRn7sk | 460 |
| 097_mu1a1_Scn2aF1Scr2_U1 | 461 |
| 098_mu1a1_Scr2_U1 | 462 |
| 099_mu1a1_Scn2aD12_U1 | 463 |
| 100_mu1a1_Scn2aB10_U1 | 464 |
| 101_hU3_Scn2aB10_hRNU1-1 | 465 |
| 102_hU3_Scn2aB10_hRNU1-2 | 466 |
| 103_hU3_Scn2aB10_hRNU1-3 | 467 |
| 104_hU3_Scn2aB10_hRNU2-1 | 468 |
| 105_hU3_Scn2aB10_hU3 | 469 |
| 106_hU3_Scn2aB10_hRNU4-1 | 470 |
| 107_hU3_Scn2aB10_hRNU5A-1 | 471 |
| 108_hU3_Scn2aB10_hRNU6-1 | 472 |
| 109_hU3_Scn2aB10_RNU7-1 | 473 |
| 110_hU3_Scn2aB10_hRNU11 | 474 |
| 111_hU3_Scn2aB10_hRNU12 | 475 |
| 112_hU3_Scn2aB10_hRN7SK | 476 |
| 113_hU3_Scn2aB10_mRnu1a1 | 477 |
| 114_hU3_Scn2aB10_mRnu1b1 | 478 |
| 115_hU3_Scn2aB10_mRnu1b6 | 479 |
| 116_hU3_Scn2aB10_mRnu2-10 | 480 |
| 117_hU3_Scn2aB10_mRnu3b1 | 481 |
| 118_hU3_Scn2aB10_mRnu5g | 482 |
| 119_hU3_Scn2aB10_mRnu6 | 483 |
| 120_hU3_Scn2aB10_mRnu7 | 484 |
| 121_hU3_Scn2aB10_mRnu11 | 485 |
| 122_hU3_Scn2aB10_mRnu12 | 486 |
| 123_hU3_Scn2aB10_mRn7sk | 487 |
| 124_hRNU4-1_Scn2aB10_hRNU1-1 | 488 |
| 125_hRNU4-1_Scn2aB10_hRNU1-2 | 489 |
| 126_hRNU4-1_Scn2aB10_hRNU1-3 | 490 |
| 127_hRNU4-1_Scn2aB10_hRNU2-1 | 491 |
| 128_hRNU4-1_Scn2aB10_hU3 | 492 |
| 129_hRNU4-1_Scn2aB10_hRNU4-1 | 493 |
| 130_hRNU4-1_Scn2aB10_hRNU5A-1 | 494 |
| 131_hRNU4-1_Scn2aB10_hRNU6-1 | 495 |
| 132_hRNU4-1_Scn2aB10_RNU7-1 | 496 |
| 133_hRNU4-1_Scn2aB10_hRNU11 | 497 |
| 134_hRNU4-1_Scn2aB10_hRNU12 | 498 |
| 135_hRNU4-1_Scn2aB10_hRN7SK | 499 |
| 136_hRNU4-1_Scn2aB10_mRnu1a1 | 500 |
| 137_hRNU4-1_Scn2aB10_mRnu1b1 | 501 |
| 138_hRNU4-1_Scn2aB10_mRnu1b6 | 502 |
| 139_hRNU4-1_Scn2aB10_mRnu2-10 | 503 |
| 140_hRNU4-1_Scn2aB10_mRnu3b1 | 504 |
| 141_hRNU4-1_Scn2aB10_mRnu5g | 505 |
| 142_hRNU4-1_Scn2aB10_mRnu6 | 506 |
| 143_hRNU4-1_Scn2aB10_mRnu7 | 507 |
| 144_hRNU4-1_Scn2aB10_mRnu11 | 508 |
| 145_hRNU4-1_Scn2aB10_mRnu12 | 509 |
| 146_hRNU4-1_Scn2aB10_mRn7sk | 510 |
| 147_hRNU5A-1_Scn2aB10_hRNU1-1 | 511 |
| 148_hRNU5A-1_Scn2aB10_hRNU1-2 | 512 |
| 149_hRNU5A-1_Scn2aB10_hRNU1-3 | 513 |
| 150_hRNU5A-1_Scn2aB10_hRNU2-1 | 514 |
| 151_hRNU5A-1_Scn2aB10_hU3 | 515 |
| 152_hRNU5A-1_Scn2aB10_hRNU4-1 | 516 |
| 153_hRNU5A-1_Scn2aB10_hRNU5A-1 | 517 |
| 154_hRNU5A-1_Scn2aB10_hRNU6-1 | 518 |
| 155_hRNU5A-1_Scn2aB10_RNU7-1 | 519 |
| 156_hRNU5A-1_Scn2aB10_hRNU11 | 520 |
| 157_hRNU5A-1_Scn2aB10_hRNU12 | 521 |
| 158_hRNU5A-1_Scn2aB10_hRN7SK | 522 |
| 159_hRNU5A-1_Scn2aB10_mRnu1a1 | 523 |
| 160_hRNU5A-1_Scn2aB10_mRnu1b1 | 524 |
| 161_hRNU5A-1_Scn2aB10_mRnu1b6 | 525 |
| 162_hRNU5A-1_Scn2aB10_mRnu2-10 | 526 |
| 163_hRNU5A-1_Scn2aB10_mRnu3b1 | 527 |
| 164_hRNU5A-1_Scn2aB10_mRnu5g | 528 |
| 165_hRNU5A-1_Scn2aB10_mRnu6 | 529 |
| 166_hRNU5A-1_Scn2aB10_mRnu7 | 530 |
| 167_hRNU5A-1_Scn2aB10_mRnu11 | 531 |
| 168_hRNU5A-1_Scn2aB10_mRnu12 | 532 |
| 169_hRNU5A-1_Scn2aB10_mRn7sk | 533 |
| 170_hRNU6-1_Scn2aB10_hRNU1-1 | 534 |
| 171_hRNU6-1_Scn2aB10_hRNU1-2 | 535 |
| 172_hRNU6-1_Scn2aB10_hRNU1-3 | 536 |
| 173_hRNU6-1_Scn2aB10_hRNU2-1 | 537 |
| 174_hRNU6-1_Scn2aB10_hU3 | 538 |
| 175_hRNU6-1_Scn2aB10_hRNU4-1 | 539 |
| 176_hRNU6-1_Scn2aB10_hRNU5A-1 | 540 |
| 177_hRNU6-1_Scn2aB10_hRNU6-1 | 541 |
| 178_hRNU6-1_Scn2aB10_RNU7-1 | 542 |
| 179_hRNU6-1_Scn2aB10_hRNU11 | 543 |
| 180_hRNU6-1_Scn2aB10_hRNU12 | 544 |
| 181_hRNU6-1_Scn2aB10_hRN7SK | 545 |
| 182_hRNU6-1_Scn2aB10_mRnu1a1 | 546 |
| 183_hRNU6-1_Scn2aB10_mRnu1b1 | 547 |
| 184_hRNU6-1_Scn2aB10_mRnu1b6 | 548 |
| 185_hRNU6-1_Scn2aB10_mRnu2-10 | 549 |
| 186_hRNU6-1_Scn2aB10_mRnu3b1 | 550 |
| 187_hRNU6-1_Scn2aB10_mRnu5g | 551 |
| 188_hRNU6-1_Scn2aB10_mRnu6 | 552 |
| 189_hRNU6-1_Scn2aB10_mRnu7 | 553 |
| 190_hRNU6-1_Scn2aB10_mRnu11 | 554 |
| 191_hRNU6-1_Scn2aB10_mRnu12 | 555 |
| 192_hRNU6-1_Scn2aB10_mRn7sk | 556 |
| 193_mu1a1_Scn2aF1Scr2_U1 | 557 |
| 194_mu1a1_Scr2_U1 | 558 |
| 195_mu1a1_Scn2aD12_U1 | 559 |
| 196_mu1a1_Scn2aB10_U1 | 560 |
| 197_RNU7-1_Scn2aB10_hRNU1-1 | 561 |
| 198_RNU7-1_Scn2aB10_hRNU1-2 | 562 |
| 199_RNU7-1_Scn2aB10_hRNU1-3 | 563 |
| 200_RNU7-1_Scn2aB10_hRNU2-1 | 564 |
| 201_RNU7-1_Scn2aB10_hU3 | 565 |
| 202_RNU7-1_Scn2aB10_hRNU4-1 | 566 |
| 203_RNU7-1_Scn2aB10_hRNU5A-1 | 567 |
| 204_RNU7-1_Scn2aB10_hRNU6-1 | 568 |
| 205_RNU7-1_Scn2aB10_RNU7-1 | 569 |
| 206_RNU7-1_Scn2aB10_hRNU11 | 570 |
| 207_RNU7-1_Scn2aB10_hRNU12 | 571 |
| 208_RNU7-1_Scn2aB10_hRN7SK | 572 |
| 209_RNU7-1_Scn2aB10_mRnu1a1 | 573 |
| 210_RNU7-1_Scn2aB10_mRnu1b1 | 574 |
| 211_RNU7-1_Scn2aB10_mRnu1b6 | 575 |
| 212_RNU7-1_Scn2aB10_mRnu2-10 | 576 |
| 213_RNU7-1_Scn2aB10_mRnu3b1 | 577 |
| 214_RNU7-1_Scn2aB10_mRnu5g | 578 |
| 215_RNU7-1_Scn2aB10_mRnu6 | 579 |
| 216_RNU7-1_Scn2aB10_mRnu7 | 580 |
| 217_RNU7-1_Scn2aB10_mRnu11 | 581 |
| 218_RNU7-1_Scn2aB10_mRnu12 | 582 |
| 219_RNU7-1_Scn2aB10_mRn7sk | 583 |
| 220_hRNU11_Scn2aB10_hRNU1-1 | 584 |
| 221_hRNU11_Scn2aB10_hRNU1-2 | 585 |
| 222_hRNU11_Scn2aB10_hRNU1-3 | 586 |
| 223_hRNU11_Scn2aB10_hRNU2-1 | 587 |
| 224_hRNU11_Scn2aB10_hU3 | 588 |
| 225_hRNU11_Scn2aB10_hRNU4-1 | 589 |
| 226_hRNU11_Scn2aB10_hRNU5A-1 | 590 |
| 227_hRNU11_Scn2aB10_hRNU6-1 | 591 |
| 228_hRNU11_Scn2aB10_RNU7-1 | 592 |
| 229_hRNU11_Scn2aB10_hRNU11 | 593 |
| 230_hRNU11_Scn2aB10_hRNU12 | 594 |
| 231_hRNU11_Scn2aB10_hRN7SK | 595 |
| 232_hRNU11_Scn2aB10_mRnu1a1 | 596 |
| 233_hRNU11_Scn2aB10_mRnu1b1 | 597 |
| 234_hRNU11_Scn2aB10_mRnu1b6 | 598 |
| 235_hRNU11_Scn2aB10_mRnu2-10 | 599 |
| 236_hRNU11_Scn2aB10_mRnu3b1 | 600 |
| 237_hRNU11_Scn2aB10_mRnu5g | 601 |
| 238_hRNU11_Scn2aB10_mRnu6 | 602 |
| 239_hRNU11_Scn2aB10_mRnu7 | 603 |
| 240_hRNU11_Scn2aB10_mRnu11 | 604 |
| 241_hRNU11_Scn2aB10_mRnu12 | 605 |
| 242_hRNU11_Scn2aB10_mRn7sk | 606 |
| 243_hRNU12_Scn2aB10_hRNU1-1 | 607 |
| 244_hRNU12_Scn2aB10_hRNU1-2 | 608 |
| 245_hRNU12_Scn2aB10_hRNU1-3 | 609 |
| 246_hRNU12_Scn2aB10_hRNU2-1 | 610 |
| 247_hRNU12_Scn2aB10_hU3 | 611 |
| 248_hRNU12_Scn2aB10_hRNU4-1 | 612 |
| 249_hRNU12_Scn2aB10_hRNU5A-1 | 613 |
| 250_hRNU12_Scn2aB10_hRNU6-1 | 614 |
| 251_hRNU12_Scn2aB10_RNU7-1 | 615 |
| 252_hRNU12_Scn2aB10_hRNU11 | 616 |
| 253_hRNU12_Scn2aB10_hRNU12 | 617 |
| 254_hRNU12_Scn2aB10_hRN7SK | 618 |
| 255_hRNU12_Scn2aB10_mRnu1a1 | 619 |
| 256_hRNU12_Scn2aB10_mRnu1b1 | 620 |
| 257_hRNU12_Scn2aB10_mRnu1b6 | 621 |
| 258_hRNU12_Scn2aB10_mRnu2-10 | 622 |
| 259_hRNU12_Scn2aB10_mRnu3b1 | 623 |
| 260_hRNU12_Scn2aB10_mRnu5g | 624 |
| 261_hRNU12_Scn2aB10_mRnu6 | 625 |
| 262_hRNU12_Scn2aB10_mRnu7 | 626 |
| 263_hRNU12_Scn2aB10_mRnu11 | 627 |
| 264_hRNU12_Scn2aB10_mRnu12 | 628 |
| 265_hRNU12_Scn2aB10_mRn7sk | 629 |
| 266_hRN7SK_Scn2aB10_hRNU1-1 | 630 |
| 267_hRN7SK_Scn2aB10_hRNU1-2 | 631 |
| 268_hRN7SK_Scn2aB10_hRNU1-3 | 632 |
| 269_hRN7SK_Scn2aB10_hRNU2-1 | 633 |
| 270_hRN7SK_Scn2aB10_hU3 | 634 |
| 271_hRN7SK_Scn2aB10_hRNU4-1 | 635 |
| 272_hRN7SK_Scn2aB10_hRNU5A-1 | 636 |
| 273_hRN7SK_Scn2aB10_hRNU6-1 | 637 |
| 274_hRN7SK_Scn2aB10_RNU7-1 | 638 |
| 275_hRN7SK_Scn2aB10_hRNU11 | 639 |
| 276_hRN7SK_Scn2aB10_hRNU12 | 640 |
| 277_hRN7SK_Scn2aB10_hRN7SK | 641 |
| 278_hRN7SK_Scn2aB10_mRnu1a1 | 642 |
| 279_hRN7SK_Scn2aB10_mRnu1b1 | 643 |
| 280_hRN7SK_Scn2aB10_mRnu1b6 | 644 |
| 281_hRN7SK_Scn2aB10_mRnu2-10 | 645 |
| 282_hRN7SK_Scn2aB10_mRnu3b1 | 646 |
| 283_hRN7SK_Scn2aB10_mRnu5g | 647 |
| 284_hRN7SK_Scn2aB10_mRnu6 | 648 |
| 285_hRN7SK_Scn2aB10_mRnu7 | 649 |
| 286_hRN7SK_Scn2aB10_mRnu11 | 650 |
| 287_hRN7SK_Scn2aB10_mRnu12 | 651 |
| 288_hRN7SK_Scn2aB10_mRn7sk | 652 |
| 289_mu1a1_Scn2aF1Scr2_U1 | 653 |
| 290_mu1a1_Scr2_U1 | 654 |
| 291_mu1a1_Scn2aD12_U1 | 655 |
| 292_mu1a1_Scn2aB10_U1 | 656 |
| 293_mRnu1a1_Scn2aB10_hRNU1-1 | 657 |
| 294_mRnu1a1_Scn2aB10_hRNU1-2 | 658 |
| 295_mRnu1a1_Scn2aB10_hRNU1-3 | 659 |
| 296_mRnu1a1_Scn2aB10_hRNU2-1 | 660 |
| 297_mRnu1a1_Scn2aB10_hU3 | 661 |
| 298_mRnu1a1_Scn2aB10_hRNU4-1 | 662 |
| 299_mRnu1a1_Scn2aB10_hRNU5A-1 | 663 |
| 300_mRnu1a1_Scn2aB10_hRNU6-1 | 664 |
| 301_mRnu1a1_Scn2aB10_RNU7-1 | 665 |
| 302_mRnu1a1_Scn2aB10_hRNU11 | 666 |
| 303_mRnu1a1_Scn2aB10_hRNU12 | 667 |
| 304_mRnu1a1_Scn2aB10_hRN7SK | 668 |
| 305_mRnu1a1_Scn2aB10_mRnu1a1 | 669 |
| 306_mRnu1a1_Scn2aB10_mRnu1b1 | 670 |
| 307_mRnu1a1_Scn2aB10_mRnu1b6 | 671 |
| 308_mRnu1a1_Scn2aB10_mRnu2-10 | 672 |
| 309_mRnu1a1_Scn2aB10_mRnu3b1 | 673 |
| 310_mRnu1a1_Scn2aB10_mRnu5g | 674 |
| 311_mRnu1a1_Scn2aB10_mRnu6 | 675 |
| 312_mRnu1a1_Scn2aB10_mRnu7 | 676 |
| 313_mRnu1a1_Scn2aB10_mRnu11 | 677 |
| 314_mRnu1a1_Scn2aB10_mRnu12 | 678 |
| 315_mRnu1a1_Scn2aB10_mRn7sk | 679 |
| 316_mRnu1b1_Scn2aB10_hRNU1-1 | 680 |
| 317_mRnu1b1_Scn2aB10_hRNU1-2 | 681 |
| 318_mRnu1b1_Scn2aB10_hRNU1-3 | 682 |
| 319_mRnu1b1_Scn2aB10_hRNU2-1 | 683 |
| 320_mRnu1b1_Scn2aB10_hU3 | 684 |
| 321_mRnu1b1_Scn2aB10_hRNU4-1 | 685 |
| 322_mRnu1b1_Scn2aB10_hRNU5A-1 | 686 |
| 323_mRnu1b1_Scn2aB10_hRNU6-1 | 687 |
| 324_mRnu1b1_Scn2aB10_RNU7-1 | 688 |
| 325_mRnu1b1_Scn2aB10_hRNU11 | 689 |
| 326_mRnu1b1_Scn2aB10_hRNU12 | 690 |
| 327_mRnu1b1_Scn2aB10_hRN7SK | 691 |
| 328_mRnu1b1_Scn2aB10_mRnu1a1 | 692 |
| 329_mRnu1b1_Scn2aB10_mRnu1b1 | 693 |
| 330_mRnu1b1_Scn2aB10_mRnu1b6 | 694 |
| 331_mRnu1b1_Scn2aB10_mRnu2-10 | 695 |
| 332_mRnu1b1_Scn2aB10_mRnu3b1 | 696 |
| 333_mRnu1b1_Scn2aB10_mRnu5g | 697 |
| 334_mRnu1b1_Scn2aB10_mRnu6 | 698 |
| 335_mRnu1b1_Scn2aB10_mRnu7 | 699 |
| 336_mRnu1b1_Scn2aB10_mRnu11 | 700 |
| 337_mRnu1b1_Scn2aB10_mRnu12 | 701 |
| 338_mRnu1b1_Scn2aB10_mRn7sk | 702 |
| 339_mRnu1b6_Scn2aB10_hRNU1-1 | 703 |
| 340_mRnu1b6_Scn2aB10_hRNU1-2 | 704 |
| 341_mRnu1b6_Scn2aB10_hRNU1-3 | 705 |
| 342_mRnu1b6_Scn2aB10_hRNU2-1 | 706 |
| 343_mRnu1b6_Scn2aB10_hU3 | 707 |
| 344_mRnu1b6_Scn2aB10_hRNU4-1 | 708 |
| 345_mRnu1b6_Scn2aB10_hRNU5A-1 | 709 |
| 346_mRnu1b6_Scn2aB10_hRNU6-1 | 710 |
| 347_mRnu1b6_Scn2aB10_RNU7-1 | 711 |
| 348_mRnu1b6_Scn2aB10_hRNU11 | 712 |
| 349_mRnu1b6_Scn2aB10_hRNU12 | 713 |
| 350_mRnu1b6_Scn2aB10_hRN7SK | 714 |
| 351_mRnu1b6_Scn2aB10_mRnu1a1 | 715 |
| 352_mRnu1b6_Scn2aB10_mRnu1b1 | 716 |
| 353_mRnu1b6_Scn2aB10_mRnu1b6 | 717 |
| 354_mRnu1b6_Scn2aB10_mRnu2-10 | 718 |
| 355_mRnu1b6_Scn2aB10_mRnu3b1 | 719 |
| 356_mRnu1b6_Scn2aB10_mRnu5g | 720 |
| 357_mRnu1b6_Scn2aB10_mRnu6 | 721 |
| 358_mRnu1b6_Scn2aB10_mRnu7 | 722 |
| 359_mRnu1b6_Scn2aB10_mRnu11 | 723 |
| 360_mRnu1b6_Scn2aB10_mRnu12 | 724 |
| 361_mRnu1b6_Scn2aB10_mRn7sk | 725 |
| 362_mRnu2-10_Scn2aB10_hRNU1-1 | 726 |
| 363_mRnu2-10_Scn2aB10_hRNU1-2 | 727 |
| 364_mRnu2-10_Scn2aB10_hRNU1-3 | 728 |
| 365_mRnu2-10_Scn2aB10_hRNU2-1 | 729 |
| 366_mRnu2-10_Scn2aB10_hU3 | 730 |
| 367_mRnu2-10_Scn2aB10_hRNU4-1 | 731 |
| 368_mRnu2-10_Scn2aB10_hRNU5A-1 | 732 |
| 369_mRnu2-10_Scn2aB10_hRNU6-1 | 733 |
| 370_mRnu2-10_Scn2aB10_RNU7-1 | 734 |
| 371_mRnu2-10_Scn2aB10_hRNU11 | 735 |
| 372_mRnu2-10_Scn2aB10_hRNU12 | 736 |
| 373_mRnu2-10_Scn2aB10_hRN7SK | 737 |
| 374_mRnu2-10_Scn2aB10_mRnu1a1 | 738 |
| 375_mRnu2-10_Scn2aB10_mRnu1b1 | 739 |
| 376_mRnu2-10_Scn2aB10_mRnu1b6 | 740 |
| 377_mRnu2-10_Scn2aB10_mRnu2-10 | 741 |
| 378_mRnu2-10_Scn2aB10_mRnu3b1 | 742 |
| 379_mRnu2-10_Scn2aB10_mRnu5g | 743 |
| 380_mRnu2-10_Scn2aB10_mRnu6 | 744 |
| 381_mRnu2-10_Scn2aB10_mRnu7 | 745 |
| 382_mRnu2-10_Scn2aB10_mRnu11 | 746 |
| 383_mRnu2-10_Scn2aB10_mRnu12 | 747 |
| 384_mRnu2-10_Scn2aB10_mRn7sk | 748 |
| 385_mu1a1_Scn2aF1Scr2_U1 | 749 |
| 386_mu1a1_Scr2_U1 | 750 |
| 387_mu1a1_Scn2aD12_U1 | 751 |
| 388_mu1a1_Scn2aB10_U1 | 752 |
| 389_mRnu3b1_Scn2aB10_hRNU1-1 | 753 |
| 390_mRnu3b1_Scn2aB10_hRNU1-2 | 754 |
| 391_mRnu3b1_Scn2aB10_hRNU1-3 | 755 |
| 392_mRnu3b1_Scn2aB10_hRNU2-1 | 756 |
| 393_mRnu3b1_Scn2aB10_hU3 | 757 |
| 394_mRnu3b1_Scn2aB10_hRNU4-1 | 758 |
| 395_mRnu3b1_Scn2aB10_hRNU5A-1 | 759 |
| 396_mRnu3b1_Scn2aB10_hRNU6-1 | 760 |
| 397_mRnu3b1_Scn2aB10_RNU7-1 | 761 |
| 398_mRnu3b1_Scn2aB10_hRNU11 | 762 |
| 399_mRnu3b1_Scn2aB10_hRNU12 | 763 |
| 400_mRnu3b1_Scn2aB10_hRN7SK | 764 |
| 401_mRnu3b1_Scn2aB10_mRnu1a1 | 765 |
| 402_mRnu3b1_Scn2aB10_mRnu1b1 | 766 |
| 403_mRnu3b1_Scn2aB10_mRnu1b6 | 767 |
| 404_mRnu3b1_Scn2aB10_mRnu2-10 | 768 |
| 405_mRnu3b1_Scn2aB10_mRnu3b1 | 769 |
| 406_mRnu3b1_Scn2aB10_mRnu5g | 770 |
| 407_mRnu3b1_Scn2aB10_mRnu6 | 771 |
| 408_mRnu3b1_Scn2aB10_mRnu7 | 772 |
| 409_mRnu3b1_Scn2aB10_mRnu11 | 773 |
| 410_mRnu3b1_Scn2aB10_mRnu12 | 774 |
| 411_mRnu3b1_Scn2aB10_mRn7sk | 775 |
| 412_mRnu5g_Scn2aB10_hRNU1-1 | 776 |
| 413_mRnu5g_Scn2aB10_hRNU1-2 | 777 |
| 414_mRnu5g_Scn2aB10_hRNU1-3 | 778 |
| 415_mRnu5g_Scn2aB10_hRNU2-1 | 779 |
| 416_mRnu5g_Scn2aB10_hU3 | 780 |
| 417_mRnu5g_Scn2aB10_hRNU4-1 | 781 |
| 418_mRnu5g_Scn2aB10_hRNU5A-1 | 782 |
| 419_mRnu5g_Scn2aB10_hRNU6-1 | 783 |
| 420_mRnu5g_Scn2aB10_RNU7-1 | 784 |
| 421_mRnu5g_Scn2aB10_hRNU11 | 785 |
| 422_mRnu5g_Scn2aB10_hRNU12 | 786 |
| 423_mRnu5g_Scn2aB10_hRN7SK | 787 |
| 424_mRnu5g_Scn2aB10_mRnu1a1 | 788 |
| 425_mRnu5g_Scn2aB10_mRnu1b1 | 789 |
| 426_mRnu5g_Scn2aB10_mRnu1b6 | 790 |
| 427_mRnu5g_Scn2aB10_mRnu2-10 | 791 |
| 428_mRnu5g_Scn2aB10_mRnu3b1 | 792 |
| 429_mRnu5g_Scn2aB10_mRnu5g | 793 |
| 430_mRnu5g_Scn2aB10_mRnu6 | 794 |
| 431_mRnu5g_Scn2aB10_mRnu7 | 795 |
| 432_mRnu5g_Scn2aB10_mRnu11 | 796 |
| 433_mRnu5g_Scn2aB10_mRnu12 | 797 |
| 434_mRnu5g_Scn2aB10_mRn7sk | 798 |
| 435_mRnu6_Scn2aB10_hRNU1-1 | 799 |
| 436_mRnu6_Scn2aB10_hRNU1-2 | 800 |
| 437_mRnu6_Scn2aB10_hRNU1-3 | 801 |
| 438_mRnu6_Scn2aB10_hRNU2-1 | 802 |
| 439_mRnu6_Scn2aB10_hU3 | 803 |
| 440_mRnu6_Scn2aB10_hRNU4-1 | 804 |
| 441_mRnu6_Scn2aB10_hRNU5A-1 | 805 |
| 442_mRnu6_Scn2aB10_hRNU6-1 | 806 |
| 443_mRnu6_Scn2aB10_RNU7-1 | 807 |
| 444_mRnu6_Scn2aB10_hRNU11 | 808 |
| 445_mRnu6_Scn2aB10_hRNU12 | 809 |
| 446_mRnu6_Scn2aB10_hRN7SK | 810 |
| 447_mRnu6_Scn2aB10_mRnu1a1 | 811 |
| 448_mRnu6_Scn2aB10_mRnu1b1 | 812 |
| 449_mRnu6_Scn2aB10_mRnu1b6 | 813 |
| 450_mRnu6_Scn2aB10_mRnu2-10 | 814 |
| 451_mRnu6_Scn2aB10_mRnu3b1 | 815 |
| 452_mRnu6_Scn2aB10_mRnu5g | 816 |
| 453_mRnu6_Scn2aB10_mRnu6 | 817 |
| 454_mRnu6_Scn2aB10_mRnu7 | 818 |
| 455_mRnu6_Scn2aB10_mRnu11 | 819 |
| 456_mRnu6_Scn2aB10_mRnu12 | 820 |
| 457_mRnu6_Scn2aB10_mRn7sk | 821 |
| 458_mRnu7_Scn2aB10_hRNU1-1 | 822 |
| 459_mRnu7_Scn2aB10_hRNU1-2 | 823 |
| 460_mRnu7_Scn2aB10_hRNU1-3 | 824 |
| 461_mRnu7_Scn2aB10_hRNU2-1 | 825 |
| 462_mRnu7_Scn2aB10_hU3 | 826 |
| 463_mRnu7_Scn2aB10_hRNU4-1 | 827 |
| 464_mRnu7_Scn2aB10_hRNU5A-1 | 828 |
| 465_mRnu7_Scn2aB10_hRNU6-1 | 829 |
| 466_mRnu7_Scn2aB10_RNU7-1 | 830 |
| 467_mRnu7_Scn2aB10_hRNU11 | 831 |
| 468_mRnu7_Scn2aB10_hRNU12 | 832 |
| 469_mRnu7_Scn2aB10_hRN7SK | 833 |
| 470_mRnu7_Scn2aB10_mRnu1a1 | 834 |
| 471_mRnu7_Scn2aB10_mRnu1b1 | 835 |
| 472_mRnu7_Scn2aB10_mRnu1b6 | 836 |
| 473_mRnu7_Scn2aB10_mRnu2-10 | 837 |
| 474_mRnu7_Scn2aB10_mRnu3b1 | 838 |
| 475_mRnu7_Scn2aB10_mRnu5g | 839 |
| 476_mRnu7_Scn2aB10_mRnu6 | 840 |
| 477_mRnu7_Scn2aB10_mRnu7 | 841 |
| 478_mRnu7_Scn2aB10_mRnu11 | 842 |
| 479_mRnu7_Scn2aB10_mRnu12 | 843 |
| 480_mRnu7_Scn2aB10_mRn7sk | 844 |
| 481_mu1a1_Scn2aF1Scr2_U1 | 845 |
| 482_mu1a1_Scr2_U1 | 846 |
| 483_mu1a1_Scn2aD12_U1 | 847 |
| 484_mu1a1_Scn2aB10_U1 | 848 |
| 485_mRnu11_Scn2aB10_hRNU1-1 | 849 |
| 486_mRnu11_Scn2aB10_hRNU1-2 | 850 |
| 487_mRnu11_Scn2aB10_hRNU1-3 | 851 |
| 488_mRnu11_Scn2aB10_hRNU2-1 | 852 |
| 489_mRnu11_Scn2aB10_hU3 | 853 |
| 490_mRnu11_Scn2aB10_hRNU4-1 | 854 |
| 491_mRnu11_Scn2aB10_hRNU5A-1 | 855 |
| 492_mRnu11_Scn2aB10_hRNU6-1 | 856 |
| 493_mRnu11_Scn2aB10_RNU7-1 | 857 |
| 494_mRnu11_Scn2aB10_hRNU11 | 858 |
| 495_mRnu11_Scn2aB10_hRNU12 | 859 |
| 496_mRnu11_Scn2aB10_hRN7SK | 860 |
| 497_mRnu11_Scn2aB10_mRnu1a1 | 861 |
| 498_mRnu11_Scn2aB10_mRnu1b1 | 862 |
| 499_mRnu11_Scn2aB10_mRnu1b6 | 863 |
| 500_mRnu11_Scn2aB10_mRnu2-10 | 864 |
| 501_mRnu11_Scn2aB10_mRnu3b1 | 865 |
| 502_mRnu11_Scn2aB10_mRnu5g | 866 |
| 503_mRnu11_Scn2aB10_mRnu6 | 867 |
| 504_mRnu11_Scn2aB10_mRnu7 | 868 |
| 505_mRnu11_Scn2aB10_mRnu11 | 869 |
| 506_mRnu11_Scn2aB10_mRnu12 | 870 |
| 507_mRnu11_Scn2aB10_mRn7sk | 871 |
| 508_mRnu12_Scn2aB10_hRNU1-1 | 872 |
| 509_mRnu12_Scn2aB10_hRNU1-2 | 873 |
| 510_mRnu12_Scn2aB10_hRNU1-3 | 874 |
| 511_mRnu12_Scn2aB10_hRNU2-1 | 875 |
| 512_mRnu12_Scn2aB10_hU3 | 876 |
| 513_mRnu12_Scn2aB10_hRNU4-1 | 877 |
| 514_mRnu12_Scn2aB10_hRNU5A-1 | 878 |
| 515_mRnu12_Scn2aB10_hRNU6-1 | 879 |
| 516_mRnu12_Scn2aB10_RNU7-1 | 880 |
| 517_mRnu12_Scn2aB10_hRNU11 | 881 |
| 518_mRnu12_Scn2aB10_hRNU12 | 882 |
| 519_mRnu12_Scn2aB10_hRN7SK | 883 |
| 520_mRnu12_Scn2aB10_mRnu1a1 | 884 |
| 521_mRnu12_Scn2aB10_mRnu1b1 | 885 |
| 522_mRnu12_Scn2aB10_mRnu1b6 | 886 |
| 523_mRnu12_Scn2aB10_mRnu2-10 | 887 |
| 524_mRnu12_Scn2aB10_mRnu3b1 | 888 |
| 525_mRnu12_Scn2aB10_mRnu5g | 889 |
| 526_mRnu12_Scn2aB10_mRnu6 | 890 |
| 527_mRnu12_Scn2aB10_mRnu7 | 891 |
| 528_mRnu12_Scn2aB10_mRnu11 | 892 |
| 529_mRnu12_Scn2aB10_mRnu12 | 893 |
| 530_mRnu12_Scn2aB10_mRn7sk | 894 |
| 531_mRn7sk_Scn2aB10_hRNU1-1 | 895 |
| 532_mRn7sk_Scn2aB10_hRNU1-2 | 896 |
| 533_mRn7sk_Scn2aB10_hRNU1-3 | 897 |
| 534_mRn7sk_Scn2aB10_hRNU2-1 | 898 |
| 535_mRn7sk_Scn2aB10_hU3 | 899 |
| 536_mRn7sk_Scn2aB10_hRNU4-1 | 900 |
| 537_mRn7sk_Scn2aB10_hRNU5A-1 | 901 |
| 538_mRn7sk_Scn2aB10_hRNU6-1 | 902 |
| 539_mRn7sk_Scn2aB10_RNU7-1 | 903 |
| 540_mRn7sk_Scn2aB10_hRNU11 | 904 |
| 541_mRn7sk_Scn2aB10_hRNU12 | 905 |
| 542_mRn7sk_Scn2aB10_hRN7SK | 906 |
| 543_mRn7sk_Scn2aB10_mRnu1a1 | 907 |
| 544_mRn7sk_Scn2aB10_mRnu1b1 | 908 |
| 545_mRn7sk_Scn2aB10_mRnu1b6 | 909 |
| 546_mRn7sk_Scn2aB10_mRnu2-10 | 910 |
| 547_mRn7sk_Scn2aB10_mRnu3b1 | 911 |
| 548_mRn7sk_Scn2aB10_mRnu5g | 912 |
| 549_mRn7sk_Scn2aB10_mRnu6 | 913 |
| 550_mRn7sk_Scn2aB10_mRnu7 | 914 |
| 551_mRn7sk_Scn2aB10_mRnu11 | 915 |
| 552_mRn7sk_Scn2aB10_mRnu12 | 916 |
| 553_mRn7sk_Scn2aB10_mRn7sk | 917 |

TABLE 7B
Example U7 Core Sequences included in a library of constructs

| | | SEQ ID |
|---|---|---|
| U7 Core Name | U7 Core Sequence | NO: |
| Scn2aF1Scr2 | aATGATAGGGACTTAGGGTGAGAAATATTCCTCTAACATTCTACAAATT | 919 |
| | TTTGGAGcaggttttctgacttcggtcggaaaacccct | |
| Scr2 | aATGATAGGGACTTAGGGTGAGATAATGGTCACAGGCGGAAGGGATCG | 920 |
| | AATCAGAGTCATGCGGAGGTGTAAATTTTTGGAGcaggttttctgacttcggtcggaa | |
| | aacccct | |
| Scn2aD12 | aATGATAGGGACTTAGGGTGAACATTACTTTGAACTTAACACTACTAG | 921 |
| | AAGAATTTTTGGAGcaggttttctgacttcggtcggaaaacccct | |
| Scn2aB10 | aATGATAGGGACTTAGGGTGATAGAAGTGCAAAGCATGACTGTCAAAT | 922 |
| | TTTTGGAGcaggttttctgacttcggtcggaaaacccct | |

TABLE 8
Library 1 Summary

| | | | | | Rep | Rep | Rep | | | ANOVA |
|---|---|---|---|---|---|---|---|---|---|---|
| Name | ID | Promoter | U7 | Terminator | 1 | 2 | 3 | Average | SD | P-value |
| 054_hRNU1- | 054 | hRNU1-3 | Scn2aB10 | hRNU2-1 | 0.64 | 0.43 | 0.25 | 0.44 | 0.19 | 0.007 |
| 3_Scn2aB10_{—} | | | | | | | | | | |
| hRNU2-1 | | | | | | | | | | |
| 043_hRNU1- | 043 | hRNU1-2 | Scn2aB10 | mRnu2-10 | 0.72 | 0.76 | 0.83 | 0.77 | 0.06 | 0.25 |
| 2_Scn2aB10_{—} | | | | | | | | | | |
| mRnu2-10 | | | | | | | | | | |
| 033_hRNU1- | 033 | hRNU1-2 | Scn2aB10 | hRNU4-1 | 0.53 | 0.90 | 1.03 | 0.82 | 0.26 | 0.35 |
| 2_Scn2aB10_{—} | | | | | | | | | | |
| hRNU4-1 | | | | | | | | | | |
| 073_hRNU1- | 073 | hRNU1-3 | Scn2aB10 | mRn7sk | 0.88 | 0.88 | 0.86 | 0.87 | 0.01 | 0.50 |
| 3_Scn2aB10_{—} | | | | | | | | | | |
| mRn7sk | | | | | | | | | | |
| 044_hRNU1- | 044 | hRNU1-2 | Scn2aB10 | mRnu3b1 | 0.88 | 0.92 | 0.91 | 0.90 | 0.02 | 0.59 |
| 2_Scn2aB10_{—} | | | | | | | | | | |
| mRnu3b1 | | | | | | | | | | |
| 097_mu1a1_{—} | 097 | mu1a1 | Scn2aF1Scr2 | U1 | 0.93 | 1.07 | 0.80 | 0.93 | 0.14 | 0.69 |
| Scn2aF1Scr2_{—} | | | | | | | | | | |
| U1 | | | | | | | | | | |
| 048_hRNU1- | 048 | hRNU1-2 | Scn2aB10 | mRnu11 | 0.82 | 1.03 | 1.07 | 0.97 | 0.13 | 0.84 |
| 2_Scn2aB10_{—} | | | | | | | | | | |
| mRnu11 | | | | | | | | | | |
| 002_mu1a1_{—} | 002 | mu1a1 | Scr2 | U1 | 1.15 | 0.81 | 0.96 | 0.97 | 0.17 | 0.84 |
| Scr2_U1 | | | | | | | | | | |
| 039_hRNU1- | 039 | hRNU1-2 | Scn2aB10 | hRN7SK | 1.31 | 1.10 | 0.59 | 1.00 | 0.37 | 0.94 |
| 2_Scn2aB10_{—} | | | | | | | | | | |
| hRN7SK | | | | | | | | | | |
| 098_mu1a1_{—} | 098 | mu1a1 | Scr2 | U1 | 1.10 | 1.02 | 0.93 | 1.02 | 0.09 | Control |
| Scr2_U1 | | | | | | | | | | |
| 050_hRNU1- | 050 | hRNU1-2 | Scn2aB10 | mRn7sk | 1.16 | 1.13 | 0.86 | 1.05 | 0.17 | 0.88 |
| 2_Scn2aB10_{—} | | | | | | | | | | |
| mRn7sk | | | | | | | | | | |
| 021_hRNU1- | 021 | hRNU1-1 | Scn2aB10 | mRnu3b1 | 0.97 | 1.08 | 1.10 | 1.05 | 0.07 | 0.88 |
| 1_Scn2aB10_{—} | | | | | | | | | | |
| mRnu3b1 | | | | | | | | | | |
| 090_hRNU2- | 090 | hRNU2-1 | Scn2aB10 | mRnu3b1 | 1.08 | 0.82 | 1.26 | 1.05 | 0.23 | 0.86 |
| 1_Scn2aB10_{—} | | | | | | | | | | |
| mRnu3b1 | | | | | | | | | | |
| 085_hRNU2- | 085 | hRNU2-1 | Scn2aB10 | hRN7SK | 1.03 | 0.84 | 1.34 | 1.07 | 0.25 | 0.80 |
| 1_Scn2aB10_{—} | | | | | | | | | | |
| hRN7SK | | | | | | | | | | |
| 016_hRNU1- | 016 | hRNU1-1 | Scn2aB10 | hRN7SK | 0.82 | 1.00 | 1.39 | 1.07 | 0.29 | 0.80 |
| 1_Scn2aB10_{—} | | | | | | | | | | |
| hRN7SK | | | | | | | | | | |
| 001_mu1a1_{—} | 001 | mu1a1 | Scn2aF1Scr2 | U1 | 0.82 | 1.10 | 1.32 | 1.08 | 0.25 | 0.77 |
| Scn2aF1Scr2_{—} | | | | | | | | | | |
| U1 | | | | | | | | | | |
| 027_hRNU1- | 027 | hRNU1-1 | Scn2aB10 | mRn7sk | 1.17 | 1.19 | 0.88 | 1.08 | 0.18 | 0.77 |
| 1_Scn2aB10_{—} | | | | | | | | | | |
| mRn7sk | | | | | | | | | | |
| 071_hRNU1- | 071 | hRNU1-3 | Scn2aB10 | mRnu11 | 1.65 | 0.92 | 0.67 | 1.08 | 0.51 | 0.77 |
| 3_Scn2aB10_{—} | | | | | | | | | | |
| mRnu11 | | | | | | | | | | |
| 036_hRNU1- | 036 | hRNU1-2 | Scn2aB10 | RNU7-1 | 0.99 | 0.90 | 1.37 | 1.09 | 0.25 | 0.74 |
| 2_Scn2aB10_{—} | | | | | | | | | | |
| RNU7-1 | | | | | | | | | | |
| 031_hRNU1- | 031 | hRNU1-2 | Scn2aB10 | hRNU2-1 | 1.19 | 1.06 | 1.06 | 1.11 | 0.08 | 0.68 |
| 2_Scn2aB10_{—} | | | | | | | | | | |
| hRNU2-1 | | | | | | | | | | |
| 062_hRNU1- | 062 | hRNU1-3 | Scn2aB10 | hRN7SK | 0.89 | 1.49 | 1.05 | 1.14 | 0.31 | 0.55 |
| 3_Scn2aB10_{—} | | | | | | | | | | |
| hRN7SK | | | | | | | | | | |
| 086_hRNU2- | 086 | hRNU2-1 | Scn2aB10 | mRnu1a1 | 1.05 | 0.79 | 1.64 | 1.16 | 0.44 | 0.50 |
| 1_Scn2aB10_{—} | | | | | | | | | | |
| mRnu1a1 | | | | | | | | | | |
| 052_hRNU1- | 052 | hRNU1-3 | Scn2aB10 | hRNU1-2 | 1.34 | 1.32 | 0.84 | 1.16 | 0.28 | 0.48 |
| 3_Scn2aB10_{—} | | | | | | | | | | |
| hRNU1-2 | | | | | | | | | | |
| 053_hRNU1- | 053 | hRNU1-3 | Scn2aB10 | hRNU1-3 | 0.67 | 1.88 | 0.96 | 1.17 | 0.63 | 0.47 |
| 3_Scn2aB10_{—} | | | | | | | | | | |
| hRNU1-3 | | | | | | | | | | |
| 067_hRNU1- | 067 | hRNU1-3 | Scn2aB10 | mRnu3b1 | 1.26 | 1.18 | 1.09 | 1.18 | 0.09 | 0.45 |
| 3_Scn2aB10_{—} | | | | | | | | | | |
| mRnu3b1 | | | | | | | | | | |
| 042_hRNU1- | 042 | hRNU1-2 | Scn2aB10 | mRnu1b6 | 1.07 | 1.19 | 1.33 | 1.20 | 0.13 | 0.40 |
| 2_Scn2aB10_{—} | | | | | | | | | | |
| mRnu1b6 | | | | | | | | | | |
| 059_hRNU1- | 059 | hRNU1-3 | Scn2aB10 | RNU7-1 | 1.40 | 1.10 | 1.13 | 1.21 | 0.16 | 0.36 |
| 3_Scn2aB10_{—} | | | | | | | | | | |
| RNU7-1 | | | | | | | | | | |
| 063_hRNU1- | 063 | hRNU1-3 | Scn2aB10 | mRnu1a1 | 1.43 | 1.20 | 1.01 | 1.21 | 0.21 | 0.35 |
| 3_Scn2aB10_{—} | | | | | | | | | | |
| mRnu1a1 | | | | | | | | | | |
| 072_hRNU1- | 072 | hRNU1-3 | Scn2aB10 | mRnu12 | 1.01 | 1.18 | 1.47 | 1.22 | 0.23 | 0.34 |
| 3_Scn2aB10_{—} | | | | | | | | | | |
| mRnu12 | | | | | | | | | | |
| 005_hRNU1- | 005 | hRNU1-1 | Scn2aB10 | hRNU1-1 | 1.39 | 1.04 | 1.25 | 1.23 | 0.18 | 0.32 |
| 1_Scn2aB10_{—} | | | | | | | | | | |
| hRNU1-1 | | | | | | | | | | |
| 081_hRNU2- | 081 | hRNU2-1 | Scn2aB10 | hRNU6-1 | 1.55 | 1.13 | 1.02 | 1.23 | 0.28 | 0.31 |
| 1_Scn2aB10_{—} | | | | | | | | | | |
| hRNU6-1 | | | | | | | | | | |
| 060_hRNU1- | 060 | hRNU1-3 | Scn2aB10 | hRNU11 | 1.21 | 1.23 | 1.26 | 1.23 | 0.03 | 0.31 |
| 3_Scn2aB10_{—} | | | | | | | | | | |
| hRNU11 | | | | | | | | | | |
| 013_hRNU1- | 013 | hRNU1-1 | Scn2aB10 | RNU7-1 | 1.28 | 1.13 | 1.31 | 1.24 | 0.10 | 0.29 |
| 1_Scn2aB10_{—} | | | | | | | | | | |
| RNU7-1 | | | | | | | | | | |
| 008_hRNU1- | 008 | hRNU1-1 | Scn2aB10 | hRNU2-1 | 1.26 | 0.84 | 1.62 | 1.24 | 0.39 | 0.29 |
| 1_Scn2aB10_{—} | | | | | | | | | | |
| hRNU2-1 | | | | | | | | | | |
| 014_hRNU1- | 014 | hRNU1-1 | Scn2aB10 | hRNU11 | 1.25 | 1.34 | 1.13 | 1.24 | 0.11 | 0.29 |
| 1_Scn2aB10_{—} | | | | | | | | | | |
| hRNU11 | | | | | | | | | | |
| 049_hRNU1- | 049 | hRNU1-2 | Scn2aB10 | mRnu12 | 1.27 | 1.26 | 1.19 | 1.24 | 0.04 | 0.29 |
| 2_Scn2aB10_{—} | | | | | | | | | | |
| mRnu12 | | | | | | | | | | |
| 032_hRNU1- | 032 | hRNU1-2 | Scn2aB10 | hU3 | 1.27 | 1.46 | 1.03 | 1.25 | 0.21 | 0.26 |
| 2_Scn2aB10_{—} | | | | | | | | | | |
| hU3 | | | | | | | | | | |
| 058_hRNU1- | 058 | hRNU1-3 | Scn2aB10 | hRNU6-1 | 1.41 | 1.06 | 1.31 | 1.26 | 0.18 | 0.25 |
| 3_Scn2aB10_{—} | | | | | | | | | | |
| hRNU6-1 | | | | | | | | | | |
| 051_hRNU1- | 051 | hRNU1-3 | Scn2aB10 | hRNU1-1 | 1.60 | 1.05 | 1.17 | 1.27 | 0.29 | 0.23 |
| 3_Scn2aB10_{—} | | | | | | | | | | |
| hRNU1-1 | | | | | | | | | | |
| 020_hRNU1- | 020 | hRNU1-1 | Scn2aB10 | mRnu2-10 | 1.27 | 1.39 | 1.15 | 1.27 | 0.12 | 0.23 |
| 1_Scn2aB10_{—} | | | | | | | | | | |
| mRnu2-10 | | | | | | | | | | |
| 064_hRNU1- | 064 | hRNU1-3 | Scn2aB10 | mRnu1b1 | 1.32 | 1.09 | 1.43 | 1.28 | 0.17 | 0.21 |
| 3_Scn2aB10_{—} | | | | | | | | | | |
| mRnu1b1 | | | | | | | | | | |
| 096_hRNU2- | 096 | hRNU2-1 | Scn2aB10 | mRn7sk | 1.27 | 1.32 | 1.25 | 1.28 | 0.04 | 0.21 |
| 1_Scn2aB10_{—} | | | | | | | | | | |
| mRn7sk | | | | | | | | | | |
| 061_hRNU1- | 061 | hRNU1-3 | Scn2aB10 | hRNU12 | 1.76 | 0.96 | 1.17 | 1.30 | 0.41 | 0.19 |
| 3_Scn2aB10_{—} | | | | | | | | | | |
| hRNU12 | | | | | | | | | | |
| 034_hRNU1- | 034 | hRNU1-2 | Scn2aB10 | hRNU5A-1 | 1.37 | 1.19 | 1.34 | 1.30 | 0.10 | 0.18 |
| 2_Scn2aB10_{—} | | | | | | | | | | |
| hRNU5A-1 | | | | | | | | | | |
| 055_hRNU1- | 055 | hRNU1-3 | Scn2aB10 | hU3 | 1.29 | 1.50 | 1.12 | 1.30 | 0.19 | 0.18 |
| 3_Scn2aB10_{—} | | | | | | | | | | |
| hU3 | | | | | | | | | | |
| 078_hRNU2- | 078 | hRNU2-1 | Scn2aB10 | hU3 | 1.42 | 1.19 | 1.32 | 1.31 | 0.12 | 0.17 |
| 1_Scn2aB10_{—} | | | | | | | | | | |
| hU3 | | | | | | | | | | |
| 012_hRNU1- | 012 | hRNU1-1 | Scn2aB10 | hRNU6-1 | 1.35 | 1.50 | 1.09 | 1.31 | 0.20 | 0.16 |
| 1_Scn2aB10_{—} | | | | | | | | | | |
| hRNU6-1 | | | | | | | | | | |
| 037_hRNU1- | 037 | hRNU1-2 | Scn2aB10 | hRNU11 | 1.53 | 1.12 | 1.30 | 1.32 | 0.21 | 0.16 |
| 2_Scn2aB10_{—} | | | | | | | | | | |
| hRNU11 | | | | | | | | | | |
| 025_hRNU1- | 025 | hRNU1-1 | Scn2aB10 | mRnu11 | 1.78 | 1.31 | 0.87 | 1.32 | 0.46 | 0.15 |
| 1_Scn2aB10_{—} | | | | | | | | | | |
| mRnu11 | | | | | | | | | | |
| 007_hRNU1- | 007 | hRNU1-1 | Scn2aB10 | hRNU1-3 | 1.44 | 1.32 | 1.21 | 1.32 | 0.11 | 0.15 |
| 1_Scn2aB10_{—} | | | | | | | | | | |
| hRNU1-3 | | | | | | | | | | |
| 077_hRNU2- | 077 | hRNU2-1 | Scn2aB10 | hRNU2-1 | 1.54 | 1.15 | 1.29 | 1.33 | 0.20 | 0.14 |
| 1_Scn2aB10_{—} | | | | | | | | | | |
| hRNU2-1 | | | | | | | | | | |
| 022_hRNU1- | 022 | hRNU1-1 | Scn2aB10 | mRnu5g | 1.33 | 1.41 | 1.24 | 1.33 | 0.08 | 0.14 |
| 1_Scn2aB10_{—} | | | | | | | | | | |
| mRnu5g | | | | | | | | | | |
| 019_hRNU1- | 019 | hRNU1-1 | Scn2aB10 | mRnu1b6 | 1.98 | 1.04 | 0.98 | 1.33 | 0.56 | 0.14 |
| 1_Scn2aB10_{—} | | | | | | | | | | |
| mRnu1b6 | | | | | | | | | | |
| 003_mu1a1_{—} | 003 | mu1a1 | Scn2aD12 | U1 | 0.95 | 1.49 | 1.59 | 1.34 | 0.35 | 0.12 |
| Scn2aD12_U1 | | | | | | | | | | |
| 065_hRNU1- | 065 | hRNU1-3 | Scn2aB10 | mRnu1b6 | 1.57 | 1.39 | 1.07 | 1.34 | 0.25 | 0.12 |
| 3_Scn2aB10_{—} | | | | | | | | | | |
| mRnu1b6 | | | | | | | | | | |
| 045_hRNU1- | 045 | hRNU1-2 | Scn2aB10 | mRnu5g | 1.42 | 1.21 | 1.40 | 1.35 | 0.11 | 0.12 |
| 2_Scn2aB10_{—} | | | | | | | | | | |
| mRnu5g | | | | | | | | | | |
| 018_hRNU1- | 018 | hRNU1-1 | Scn2aB10 | mRnu1b1 | 1.75 | 1.38 | 0.93 | 1.35 | 0.41 | 0.11 |
| 1_Scn2aB10_{—} | | | | | | | | | | |
| mRnu1b1 | | | | | | | | | | |
| 070_hRNU1- | 070 | hRNU1-3 | Scn2aB10 | mRnu7 | 1.59 | 1.26 | 1.21 | 1.35 | 0.21 | 0.11 |
| 3_Scn2aB10_{—} | | | | | | | | | | |
| mRnu7 | | | | | | | | | | |
| 006_hRNU1- | 006 | hRNU1-1 | Scn2aB10 | hRNU1-2 | 0.98 | 1.92 | 1.16 | 1.36 | 0.50 | 0.11 |
| 1_Scn2aB10_{—} | | | | | | | | | | |
| hRNU1-2 | | | | | | | | | | |
| 024_hRNU1- | 024 | hRNU1-1 | Scn2aB10 | mRnu7 | 1.54 | 1.43 | 1.11 | 1.36 | 0.22 | 0.11 |
| 1_Scn2aB10_{—} | | | | | | | | | | |
| mRnu7 | | | | | | | | | | |
| 035_hRNU1- | 035 | hRNU1-2 | Scn2aB10 | hRNU6-1 | 1.48 | 1.47 | 1.16 | 1.37 | 0.18 | 0.10 |
| 2_Scn2aB10_{—} | | | | | | | | | | |
| hRNU6-1 | | | | | | | | | | |
| 079_hRNU2- | 079 | hRNU2-1 | Scn2aB10 | hRNU4-1 | 1.44 | 1.40 | 1.34 | 1.39 | 0.05 | 0.08 |
| 1_Scn2aB10_{—} | | | | | | | | | | |
| hRNU4-1 | | | | | | | | | | |
| 040_hRNU1- | 040 | hRNU1-2 | Scn2aB10 | mRnu1a1 | 1.21 | 1.28 | 1.72 | 1.41 | 0.27 | 0.07 |
| 2_Scn2aB10_{—} | | | | | | | | | | |
| mRnu1a1 | | | | | | | | | | |
| 015_hRNU1- | 015 | hRNU1-1 | Scn2aB10 | hRNU12 | 1.43 | 1.45 | 1.34 | 1.41 | 0.06 | 0.07 |
| 1_Scn2aB10_{—} | | | | | | | | | | |
| hRNU12 | | | | | | | | | | |
| 095_hRNU2- | 095 | hRNU2-1 | Scn2aB10 | mRnu12 | 1.61 | 1.12 | 1.55 | 1.43 | 0.27 | 0.05 |
| 1_Scn2aB10_{—} | | | | | | | | | | |
| mRnu12 | | | | | | | | | | |
| 057_hRNU1- | 057 | hRNU1-3 | Scn2aB10 | hRNU5A-1 | 1.35 | 1.48 | 1.45 | 1.43 | 0.07 | 0.05 |
| 3_Scn2aB10_{—} | | | | | | | | | | |
| hRNU5A-1 | | | | | | | | | | |
| 089_hRNU2- | 089 | hRNU2-1 | Scn2aB10 | mRnu2-10 | 1.53 | 1.71 | 1.11 | 1.45 | 0.31 | 0.04 |
| 1_Scn2aB10_{—} | | | | | | | | | | |
| mRnu2-10 | | | | | | | | | | |
| 030_hRNU1- | 030 | hRNU1-2 | Scn2aB10 | hRNU1-3 | 1.10 | 1.61 | 1.66 | 1.45 | 0.31 | 0.04 |
| 2_Scn2aB10_{—} | | | | | | | | | | |
| hRNU1-3 | | | | | | | | | | |
| 066_hRNU1- | 066 | hRNU1-3 | Scn2aB10 | mRnu2-10 | 1.71 | 1.65 | 1.04 | 1.47 | 0.37 | 0.03 |
| 3_Scn2aB10_{—} | | | | | | | | | | |
| mRnu2-10 | | | | | | | | | | |
| 038_hRNU1- | 038 | hRNU1-2 | Scn2aB10 | hRNU12 | 1.62 | 1.33 | 1.47 | 1.47 | 0.14 | 0.03 |
| 2_Scn2aB10_{—} | | | | | | | | | | |
| hRNU12 | | | | | | | | | | |
| 029_hRNU1- | 029 | hRNU1-2 | Scn2aB10 | hRNU1-2 | 1.68 | 1.28 | 1.49 | 1.48 | 0.20 | 0.03 |
| 2_Scn2aB10_{—} | | | | | | | | | | |
| hRNU1-2 | | | | | | | | | | |
| 082_hRNU2- | 082 | hRNU2-1 | Scn2aB10 | RNU7-1 | 1.45 | 2.18 | 0.85 | 1.49 | 0.67 | 0.03 |
| 1_Scn2aB10_{—} | | | | | | | | | | |
| RNU7-1 | | | | | | | | | | |
| 076_hRNU2- | 076 | hRNU2-1 | Scn2aB10 | hRNU1-3 | 1.67 | 1.49 | 1.35 | 1.50 | 0.16 | 0.02 |
| 1_Scn2aB10_{—} | | | | | | | | | | |
| hRNU1-3 | | | | | | | | | | |
| 041_hRNU1- | 041 | hRNU1-2 | Scn2aB10 | mRnu1b1 | 1.37 | 1.73 | 1.42 | 1.51 | 0.19 | 0.02 |
| 2_Scn2aB10_{—} | | | | | | | | | | |
| mRnu1b1 | | | | | | | | | | |
| 094_hRNU2- | 094 | hRNU2-1 | Scn2aB10 | mRnu11 | 1.54 | 1.58 | 1.44 | 1.52 | 0.07 | 0.02 |
| 1_Scn2aB10_{—} | | | | | | | | | | |
| mRnu11 | | | | | | | | | | |
| 088_hRNU2- | 088 | hRNU2-1 | Scn2aB10 | mRnu1b6 | 1.49 | 1.49 | 1.58 | 1.52 | 0.05 | 0.02 |
| 1_Scn2aB10_{—} | | | | | | | | | | |
| mRnu1b6 | | | | | | | | | | |
| 068_hRNU1- | 068 | hRNU1-3 | Scn2aB10 | mRnu5g | 1.13 | 1.74 | 1.70 | 1.52 | 0.34 | 0.02 |
| 3_Scn2aB10_{—} | | | | | | | | | | |
| mRnu5g | | | | | | | | | | |
| 087_hRNU2- | 087 | hRNU2-1 | Scn2aB10 | mRnu1b1 | 1.23 | 1.79 | 1.55 | 1.53 | 0.28 | 0.02 |
| 1_Scn2aB10_{—} | | | | | | | | | | |
| mRnu1b1 | | | | | | | | | | |
| 080_hRNU2- | 080 | hRNU2-1 | Scn2aB10 | hRNU5A-1 | 1.88 | 1.58 | 1.14 | 1.54 | 0.37 | 0.02 |
| 1_Scn2aB10_{—} | | | | | | | | | | |
| hRNU5A-1 | | | | | | | | | | |
| 047_hRNU1- | 047 | hRNU1-2 | Scn2aB10 | mRnu7 | 1.26 | 1.63 | 1.72 | 1.54 | 0.24 | 0.01 |
| 2_Scn2aB10_{—} | | | | | | | | | | |
| mRnu7 | | | | | | | | | | |
| 056_hRNU1- | 056 | hRNU1-3 | Scn2aB10 | hRNU4-1 | 1.59 | 1.83 | 1.19 | 1.54 | 0.32 | 0.01 |
| 3_Scn2aB10_{—} | | | | | | | | | | |
| hRNU4-1 | | | | | | | | | | |
| 011_hRNU1- | 011 | hRNU1-1 | Scn2aB10 | hRNU5A-1 | 1.30 | 2.09 | 1.24 | 1.54 | 0.48 | 0.01 |
| 1_Scn2aB10_{—} | | | | | | | | | | |
| hRNU5A-1 | | | | | | | | | | |
| 075_hRNU2- | 075 | hRNU2-1 | Scn2aB10 | hRNU1-2 | 1.57 | 1.52 | 1.56 | 1.55 | 0.03 | 0.01 |
| 1_Scn2aB10_{—} | | | | | | | | | | |
| hRNU1-2 | | | | | | | | | | |
| 093_hRNU2- | 093 | hRNU2-1 | Scn2aB10 | mRnu7 | 1.38 | 1.88 | 1.39 | 1.55 | 0.28 | 0.01 |
| 1_Scn2aB10_{—} | | | | | | | | | | |
| mRnu7 | | | | | | | | | | |
| 009_hRNU1- | 009 | hRNU1-1 | Scn2aB10 | hU3 | 1.82 | 1.37 | 1.46 | 1.55 | 0.24 | 0.01 |
| 1_Scn2aB10_{—} | | | | | | | | | | |
| hU3 | | | | | | | | | | |
| 084_hRNU2- | 084 | hRNU2-1 | Scn2aB10 | hRNU12 | 1.68 | 1.50 | 1.47 | 1.55 | 0.11 | 0.01 |
| 1_Scn2aB10_{—} | | | | | | | | | | |
| hRNU12 | | | | | | | | | | |
| 083_hRNU2- | 083 | hRNU2-1 | Scn2aB10 | hRNU11 | 1.42 | 1.87 | 1.44 | 1.58 | 0.25 | 0.009 |
| 1_Scn2aB10_{—} | | | | | | | | | | |
| hRNU11 | | | | | | | | | | |
| 026_hRNU1- | 026 | hRNU1-1 | Scn2aB10 | mRnu12 | 1.30 | 1.68 | 1.91 | 1.63 | 0.31 | 0.004 |
| 1_Scn2aB10_{—} | | | | | | | | | | |
| mRnu12 | | | | | | | | | | |
| 074_hRNU2- | 074 | hRNU2-1 | Scn2aB10 | hRNU1-1 | 1.60 | 1.71 | 1.83 | 1.71 | 0.11 | 0.001 |
| 1_Scn2aB10_{—} | | | | | | | | | | |
| hRNU1-1 | | | | | | | | | | |
| 099_mu1a1_{—} | 099 | mu1a1 | Scn2aD12 | U1 | 2.02 | 1.67 | 1.50 | 1.73 | 0.27 | <0.001 |
| Scn2aD12_U1 | | | | | | | | | | |
| 010_hRNU1- | 010 | hRNU1-1 | Scn2aB10 | hRNU4-1 | 1.61 | 1.74 | 1.91 | 1.75 | 0.15 | <0.001 |
| 1_Scn2aB10_{—} | | | | | | | | | | |
| hRNU4-1 | | | | | | | | | | |
| 004_mu1a1_{—} | 004 | mu1a1 | Scn2aB10 | U1 | 1.57 | 2.11 | 1.62 | 1.77 | 0.30 | <0.001 |
| Scn2aB10_U1 | | | | | | | | | | |
| 028_hRNU1- | 028 | hRNU1-2 | Scn2aB10 | hRNU1-1 | 1.72 | 1.55 | 2.04 | 1.77 | 0.25 | <0.001 |
| 2_Scn2aB10_{—} | | | | | | | | | | |
| hRNU1-1 | | | | | | | | | | |
| 091_hRNU2- | 091 | hRNU2-1 | Scn2aB10 | mRnu5g | 1.50 | 2.16 | 1.87 | 1.84 | 0.33 | <0.001 |
| 1_Scn2aB10_{—} | | | | | | | | | | |
| mRnu5g | | | | | | | | | | |
| 017_hRNU1- | 017 | hRNU1-1 | Scn2aB10 | mRnu1a1 | 2.02 | 1.84 | 1.71 | 1.86 | 0.16 | <0.001 |
| 1_Scn2aB10_{—} | | | | | | | | | | |
| mRnu1a1 | | | | | | | | | | |

TABLE 9
Library 2 Summary

| Name | Rep1 | Rep2 | Rep3 | Average | SD | P-Value |
|---|---|---|---|---|---|---|
| 114_hU3_Scn2aB10_mRnu1b1 | 0.70 | 0.99 | 1.20 | 0.96 | 0.25 | 0.91 |
| 194_mu1a1_Scr2_U1 | 1.00 | 1.00 | 1.00 | 1.00 | 0.00 | Control |
| 113_hU3_Scn2aB10_mRnu1a1 | 0.89 | 0.89 | 1.26 | 1.01 | 0.22 | 0.97 |
| 178_hRNU6- | 0.95 | 0.98 | 1.18 | 1.04 | 0.12 | 0.91 |
| 1_Scn2aB10_RNU7-1 | | | | | | |
| 172_hRNU6- | 0.83 | 0.96 | 1.38 | 1.06 | 0.29 | 0.86 |
| 1_Scn2aB10_hRNU1-3 | | | | | | |
| 104_hU3_Scn2aB10_hRNU2-1 | 0.47 | 1.25 | 1.49 | 1.07 | 0.53 | 0.82 |
| 179_hRNU6- | 0.89 | 0.97 | 1.38 | 1.08 | 0.26 | 0.8 |
| 1_Scn2aB10_hRNU11 | | | | | | |
| 111_hU3_Scn2aB10_hRNU12 | 1.06 | 0.90 | 1.30 | 1.08 | 0.20 | 0.78 |
| 108_hU3_Scn2aB10_hRNU6-1 | 0.86 | 1.19 | 1.23 | 1.09 | 0.20 | 0.77 |
| 117_hU3_Scn2aB10_mRnu3b1 | 0.66 | 1.05 | 1.57 | 1.09 | 0.45 | 0.77 |
| 112_hU3_Scn2aB10_hRN7SK | 0.95 | 1.07 | 1.28 | 1.10 | 0.17 | 0.75 |
| 110_hU3_Scn2aB10_hRNU11 | 1.14 | 0.70 | 1.55 | 1.13 | 0.43 | 0.68 |
| 115_hU3_Scn2aB10_mRnu1b6 | 1.00 | 0.84 | 1.59 | 1.14 | 0.40 | 0.65 |
| 106_hU3_Scn2aB10_hRNU4-1 | 1.12 | 1.03 | 1.42 | 1.19 | 0.20 | 0.54 |
| 103_hU3_Scn2aB10_hRNU1-3 | 1.03 | 1.27 | 1.29 | 1.19 | 0.15 | 0.53 |
| 189_hRNU6- | 0.77 | 1.14 | 1.74 | 1.22 | 0.49 | 0.49 |
| 1_Scn2aB10_mRnu7 | | | | | | |
| 101_hU3_Scn2aB10_hRNU1-1 | 0.88 | 1.17 | 1.63 | 1.23 | 0.38 | 0.47 |
| 109_hU3_Scn2aB10_RNU7-1 | 1.14 | 0.94 | 1.61 | 1.23 | 0.34 | 0.46 |
| 123_hU3_Scn2aB10_mRn7sk | 1.08 | 1.47 | 1.24 | 1.27 | 0.19 | 0.4 |
| 122_hU3_Scn2aB10_mRnu12 | 0.93 | 1.47 | 1.41 | 1.27 | 0.30 | 0.39 |
| 181_hRNU6- | 0.99 | 1.08 | 1.82 | 1.30 | 0.46 | 0.34 |
| 1_Scn2aB10_hRN7SK | | | | | | |
| 163_hRNU5A- | 1.12 | 1.23 | 1.59 | 1.31 | 0.25 | 0.32 |
| 1_Scn2aB10_mRnu3b1 | | | | | | |
| 169_hRNU5A- | 1.06 | 1.09 | 1.80 | 1.32 | 0.42 | 0.31 |
| 1_Scn2aB10_mRn7sk | | | | | | |
| 162_hRNU5A- | 1.43 | 1.49 | 1.07 | 1.33 | 0.22 | 0.29 |
| 1_Scn2aB10_mRnu2-10 | | | | | | |
| 102_hU3_Scn2aB10_hRNU1-2 | 1.08 | 1.55 | 1.37 | 1.33 | 0.23 | 0.29 |
| 191_hRNU6- | 0.93 | 1.48 | 1.59 | 1.33 | 0.36 | 0.29 |
| 1_Scn2aB10_mRnu12 | | | | | | |
| 186_hRNU6- | 1.23 | 1.23 | 1.58 | 1.34 | 0.20 | 0.27 |
| 1_Scn2aB10_mRnu3b1 | | | | | | |
| 192_hRNU6- | 1.07 | 1.08 | 1.94 | 1.36 | 0.50 | 0.25 |
| 1_Scn2aB10_mRn7sk | | | | | | |
| 107_hU3_Scn2aB10_hRNU5A-1 | 1.24 | 0.97 | 1.92 | 1.38 | 0.49 | 0.23 |
| 200_RNU7- | 1.52 | 1.27 | 1.44 | 1.41 | 0.13 | 0.19 |
| 1_Scn2aB10_hRNU2-1 | | | | | | |
| 121_hU3_Scn2aB10_mRnu11 | 1.09 | 1.45 | 1.71 | 1.42 | 0.31 | 0.18 |
| 133_hRNU4- | 0.94 | 2.29 | 1.04 | 1.42 | 0.75 | 0.18 |
| 1_Scn2aB10_hRNU11 | | | | | | |
| 116_hU3_Scn2aB10_mRnu2-10 | 1.27 | 1.53 | 1.47 | 1.42 | 0.14 | 0.18 |
| 193_mu1a1_Scn2aF1Scr2_U1 | 1.57 | 1.05 | 1.70 | 1.44 | 0.34 | 0.16 |
| 175_hRNU6- | 1.16 | 1.24 | 1.95 | 1.45 | 0.44 | 0.15 |
| 1_Scn2aB10_hRNU4-1 | | | | | | |
| 187_hRNU6- | 1.19 | 1.78 | 1.47 | 1.48 | 0.29 | 0.13 |
| 1_Scn2aB10_mRnu5g | | | | | | |
| 146_hRNU4- | 1.35 | 1.50 | 1.58 | 1.48 | 0.12 | 0.13 |
| 1_Scn2aB10_mRn7sk | | | | | | |
| 190_hRNU6- | 1.17 | 1.76 | 1.54 | 1.49 | 0.30 | 0.12 |
| 1_Scn2aB10_mRnu11 | | | | | | |
| 139_hRNU4- | 1.45 | 1.76 | 1.26 | 1.49 | 0.25 | 0.12 |
| 1_Scn2aB10_mRnu2-10 | | | | | | |
| 120_hU3_Scn2aB10_mRnu7 | 1.51 | 1.31 | 1.67 | 1.50 | 0.18 | 0.11 |
| 182_hRNU6- | 1.14 | 1.50 | 1.86 | 1.50 | 0.36 | 0.11 |
| 1_Scn2aB10_mRnu1a1 | | | | | | |
| 177_hRNU6- | 0.70 | 1.52 | 2.34 | 1.52 | 0.82 | 0.1 |
| 1_Scn2aB10_hRNU6-1 | | | | | | |
| 180_hRNU6- | 0.92 | 1.61 | 2.13 | 1.55 | 0.61 | 0.08 |
| 1_Scn2aB10_hRNU12 | | | | | | |
| 176_hRNU6- | 1.36 | 1.17 | 2.18 | 1.57 | 0.54 | 0.07 |
| 1_Scn2aB10_hRNU5A-1 | | | | | | |
| 183_hRNU6- | 0.90 | 1.16 | 2.77 | 1.6 | 1.02 | 0.05 |
| 1_Scn2aB10_mRnu1b1 | | | | | | |
| 144_hRNU4- | 1.35 | 1.53 | 1.95 | 1.6 | 0.31 | 0.05 |
| 1_Scn2aB10_mRnu11 | | | | | | |
| 127_hRNU4- | 1.35 | 1.76 | 1.75 | 1.62 | 0.23 | 0.05 |
| 1_Scn2aB10_hRNU2-1 | | | | | | |
| 161_hRNU5A- | 1.60 | 1.21 | 2.13 | 1.65 | 0.46 | 0.04 |
| 1_Scn2aB10_mRnu1b6 | | | | | | |
| 140_hRNU4- | 1.37 | 1.52 | 2.12 | 1.67 | 0.40 | 0.03 |
| 1_Scn2aB10_mRnu3b1 | | | | | | |
| 174_hRNU6-1_Scn2aB10_hU3 | 1.45 | 1.47 | 2.13 | 1.68 | 0.39 | 0.03 |
| 185_hRNU6- | 1.07 | 1.97 | 2.02 | 1.69 | 0.53 | 0.03 |
| 1_Scn2aB10_mRnu2-10 | | | | | | |
| 135_hRNU4- | 1.13 | 1.36 | 2.58 | 1.69 | 0.78 | 0.03 |
| 1_Scn2aB10_hRN7SK | | | | | | |
| 150_hRNU5A- | 1.36 | 1.68 | 2.05 | 1.70 | 0.34 | 0.03 |
| 1_Scn2aB10_hRNU2-1 | | | | | | |
| 131_hRNU4- | 1.14 | 2.08 | 1.90 | 1.71 | 0.50 | 0.02 |
| 1_Scn2aB10_hRNU6-1 | | | | | | |
| 173_hRNU6- | 1.67 | 1.92 | 1.53 | 1.71 | 0.20 | 0.02 |
| 1_Scn2aB10_hRNU2-1 | | | | | | |
| 164_hRNU5A- | 1.09 | 2.14 | 1.89 | 1.71 | 0.55 | 0.02 |
| 1_Scn2aB10_mRnu5g | | | | | | |
| 145_hRNU4- | 1.01 | 1.59 | 2.52 | 1.71 | 0.76 | 0.02 |
| 1_Scn2aB10_mRnu12 | | | | | | |
| 199_RNU7- | 1.50 | 1.37 | 2.28 | 1.72 | 0.49 | 0.02 |
| 1_Scn2aB10_hRNU1-3 | | | | | | |
| 201_RNU7-1_Scn2aB10_hU3 | 1.64 | 1.34 | 2.17 | 1.72 | 0.42 | 0.02 |
| 170_hRNU6- | 1.33 | 2.00 | 1.83 | 1.72 | 0.35 | 0.02 |
| 1_Scn2aB10_hRNU1-1 | | | | | | |
| 184_hRNU6- | 1.18 | 1.61 | 2.43 | 1.74 | 0.63 | 0.02 |
| 1_Scn2aB10_mRnu1b6 | | | | | | |
| 137_hRNU4- | 1.53 | 1.43 | 2.34 | 1.77 | 0.50 | 0.01 |
| 1_Scn2aB10_mRnu1b1 | | | | | | |
| 198_RNU7- | 1.06 | 1.35 | 2.97 | 1.79 | 1.03 | 0.01 |
| 1_Scn2aB10_hRNU1-2 | | | | | | |
| 118_hU3_Scn2aB10_mRnu5g | 1.22 | 2.01 | 2.18 | 1.80 | 0.51 | 0.01 |
| 196_mu1a1_Scn2aB10_U1 | 1.45 | 1.55 | 2.48 | 1.83 | 0.57 | 0.009 |
| 158_hRNU5A- | 1.10 | 1.87 | 2.53 | 1.83 | 0.72 | 0.008 |
| 1_Scn2aB10_hRN7SK | | | | | | |
| 154_hRNU5A- | 1.82 | 1.61 | 2.08 | 1.84 | 0.24 | 0.008 |
| 1_Scn2aB10_hRNU6-1 | | | | | | |
| 149_hRNU5A- | 1.34 | 1.67 | 2.64 | 1.88 | 0.68 | 0.005 |
| 1_Scn2aB10_hRNU1-3 | | | | | | |
| 124_hRNU4- | 1.55 | 2.30 | 1.85 | 1.90 | 0.38 | 0.004 |
| 1_Scn2aB10_hRNU1-1 | | | | | | |
| 171_hRNU6- | 1.72 | 1.37 | 2.68 | 1.93 | 0.68 | 0.004 |
| 1_Scn2aB10_hRNU1-2 | | | | | | |
| 138_hRNU4- | 1.38 | 2.11 | 2.42 | 1.97 | 0.53 | 0.002 |
| 1_Scn2aB10_mRnu1b6 | | | | | | |
| 152_hRNU5A- | 2.47 | 1.90 | 1.58 | 1.99 | 0.45 | 0.002 |
| 1_Scn2aB10_hRNU4-1 | | | | | | |
| 130_hRNU4- | 1.68 | 2.14 | 2.16 | 1.99 | 0.27 | 0.002 |
| 1_Scn2aB10_hRNU5A-1 | | | | | | |
| 132_hRNU4- | 1.10 | 2.25 | 2.62 | 1.99 | 0.79 | 0.002 |
| 1_Scn2aB10_RNU7-1 | | | | | | |
| 156_hRNU5A- | 1.58 | 1.91 | 2.53 | 2.00 | 0.48 | 0.001 |
| 1_Scn2aB10_hRNU11 | | | | | | |
| 168_hRNU5A- | 2.10 | 1.81 | 2.14 | 2.01 | 0.18 | 0.001 |
| 1_Scn2aB10_mRnu12 | | | | | | |
| 126_hRNU4- | 0.89 | 2.60 | 2.68 | 2.05 | 1.01 | <0.001 |
| 1_Scn2aB10_hRNU1-3 | | | | | | |
| 136_hRNU4- | 2.31 | 1.44 | 2.49 | 2.08 | 0.57 | <0.001 |
| 1_Scn2aB10_mRnu1a1 | | | | | | |
| 143_hRNU4- | 2.24 | 1.51 | 2.53 | 2.09 | 0.53 | <0.001 |
| 1_Scn2aB10_mRnu7 | | | | | | |
| 141_hRNU4- | 2.12 | 2.06 | 2.12 | 2.10 | 0.03 | <0.001 |
| 1_Scn2aB10_mRnu5g | | | | | | |
| 160_hRNU5A- | 1.17 | 2.63 | 2.60 | 2.13 | 0.83 | <0.001 |
| 1_Scn2aB10_mRnu1b1 | | | | | | |
| 148_hRNU5A- | 1.31 | 2.75 | 2.49 | 2.18 | 0.77 | <0.001 |
| 1_Scn2aB10_hRNU1-2 | | | | | | |
| 125_hRNU4- | 1.64 | 2.48 | 2.44 | 2.19 | 0.47 | <0.001 |
| 1_Scn2aB10_hRNU1-2 | | | | | | |
| 100_mu1a1_Scn2aB10 U1 | 2.15 | 1.40 | 3.08 | 2.21 | 0.84 | <0.001 |
| 134_hRNU4- | 2.03 | 2.34 | 2.34 | 2.24 | 0.18 | <0.001 |
| 1_Scn2aB10_hRNU12 | | | | | | |
| 129_hRNU4- | 1.63 | 1.75 | 3.40 | 2.26 | 0.99 | <0.001 |
| 1_Scn2aB10_hRNU4-1 | | | | | | |
| 128_hRNU4-1_Scn2aB10_hU3 | 1.67 | 2.47 | 2.74 | 2.29 | 0.55 | <0.001 |
| 197_RNU7- | 1.50 | 2.31 | 3.26 | 2.36 | 0.88 | <0.001 |
| 1_Scn2aB10_hRNU1-1 | | | | | | |
| 159_hRNU5A- | 1.56 | 3.04 | 2.63 | 2.41 | 0.77 | <0.001 |
| 1_Scn2aB10_mRnu1a1 | | | | | | |
| 153_hRNU5A- | 2.89 | 1.60 | 2.84 | 2.44 | 0.73 | <0.001 |
| 1_Scn2aB10_hRNU5A-1 | | | | | | |
| 195 mu1a1 Scn2aD12_U1 | 1.75 | 2.06 | 3.58 | 2.47 | 0.98 | <0.001 |
| 157_hRNU5A- | 2.12 | 2.75 | 2.61 | 2.49 | 0.33 | <0.001 |
| 1_Scn2aB10_hRNU12 | | | | | | |
| 166_hRNU5A- | 1.85 | 2.95 | 3.27 | 2.69 | 0.74 | <0.001 |
| 1_Scn2aB10_mRnu7 | | | | | | |

TABLE 10
Library 3 Summary

| | | | | | | | | | | P-value |
|---|---|---|---|---|---|---|---|---|---|---|
| Name | ID | Promoter | U7 | Terminator | Rep1 | Rep2 | Rep3 | Average | SD | (ANOVA) |
| 259_hRNU12_ | 259 | hRNU12 | Scn2aB10 | mRnu3b1 | 0.77 | 0.63 | 0.70 | 0.70 | 0.07 | 0.15 |
| Scn2aB10_ | | | | | | | | | | |
| mRnu3b1 | | | | | | | | | | |
| 282_hRN7SK_ | 282 | hRN7SK | Scn2aB10 | mRnu3b1 | 1.00 | 0.83 | 0.85 | 0.89 | 0.09 | 0.79 |
| Scn2aB10_ | | | | | | | | | | |
| mRnu3b1 | | | | | | | | | | |
| 285_hRN7SK_ | 285 | hRN7SK | Scn2aB10 | mRnu7 | 1.03 | 0.80 | 0.86 | 0.90 | 0.12 | 0.81 |
| Scn2aB10_ | | | | | | | | | | |
| mRnu7 | | | | | | | | | | |
| 274_hRN7SK_ | 274 | hRN7SK | Scn2aB10 | RNU7-1 | 0.93 | 1.00 | 0.78 | 0.90 | 0.11 | 0.84 |
| Scn2aB10_ | | | | | | | | | | |
| RNU7-1 | | | | | | | | | | |
| 250_hRNU12_ | 250 | hRNU12 | Scn2aB10 | hRNU6-1 | 0.98 | 0.89 | 0.86 | 0.91 | 0.06 | 0.87 |
| Scn2aB10_ | | | | | | | | | | |
| hRNU6-1 | | | | | | | | | | |
| 290_mu1a1_ | 290 | mu1a1 | Scr2 | U1 | 0.99 | 1.06 | 0.76 | 0.94 | 0.15 | Control |
| Scr2_U1 | | | | | | | | | | |
| 251_hRNU12_ | 251 | hRNU12 | Scn2aB10 | RNU7-1 | 0.89 | 1.13 | 0.89 | 0.97 | 0.14 | 0.84 |
| Scn2aB10_ | | | | | | | | | | |
| RNU7-1 | | | | | | | | | | |
| 267_hRN7SK_ | 267 | hRN7SK | Scn2aB10 | hRNU1-2 | 1.06 | 1.03 | 0.92 | 1.00 | 0.07 | 0.69 |
| Scn2aB10_ | | | | | | | | | | |
| hRNU1-2 | | | | | | | | | | |
| 254_hRNU12_ | 254 | hRNU12 | Scn2aB10 | hRN7SK | 0.88 | 1.22 | 0.91 | 1.00 | 0.18 | 0.69 |
| Scn2aB10_ | | | | | | | | | | |
| hRN7SK | | | | | | | | | | |
| 273_hRN7SK_ | 273 | hRN7SK | Scn2aB10 | hRNU6-1 | 1.14 | 0.99 | 0.97 | 1.03 | 0.09 | 0.56 |
| Scn2aB10_ | | | | | | | | | | |
| hRNU6-1 | | | | | | | | | | |
| 268_hRN7SK_ | 268 | hRN7SK | Scn2aB10 | hRNU1-3 | 1.02 | 0.99 | 1.15 | 1.05 | 0.09 | 0.48 |
| Scn2aB10_ | | | | | | | | | | |
| hRNU1-3 | | | | | | | | | | |
| 271_hRN7SK_ | 271 | hRN7SK | Scn2aB10 | hRNU4-1 | 1.07 | 1.14 | 0.97 | 1.06 | 0.09 | 0.46 |
| Scn2aB10_ | | | | | | | | | | |
| hRNU4-1 | | | | | | | | | | |
| 289_mu1a1_ | 289 | mu1a1 | Scn2aF1Scr2 | U1 | 1.01 | 0.94 | 1.24 | 1.06 | 0.15 | 0.44 |
| Scn2aF1Scr2_ | | | | | | | | | | |
| U1 | | | | | | | | | | |
| 275_hRN7SK_ | 275 | hRN7SK | Scn2aB10 | hRNU11 | 1.03 | 1.11 | 1.10 | 1.08 | 0.05 | 0.39 |
| Scn2aB10_ | | | | | | | | | | |
| hRNU11 | | | | | | | | | | |
| 266_hRN7SK_ | 266 | hRN7SK | Scn2aB10 | hRNU1-1 | 1.17 | 1.02 | 1.05 | 1.08 | 0.08 | 0.39 |
| Scn2aB10_ | | | | | | | | | | |
| hRNU1-1 | | | | | | | | | | |
| 247_hRNU12_ | 247 | hRNU12 | Scn2aB10 | hU3 | 0.82 | 1.26 | 1.17 | 1.09 | 0.23 | 0.38 |
| Scn2aB10_hU3 | | | | | | | | | | |
| 257_hRNU12_ | 257 | hRNU12 | Scn2aB10 | mRnu1b6 | 0.98 | 1.09 | 1.19 | 1.09 | 0.11 | 0.37 |
| Scn2aB10_ | | | | | | | | | | |
| mRnu1b6 | | | | | | | | | | |
| 283_hRN7SK_ | 283 | hRN7SK | Scn2aB10 | mRnu5g | 0.99 | 1.30 | 1.04 | 1.11 | 0.17 | 0.3 |
| Scn2aB10_ | | | | | | | | | | |
| mRnu5g | | | | | | | | | | |
| 264_hRNU12_ | 264 | hRNU12 | Scn2aB10 | mRnu12 | 1.18 | 1.11 | 1.04 | 1.11 | 0.07 | 0.3 |
| Scn2aB10_ | | | | | | | | | | |
| mRnu12 | | | | | | | | | | |
| 246_hRNU12_ | 246 | hRNU12 | Scn2aB10 | hRNU2-1 | 1.15 | 1.13 | 1.07 | 1.12 | 0.04 | 0.28 |
| Scn2aB10_ | | | | | | | | | | |
| hRNU2-1 | | | | | | | | | | |
| 249_hRNU12_ | 249 | hRNU12 | Scn2aB10 | hRNU5A-1 | 1.0 | 1.27 | 1.05 | 1.12 | 0.13 | 0.27 |
| Scn2aB10_ | | | | | | | | | | |
| hRNU5A-1 | | | | | | | | | | |
| 248_hRNU12_ | 248 | hRNU12 | Scn2aB10 | hRNU4-1 | 1.01 | 1.19 | 1.17 | 1.13 | 0.10 | 0.26 |
| Scn2aB10_ | | | | | | | | | | |
| hRNU4-1 | | | | | | | | | | |
| 280_hRN7SK_ | 280 | hRN7SK | Scn2aB10 | mRnu1b6 | 1.16 | 1.16 | 1.07 | 1.13 | 0.05 | 0.24 |
| Scn2aB10_ | | | | | | | | | | |
| mRnu1b6 | | | | | | | | | | |
| 265_hRNU12_ | 265 | hRNU12 | Scn2aB10 | mRn7sk | 1.13 | 1.20 | 1.06 | 1.13 | 0.07 | 0.24 |
| Scn2aB10_ | | | | | | | | | | |
| mRn7sk | | | | | | | | | | |
| 256_hRNU12_ | 256 | hRNU12 | Scn2aB10 | mRnu1b1 | 1.01 | 1.14 | 1.26 | 1.14 | 0.12 | 0.23 |
| Scn2aB10_ | | | | | | | | | | |
| mRnu1b1 | | | | | | | | | | |
| 281_hRN7SK_ | 281 | hRN7SK | Scn2aB10 | mRnu2-10 | 1.31 | 1.01 | 1.10 | 1.14 | 0.16 | 0.22 |
| Scn2aB10_ | | | | | | | | | | |
| mRnu2-10 | | | | | | | | | | |
| 278_hRN7SK_ | 278 | hRN7SK | Scn2aB10 | mRnu1a1 | 1.02 | 1.22 | 1.19 | 1.15 | 0.11 | 0.21 |
| Scn2aB10_ | | | | | | | | | | |
| mRnu1a1 | | | | | | | | | | |
| 276_hRN7SK_ | 276 | hRN7SK | Scn2aB10 | hRNU12 | 1.35 | 1.32 | 0.82 | 1.16 | 0.30 | 0.17 |
| Scn2aB10_ | | | | | | | | | | |
| hRNU12 | | | | | | | | | | |
| 263_hRNU12_ | 263 | hRNU12 | Scn2aB10 | mRnu11 | 1.25 | 1.25 | 0.99 | 1.16 | 0.15 | 0.17 |
| Scn2aB10_ | | | | | | | | | | |
| mRnu11 | | | | | | | | | | |
| 242_hRNU11_ | 242 | hRNU11 | Scn2aB10 | mRn7sk | 1.12 | 1.20 | 1.29 | 1.21 | 0.09 | 0.11 |
| Scn2aB10_ | | | | | | | | | | |
| mRn7sk | | | | | | | | | | |
| 213 RNU7- | 213 | RNU7-1 | Scn2aB10 | mRnu3b1 | 1.26 | 1.17 | 1.20 | 1.21 | 0.05 | 0.1 |
| 1_Scn2aB10_ | | | | | | | | | | |
| mRnu3b1 | | | | | | | | | | |
| 258_hRNU12_ | 258 | hRNU12 | Scn2aB10 | mRnu2-10 | 1.21 | 1.44 | 1.00 | 1.21 | 0.22 | 0.09 |
| Scn2aB10_ | | | | | | | | | | |
| mRnu2-10 | | | | | | | | | | |
| 287_hRN7SK_ | 287 | hRN7SK | Scn2aB10 | mRnu12 | 1.10 | 1.52 | 1.03 | 1.22 | 0.27 | 0.09 |
| Scn2aB10_ | | | | | | | | | | |
| mRnu12 | | | | | | | | | | |
| 279_hRN7SK_ | 279 | hRN7SK | Scn2aB10 | mRnu1b1 | 1.31 | 1.14 | 1.20 | 1.22 | 0.09 | 0.09 |
| Scn2aB10_ | | | | | | | | | | |
| mRnu1b1 | | | | | | | | | | |
| 270_hRN7SK_ | 270 | hRN7SK | Scn2aB10 | hU3 | 1.10 | 1.50 | 1.07 | 1.22 | 0.24 | 0.08 |
| Scn2aB10_hU3 | | | | | | | | | | |
| 252_hRNU12_ | 252 | hRNU12 | Scn2aB10 | hRNU11 | 1.18 | 1.40 | 1.16 | 1.25 | 0.13 | 0.06 |
| Scn2aB10_ | | | | | | | | | | |
| hRNU11 | | | | | | | | | | |
| 231_hRNU11_ | 231 | hRNU11 | Scn2aB10 | hRN7SK | 1.26 | 1.27 | 1.23 | 1.25 | 0.02 | 0.06 |
| Scn2aB10_ | | | | | | | | | | |
| hRN7SK | | | | | | | | | | |
| 272_hRN7SK_ | 272 | hRN7SK | Scn2aB10 | hRNU5A-1 | 1.10 | 1.35 | 1.36 | 1.27 | 0.15 | 0.05 |
| Scn2aB10_ | | | | | | | | | | |
| hRNU5A-1 | | | | | | | | | | |
| 227_hRNU11_ | 227 | hRNU11 | Scn2aB10 | hRNU6-1 | 1.24 | 1.43 | 1.19 | 1.29 | 0.13 | 0.04 |
| Scn2aB10_ | | | | | | | | | | |
| hRNU6-1 | | | | | | | | | | |
| 205 RNU7- | 205 | RNU7-1 | Scn2aB10 | RNU7-1 | 1.11 | 1.62 | 1.14 | 1.29 | 0.29 | 0.03 |
| 1_Scn2aB10_ | | | | | | | | | | |
| RNU7-1 | | | | | | | | | | |
| 277_hRN7SK_ | 277 | hRN7SK | Scn2aB10 | hRN7SK | 1.10 | 1.46 | 1.32 | 1.29 | 0.18 | 0.03 |
| Scn2aB10_ | | | | | | | | | | |
| hRN7SK | | | | | | | | | | |
| 255_hRNU12_ | 255 | hRNU12 | Scn2aB10 | mRnu1a1 | 1.47 | 1.20 | 1.22 | 1.30 | 0.15 | 0.03 |
| Scn2aB10_ | | | | | | | | | | |
| mRnu1a1 | | | | | | | | | | |
| 207_RNU7- | 207 | RNU7-1 | Scn2aB10 | hRNU12 | 1.29 | 1.44 | 1.21 | 1.31 | 0.12 | 0.02 |
| 1_Scn2aB10_ | | | | | | | | | | |
| hRNU12 | | | | | | | | | | |
| 245_hRNU12_ | 245 | hRNU12 | Scn2aB10 | hRNU1-3 | 1.44 | 1.14 | 1.40 | 1.32 | 0.16 | 0.02 |
| Scn2aB10_ | | | | | | | | | | |
| hRNU1-3 | | | | | | | | | | |
| 243_hRNU12_ | 243 | hRNU12 | Scn2aB10 | hRNU1-1 | 1.54 | 1.10 | 1.37 | 1.34 | 0.22 | 0.02 |
| Scn2aB10_ | | | | | | | | | | |
| hRNU1-1 | | | | | | | | | | |
| 235_hRNU11_ | 235 | hRNU11 | Scn2aB10 | mRnu2-10 | 1.20 | 1.71 | 1.15 | 1.35 | 0.31 | 0.01 |
| Scn2aB10_ | | | | | | | | | | |
| mRnu2-10 | | | | | | | | | | |
| 204 RNU7- | 204 | RNU7-1 | Scn2aB10 | hRNU6-1 | 1.32 | 1.56 | 1.18 | 1.35 | 0.19 | 0.01 |
| 1_Scn2aB10_ | | | | | | | | | | |
| hRNU6-1 | | | | | | | | | | |
| 208 RNU7- | 208 | RNU7-1 | Scn2aB10 | hRN7SK | 1.46 | 1.39 | 1.21 | 1.35 | 0.13 | 0.01 |
| 1_Scn2aB10_ | | | | | | | | | | |
| hRN7SK | | | | | | | | | | |
| 288_hRN7SK_ | 288 | hRN7SK | Scn2aB10 | mRn7sk | 1.49 | 1.03 | 1.65 | 1.39 | 0.32 | 0.007 |
| Scn2aB10_ | | | | | | | | | | |
| mRn7sk | | | | | | | | | | |
| 239_hRNU11_ | 239 | hRNU11 | Scn2aB10 | mRnu7 | 1.49 | 1.42 | 1.27 | 1.39 | 0.11 | 0.006 |
| Scn2aB10_ | | | | | | | | | | |
| mRnu7 | | | | | | | | | | |
| 300_mRnu1a1_ | 300 | mRnu1a1 | Scn2aB10 | hRNU6-1 | 1.35 | 1.53 | 1.31 | 1.40 | 0.12 | 0.006 |
| Scn2aB10_ | | | | | | | | | | |
| hRNU6-1 | | | | | | | | | | |
| 240_hRNU11_ | 240 | hRNU11 | Scn2aB10 | mRnu11 | 1.23 | 1.57 | 1.50 | 1.43 | 0.18 | 0.003 |
| Scn2aB10_ | | | | | | | | | | |
| mRnu11 | | | | | | | | | | |
| 218_RNU7- | 218 | RNU7-1 | Scn2aB10 | mRnu12 | 1.72 | 1.31 | 1.29 | 1.44 | 0.24 | 0.003 |
| 1_Scn2aB10_ | | | | | | | | | | |
| mRnu12 | | | | | | | | | | |
| 234_hRNU11_ | 234 | hRNU11 | Scn2aB10 | mRnu1b6 | 1.61 | 1.43 | 1.30 | 1.45 | 0.16 | 0.002 |
| Scn2aB10_ | | | | | | | | | | |
| mRnu1b6 | | | | | | | | | | |
| 214_RNU7- | 214 | RNU7-1 | Scn2aB10 | mRnu5g | 1.25 | 1.63 | 1.46 | 1.45 | 0.19 | 0.002 |
| 1_Scn2aB10_ | | | | | | | | | | |
| mRnu5g | | | | | | | | | | |
| 269_hRN7SK_ | 269 | hRN7SK | Scn2aB10 | hRNU2-1 | 1.53 | 1.36 | 1.46 | 1.45 | 0.09 | 0.002 |
| Scn2aB10_ | | | | | | | | | | |
| hRNU2-1 | | | | | | | | | | |
| 212_RNU7- | 212 | RNU7-1 | Scn2aB10 | mRnu2-10 | 1.33 | 1.83 | 1.21 | 1.46 | 0.33 | 0.002 |
| 1_Scn2aB10_ | | | | | | | | | | |
| mRnu2-10 | | | | | | | | | | |
| 260_hRNU12_ | 260 | hRNU12 | Scn2aB10 | mRnu5g | 1.28 | 1.91 | 1.21 | 1.47 | 0.39 | 0.002 |
| Scn2aB10_ | | | | | | | | | | |
| mRnu5g | | | | | | | | | | |
| 219_RNU7- | 219 | RNU7-1 | Scn2aB10 | mRn7sk | 1.80 | 1.21 | 1.39 | 1.47 | 0.30 | 0.002 |
| 1_Scn2aB10_ | | | | | | | | | | |
| mRn7sk | | | | | | | | | | |
| 236_hRNU11_ | 236 | hRNU11 | Scn2aB10 | mRnu3b1 | 1.46 | 1.57 | 1.38 | 1.47 | 0.10 | 0.001 |
| Scn2aB10_ | | | | | | | | | | |
| mRnu3b1 | | | | | | | | | | |
| 228_hRNU11_ | 228 | hRNU11 | Scn2aB10 | RNU7-1 | 1.34 | 1.61 | 1.49 | 1.48 | 0.14 | 0.001 |
| Scn2aB10_ | | | | | | | | | | |
| RNU7-1 | | | | | | | | | | |
| 241_hRNU11_ | 241 | hRNU11 | Scn2aB10 | mRnu12 | 1.39 | 1.63 | 1.43 | 1.49 | 0.13 | 0.001 |
| Scn2aB10_ | | | | | | | | | | |
| mRnu12 | | | | | | | | | | |
| 237_hRNU11_ | 237 | hRNU11 | Scn2aB10 | mRnu5g | 1.67 | 1.30 | 1.51 | 1.49 | 0.19 | <0.001 |
| Scn2aB10_ | | | | | | | | | | |
| mRnu5g | | | | | | | | | | |
| 206_RNU7- | 206 | RNU7-1 | Scn2aB10 | hRNU11 | 1.46 | 1.53 | 1.50 | 1.50 | 0.04 | <0.001 |
| 1_Scn2aB10_ | | | | | | | | | | |
| hRNU11 | | | | | | | | | | |
| 217_RNU7- | 217 | RNU7-1 | Scn2aB10 | mRnu11 | 1.40 | 1.77 | 1.41 | 1.53 | 0.21 | <0.001 |
| 1_Scn2aB10_ | | | | | | | | | | |
| mRnu11 | | | | | | | | | | |
| 229_hRNU11_ | 229 | hRNU11 | Scn2aB10 | hRNU11 | 1.44 | 1.78 | 1.43 | 1.55 | 0.20 | <0.001 |
| Scn2aB10_ | | | | | | | | | | |
| hRNU11 | | | | | | | | | | |
| 233_hRNU11_ | 233 | hRNU11 | Scn2aB10 | mRnu1b1 | 1.42 | 1.79 | 1.54 | 1.58 | 0.19 | <0.001 |
| Scn2aB10_ | | | | | | | | | | |
| mRnu1b1 | | | | | | | | | | |
| 296_mRnu1a1 | 296 | mRnu1a1 | Scn2aB10 | hRNU2-1 | 1.49 | 1.88 | 1.39 | 1.59 | 0.26 | <0.001 |
| Scn2aB10_ | | | | | | | | | | |
| hRNU2-1 | | | | | | | | | | |
| 211_RNU7- | 211 | RNU7-1 | Scn2aB10 | mRnu1b6 | 1.70 | 1.32 | 1.76 | 1.59 | 0.24 | <0.001 |
| 1_Scn2aB10 | | | | | | | | | | |
| mRnu1b6 | | | | | | | | | | |
| 202_RNU7- | 202 | RNU7-1 | Scn2aB10 | hRNU4-1 | 1.28 | 1.69 | 1.84 | 1.60 | 0.29 | <0.001 |
| 1_Scn2aB10_ | | | | | | | | | | |
| hRNU4-1 | | | | | | | | | | |
| 244_hRNU12_ | 244 | hRNU12 | Scn2aB10 | hRNU1-2 | 1.59 | 1.54 | 1.70 | 1.6 | 0.08 | <0.001 |
| Scn2aB10_ | | | | | | | | | | |
| hRNU1-2 | | | | | | | | | | |
| 223_hRNU11_ | 223 | hRNU11 | Scn2aB10 | hRNU2-1 | 1.71 | 1.56 | 1.64 | 1.63 | 0.07 | <0.001 |
| Scn2aB10_ | | | | | | | | | | |
| hRNU2-1 | | | | | | | | | | |
| 209 RNU7- | 209 | RNU7-1 | Scn2aB10 | mRnu1a1 | 1.40 | 1.80 | 1.76 | 1.65 | 0.22 | <0.001 |
| 1_Scn2aB10_ | | | | | | | | | | |
| mRnu1a1 | | | | | | | | | | |
| 225_hRNU11_ | 225 | hRNU11 | Scn2aB10 | hRNU4-1 | 1.43 | 1.51 | 2.04 | 1.66 | 0.33 | <0.001 |
| Scn2aB10_ | | | | | | | | | | |
| hRNU4-1 | | | | | | | | | | |
| 230_hRNU11_ | 230 | hRNU11 | Scn2aB10 | hRNU12 | 1.49 | 1.53 | 2.01 | 1.67 | 0.29 | <0.001 |
| Scn2aB10_ | | | | | | | | | | |
| hRNU12 | | | | | | | | | | |
| 293_mRnu1a1_ | 293 | mRnu1a1 | Scn2aB10 | hRNU1-1 | 1.42 | 1.87 | 1.74 | 1.68 | 0.23 | <0.001 |
| Scn2aB10_ | | | | | | | | | | |
| hRNU1-1 | | | | | | | | | | |
| 294_mRnu1a1_ | 294 | mRnu1a1 | Scn2aB10 | hRNU1-2 | 1.78 | 1.49 | 1.82 | 1.70 | 0.18 | <0.001 |
| Scn2aB10_ | | | | | | | | | | |
| hRNU1-2 | | | | | | | | | | |
| 291 mu1a1_ | 291 | mu1a1 | Scn2aD12 | U1 | 1.78 | 1.85 | 1.50 | 1.71 | 0.19 | <0.001 |
| Scn2aD 12 U1 | | | | | | | | | | |
| 216 RNU7- | 216 | RNU7-1 | Scn2aB10 | mRnu7 | 1.62 | 1.53 | 2.02 | 1.72 | 0.26 | <0.001 |
| 1_Scn2aB10_ | | | | | | | | | | |
| mRnu7 | | | | | | | | | | |
| 221_hRNU11_ | 221 | hRNU11 | Scn2aB10 | hRNU1-2 | 1.83 | 1.75 | 1.59 | 1.72 | 0.12 | <0.001 |
| Scn2aB10_ | | | | | | | | | | |
| hRNU1-2 | | | | | | | | | | |
| 297_mRnu1a1_ | 297 | mRnu1a1 | Scn2aB10 | hU3 | 1.88 | 1.60 | 1.69 | 1.72 | 0.14 | <0.001 |
| Scn2aB10_hU3 | | | | | | | | | | |
| 299_mRnu1a1_ | 299 | mRnu1a1 | Scn2aB10 | hRNU5A-1 | 2.06 | 1.68 | 1.49 | 1.74 | 0.29 | <0.001 |
| Scn2aB10_ | | | | | | | | | | |
| hRNU5A-1 | | | | | | | | | | |
| 222_hRNU11_ | 222 | hRNU11 | Scn2aB10 | hRNU1-3 | 1.55 | 2.06 | 1.72 | 1.78 | 0.26 | <0.001 |
| Scn2aB10_ | | | | | | | | | | |
| hRNU1-3 | | | | | | | | | | |
| 232_hRNU11_ | 232 | hRNU11 | Scn2aB10 | mRnu1a1 | 2.24 | 1.62 | 1.54 | 1.80 | 0.38 | <0.001 |
| Scn2aB10_ | | | | | | | | | | |
| mRnu1a1 | | | | | | | | | | |
| 298_mRnu1a1_ | 298 | mRnu1a1 | Scn2aB10 | hRNU4-1 | 2.06 | 1.52 | 1.99 | 1.86 | 0.29 | <0.001 |
| Scn2aB10_ | | | | | | | | | | |
| hRNU4-1 | | | | | | | | | | |
| 226_hRNU11 | 226 | hRNU11 | Scn2aB10 | hRNU5A-1 | 2.0 | 1.74 | 1.79 | 1.86 | 0.16 | <0.001 |
| Scn2aB10_ | | | | | | | | | | |
| hRNU5A-1 | | | | | | | | | | |
| 224_hRNU11 | 224 | hRNU11 | Scn2aB10 | hU3 | 1.75 | 2.06 | 1.87 | 1.89 | 0.15 | <0.001 |
| Scn2aB10_hU3 | | | | | | | | | | |
| 220_hRNU11_ | 220 | hRNU11 | Scn2aB10 | hRNU1-1 | 2.1 | 1.79 | 1.77 | 1.89 | 0.19 | <0.001 |
| Scn2aB10_ | | | | | | | | | | |
| hRNU1-1 | | | | | | | | | | |
| 295_mRnu1a1_ | 295 | mRnu1a1 | Scn2aB10 | hRNU1-3 | 2.09 | 2.21 | 1.51 | 1.93 | 0.38 | <0.001 |
| Scn2aB10_ | | | | | | | | | | |
| hRNU1-3 | | | | | | | | | | |
| 301_mRnu1a1_ | 301 | mRnu1a1 | Scn2aB10 | RNU7-1 | 2.03 | 1.95 | 1.82 | 1.94 | 0.11 | <0.001 |
| Scn2aB10_ | | | | | | | | | | |
| RNU7-1 | | | | | | | | | | |
| 203_RNU7- | 203 | RNU7-1 | Scn2aB10 | hRNU5A-1 | 2.54 | 2.11 | 1.78 | 2.14 | 0.38 | <0.001 |
| 1_Scn2aB10_ | | | | | | | | | | |
| hRNU5A-1 | | | | | | | | | | |

TABLE 11
Library 4 Summary

| | | | | | New4- | New4- | New4- | Aver- | | P-value |
|---|---|---|---|---|---|---|---|---|---|---|
| Name | ID | Promoter | U7 | Terminator | 1 | 2 | 3 | age | SD | (ANOVA) |
| 347_mRnu1b6_ | 347 | mRnu1b6 | Scn2aB10 | RNU7-1 | 1.09 | 0.81 | 0.86 | 0.92 | 0.15 | 0.64 |
| Scn2aB10_ | | | | | | | | | | |
| RNU7-1 | | | | | | | | | | |
| 385_mu1a1 | 385 | mu1a1 | Scn2aF1Scr2 | U1 | 1.01 | 0.90 | 1.09 | 1.00 | 0.09 | Control |
| Scn2aF1Scr2_U1 | | | | | | | | | | |
| 386_mu1a1_ | 386 | mu1a1 | Scr2 | U1 | 0.99 | 1.10 | 0.91 | 1.00 | 0.09 | >0.99 |
| Scr2_U1 | | | | | | | | | | |
| 327_mRnu1b1 | 327 | mRnu1b1 | Scn2aB10 | hRN7SK | 1.31 | 1.16 | 1.19 | 1.22 | 0.08 | 0.2 |
| Scn2aB10_ | | | | | | | | | | |
| hRN7SK | | | | | | | | | | |
| 346_mRnu1b6 | 346 | mRnu1b6 | Scn2aB10 | hRNU6-1 | 1.32 | 1.24 | 1.12 | 1.22 | 0.10 | 0.18 |
| Scn2aB10_ | | | | | | | | | | |
| hRNU6-1 | | | | | | | | | | |
| 338_mRnu1b1 | 338 | mRnu1b1 | Scn2aB10 | mRn7sk | 1.48 | 1.04 | 1.35 | 1.29 | 0.23 | 0.09 |
| Scn2aB10_ | | | | | | | | | | |
| mRn7sk | | | | | | | | | | |
| 323_mRnu1b1 | 323 | mRnu1b1 | Scn2aB10 | hRNU6-1 | 1.44 | 1.03 | 1.47 | 1.31 | 0.24 | 0.07 |
| Scn2aB10_ | | | | | | | | | | |
| hRNU6-1 | | | | | | | | | | |
| 355_mRnu1b6 | 355 | mRnu1b6 | Scn2aB10 | mRnu3b1 | 0.87 | 2.13 | 1.01 | 1.3 | 0.69 | 0.05 |
| Scn2aB10_ | | | | | | | | | | |
| mRnu3b1 | | | | | | | | | | |
| 332_mRnu1b1 | 332 | mRnu1b1 | Scn2aB10 | mRnu3b1 | 1.51 | 1.10 | 1.40 | 1.34 | 0.21 | 0.05 |
| Scn2aB10_ | | | | | | | | | | |
| mRnu3b1 | | | | | | | | | | |
| 356_mRnu1b6 | 356 | mRnu1b6 | Scn2aB10 | mRnu5g | 1.68 | 0.91 | 1.44 | 1.34 | 0.39 | 0.04 |
| Scn2aB10_ | | | | | | | | | | |
| mRnu5g | | | | | | | | | | |
| 350_mRnu1b6 | 350 | mRnu1b6 | Scn2aB10 | hRN7SK | 1.40 | 1.33 | 1.44 | 1.39 | 0.06 | 0.02 |
| Scn2aB10_ | | | | | | | | | | |
| hRN7SK | | | | | | | | | | |
| 309_mRnu1a1 | 309 | mRnu1a1 | Scn2aB10 | mRnu3b1 | 1.55 | 1.2 | 1.39 | 1.39 | 0.16 | 0.02 |
| Scn2aB10_ | | | | | | | | | | |
| mRnu3b1 | | | | | | | | | | |
| 384_mRnu2- | 384 | mRnu2-10 | Scn2aB10 | mRn7sk | 1.58 | 1.28 | 1.40 | 1.42 | 0.15 | 0.01 |
| 10_Scn2aB10_ | | | | | | | | | | |
| mRn7sk | | | | | | | | | | |
| 315_mRnu1a1 | 315 | mRnu1a1 | Scn2aB10 | mRn7sk | 1.57 | 1.11 | 1.59 | 1.42 | 0.27 | 0.01 |
| Scn2aB10_ | | | | | | | | | | |
| mRn7sk | | | | | | | | | | |
| 319_mRnu1b1 | 319 | mRnu1b1 | Scn2aB10 | hRNU2-1 | 1.42 | 1.33 | 1.53 | 1.43 | 0.10 | 0.01 |
| Scn2aB10_ | | | | | | | | | | |
| hRNU2-1 | | | | | | | | | | |
| 378_mRnu2- | 378 | mRnu2-10 | Scn2aB10 | mRnu3b1 | 1.56 | 1.25 | 1.54 | 1.45 | 0.17 | 0.008 |
| 10_Scn2aB10_ | | | | | | | | | | |
| mRnu3b1 | | | | | | | | | | |
| 337_mRnu1b1 | 337 | mRnu1b1 | Scn2aB10 | mRnu12 | 1.59 | 1.27 | 1.55 | 1.47 | 0.18 | 0.006 |
| Scn2aB10_ | | | | | | | | | | |
| mRnu12 | | | | | | | | | | |
| 400_mRnu3b1 | 400 | mRnu3b1 | Scn2aB10 | hRN7SK | 1.74 | 1.34 | 1.35 | 1.48 | 0.23 | 0.005 |
| Scn2aB10_ | | | | | | | | | | |
| hRN7SK | | | | | | | | | | |
| 324_mRnu1b1 | 324 | mRnu1b1 | Scn2aB10 | RNU7-1 | 1.57 | 1.29 | 1.59 | 1.48 | 0.17 | 0.005 |
| Scn2aB10_ | | | | | | | | | | |
| RNU7-1 | | | | | | | | | | |
| 316_mRnu1b1 | 316 | mRnu1b1 | Scn2aB10 | hRNU1-1 | 1.56 | 1.49 | 1.41 | 1.49 | 0.07 | 0.004 |
| Scn2aB10_ | | | | | | | | | | |
| hRNU1-1 | | | | | | | | | | |
| 351_mRnu1b6 | 351 | mRnu1b6 | Scn2aB10 | mRnu1a1 | 1.24 | 1.55 | 1.68 | 1.49 | 0.23 | 0.004 |
| Scn2aB10_ | | | | | | | | | | |
| mRnu1a1 | | | | | | | | | | |
| 354_mRnu1b6 | 354 | mRnu1b6 | Scn2aB10 | mRnu2-10 | 1.62 | 1.40 | 1.50 | 1.50 | 0.11 | 0.003 |
| Scn2aB10_ | | | | | | | | | | |
| mRnu2-10 | | | | | | | | | | |
| 331_mRnu1b1 | 331 | mRnu1b1 | Scn2aB10 | mRnu2-10 | 1.71 | 1.42 | 1.50 | 1.55 | 0.15 | 0.002 |
| Scn2aB10_ | | | | | | | | | | |
| mRnu2-10 | | | | | | | | | | |
| 373_mRnu2- | 373 | mRnu2-10 | Scn2aB10 | hRN7SK | 1.66 | 1.30 | 1.7 | 1.56 | 0.22 | 0.001 |
| 10_Scn2aB10_ | | | | | | | | | | |
| hRN7SK | | | | | | | | | | |
| 317_mRnu1b1 | 317 | mRnu1b1 | Scn2aB10 | hRNU1-2 | 1.93 | 1.38 | 1.42 | 1.57 | 0.31 | <0.001 |
| Scn2aB10_hRN | | | | | | | | | | |
| U1-2 | | | | | | | | | | |
| 361_mRnu1b6 | 361 | mRnu1b6 | Scn2aB10 | mRn7sk | 1.85 | 1.29 | 1.61 | 1.58 | 0.28 | <0.001 |
| Scn2aB10_ | | | | | | | | | | |
| mRn7sk | | | | | | | | | | |
| 387_mu1a1 | 387 | mu1a1 | Scn2aD12 | U1 | 1.74 | 1.41 | 1.64 | 1.60 | 0.17 | <0.001 |
| Scn2aD12_U1 | | | | | | | | | | |
| 325_mRnu1b1 | 325 | mRnu1b1 | Scn2aB10 | hRNU11 | 1.77 | 1.38 | 1.66 | 1.60 | 0.20 | <0.001 |
| Scn2aB10_ | | | | | | | | | | |
| hRNU11 | | | | | | | | | | |
| 304_mRnu1a1 | 304 | mRnu1a1 | Scn2aB10 | hRN7SK | 1.65 | 1.39 | 1.77 | 1.60 | 0.19 | <0.001 |
| Scn2aB10_ | | | | | | | | | | |
| hRN7SK | | | | | | | | | | |
| 396_mRnu3b1 | 396 | mRnu3b1 | Scn2aB10 | hRNU6-1 | 1.68 | 1.37 | 1.80 | 1.61 | 0.22 | <0.001 |
| Scn2aB10_ | | | | | | | | | | |
| hRNU6-1 | | | | | | | | | | |
| 342_mRnu1b6 | 342 | mRnu1b6 | Scn2aB10 | hRNU2-1 | 1.86 | 1.25 | 1.74 | 1.62 | 0.32 | <0.001 |
| Scn2aB10_ | | | | | | | | | | |
| hRNU2-1 | | | | | | | | | | |
| 381_mRnu2- | 381 | mRnu2-10 | Scn2aB10 | mRnu7 | 1.72 | 1.41 | 1.73 | 1.62 | 0.18 | <0.001 |
| 10_Scn2aB10_ | | | | | | | | | | |
| mRnu7 | | | | | | | | | | |
| 369_mRnu2- | 369 | mRnu2-10 | Scn2aB10 | hRNU6-1 | 1.66 | 1.52 | 1.7 | 1.63 | 0.10 | <0.001 |
| 10_Scn2aB10_ | | | | | | | | | | |
| hRNU6-1 | | | | | | | | | | |
| 335_mRnu1b1 | 335 | mRnu1b1 | Scn2aB10 | mRnu7 | 1.87 | 1.50 | 1.56 | 1.64 | 0.20 | <0.001 |
| Scn2aB10_ | | | | | | | | | | |
| mRnu7 | | | | | | | | | | |
| 330_mRnu1b1 | 330 | mRnu1b1 | Scn2aB10 | mRnu1b6 | 1.89 | 1.54 | 1.52 | 1.65 | 0.21 | <0.001 |
| Scn2aB10_ | | | | | | | | | | |
| mRnu1b6 | | | | | | | | | | |
| 352_mRnu1b6 | 352 | mRnu1b6 | Scn2aB10 | mRnu1b1 | 1.69 | 1.54 | 1.7 | 1.66 | 0.11 | <0.001 |
| Scn2aB10_ | | | | | | | | | | |
| mRnu1b1 | | | | | | | | | | |
| 382_mRnu2- | 382 | mRnu2-10 | Scn2aB10 | mRnu11 | 1.94 | 1.40 | 1.6 | 1.67 | 0.27 | <0.001 |
| 10_Scn2aB10_ | | | | | | | | | | |
| mRnu11 | | | | | | | | | | |
| 344_mRnu1b6 | 344 | mRnu1b6 | Scn2aB10 | hRNU4-1 | 0.99 | 1.58 | 2.44 | 1.67 | 0.73 | <0.001 |
| Scn2aB10_ | | | | | | | | | | |
| hRNU4-1 | | | | | | | | | | |
| 336_mRnu1b1 | 336 | mRnu1b1 | Scn2aB10 | mRnu11 | 1.89 | 1.41 | 1.74 | 1.68 | 0.25 | <0.001 |
| Scn2aB10_ | | | | | | | | | | |
| mRnu11 | | | | | | | | | | |
| 371_mRnu2- | 371 | mRnu2-10 | Scn2aB10 | hRNU11 | 1.87 | 1.55 | 1.62 | 1.68 | 0.17 | <0.001 |
| 10_Scn2aB10 | | | | | | | | | | |
| hRNU11 | | | | | | | | | | |
| 326_mRnu1b1 | 326 | mRnu1b1 | Scn2aB10 | hRNU12 | 1.43 | 1.89 | 1.82 | 1.71 | 0.24 | <0.001 |
| Scn2aB10_ | | | | | | | | | | |
| hRNU12 | | | | | | | | | | |
| 343_mRnu1b6 | 343 | mRnu1b6 | Scn2aB10 | hU3 | 2.11 | 1.39 | 1.65 | 1.72 | 0.36 | <0.001 |
| Scn2aB10_hU3 | | | | | | | | | | |
| 318_mRnu1b1 | 318 | mRnu1b1 | Scn2aB10 | hRNU1-3 | 1.84 | 1.63 | 1.69 | 1.72 | 0.11 | <0.001 |
| Scn2aB10_ | | | | | | | | | | |
| hRNU1-3 | | | | | | | | | | |
| 358_mRnu1b6 | 358 | mRnu1b6 | Scn2aB10 | mRnu7 | 1.88 | 1.56 | 1.75 | 1.73 | 0.16 | <0.001 |
| Scn2aB10_ | | | | | | | | | | |
| mRnu7 | | | | | | | | | | |
| 383_mRnu2- | 383 | mRnu2-10 | Scn2aB10 | mRnu12 | 2.04 | 1.43 | 1.73 | 1.73 | 0.31 | <0.001 |
| 10_Scn2aB10_ | | | | | | | | | | |
| mRnu12 | | | | | | | | | | |
| 308_mRnu1a1 | 308 | mRnu1a1 | Scn2aB10 | mRnu2-10 | 1.71 | 1.65 | 1.84 | 1.73 | 0.10 | <0.001 |
| Scn2aB10_ | | | | | | | | | | |
| mRnu2-10 | | | | | | | | | | |
| 376_mRnu2- | 376 | mRnu2-10 | Scn2aB10 | mRnu1b6 | 1.94 | 1.57 | 1.70 | 1.74 | 0.19 | <0.001 |
| 10_Scn2aB10_ | | | | | | | | | | |
| mRnu1b6 | | | | | | | | | | |
| 370_mRnu2- | 370 | mRnu2-10 | Scn2aB10 | RNU7-1 | 1.88 | 1.49 | 1.84 | 1.74 | 0.21 | <0.001 |
| 10_Scn2aB10_ | | | | | | | | | | |
| RNU7-1 | | | | | | | | | | |
| 333_mRnu1b1_ | 333 | mRnu1b1 | Scn2aB10 | mRnu5g | 2.13 | 1.39 | 1.73 | 1.75 | 0.37 | <0.001 |
| Scn2aB10_ | | | | | | | | | | |
| mRnu5g | | | | | | | | | | |
| 366_mRnu2- | 366 | mRnu2-10 | Scn2aB10 | hU3 | 1.88 | 1.63 | 1.76 | 1.76 | 0.13 | <0.001 |
| 10_Scn2aB10_ | | | | | | | | | | |
| hU3 | | | | | | | | | | |
| 321_mRnu1b1_ | 321 | mRnu1b1 | Scn2aB10 | hRNU4-1 | 1.99 | 1.55 | 1.74 | 1.76 | 0.22 | <0.001 |
| Scn2aB10_ | | | | | | | | | | |
| hRNU4-1 | | | | | | | | | | |
| 314_mRnu1a1_ | 314 | mRnu1a1 | Scn2aB10 | mRnu12 | 1.79 | 1.79 | 1.71 | 1.77 | 0.04 | <0.001 |
| Scn2aB10_ | | | | | | | | | | |
| mRnu12 | | | | | | | | | | |
| 389_mRnu3b1_ | 389 | mRnu3b1 | Scn2aB10 | hRNU1-1 | 1.87 | 1.76 | 1.75 | 1.79 | 0.07 | <0.001 |
| Scn2aB10_ | | | | | | | | | | |
| hRNU1-1 | | | | | | | | | | |
| 302_mRnu1a1_ | 302 | mRnu1a1 | Scn2aB10 | hRNU11 | 1.73 | 1.91 | 1.75 | 1.80 | 0.10 | <0.001 |
| Scn2aB10_ | | | | | | | | | | |
| hRNU11 | | | | | | | | | | |
| 393_mRnu3b1_ | 393 | mRnu3b1 | Scn2aB10 | hU3 | 1.86 | 1.95 | 1.59 | 1.80 | 0.18 | <0.001 |
| Scn2aB10_hU3 | | | | | | | | | | |
| 320_mRnu1b1_ | 320 | mRnu1b1 | Scn2aB10 | hU3 | 2.01 | 1.67 | 1.73 | 1.80 | 0.18 | <0.001 |
| Scn2aB10_hU3 | | | | | | | | | | |
| 364_mRnu2- | 364 | mRnu2-10 | Scn2aB10 | hRNU1-3 | 1.86 | 1.66 | 1.91 | 1.81 | 0.13 | <0.001 |
| 10_Scn2aB10_ | | | | | | | | | | |
| hRNU1-3 | | | | | | | | | | |
| 399_mRnu3b1_ | 399 | mRnu3b1 | Scn2aB10 | hRNU12 | 1.96 | 1.58 | 1.88 | 1.81 | 0.20 | <0.001 |
| Scn2aB10_ | | | | | | | | | | |
| hRNU12 | | | | | | | | | | |
| 365_mRnu2- | 365 | mRnu2-10 | Scn2aB10 | hRNU2-1 | 1.97 | 1.61 | 1.86 | 1.81 | 0.19 | <0.001 |
| 10_Scn2aB10_ | | | | | | | | | | |
| hRNU2-1 | | | | | | | | | | |
| 360_mRnu1b6_ | 360 | mRnu1b6 | Scn2aB10 | mRnu12 | 2.13 | 1.60 | 1.71 | 1.81 | 0.28 | <0.001 |
| Scn2aB10_ | | | | | | | | | | |
| mRnu12 | | | | | | | | | | |
| 349_mRnu1b6_ | 349 | mRnu1b6 | Scn2aB10 | hRNU12 | 2.05 | 1.49 | 1.91 | 1.82 | 0.29 | <0.001 |
| Scn2aB10_ | | | | | | | | | | |
| hRNU12 | | | | | | | | | | |
| 322_mRnu1b1_ | 322 | mRnu1b1 | Scn2aB10 | hRNU5A-1 | 2.57 | 1.59 | 1.29 | 1.82 | 0.67 | <0.001 |
| Scn2aB10_ | | | | | | | | | | |
| hRNU5A-1 | | | | | | | | | | |
| 397_mRnu3b1_ | 397 | mRnu3b1 | Scn2aB10 | RNU7-1 | 1.90 | 1.83 | 1.75 | 1.83 | 0.07 | <0.001 |
| Scn2aB10_ | | | | | | | | | | |
| RNU7-1 | | | | | | | | | | |
| 363_mRnu2- | 363 | mRnu2-10 | Scn2aB10 | hRNU1-2 | 2.10 | 1.55 | 1.86 | 1.83 | 0.27 | <0.001 |
| 10_Scn2aB10_ | | | | | | | | | | |
| hRNU1-2 | | | | | | | | | | |
| 375_mRnu2- | 375 | mRnu2-10 | Scn2aB10 | mRnu1b1 | 1.86 | 1.64 | 2.00 | 1.84 | 0.18 | <0.001 |
| 10_Scn2aB10_ | | | | | | | | | | |
| mRnu1b1 | | | | | | | | | | |
| 310_mRnu1a1_ | 310 | mRnu1a1 | Scn2aB10 | mRnu5g | 1.81 | 1.67 | 2.03 | 1.84 | 0.18 | <0.001 |
| Scn2aB10_ | | | | | | | | | | |
| mRnu5g | | | | | | | | | | |
| 305_mRnu1a1_ | 305 | mRnu1a1 | Scn2aB10 | mRnu1a1 | 1.87 | 1.74 | 1.90 | 1.84 | 0.09 | <0.001 |
| Scn2aB10_ | | | | | | | | | | |
| mRnu1a1 | | | | | | | | | | |
| 306_mRnu1a1_ | 306 | mRnu1a1 | Scn2aB10 | mRnu1b1 | 1.98 | 1.90 | 1.66 | 1.85 | 0.17 | <0.001 |
| Scn2aB10_ | | | | | | | | | | |
| mRnu1b1 | | | | | | | | | | |
| 391_mRnu3b1_ | 391 | mRnu3b1 | Scn2aB10 | hRNU1-3 | 1.96 | 1.76 | 1.88 | 1.87 | 0.10 | <0.001 |
| Scn2aB10_ | | | | | | | | | | |
| hRNU1-3 | | | | | | | | | | |
| 339_mRnu1b6_ | 339 | mRnu1b6 | Scn2aB10 | hRNU1-1 | 2.02 | 1.62 | 1.95 | 1.87 | 0.21 | <0.001 |
| Scn2aB10_ | | | | | | | | | | |
| hRNU1-1 | | | | | | | | | | |
| 341_mRnu1b6_ | 341 | mRnu1b6 | Scn2aB10 | hRNU1-3 | 2.19 | 1.55 | 1.86 | 1.87 | 0.32 | <0.001 |
| Scn2aB10_ | | | | | | | | | | |
| hRNU1-3 | | | | | | | | | | |
| 307_mRnu1a1_ | 307 | mRnu1a1 | Scn2aB10 | mRnu1b6 | 1.59 | 1.98 | 2.05 | 1.87 | 0.25 | <0.001 |
| Scn2aB10_ | | | | | | | | | | |
| mRnu1b6 | | | | | | | | | | |
| 392_mRnu3b1_ | 392 | mRnu3b1 | Scn2aB10 | hRNU2-1 | 2.08 | 1.62 | 1.93 | 1.88 | 0.24 | <0.001 |
| Scn2aB10_ | | | | | | | | | | |
| hRNU2-1 | | | | | | | | | | |
| 367_mRnu2- | 367 | mRnu2-10 | Scn2aB10 | hRNU4-1 | 2.19 | 1.66 | 1.79 | 1.88 | 0.28 | <0.001 |
| 10_Scn2aB10_ | | | | | | | | | | |
| hRNU4-1 | | | | | | | | | | |
| 312_mRnu1a1_ | 312 | mRnu1a1 | Scn2aB10 | mRnu7 | 2.34 | 1.69 | 1.69 | 1.91 | 0.37 | <0.001 |
| Scn2aB10_ | | | | | | | | | | |
| mRnu7 | | | | | | | | | | |
| 328_mRnu1b1_ | 328 | mRnu1b1 | Scn2aB10 | mRnu1a1 | 2.35 | 1.72 | 1.65 | 1.91 | 0.39 | <0.001 |
| Scn2aB10__ | | | | | | | | | | |
| mRnu1a1 | | | | | | | | | | |
| 388_mu1a1_ | 388 | mu1a1 | Scn2aB10 | U1 | 2.19 | 1.70 | 1.86 | 1.92 | 0.25 | <0.001 |
| Scn2aB10_U1 | | | | | | | | | | |
| 348_mRnu1b6_ | 348 | mRnu1b6 | Scn2aB10 | hRNU11 | 2.10 | 1.7 | 2.03 | 1.95 | 0.19 | <0.001 |
| Scn2aB10_ | | | | | | | | | | |
| hRNU11 | | | | | | | | | | |
| 401_mRnu3b1_ | 401 | mRnu3b1 | Scn2aB10 | mRnu1a1 | 2.15 | 1.73 | 1.98 | 1.96 | 0.21 | <0.001 |
| Scn2aB10_ | | | | | | | | | | |
| mRnu1a1 | | | | | | | | | | |
| 340_mRnu1b6_ | 340 | mRnu1b6 | Scn2aB10 | hRNU1-2 | 2.09 | 1.67 | 2.12 | 1.96 | 0.25 | <0.001 |
| Scn2aB10_ | | | | | | | | | | |
| hRNU1-2 | | | | | | | | | | |
| 362_mRnu2- | 362 | mRnu2-10 | Scn2aB10 | hRNU1-1 | 2.40 | 1.73 | 1.77 | 1.97 | 0.38 | <0.001 |
| 10_Scn2aB10_ | | | | | | | | | | |
| hRNU1-1 | | | | | | | | | | |
| 359_mRnu1b6_ | 359 | mRnu1b6 | Scn2aB10 | mRnu11 | 2.10 | 1.74 | 2.11 | 1.98 | 0.21 | <0.001 |
| Scn2aB10_ | | | | | | | | | | |
| mRnu11 | | | | | | | | | | |
| 353_mRnu1b6_ | 353 | mRnu1b6 | Scn2aB10 | mRnu1b6 | 2.22 | 1.72 | 2.0 | 1.98 | 0.25 | <0.001 |
| Scn2aB10_ | | | | | | | | | | |
| mRnu1b6 | | | | | | | | | | |
| 329_mRnu1b1_ | 329 | mRnu1b1 | Scn2aB10 | mRnu1b1 | 2.25 | 1.79 | 1.93 | 1.99 | 0.24 | <0.001 |
| Scn2aB10_ | | | | | | | | | | |
| mRnu1b1 | | | | | | | | | | |
| 303_mRnu1a1_ | 303 | mRnu1a1 | Scn2aB10 | hRNU12 | 2.04 | 1.81 | 2.23 | 2.03 | 0.21 | <0.001 |
| Scn2aB10_ | | | | | | | | | | |
| hRNU12 | | | | | | | | | | |
| 377_mRnu2- | 377 | mRnu2-10 | Scn2aB10 | mRnu2-10 | 2.08 | 1.88 | 2.12 | 2.03 | 0.13 | <0.001 |
| 10_Scn2aB10_ | | | | | | | | | | |
| mRnu2-10 | | | | | | | | | | |
| 313_mRnu1a1_ | 313 | mRnu1a1 | Scn2aB10 | mRnu11 | 2.34 | 1.74 | 2.03 | 2.04 | 0.30 | <0.001 |
| Scn2aB10_ | | | | | | | | | | |
| mRnu11 | | | | | | | | | | |
| 345_mRnu1b6_ | 345 | mRnu1b6 | Scn2aB10 | hRNU5A-1 | 2.23 | 1.91 | 2.07 | 2.07 | 0.16 | <0.001 |
| Scn2aB10_ | | | | | | | | | | |
| hRNU5A-1 | | | | | | | | | | |
| 394_mRnu3b1_ | 394 | mRnu3b1 | Scn2aB10 | hRNU4-1 | 2.25 | 1.92 | 2.08 | 2.08 | 0.16 | <0.001 |
| Scn2aB10_ | | | | | | | | | | |
| hRNU4-1 | | | | | | | | | | |
| 368_mRnu2- | 368 | mRnu2-10 | Scn2aB10 | hRNU5A-1 | 2.50 | 1.93 | 1.86 | 2.10 | 0.35 | <0.001 |
| 10_Scn2aB10_ | | | | | | | | | | |
| hRNU5A-1 | | | | | | | | | | |
| 374_mRnu2- | 374 | mRnu2-10 | Scn2aB10 | mRnu1a1 | 2.25 | 1.72 | 2.39 | 2.12 | 0.36 | <0.001 |
| 10_Scn2aB10_ | | | | | | | | | | |
| mRnu1a1 | | | | | | | | | | |
| 398_mRnu3b1_ | 398 | mRnu3b1 | Scn2aB10 | hRNU11 | 2.18 | 2.14 | 2.07 | 2.13 | 0.06 | <0.001 |
| Scn2aB10_ | | | | | | | | | | |
| hRNU11 | | | | | | | | | | |
| 372_mRnu2- | 372 | mRnu2-10 | Scn2aB10 | hRNU12 | 2.43 | 1.77 | 2.29 | 2.16 | 0.35 | <0.001 |
| 10_Scn2aB10_ | | | | | | | | | | |
| hRNU12 | | | | | | | | | | |
| 390_mRnu3b1_ | 390 | mRnu3b1 | Scn2aB10 | hRNU1-2 | 2.39 | 1.89 | 2.24 | 2.17 | 0.25 | <0.001 |
| Scn2aB10_ | | | | | | | | | | |
| hRNU1-2 | | | | | | | | | | |
| 379_mRnu2- | 379 | mRnu2-10 | Scn2aB10 | mRnu5g | 2.69 | 1.85 | 1.98 | 2.17 | 0.45 | <0.001 |
| 10_Scn2aB10_ | | | | | | | | | | |
| mRnu5g | | | | | | | | | | |
| 395_mRnu3b1_ | 395 | mRnu3b1 | Scn2aB10 | hRNU5A-1 | 1.88 | 2.25 | 2.68 | 2.27 | 0.40 | <0.001 |
| Scn2aB10_ | | | | | | | | | | |
| hRNU5A-1 | | | | | | | | | | |

TABLE 12
Library 5 Summary

| | | | | | | | | | | P-value |
|---|---|---|---|---|---|---|---|---|---|---|
| Name | ID | Promoter | U7 | Terminator | Rep1 | Rep2 | Rep3 | Average | SD | (ANOVA) |
| 448_mRnu6_ | 448 | mRnu6 | Scn2aB10 | mRnu1b1 | 0.74 | 0.82 | 0.82 | 0.79 | 0.05 | 0.28 |
| Scn2aB10_ | | | | | | | | | | |
| mRnu1b1 | | | | | | | | | | |
| 444_mRnu6_ | 444 | mRnu6 | Scn2aB10 | hRNU11 | 0.84 | 0.74 | 0.90 | 0.83 | 0.08 | 0.40 |
| Scn2aB10_ | | | | | | | | | | |
| hRNU11 | | | | | | | | | | |
| 446_mRnu6_ | 446 | mRnu6 | Scn2aB10 | hRN7SK | 0.80 | 0.91 | 0.86 | 0.86 | 0.05 | 0.54 |
| Scn2aB10_ | | | | | | | | | | |
| hRN7SK | | | | | | | | | | |
| 454_mRnu6 | 454 | mRnu6 | Scn2aB10 | mRnu7 | 0.85 | 0.90 | 0.82 | 0.86 | 0.04 | 0.54 |
| Scn2aB10_ | | | | | | | | | | |
| mRnu7 | | | | | | | | | | |
| 442_mRnu6_ | 442 | mRnu6 | Scn2aB10 | hRNU6-1 | 0.80 | 0.88 | 0.91 | 0.86 | 0.05 | 0.58 |
| Scn2aB10_ | | | | | | | | | | |
| hRNU6-1 | | | | | | | | | | |
| 457_mRnu6_ | 457 | mRnu6 | Scn2aB10 | mRn7sk | 0.82 | 0.97 | 0.91 | 0.90 | 0.07 | 0.78 |
| Scn2aB10_ | | | | | | | | | | |
| mRn7sk | | | | | | | | | | |
| 450_mRnu6_ | 450 | mRnu6 | Scn2aB10 | mRnu2-10 | 0.88 | 0.96 | 0.88 | 0.91 | 0.04 | 0.82 |
| Scn2aB10_ | | | | | | | | | | |
| mRnu2-10 | | | | | | | | | | |
| 449_mRnu6_ | 449 | mRnu6 | Scn2aB10 | mRnu1b6 | 0.95 | 0.92 | 0.89 | 0.92 | 0.03 | 0.90 |
| Scn2aB10_ | | | | | | | | | | |
| mRnu1b6 | | | | | | | | | | |
| 436_mRnu6_ | 436 | mRnu6 | Scn2aB10 | hRNU1-2 | 0.89 | 0.97 | 0.90 | 0.92 | 0.05 | 0.90 |
| Scn2aB10_ | | | | | | | | | | |
| hRNU1-2 | | | | | | | | | | |
| 455_mRnu6_ | 455 | mRnu6 | Scn2aB10 | mRnu11 | 0.89 | 0.85 | 1.02 | 0.92 | 0.09 | 0.90 |
| Scn2aB10_ | | | | | | | | | | |
| mRnu11 | | | | | | | | | | |
| 482 mu1a1 | 482 | mu1a1 | Scr2 | U1 | 1.02 | 0.93 | 0.86 | 0.94 | 0.08 | Control |
| Scr2_U1 | | | | | | | | | | |
| 445_mRnu6_ | 445 | mRnu6 | Scn2aB10 | hRNU12 | 0.86 | 1.01 | 0.95 | 0.94 | 0.07 | 0.98 |
| Scn2aB10_ | | | | | | | | | | |
| hRNU12 | | | | | | | | | | |
| 451_mRnu6_ | 451 | mRnu6 | Scn2aB10 | mRnu3b1 | 0.81 | 0.94 | 1.15 | 0.97 | 0.17 | 0.82 |
| Scn2aB10_ | | | | | | | | | | |
| mRnu3b1 | | | | | | | | | | |
| 452_mRnu6_ | 452 | mRnu6 | Scn2aB10 | mRnu5g | 1.07 | 0.91 | 0.93 | 0.97 | 0.09 | 0.80 |
| Scn2aB10_ | | | | | | | | | | |
| mRnu5g | | | | | | | | | | |
| 447_mRnu6_ | 447 | mRnu6 | Scn2aB10 | mRnu1a1 | 1.08 | 0.91 | 0.93 | 0.97 | 0.09 | 0.78 |
| Scn2aB10_ | | | | | | | | | | |
| mRnu1a1 | | | | | | | | | | |
| 440_mRnu6_ | 440 | mRnu6 | Scn2aB10 | hRNU4-1 | 1.05 | 1.02 | 0.94 | 1.01 | 0.06 | 0.61 |
| Scn2aB10_ | | | | | | | | | | |
| hRNU4-1 | | | | | | | | | | |
| 456_mRnu6_ | 456 | mRnu6 | Scn2aB10 | mRnu12 | 1.01 | 1.12 | 0.89 | 1.01 | 0.12 | 0.59 |
| Scn2aB10_ | | | | | | | | | | |
| mRnu12 | | | | | | | | | | |
| 443_mRnu6_ | 443 | mRnu6 | Scn2aB10 | RNU7-1 | 0.84 | 1.29 | 0.96 | 1.03 | 0.24 | 0.48 |
| Scn2aB10_ | | | | | | | | | | |
| RNU7-1 | | | | | | | | | | |
| 435_mRnu6_ | 435 | mRnu6 | Scn2aB10 | hRNU1-1 | 0.93 | 1.00 | 1.18 | 1.04 | 0.13 | 0.45 |
| Scn2aB10_ | | | | | | | | | | |
| hRNU1-1 | | | | | | | | | | |
| 438_mRnu6_ | 438 | mRnu6 | Scn2aB10 | hRNU2-1 | 0.95 | 1.30 | 0.92 | 1.06 | 0.21 | 0.36 |
| Scn2aB10_ | | | | | | | | | | |
| hRNU2-1 | | | | | | | | | | |
| 439_mRnu6_ | 439 | mRnu6 | Scn2aB10 | hU3 | 1.07 | 1.14 | 0.98 | 1.06 | 0.08 | 0.34 |
| Scn2aB10_hU3 | | | | | | | | | | |
| 481_mu1a1_ | 481 | mu1a1 | Scn2aF1Scr2 | U1 | 0.98 | 1.07 | 1.14 | 1.06 | 0.08 | 0.34 |
| Scn2aF1Scr2_ | | | | | | | | | | |
| U1 | | | | | | | | | | |
| 437_mRnu6_ | 437 | mRnu6 | Scn2aB10 | hRNU1-3 | 1.01 | 1.09 | 1.11 | 1.07 | 0.05 | 0.31 |
| Scn2aB10_ | | | | | | | | | | |
| hRNU1-3 | | | | | | | | | | |
| 441_mRnu6_ | 441 | mRnu6 | Scn2aB10 | hRNU5A-1 | 1.23 | 1.04 | 0.97 | 1.08 | 0.13 | 0.28 |
| Scn2aB10_ | | | | | | | | | | |
| hRNU5A-1 | | | | | | | | | | |
| 419_mRnu5g_ | 419 | mRnu5g | Scn2aB10 | hRNU6-1 | 1.10 | 1.11 | 1.21 | 1.14 | 0.06 | 0.12 |
| Scn2aB10_ | | | | | | | | | | |
| hRNU6-1 | | | | | | | | | | |
| 500_mRnu11_ | 500 | mRnu11 | Scn2aB10 | mRnu2-10 | 1.26 | 1.16 | 1.08 | 1.17 | 0.09 | 0.08 |
| Scn2aB10_ | | | | | | | | | | |
| mRnu2-10 | | | | | | | | | | |
| 492_mRnu11 | 492 | mRnu11 | Scn2aB10 | hRNU6-1 | 1.29 | 1.29 | 0.97 | 1.18 | 0.18 | 0.06 |
| Scn2aB10_ | | | | | | | | | | |
| hRNU6-1 | | | | | | | | | | |
| 420_mRnu5g_ | 420 | mRnu5g | Scn2aB10 | RNU7-1 | 1.27 | 1.15 | 1.23 | 1.22 | 0.06 | 0.03 |
| Scn2aB10_ | | | | | | | | | | |
| RNU7-1 | | | | | | | | | | |
| 465_mRnu7_ | 465 | mRnu7 | Scn2aB10 | hRNU6-1 | 1.16 | 1.43 | 1.09 | 1.23 | 0.18 | 0.03 |
| Scn2aB10_ | | | | | | | | | | |
| hRNU6-1 | | | | | | | | | | |
| 477_mRnu7_ | 477 | mRnu7 | Scn2aB10 | mRnu7 | 1.10 | 1.34 | 1.34 | 1.26 | 0.14 | 0.01 |
| Scn2aB10_ | | | | | | | | | | |
| mRnu7 | | | | | | | | | | |
| 470_mRnu7_ | 470 | mRnu7 | Scn2aB10 | mRnu1a1 | 1.24 | 1.18 | 1.36 | 1.26 | 0.09 | 0.01 |
| Scn2aB10_ | | | | | | | | | | |
| mRnu1a1 | | | | | | | | | | |
| 428_mRnu5g_ | 428 | mRnu5g | Scn2aB10 | mRnu3b1 | 1.33 | 1.17 | 1.29 | 1.26 | 0.08 | 0.01 |
| Scn2aB10_ | | | | | | | | | | |
| mRnu3b1 | | | | | | | | | | |
| 474_mRnu7_ | 474 | mRnu7 | Scn2aB10 | mRnu3b1 | 1.23 | 1.45 | 1.18 | 1.29 | 0.14 | 0.008 |
| Scn2aB10_ | | | | | | | | | | |
| mRnu3b1 | | | | | | | | | | |
| 421_mRnu5g_ | 421 | mRnu5g | Scn2aB10 | hRNU11 | 1.30 | 1.22 | 1.35 | 1.29 | 0.06 | 0.008 |
| Scn2aB10_ | | | | | | | | | | |
| hRNU11 | | | | | | | | | | |
| 469_mRnu7_ | 469 | mRnu7 | Scn2aB10 | hRN7SK | 1.33 | 1.24 | 1.34 | 1.31 | 0.05 | 0.006 |
| Scn2aB10_ | | | | | | | | | | |
| hRN7SK | | | | | | | | | | |
| 473_mRnu7_ | 473 | mRnu7 | Scn2aB10 | mRnu2-10 | 1.31 | 1.19 | 1.44 | 1.31 | 0.12 | 0.005 |
| Scn2aB10_ | | | | | | | | | | |
| mRnu2-10 | | | | | | | | | | |
| 462_mRnu7_ | 462 | mRnu7 | Scn2aB10 | hU3 | 1.33 | 1.39 | 1.23 | 1.32 | 0.08 | 0.004 |
| Scn2aB10_hU3 | | | | | | | | | | |
| 431_mRnu5g_ | 431 | mRnu5g | Scn2aB10 | mRnu7 | 1.19 | 1.39 | 1.42 | 1.34 | 0.12 | 0.003 |
| Scn2aB10_ | | | | | | | | | | |
| mRnu7 | | | | | | | | | | |
| 493_mRnu11_ | 493 | mRnu11 | Scn2aB10 | RNU7-1 | 1.20 | 1.46 | 1.40 | 1.35 | 0.14 | 0.002 |
| Scn2aB10_ | | | | | | | | | | |
| RNU7-1 | | | | | | | | | | |
| 427_mRnu5g_ | 427 | mRnu5g | Scn2aB10 | mRnu2-10 | 1.50 | 0.94 | 1.65 | 1.36 | 0.38 | 0.001 |
| Scn2aB10_ | | | | | | | | | | |
| mRnu2-10 | | | | | | | | | | |
| 501_mRnu11_ | 501 | mRnu11 | Scn2aB10 | mRnu3b1 | 1.37 | 1.55 | 1.19 | 1.37 | 0.18 | 0.001 |
| Scn2aB10_ | | | | | | | | | | |
| mRnu3b1 | | | | | | | | | | |
| 425_mRnu5g_ | 425 | mRnu5g | Scn2aB10 | mRnu1b1 | 1.21 | 1.45 | 1.45 | 1.37 | 0.14 | 0.001 |
| Scn2aB10_ | | | | | | | | | | |
| mRnu1b1 | | | | | | | | | | |
| 461_mRnu7_ | 461 | mRnu7 | Scn2aB10 | hRNU2-1 | 1.33 | 1.39 | 1.40 | 1.37 | 0.04 | 0.001 |
| Scn2aB10_ | | | | | | | | | | |
| hRNU2-1 | | | | | | | | | | |
| 424_mRnu5g_ | 424 | mRnu5g | Scn2aB10 | mRnu1a1 | 1.35 | 1.43 | 1.39 | 1.39 | 0.04 | <0.001 |
| Scn2aB10__ | | | | | | | | | | |
| mRnu1a1 | | | | | | | | | | |
| 472_mRnu7_ | 472 | mRnu7 | Scn2aB10 | mRnu1b6 | 1.30 | 1.47 | 1.42 | 1.39 | 0.09 | <0.001 |
| Scn2aB10_ | | | | | | | | | | |
| mRnu1b6 | | | | | | | | | | |
| 499_mRnu11_ | 499 | mRnu11 | Scn2aB10 | mRnu1b6 | 1.28 | 1.24 | 1.66 | 1.39 | 0.23 | <0.001 |
| Scn2aB10_ | | | | | | | | | | |
| mRnu1b6 | | | | | | | | | | |
| 402_mRnu3b1_ | 402 | mRnu3b1 | Scn2aB10 | mRnu1b1 | 1.30 | 1.47 | 1.46 | 1.41 | 0.09 | <0.001 |
| Scn2aB10_ | | | | | | | | | | |
| mRnu1b1 | | | | | | | | | | |
| 432_mRnu5g_ | 432 | mRnu5g | Scn2aB10 | mRnu11 | 1.23 | 1.57 | 1.44 | 1.41 | 0.17 | <0.001 |
| Scn2aB10_ | | | | | | | | | | |
| mRnu11 | | | | | | | | | | |
| 414_mRnu5g_ | 414 | mRnu5g | Scn2aB10 | hRNU1-3 | 1.26 | 1.38 | 1.61 | 1.41 | 0.18 | <0.001 |
| Scn2aB10_ | | | | | | | | | | |
| hRNU1-3 | | | | | | | | | | |
| 423_mRnu5g_ | 423 | mRnu5g | Scn2aB10 | hRN7SK | 1.32 | 1.66 | 1.27 | 1.42 | 0.21 | <0.001 |
| Scn2aB10_ | | | | | | | | | | |
| hRN7SK | | | | | | | | | | |
| 434_mRnu5g_ | 434 | mRnu5g | Scn2aB10 | mRn7sk | 1.49 | 1.30 | 1.51 | 1.43 | 0.12 | <0.001 |
| Scn2aB10_ | | | | | | | | | | |
| mRn7sk | | | | | | | | | | |
| 466_mRnu7_ | 466 | mRnu7 | Scn2aB10 | RNU7-1 | 1.16 | 1.85 | 1.29 | 1.44 | 0.37 | <0.001 |
| Scn2aB10_ | | | | | | | | | | |
| RNU7-1 | | | | | | | | | | |
| 412_mRnu5g_ | 412 | mRnu5g | Scn2aB10 | hRNU1-1 | 1.38 | 1.56 | 1.43 | 1.46 | 0.09 | <0.001 |
| Scn2aB10_ | | | | | | | | | | |
| hRNU1-1 | | | | | | | | | | |
| 411_mRnu3b1_ | 411 | mRnu3b1 | Scn2aB10 | mRn7sk | 1.42 | 1.42 | 1.54 | 1.46 | 0.07 | <0.001 |
| Scn2aB10_ | | | | | | | | | | |
| mRn7sk | | | | | | | | | | |
| 479_mRnu7 | 479 | mRnu7 | Scn2aB10 | mRnu12 | 1.45 | 1.51 | 1.45 | 1.47 | 0.03 | <0.001 |
| Scn2aB10_ | | | | | | | | | | |
| mRnu12 | | | | | | | | | | |
| 498_mRnu11_ | 498 | mRnu11 | Scn2aB10 | mRnu1b1 | 1.38 | 1.65 | 1.39 | 1.47 | 0.15 | <0.001 |
| Scn2aB10_ | | | | | | | | | | |
| mRnu1b1 | | | | | | | | | | |
| 429_mRnu5g_ | 429 | mRnu5g | Scn2aB10 | mRnu5g | 1.58 | 1.35 | 1.52 | 1.48 | 0.12 | <0.001 |
| Scn2aB10_ | | | | | | | | | | |
| mRnu5g | | | | | | | | | | |
| 433_mRnu5g_ | 433 | mRnu5g | Scn2aB10 | mRnu12 | 1.51 | 1.45 | 1.50 | 1.49 | 0.03 | <0.001 |
| Scn2aB10_ | | | | | | | | | | |
| mRnu12 | | | | | | | | | | |
| 471_mRnu7_ | 471 | mRnu7 | Scn2aB10 | mRnu1b1 | 1.52 | 1.33 | 1.63 | 1.49 | 0.15 | <0.001 |
| Scn2aB10_ | | | | | | | | | | |
| mRnu1b1 | | | | | | | | | | |
| 458_mRnu7_ | 458 | mRnu7 | Scn2aB10 | hRNU1-1 | 1.43 | 1.68 | 1.38 | 1.50 | 0.16 | <0.001 |
| Scn2aB10_ | | | | | | | | | | |
| hRNU1-1 | | | | | | | | | | |
| 459_mRnu7_ | 459 | mRnu7 | Scn2aB10 | hRNU1-2 | 1.36 | 1.73 | 1.40 | 1.50 | 0.20 | <0.001 |
| Scn2aB10_ | | | | | | | | | | |
| hRNU1-2 | | | | | | | | | | |
| 478_mRnu7_ | 478 | mRnu7 | Scn2aB10 | mRnu11 | 1.54 | 1.35 | 1.61 | 1.50 | 0.13 | <0.001 |
| Scn2aB10_ | | | | | | | | | | |
| mRnu11 | | | | | | | | | | |
| 417_mRnu5g_ | 417 | mRnu5g | Scn2aB10 | hRNU4-1 | 1.22 | 1.60 | 1.70 | 1.51 | 0.25 | <0.001 |
| Scn2aB10_ | | | | | | | | | | |
| hRNU4-1 | | | | | | | | | | |
| 422_mRnu5g_ | 422 | mRnu5g | Scn2aB10 | hRNU12 | 1.43 | 1.57 | 1.56 | 1.52 | 0.08 | <0.001 |
| Scn2aB10_ | | | | | | | | | | |
| hRNU12 | | | | | | | | | | |
| 496_mRnu11_ | 496 | mRnu11 | Scn2aB10 | hRN7SK | 1.57 | 1.39 | 1.63 | 1.53 | 0.12 | <0.001 |
| Scn2aB10_ | | | | | | | | | | |
| hRN7SK | | | | | | | | | | |
| 416_mRnu5g_ | 416 | mRnu5g | Scn2aB10 | hU3 | 1.40 | 1.44 | 1.76 | 1.53 | 0.19 | <0.001 |
| Scn2aB10_hU3 | | | | | | | | | | |
| 408_mRnu3b1_ | 408 | mRnu3b1 | Scn2aB10 | mRnu7 | 1.28 | 1.65 | 1.67 | 1.54 | 0.22 | <0.001 |
| Scn2aB10_ | | | | | | | | | | |
| mRnu7 | | | | | | | | | | |
| 486_mRnu11_ | 486 | mRnu11 | Scn2aB10 | hRNU1-2 | 1.34 | 1.64 | 1.64 | 1.54 | 0.17 | <0.001 |
| Scn2aB10_ | | | | | | | | | | |
| hRNU1-2 | | | | | | | | | | |
| 475_mRnu7_ | 475 | mRnu7 | Scn2aB10 | mRnu5g | 1.53 | 1.6 | 1.46 | 1.55 | 0.11 | <0.001 |
| Scn2aB10_ | | | | | | | | | | |
| mRnu5g | | | | | | | | | | |
| 491_mRnu11_ | 491 | mRnu11 | Scn2aB10 | hRNU5A-1 | 1.43 | 1.71 | 1.55 | 1.56 | 0.14 | <0.001 |
| Scn2aB10_ | | | | | | | | | | |
| hRNU5A-1 | | | | | | | | | | |
| 460_mRnu7_ | 460 | mRnu7 | Scn2aB10 | hRNU1-3 | 1.37 | 1.73 | 1.63 | 1.58 | 0.18 | <0.001 |
| Scn2aB10_ | | | | | | | | | | |
| hRNU1-3 | | | | | | | | | | |
| 410_mRnu3b1_ | 410 | mRnu3b1 | Scn2aB10 | mRnu12 | 1.54 | 1.54 | 1.66 | 1.58 | 0.07 | <0.001 |
| Scn2aB10_ | | | | | | | | | | |
| mRnu12 | | | | | | | | | | |
| 494_mRnu11_ | 494 | mRnu11 | Scn2aB10 | hRNU11 | 1.60 | 1.7 | 1.60 | 1.63 | 0.0 | <0.001 |
| Scn2aB10_ | | | | | | | | | | |
| hRNU11 | | | | | | | | | | |
| 467_mRnu7_ | 467 | mRnu7 | Scn2aB10 | hRNU11 | 1.66 | 1.65 | 1.68 | 1.66 | 0.02 | <0.001 |
| Scn2aB10_ | | | | | | | | | | |
| hRNU11 | | | | | | | | | | |
| 485_mRnu11_ | 485 | mRnu11 | Scn2aB10 | hRNU1-1 | 1.38 | 1.84 | 1.79 | 1.67 | 0.25 | <0.001 |
| Scn2aB10_ | | | | | | | | | | |
| hRNU1-1 | | | | | | | | | | |
| 490_mRnu11_ | 490 | mRnu11 | Scn2aB10 | hRNU4-1 | 1.65 | 1.84 | 1.52 | 1.67 | 0.16 | <0.001 |
| Scn2aB10_ | | | | | | | | | | |
| hRNU4-1 | | | | | | | | | | |
| 413_mRnu5g_ | 413 | mRnu5g | Scn2aB10 | hRNU1-2 | 1.71 | 2.00 | 1.34 | 1.68 | 0.34 | <0.001 |
| Scn2aB10_ | | | | | | | | | | |
| hRNU1-2 | | | | | | | | | | |
| 409_mRnu3b1_ | 409 | mRnu3b1 | Scn2aB10 | mRnu11 | 1.50 | 1.66 | 1.89 | 1.69 | 0.20 | <0.001 |
| Scn2aB10_ | | | | | | | | | | |
| mRnu11 | | | | | | | | | | |
| 488_mRnu11_ | 488 | mRnu11 | Scn2aB10 | hRNU2-1 | 1.75 | 1.78 | 1.59 | 1.71 | 0.10 | <0.001 |
| Scn2aB10_ | | | | | | | | | | |
| hRNU2-1 | | | | | | | | | | |
| 495_mRnu11_ | 495 | mRnu11 | Scn2aB10 | hRNU12 | 1.92 | 1.81 | 1.44 | 1.72 | 0.25 | <0.001 |
| Scn2aB10_ | | | | | | | | | | |
| hRNU12 | | | | | | | | | | |
| 415_mRnu5g_ | 415 | mRnu5g | Scn2aB10 | hRNU2-1 | 1.80 | 1.83 | 1.58 | 1.74 | 0.14 | <0.001 |
| Scn2aB10_ | | | | | | | | | | |
| hRNU2-1 | | | | | | | | | | |
| 426_mRnu5g_ | 426 | mRnu5g | Scn2aB10 | mRnu1b6 | 1.69 | 1.84 | 1.69 | 1.74 | 0.08 | <0.001 |
| Scn2aB10_ | | | | | | | | | | |
| mRnu1b6 | | | | | | | | | | |
| 483 mu1a1_ | 483 | mu1a1 | Scn2aD12 | U1 | 2.02 | 1.74 | 1.47 | 1.74 | 0.27 | <0.001 |
| Scn2aD12_U1 | | | | | | | | | | |
| 484 mu1a1_ | 484 | mu1a1 | Scn2aB10 | U1 | 1.66 | 1.78 | 1.82 | 1.76 | 0.09 | <0.001 |
| Scn2aB10_U1 | | | | | | | | | | |
| 464_mRnu7_ | 464 | mRnu7 | Scn2aB10 | hRNU5A-1 | 1.73 | 1.81 | 1.82 | 1.79 | 0.05 | <0.001 |
| Scn2aB10_ | | | | | | | | | | |
| hRNU5A-1 | | | | | | | | | | |
| 463_mRnu7_ | 463 | mRnu7 | Scn2aB10 | hRNU4-1 | 1.63 | 2.01 | 1.83 | 1.82 | 0.19 | <0.001 |
| Scn2aB10_ | | | | | | | | | | |
| hRNU4-1 | | | | | | | | | | |
| 406_mRnu3b1_ | 406 | mRnu3b1 | Scn2aB10 | mRnu5g | 1.75 | 2.03 | 1.72 | 1.83 | 0.17 | <0.001 |
| Scn2aB10_ | | | | | | | | | | |
| mRnu5g | | | | | | | | | | |
| 468_mRnu7_ | 468 | mRnu7 | Scn2aB10 | hRNU12 | 1.36 | 2.21 | 1.94 | 1.84 | 0.44 | <0.001 |
| Scn2aB10_ | | | | | | | | | | |
| hRNU12 | | | | | | | | | | |
| 405_mRnu3b1_ | 405 | mRnu3b1 | Scn2aB10 | mRnu3b1 | 1.72 | 1.75 | 2.10 | 1.86 | 0.21 | <0.001 |
| Scn2aB10_ | | | | | | | | | | |
| mRnu3b1 | | | | | | | | | | |
| 404_mRnu3b1_ | 404 | mRnu3b1 | Scn2aB10 | mRnu2-10 | 1.56 | 2.19 | 1.96 | 1.90 | 0.32 | <0.001 |
| Scn2aB10_ | | | | | | | | | | |
| mRnu2-10 | | | | | | | | | | |
| 418_mRnu5g_ | 418 | mRnu5g | Scn2aB10 | hRNU5A-1 | 1.68 | 2.71 | 1.63 | 2.01 | 0.61 | <0.001 |
| Scn2aB10_ | | | | | | | | | | |
| hRNU5A-1 | | | | | | | | | | |

TABLE 13
Library 6 Summary

| | | | | | | | | | | ANOVA |
|---|---|---|---|---|---|---|---|---|---|---|
| Name | ID | Promoter | U7 | Terminator | Rep1 | Rep2 | Rep3 | Average | SD | P-value |
| 2A12_194_ | 194 | mu1a1 | Scr2 | U1 | 0.83 | 0.81 | 0.82 | 0.82 | 0.01 | 0.24 |
| mu1a1_Scr2_U1 | | | | | | | | | | |
| 530_mRnu12_ | 530 | mRnu12 | Scn2aB10 | mRn7sk | 0.96 | 0.78 | 0.94 | 0.89 | 0.10 | 0.55 |
| Scn2aB10_ | | | | | | | | | | |
| mRn7sk | | | | | | | | | | |
| 515_mRnu12_ | 515 | mRnu12 | Scn2aB10 | hRNU6-1 | 0.92 | 0.88 | 0.95 | 0.92 | 0.03 | 0.68 |
| Scn2aB10_ | | | | | | | | | | |
| hRNU6-1 | | | | | | | | | | |
| 546_mRn7sk_ | 546 | mRn7sk | Scn2aB10 | mRnu2-10 | 0.73 | 1.05 | 1.10 | 0.96 | 0.20 | 0.94 |
| Scn2aB10_ | | | | | | | | | | |
| mRnu2-10 | | | | | | | | | | |
| 2H11_193_ | 193 | mu1a1 | Scn2aF1Scr2 | U1 | 1.03 | 0.86 | 1.02 | 0.97 | 0.10 | Control |
| mu1a1_ | | | | | | | | | | |
| Scn2aF1Scr2_U1 | | | | | | | | | | |
| 535_mRn7sk | 535 | mRn7sk | Scn2aB10 | hU3 | 1.08 | 1.06 | 0.99 | 1.04 | 0.04 | 0.57 |
| Scn2aB10_hU3 | | | | | | | | | | |
| 542_mRn7sk_ | 542 | mRn7sk | Scn2aB10 | hRN7SK | 1.05 | 1.03 | 1.11 | 1.06 | 0.05 | 0.47 |
| Scn2aB10_ | | | | | | | | | | |
| hRN7SK | | | | | | | | | | |
| 548_mRn7sk_ | 548 | mRn7sk | Scn2aB10 | mRnu5g | 1.08 | 0.95 | 1.20 | 1.07 | 0.12 | 0.4 |
| Scn2aB10_ | | | | | | | | | | |
| mRnu5g | | | | | | | | | | |
| 524_mRnu12_ | 524 | mRnu12 | Scn2aB10 | mRnu3b1 | 1.21 | 0.97 | 1.09 | 1.09 | 0.12 | 0.35 |
| Scn2aB10_ | | | | | | | | | | |
| mRnu3b1 | | | | | | | | | | |
| 539_mRn7sk_ | 539 | mRn7sk | Scn2aB10 | RNU7-1 | 1.16 | 0.83 | 1.27 | 1.09 | 0.23 | 0.36 |
| Scn2aB10_ | | | | | | | | | | |
| RNU7-1 | | | | | | | | | | |
| 534_mRn7sk_ | 534 | mRn7sk | Scn2aB10 | hRNU2-1 | 1.27 | 0.90 | 1.12 | 1.09 | 0.19 | 0.32 |
| Scn2aB10_ | | | | | | | | | | |
| hRNU2-1 | | | | | | | | | | |
| 545_mRn7sk_ | 545 | mRn7sk | Scn2aB10 | mRnu1b6 | 1.39 | 1.14 | 0.80 | 1.11 | 0.29 | 0.27 |
| Scn2aB10_ | | | | | | | | | | |
| mRnu1b6 | | | | | | | | | | |
| 538_mRn7sk_ | 538 | mRn7sk | Scn2aB10 | hRNU6-1 | 1.18 | 0.94 | 1.24 | 1.12 | 0.16 | 0.24 |
| Scn2aB10_ | | | | | | | | | | |
| hRNU6-1 | | | | | | | | | | |
| 541_mRn7sk_ | 541 | mRn7sk | Scn2aB10 | hRNU12 | 1.17 | 0.94 | 1.25 | 1.12 | 0.16 | 0.24 |
| Scn2aB10_ | | | | | | | | | | |
| hRNU12 | | | | | | | | | | |
| 532_mRn7sk_ | 532 | mRn7sk | Scn2aB10 | hRNU1-2 | 1.24 | 0.98 | 1.18 | 1.13 | 0.14 | 0.2 |
| Scn2aB10_ | | | | | | | | | | |
| hRNU1-2 | | | | | | | | | | |
| 531_mRn7sk_ | 531 | mRn7sk | Scn2aB10 | hRNU1-1 | 1.19 | 1.13 | 1.13 | 1.15 | 0.03 | 0.16 |
| Scn2aB10_ | | | | | | | | | | |
| hRNU1-1 | | | | | | | | | | |
| 553_mRn7sk_ | 553 | mRn7sk | Scn2aB10 | mRn7sk | 0.97 | 1.26 | 1.33 | 1.18 | 0.19 | 0.09 |
| Scn2aB10_ | | | | | | | | | | |
| mRn7sk | | | | | | | | | | |
| 537_mRn7sk_ | 537 | mRn7sk | Scn2aB10 | hRNU5A-1 | 1.15 | 1.09 | 1.38 | 1.21 | 0.15 | 0.07 |
| Scn2aB10_ | | | | | | | | | | |
| hRNU5A-1 | | | | | | | | | | |
| 1A1_001_mu1a1_ | 001 | mu1a1 | Scn2aF1Scr2 | U1 | 1.14 | 1.33 | 1.15 | 1.21 | 0.10 | 0.07 |
| Scn2aF1Scr2_ | | | | | | | | | | |
| U1 | | | | | | | | | | |
| 544_mRn7sk_ | 544 | mRn7sk | Scn2aB10 | mRnu1b1 | 1.18 | 1.09 | 1.36 | 1.21 | 0.14 | 0.06 |
| Scn2aB10_ | | | | | | | | | | |
| mRnu1b1 | | | | | | | | | | |
| 523_mRnu12_ | 523 | mRnu12 | Scn2aB10 | mRnu2-10 | 1.35 | 1.04 | 1.24 | 1.21 | 0.16 | 0.06 |
| Scn2aB10_ | | | | | | | | | | |
| mRnu2-10 | | | | | | | | | | |
| 552_mRn7sk_ | 552 | mRn7sk | Scn2aB10 | mRnu12 | 1.22 | 1.03 | 1.42 | 1.22 | 0.20 | 0.05 |
| Scn2aB10_ | | | | | | | | | | |
| mRnu12 | | | | | | | | | | |
| 543_mRn7sk_ | 543 | mRn7sk | Scn2aB10 | mRnu1a1 | 1.22 | 1.17 | 1.28 | 1.22 | 0.05 | 0.05 |
| Scn2aB10_ | | | | | | | | | | |
| mRnu1a1 | | | | | | | | | | |
| 516_mRnu12_ | 516 | mRnu12 | Scn2aB10 | RNU7-1 | 1.17 | 1.15 | 1.38 | 1.23 | 0.13 | 0.04 |
| Scn2aB10_ | | | | | | | | | | |
| RNU7-1 | | | | | | | | | | |
| 529_mRnu12_ | 529 | mRnu12 | Scn2aB10 | mRnu12 | 1.36 | 1.03 | 1.30 | 1.23 | 0.18 | 0.04 |
| Scn2aB10_ | | | | | | | | | | |
| mRnu12 | | | | | | | | | | |
| 525_mRnu12_ | 525 | mRnu12 | Scn2aB10 | mRnu5g | 1.36 | 1.29 | 1.12 | 1.26 | 0.12 | 0.03 |
| Scn2aB10_ | | | | | | | | | | |
| mRnu5g | | | | | | | | | | |
| 533_mRn7sk_ | 533 | mRn7sk | Scn2aB10 | hRNU1-3 | 1.19 | 1.27 | 1.33 | 1.26 | 0.07 | 0.02 |
| Scn2aB10_ | | | | | | | | | | |
| hRNU1-3 | | | | | | | | | | |
| 540_mRn7sk_ | 540 | mRn7sk | Scn2aB10 | hRNU11 | 1.20 | 1.01 | 1.63 | 1.28 | 0.32 | 0.02 |
| Scn2aB10_ | | | | | | | | | | |
| hRNU11 | | | | | | | | | | |
| 512_mRnu12_ | 512 | mRnu12 | Scn2aB10 | hU3 | 1.31 | 1.16 | 1.47 | 1.32 | 0.15 | 0.008 |
| Scn2aB10_hU3 | | | | | | | | | | |
| 519_mRnu12_ | 519 | mRnu12 | Scn2aB10 | hRN7SK | 1.47 | 1.35 | 1.16 | 1.33 | 0.16 | 0.006 |
| Scn2aB10_ | | | | | | | | | | |
| hRN7SK | | | | | | | | | | |
| 508_mRnu12_ | 508 | mRnu12 | Scn2aB10 | hRNU1-1 | 1.46 | 1.22 | 1.33 | 1.34 | 0.12 | 0.005 |
| Scn2aB10_ | | | | | | | | | | |
| hRNU1-1 | | | | | | | | | | |
| 2C7_154_ | 154 | hRNU5A- | Scn2aB10 | hRNU6-1 | 1.45 | 1.30 | 1.26 | 1.34 | 0.10 | 0.005 |
| hRNU5A- | | 1 | | | | | | | | |
| 1_Scn2aB10_ | | | | | | | | | | |
| hRNU6-1 | | | | | | | | | | |
| 1C5_036_ | 036 | hRNU1-2 | Scn2aB10 | RNU7-1 | 1.36 | 1.30 | 1.40 | 1.35 | 0.05 | 0.003 |
| hRNU1- | | | | | | | | | | |
| 2_Scn2aB10_ | | | | | | | | | | |
| RNU7-1 | | | | | | | | | | |
| 517_mRnu12_ | 517 | mRnu12 | Scn2aB10 | hRNU11 | 1.62 | 1.1. | 1.32 | 1.36 | 0.25 | 0.003 |
| Scn2aB10_ | | | | | | | | | | |
| hRNU11 | | | | | | | | | | |
| 1D7_054_ | 054 | hRNU1-3 | Scn2aB10 | hRNU2-1 | 1.49 | 1.22 | 1.44 | 1.38 | 0.14 | 0.001 |
| hRNU1- | | | | | | | | | | |
| 3_Scn2aB10_ | | | | | | | | | | |
| hRNU2-1 | | | | | | | | | | |
| 513_mRnu12_ | 513 | mRnu12 | Scn2aB10 | hRNU4-1 | 1.34 | 1.26 | 1.56 | 1.39 | 0.16 | 0.001 |
| Scn2aB10_ | | | | | | | | | | |
| hRNU4-1 | | | | | | | | | | |
| 528_mRnu12_ | 528 | mRnu12 | Scn2aB10 | mRnul1 | 1.36 | 1.35 | 1.46 | 1.39 | 0.06 | 0.001 |
| Scn2aB10_ | | | | | | | | | | |
| mRnu11 | | | | | | | | | | |
| 511_mRnu12_ | 511 | mRnu12 | Scn2aB10 | hRNU2-1 | 1.57 | 1.36 | 1.28 | 1.40 | 0.15 | <0.001 |
| Scn2aB10_ | | | | | | | | | | |
| hRNU2-1 | | | | | | | | | | |
| 509_mRnu12_ | 509 | mRnu12 | Scn2aB10 | hRNU1-2 | 1.27 | 1.45 | 1.48 | 1.40 | 0.12 | <0.001 |
| Scn2aB10_ | | | | | | | | | | |
| hRNU1-2 | | | | | | | | | | |
| 510_mRnu12_ | 510 | mRnu12 | Scn2aB10 | hRNU1-3 | 1.47 | 1.40 | 1.41 | 1.43 | 0.04 | <0.001 |
| Scn2aB10_ | | | | | | | | | | |
| hRNU1-3 | | | | | | | | | | |
| 521_mRnu12_ | 521 | mRnu12 | Scn2aB10 | mRnu1b1 | 1.48 | 1.37 | 1.45 | 1.43 | 0.06 | <0.001 |
| Scn2aB10_ | | | | | | | | | | |
| mRnu1b1 | | | | | | | | | | |
| 2B12_195_mu1a1_ | 195 | mu1a1 | Scn2aD12 | U1 | 1.52 | 1.35 | 1.44 | 1.44 | 0.08 | <0.001 |
| Scn2aD12_ | | | | | | | | | | |
| U1 | | | | | | | | | | |
| 518_mRnu12_ | 518 | mRnu12 | Scn2aB10 | hRNU12 | 1.41 | 1.22 | 1.69 | 1.4 | 0.24 | <0.001 |
| Scn2aB10_ | | | | | | | | | | |
| hRNU12 | | | | | | | | | | |
| 506_mRnu11_ | 506 | mRnu11 | Scn2aB10 | mRnu12 | 1.31 | 1.59 | 1.68 | 1.53 | 0.20 | <0.001 |
| Scn2aB10_ | | | | | | | | | | |
| mRnu12 | | | | | | | | | | |
| 514_mRnu12_ | 514 | mRnu12 | Scn2aB10 | hRNU5A-1 | 2.02 | 1.12 | 1.45 | 1.53 | 0.46 | <0.001 |
| Scn2aB10_ | | | | | | | | | | |
| hRNU5A-1 | | | | | | | | | | |
| 527_mRnu12_ | 527 | mRnu12 | Scn2aB10 | mRnu7 | 1.17 | 1.80 | 1.65 | 1.54 | 0.33 | <0.001 |
| Scn2aB10_ | | | | | | | | | | |
| mRnu7 | | | | | | | | | | |
| 2C12_196_ | 196 | mu1a1 | Scn2aB10 | U1 | 1.54 | 1.4 | 1.68 | 1.54 | 0.13 | <0.001 |
| mu1a1_ | | | | | | | | | | |
| Scn2aB10_U1 | | | | | | | | | | |
| 507_mRnu11_ | 507 | mRnu11 | Scn2aB10 | mRn7sk | 1.38 | 1.57 | 1.74 | 1.57 | 0.18 | <0.001 |
| Scn2aB10_ | | | | | | | | | | |
| mRn7sk | | | | | | | | | | |
| 522_mRnu12_ | 522 | mRnu12 | Scn2aB10 | mRnu1b6 | 1.51 | 1.50 | 1.75 | 1.59 | 0.14 | <0.001 |
| Scn2aB10_ | | | | | | | | | | |
| mRnu1b6 | | | | | | | | | | |
| 1B7_052_ | 052 | hRNU1-3 | Scn2aB10 | hRNU1-2 | 1.77 | 1.45 | 1.66 | 1.62 | 0.17 | <0.001 |
| hRNU1- | | | | | | | | | | |
| 3_Scn2aB10_ | | | | | | | | | | |
| hRNU1-2 | | | | | | | | | | |
| 520_mRnu12_ | 520 | mRnu12 | Scn2aB10 | mRnu1a1 | 1.64 | 1.63 | 1.62 | 1.63 | 0.01 | <0.001 |
| Scn2aB10_ | | | | | | | | | | |
| mRnu1a1 | | | | | | | | | | |
| 1B11_084_ | 084 | hRNU2-1 | Scn2aB10 | hRNU12 | 1.89 | 1.42 | 1.75 | 1.69 | 0.24 | <0.001 |
| hRNU2- | | | | | | | | | | |
| 1_Scn2aB10_ | | | | | | | | | | |
| hRNU12 | | | | | | | | | | |
| 2C4_128_ | 128 | hRNU4-1 | Scn2aB10 | hU3 | 1.69 | 1.45 | 2.08 | 1.74 | 0.31 | <0.001 |
| hRNU4- | | | | | | | | | | |
| 1_Scn2aB10_ | | | | | | | | | | |
| hU3 | | | | | | | | | | |
| 505_mRnu11_ | 505 | mRnu11 | Scn2aB10 | mRnu11 | 1.75 | 1.63 | 1.93 | 1.77 | 0.15 | <0.001 |
| Scn2aB10_ | | | | | | | | | | |
| mRnu11 | | | | | | | | | | |
| 2E7 156_ | 156 | hRNU5A- | Scn2aB10 | hRNU11 | 1.97 | 1.60 | 2.00 | 1.86 | 0.22 | <0.001 |
| hRNU5A- | | 1 | | | | | | | | |
| 1_Scn2aB10_ | | | | | | | | | | |
| hRNU11 | | | | | | | | | | |
| 1D1_004_mu1a1_ | 004 | mu1a1 | Scn2aB10 | U1 | 1.99 | 1.73 | 1.86 | 1.86 | 0.13 | <0.001 |
| Scn2aB10_ | | | | | | | | | | |
| U1 | | | | | | | | | | |
| 3B7_253_ | 253 | hRNU12 | Scn2aB10 | hRNU12 | 1.92 | 1.64 | 2.06 | 1.87 | 0.22 | <0.001 |
| hRNU12_ | | | | | | | | | | |
| Scn2aB10hRNU12 | | | | | | | | | | |
| 2A1_100_mu1a1_ | 100 | mu1a1 | Scn2aB10 | U1 | 1.86 | 1.80 | 2.05 | 1.90 | 0.13 | <0.001 |
| Scn2aB10_U1 | | | | | | | | | | |
| 504_mRnu11_ | 504 | mRnu11 | Scn2aB10 | mRnu7 | 1.82 | 2.03 | 1.94 | 1.93 | 0.10 | <0.001 |
| Scn2aB10_ | | | | | | | | | | |
| mRnu7 | | | | | | | | | | |
| 2A7_152_hRN | 152 | hRNU5A- | Scn2aB10 | hRNU4-1 | 2.03 | 1.64 | 2.15 | 1.94 | 0.27 | <0.001 |
| U5A- | | 1 | | | | | | | | |
| 1_Scn2aB10_ | | | | | | | | | | |
| hRNU4-1 | | | | | | | | | | |
| 2B6_144_hRN | 144 | hRNU4-1 | Scn2aB10 | mRnu11 | 2.18 | 1.79 | 1.90 | 1.96 | 0.20 | <0.001 |
| U4- | | | | | | | | | | |
| 1_Scn2aB10_ | | | | | | | | | | |
| mRnu11 | | | | | | | | | | |
| 2F8_166_hRN | 166 | hRNU5A- | Scn2aB10 | mRnu7 | 1.79 | 1.90 | 2.24 | 1.98 | 0.23 | <0.001 |
| U5A- | | 1 | | | | | | | | |
| 1_Scn2aB10_ | | | | | | | | | | |
| mRnu7 | | | | | | | | | | |
| 1E7_055_hRN | 055 | hRNU1-3 | Scn2aB10 | hU3 | 2.01 | 2.10 | 2.05 | 2.06 | 0.04 | <0.001 |
| U1- | | | | | | | | | | |
| 3_Scn2aB10_ | | | | | | | | | | |
| hU3 | | | | | | | | | | |
| 1A12_091_ | 091 | hRNU2-1 | Scn2aB10 | mRnu5g | 2.18 | 1.89 | 2.11 | 2.06 | 0.15 | <0.001 |
| hRNU2- | | | | | | | | | | |
| 1_Scn2aB10_ | | | | | | | | | | |
| mRnu5g | | | | | | | | | | |
| 3B8_262_ | 262 | hRNU12 | Scn2aB10 | mRnu7 | 2.02 | 1.90 | 2.30 | 2.07 | 0.21 | <0.001 |
| hRNU12 | | | | | | | | | | |
| Scn2aB10_mRnu7 | | | | | | | | | | |
| 502_mRnu11_ | 502 | mRnu11 | Scn2aB10 | mRnu5g | 1.86 | 1.92 | 2.50 | 2.09 | 0.36 | <0.001 |
| Scn2aB10_ | | | | | | | | | | |
| mRnu5g | | | | | | | | | | |
| 1E6_047_hRN | 047 | hRNU1-2 | Scn2aB10 | mRnu7 | 2.06 | 1.98 | 2.42 | 2.16 | 0.24 | <0.001 |
| U1- | | | | | | | | | | |
| 2_Scn2aB10_ | | | | | | | | | | |
| mRnu7 | | | | | | | | | | |
| 3A6_244_hRNU12_ | 244 | hRNU12 | Scn2aB10 | hRNU1-2 | 2.69 | 2.47 | 2.35 | 2.50 | 0.17 | <0.001 |
| Scn2aB10_ | | | | | | | | | | |
| hRNU1-2 | | | | | | | | | | |

### Example 10: Example snRNA Sequences and Example Expression Construct Sequences

Tables 14A-14C include some example snRNA sequences. Some such RNA sequences may be useful in a composition or method herein. A DNA sequence encoding an snRNA sequence of any of Tables 14A-14C may have a similar sequence but have Ts instead of Us. Some such DNA sequences may be useful in a composition or method herein, such as in an expression construct or vector, or in a method of treatment that includes administering a composition comprising the expression construct or vector.

TABLE 14A
DNA Example U7 snRNA sequences targeting SCN1A mRNA-corresponding with
constructs in Table 2A

| Sequence | | SEQ |
|---|---|---|
| Name | snRNA Sequences (5′ to 3′) | ID NO: |
| Scramble-01 | AUGAUAGGGACUUAGGGUGAGCACCCCAUCCCCCACUACAAAUUUUUG | 923 |
| | GAGcaggUUUUcUgacUUcggUcggaaaaccccU | |
| N5-01 | AUGAUAGGGACUUAGGGUGAAACCACCGCUCCACCCCAUCAAUUUUUG | 924 |
| | GAGcaggUUUUcUgacUUcggUcggaaaaccccU | |
| N5-02 | AUGAUAGGGACUUAGGGUGACAAGUUGGAGCAAGAUUAUCAAUUUUUG | 925 |
| | GAGcaggUUUUcUgacUUcggUcggaaaaccccU | |
| N5-03 | AUGAUAGGGACUUAGGGUGACUAUAUAAAAUAGAAAUAUAAAUUUUU | 926 |
| | GGAGcaggUUUUcUgacUUcggUcggaaaaccccU | |
| N5-04 | AUGAUAGGGACUUAGGGUGAUAGUUUAUUAUUAGUUAGAAAAUUUUU | 927 |
| | GGAGcaggUUUUcUgacUUcggUcggaaaaccccU | |
| N5-05 | AUGAUAGGGACUUAGGGUGAGAGGAACCACCGCUCCACCCCAUCAAUU | 928 |
| | UUUGGAGcaggUUUUcUgacUUcggUcggaaaaccccU | |
| N5-06 | AUGAUAGGGACUUAGGGUGACAUCCAAGUUGGAGCAAGAUUAUCAAUU | 929 |
| | UUUGGAGcaggUUUUcUgacUUcggUcggaaaaccccU | |
| N5-07 | AUGAUAGGGACUUAGGGUGAUAUCCUAUAUAAAAUAGAAAUAUAAAU | 930 |
| | UUUUGGAGcaggUUUUcUgacUUcggUcggaaaaccccU | |
| N5-08 | AUGAUAGGGACUUAGGGUGAUAUAUAGUUUAUUAUUAGUUAGAAAAU | 931 |
| | UUUUGGAGcaggUUUUcUgacUUcggUcggaaaaccccU | |
| N5-09 | AUGAUAGGGACUUAGGGUGAGGGAGGAACCACCGCUCCACCCCAUCAA | 932 |
| | UUUUUGGAGcaggUUUUcUgacUUcggUcggaaaaccccU | |
| N5-10 | AUGAUAGGGACUUAGGGUGACCCAUCCAAGUUGGAGCAAGAUUAUCAA | 933 |
| | UUUUUGGAGcaggUUUUcUgacUUcggUcggaaaaccccU | |
| N5-11 | AUGAUAGGGACUUAGGGUGAAUUAUCCUAUAUAAAAUAGAAAUAUAA | 934 |
| | AUUUUUGGAGcaggUUUUcUgacUUcggUcggaaaaccccU | |
| N5-12 | AUGAUAGGGACUUAGGGUGAAAUAUAUAGUUUAUUAUUAGUUAGAAA | 935 |
| | AUUUUUGGAGcaggUUUUcUgacUUcggUcggaaaaccccU | |
| N5-13 | AUGAUAGGGACUUAGGGUGAUGAGGGGAGGAACCACCGCUCCACCCCA | 936 |
| | UCAAUUUUUGGAGcaggUUUUcUgacUUcggUcggaaaaccccU | |
| N5-14 | AUGAUAGGGACUUAGGGUGACCACCCCAUCCAAGUUGGAGCAAGAUUA | 937 |
| | UCAAUUUUUGGAGcaggUUUUcUgacUUcggUcggaaaaccccU | |
| N5-15 | AUGAUAGGGACUUAGGGUGACAAGAUUAUCCUAUAUAAAAUAGAAAUA | 938 |
| | UAAAUUUUUGGAGcaggUUUUcUgacUUcggUcggaaaaccccU | |
| N5-16 | AUGAUAGGGACUUAGGGUGAUAGAAAUAUAUAGUUUAUUAUUAGUUA | 939 |
| | GAAAAUUUUUGGAGcaggUUUUcUgacUUcggUcggaaaaccccU | |
| N3-01 | AUGAUAGGGACUUAGGGUGAAGUGCAAGGAUUAAAGGUAGAAUUUUU | 940 |
| | GGAGcaggUUUUcUgacUUcggUcggaaaaccccU | |
| N3-02 | AUGAUAGGGACUUAGGGUGACAAAAGGGGUAAUACAGUACAAUUUUUG | 941 |
| | GAGcaggUUUUcUgacUUcggUcggaaaaccccU | |
| N3-03 | AUGAUAGGGACUUAGGGUGACCAUAAUAAAGGGCUGAGGGAAUUUUUG | 942 |
| | GAGcaggUUUUcUgacUUcggUcggaaaaccccU | |
| N3-04 | AUGAUAGGGACUUAGGGUGAGAGGAACCACCGCUCCACCCAAUUUUUG | 943 |
| | GAGcaggUUUUcUgacUUcggUcggaaaaccccU | |
| N3-05 | AUGAUAGGGACUUAGGGUGAUCACAGUGCAAGGAUUAAAGGUAGAAUU | 944 |
| | UUUGGAGcaggUUUUcUgacUUcggUcggaaaaccccU | |
| N3-06 | AUGAUAGGGACUUAGGGUGAGUAGCAAAAGGGGUAAUACAGUACAAUU | 945 |
| | UUUGGAGcaggUUUUcUgacUUcggUcggaaaaccccU | |
| N3-07 | AUGAUAGGGACUUAGGGUGAGUACCCAUAAUAAAGGGCUGAGGGAAUU | 946 |
| | UUUGGAGcaggUUUUcUgacUUcggUcggaaaaccccU | |
| N3-08 | AUGAUAGGGACUUAGGGUGAAGGGGAGGAACCACCGCUCCACCCAAUU | 947 |
| | UUUGGAGcaggUUUUcUgacUUcggUcggaaaaccccU | |
| N3-09 | AUGAUAGGGACUUAGGGUGAAGUCACAGUGCAAGGAUUAAAGGUAGAA | 948 |
| | UUUUUGGAGcaggUUUUcUgacUUcggUcggaaaaccccU | |
| N3-10 | AUGAUAGGGACUUAGGGUGAAGGUAGCAAAAGGGGUAAUACAGUACAA | 949 |
| | UUUUUGGAGcaggUUUUcUgacUUcggUcggaaaaccccU | |
| N3-11 | AUGAUAGGGACUUAGGGUGACAGUACCCAUAAUAAAGGGCUGAGGGAA | 950 |
| | UUUUUGGAGcaggUUUUcUgacUUcggUcggaaaaccccU | |
| N3-12 | AUGAUAGGGACUUAGGGUGAUGAGGGGAGGAACCACCGCUCCACCCAA | 951 |
| | UUUUUGGAGcaggUUUUcUgacUUcggUcggaaaaccccU | |
| N3-13 | AUGAUAGGGACUUAGGGUGACAUAAGUCACAGUGCAAGGAUUAAAGGU | 952 |
| | AGAAUUUUUGGAGcaggUUUUcUgacUUcggUcggaaaaccccU | |
| N3-14 | AUGAUAGGGACUUAGGGUGAUUAAAGGUAGCAAAAGGGGUAAUACAG | 953 |
| | UACAAUUUUUGGAGcaggUUUUcUgacUUcggUcggaaaaccccU | |
| N3-15 | AUGAUAGGGACUUAGGGUGAAAUACAGUACCCAUAAUAAAGGGCUGAG | 954 |
| | GGAAUUUUUGGAGcaggUUUUcUgacUUcggUcggaaaaccccU | |
| N3-16 | AUGAUAGGGACUUAGGGUGAGGGCUGAGGGGAGGAACCACCGCUCCAC | 955 |
| | CCAAUUUUUGGAGcaggUUUUcUgacUUcggUcggaaaaccccU | |
| N5-ESSalt1- | UUUGUUCCGUGGGUGGUUUAAACCACCGCUCCACCCCAUCAAUUUUUG | 956 |
| 01 | GAGcaggUUUUcUgacUUcggUcggaaaaccccU | |
| N5-ESSalt1- | UUUGUUCCGUGGGUGGUUUACAAGUUGGAGCAAGAUUAUCAAUUUUUG | 957 |
| 02 | GAGcaggUUUUcUgacUUcggUcggaaaaccccU | |
| N5-ESSalt1- | UUUGUUCCGUGGGUGGUUUACUAUAUAAAAUAGAAAUAUAAAUUUUU | 958 |
| 03 | GGAGcaggUUUUcUgacUUcggUcggaaaaccccU | |
| N5-ESSalt1- | UUUGUUCCGUGGGUGGUUUAUAGUUUAUUAUUAGUUAGAAAAUUUUU | 959 |
| 04 | GGAGcaggUUUUcUgacUUcggUcggaaaaccccU | |
| N5-ESSalt1- | UUUGUUCCGUGGGUGGUUUAGAGGAACCACCGCUCCACCCCAUCAAUU | 960 |
| 05 | UUUGGAGcaggUUUUcUgacUUcggUcggaaaaccccU | |
| N5-ESSalt1- | UUUGUUCCGUGGGUGGUUUACAUCCAAGUUGGAGCAAGAUUAUCAAUU | 961 |
| 06 | UUUGGAGcaggUUUUcUgacUUcggUcggaaaaccccU | |
| N5-ESSalt1- | UUUGUUCCGUGGGUGGUUUAUAUCCUAUAUAAAAUAGAAAUAUAAAUU | 962 |
| 07 | UUUGGAGcaggUUUUcUgacUUcggUcggaaaaccccU | |
| N5-ESSalt1- | UUUGUUCCGUGGGUGGUUUAUAUAUAGUUUAUUAUUAGUUAGAAAAU | 963 |
| 08 | UUUUGGAGcaggUUUUcUgacUUcggUcggaaaaccccU | |
| N5-ESSalt1- | UUUGUUCCGUGGGUGGUUUAGGGAGGAACCACCGCUCCACCCCAUCAA | 964 |
| 09 | UUUUUGGAGcaggUUUUcUgacUUcggUcggaaaaccccU | |
| Scramble-02 | UUUGUUCCGUGGGUGGUUUAGGAGAAUCACCUUAUAGGAUAAUUUUUG | 965 |
| | GAGcaggUUUUcUgacUUcggUcggaaaaccccU | |
| N5-ESSalt1- | UUUGUUCCGUGGGUGGUUUACCCAUCCAAGUUGGAGCAAGAUUAUCAA | 966 |
| 10 | UUUUUGGAGcaggUUUUcUgacUUcggUcggaaaaccccU | |
| N5-ESSalt1- | UUUGUUCCGUGGGUGGUUUAAUUAUCCUAUAUAAAAUAGAAAUAUAAA | 967 |
| 11 | UUUUUGGAGcaggUUUUcUgacUUcggUcggaaaaccccU | |
| N5-ESSalt1- | UUUGUUCCGUGGGUGGUUUAAAUAUAUAGUUUAUUAUUAGUUAGAAA | 968 |
| 12 | AUUUUUGGAGcaggUUUUcUgacUUcggUcggaaaaccccU | |
| N5-ESSalt1- | UUUGUUCCGUGGGUGGUUUAUGAGGGGAGGAACCACCGCUCCACCCCA | 969 |
| 13 | UCAAUUUUUGGAGcaggUUUUcUgacUUcggUcggaaaaccccU | |
| N5-ESSalt1- | UUUGUUCCGUGGGUGGUUUACCACCCCAUCCAAGUUGGAGCAAGAUUA | 970 |
| 14 | UCAAUUUUUGGAGcaggUUUUcUgacUUcggUcggaaaaccccU | |
| N5-ESSalt1- | UUUGUUCCGUGGGUGGUUUACAAGAUUAUCCUAUAUAAAAUAGAAAUA | 971 |
| 15 | UAAAUUUUUGGAGcaggUUUUcUgacUUcggUcggaaaaccccU | |
| N5-ESSalt1- | UUUGUUCCGUGGGUGGUUUAUAGAAAUAUAUAGUUUAUUAUUAGUUA | 972 |
| 16 | GAAAAUUUUUGGAGcaggUUUUcUgacUUcggUcggaaaaccccU | |
| N3-ESSalt1- | UUUGUUCCGUGGGUGGUUUAAGUGCAAGGAUUAAAGGUAGAAUUUUU | 973 |
| 01 | GGAGcaggUUUUcUgacUUcggUcggaaaaccccU | |
| N3-ESSalt1- | UUUGUUCCGUGGGUGGUUUACAAAAGGGGUAAUACAGUACAAUUUUUG | 974 |
| 02 | GAGcaggUUUUcUgacUUcggUcggaaaaccccU | |
| N3-ESSalt1- | UUUGUUCCGUGGGUGGUUUACCAUAAUAAAGGGCUGAGGGAAUUUUUG | 975 |
| 03 | GAGcaggUUUUcUgacUUcggUcggaaaaccccU | |
| N3-ESSalt1- | UUUGUUCCGUGGGUGGUUUAGAGGAACCACCGCUCCACCCAAUUUUUG | 976 |
| 04 | GAGcaggUUUUcUgacUUcggUcggaaaaccccU | |
| Scramble-03 | UUUGUUCCGUGGGUGGUUUAGAAUAAUAACAUAAUAAUAUAAUUUUU | 977 |
| | GGAGcaggUUUUcUgacUUcggUcggaaaaccccU | |
| N3-ESSalt1- | UUUGUUCCGUGGGUGGUUUAUCACAGUGCAAGGAUUAAAGGUAGAAUU | 978 |
| 05 | UUUGGAGcaggUUUUcUgacUUcggUcggaaaaccccU | |
| N3-ESSalt1- | UUUGUUCCGUGGGUGGUUUAGUAGCAAAAGGGGUAAUACAGUACAAUU | 979 |
| 06 | UUUGGAGcaggUUUUcUgacUUcggUcggaaaaccccU | |
| N3-ESSalt1- | UUUGUUCCGUGGGUGGUUUAGUACCCAUAAUAAAGGGCUGAGGGAAUU | 980 |
| 07 | UUUGGAGcaggUUUUcUgacUUcggUcggaaaaccccU | |
| N3-ESSalt1- | UUUGUUCCGUGGGUGGUUUAAGGGGAGGAACCACCGCUCCACCCAAUU | 981 |
| 08 | UUUGGAGcaggUUUUcUgacUUcggUcggaaaaccccU | |
| N3-ESSalt1- | UUUGUUCCGUGGGUGGUUUAAGUCACAGUGCAAGGAUUAAAGGUAGAA | 982 |
| 09 | UUUUUGGAGcaggUUUUcUgacUUcggUcggaaaaccccU | |
| N3-ESSalt1- | UUUGUUCCGUGGGUGGUUUAAGGUAGCAAAAGGGGUAAUACAGUACAA | 983 |
| 10 | UUUUUGGAGcaggUUUUcUgacUUcggUcggaaaaccccU | |
| N3-ESSalt1- | UUUGUUCCGUGGGUGGUUUACAGUACCCAUAAUAAAGGGCUGAGGGAA | 984 |
| 11 | UUUUUGGAGcaggUUUUcUgacUUcggUcggaaaaccccU | |
| N3-ESSalt1- | UUUGUUCCGUGGGUGGUUUAUGAGGGGAGGAACCACCGCUCCACCCAA | 985 |
| 12 | UUUUUGGAGcaggUUUUcUgacUUcggUcggaaaaccccU | |
| N3-ESSalt1- | UUUGUUCCGUGGGUGGUUUACAUAAGUCACAGUGCAAGGAUUAAAGGU | 986 |
| 13 | AGAAUUUUUGGAGcaggUUUUcUgacUUcggUcggaaaaccccU | |
| N3-ESSalt1- | UUUGUUCCGUGGGUGGUUUAUUAAAGGUAGCAAAAGGGGUAAUACAGU | 987 |
| 14 | ACAAUUUUUGGAGcaggUUUUcUgacUUcggUcggaaaaccccU | |
| N3-ESSalt1- | UUUGUUCCGUGGGUGGUUUAAAUACAGUACCCAUAAUAAAGGGCUGAG | 988 |
| 15 | GGAAUUUUUGGAGcaggUUUUcUgacUUcggUcggaaaaccccU | |
| N3-ESSalt1- | UUUGUUCCGUGGGUGGUUUAGGGCUGAGGGGAGGAACCACCGCUCCAC | 989 |
| 16 | CCAAUUUUUGGAGcaggUUUUcUgacUUcggUcggaaaaccccU | |
| N5-ESSalt2- | UGGGGGGAGGUAGGUAGGUAAACCACCGCUCCACCCCAUCAAUUUUUG | 990 |
| 01 | GAGcaggUUUUcUgacUUcggUcggaaaaccccU | |
| N5-ESSalt2- | UGGGGGGAGGUAGGUAGGUACAAGUUGGAGCAAGAUUAUCAAUUUUU | 991 |
| 02 | GGAGcaggUUUUcUgacUUcggUcggaaaaccccU | |
| N5-ESSalt2- | UGGGGGGAGGUAGGUAGGUACUAUAUAAAAUAGAAAUAUAAAUUUUU | 992 |
| 03 | GGAGcaggUUUUcUgacUUcggUcggaaaaccccU | |
| N5-ESSalt2- | UGGGGGGAGGUAGGUAGGUAUAGUUUAUUAUUAGUUAGAAAAUUUUU | 993 |
| 04 | GGAGcaggUUUUcUgacUUcggUcggaaaaccccU | |
| N5-ESSalt2- | UGGGGGGAGGUAGGUAGGUAGAGGAACCACCGCUCCACCCCAUCAAUU | 994 |
| 05 | UUUGGAGcaggUUUUcUgacUUcggUcggaaaaccccU | |
| N5-ESSalt2- | UGGGGGGAGGUAGGUAGGUACAUCCAAGUUGGAGCAAGAUUAUCAAUU | 995 |
| 06 | UUUGGAGcaggUUUUcUgacUUcggUcggaaaaccccU | |
| N5-ESSalt2- | UGGGGGGAGGUAGGUAGGUAUAUCCUAUAUAAAAUAGAAAUAUAAAU | 996 |
| 07 | UUUUGGAGcaggUUUUcUgacUUcggUcggaaaaccccU | |
| N5-ESSalt2- | UGGGGGGAGGUAGGUAGGUAUAUAUAGUUUAUUAUUAGUUAGAAAAU | 997 |
| 08 | UUUUGGAGcaggUUUUcUgacUUcggUcggaaaaccccU | |
| N5-ESSalt2- | UGGGGGGAGGUAGGUAGGUAGGGAGGAACCACCGCUCCACCCCAUCAA | 998 |
| 09 | UUUUUGGAGcaggUUUUcUgacUUcggUcggaaaaccccU | |
| N5-ESSalt2- | UGGGGGGAGGUAGGUAGGUACCCAUCCAAGUUGGAGCAAGAUUAUCAA | 999 |
| 10 | UUUUUGGAGcaggUUUUcUgacUUcggUcggaaaaccccU | |
| N5-ESSalt2- | UGGGGGGAGGUAGGUAGGUAAUUAUCCUAUAUAAAAUAGAAAUAUAA | 1000 |
| 11 | AUUUUUGGAGcaggUUUUcUgacUUcggUcggaaaaccccU | |
| N5-ESSalt2- | UGGGGGGAGGUAGGUAGGUAUGAGGGGAGGAACCACCGCUCCACCCCA | 1001 |
| 13 | UCAAUUUUUGGAGcaggUUUUcUgacUUcggUcggaaaaccccU | |
| N5-ESSalt2- | UGGGGGGAGGUAGGUAGGUACCACCCCAUCCAAGUUGGAGCAAGAUUA | 1002 |
| 14 | UCAAUUUUUGGAGcaggUUUUcUgacUUcggUcggaaaaccccU | |
| N5-ESSalt2- | UGGGGGGAGGUAGGUAGGUACAAGAUUAUCCUAUAUAAAAUAGAAAU | 1003 |
| 15 | AUAAAUUUUUGGAGcaggUUUUcUgacUUcggUcggaaaaccccU | |
| N3-ESSalt2- | UGGGGGGAGGUAGGUAGGUAAGUGCAAGGAUUAAAGGUAGAAUUUUU | 1004 |
| 01 | GGAGcaggUUUUcUgacUUcggUcggaaaaccccU | |
| N3-ESSalt2- | UGGGGGGAGGUAGGUAGGUACAAAAGGGGUAAUACAGUACAAUUUUU | 1005 |
| 02 | GGAGcaggUUUUcUgacUUcggUcggaaaaccccU | |
| N3-ESSalt2- | UGGGGGGAGGUAGGUAGGUACCAUAAUAAAGGGCUGAGGGAAUUUUU | 1006 |
| 03 | GGAGcaggUUUUcUgacUUcggUcggaaaaccccU | |
| N3-ESSalt2- | UGGGGGGAGGUAGGUAGGUAGAGGAACCACCGCUCCACCCAAUUUUUG | 1007 |
| 04 | GAGcaggUUUUcUgacUUcggUcggaaaaccccU | |
| N3-ESSalt2- | UGGGGGGAGGUAGGUAGGUAUCACAGUGCAAGGAUUAAAGGUAGAAU | 1008 |
| 05 | UUUUGGAGcaggUUUUcUgacUUcggUcggaaaaccccU | |
| N3-ESSalt2- | UGGGGGGAGGUAGGUAGGUAGUAGCAAAAGGGGUAAUACAGUACAAU | 1009 |
| 06 | UUUUGGAGcaggUUUUcUgacUUcggUcggaaaaccccU | |
| N3-ESSalt2- | UGGGGGGAGGUAGGUAGGUAGUACCCAUAAUAAAGGGCUGAGGGAAUU | 1010 |
| 07 | UUUGGAGcaggUUUUcUgacUUcggUcggaaaaccccU | |
| N3-ESSalt2- | UGGGGGGAGGUAGGUAGGUAAGGGGAGGAACCACCGCUCCACCCAAUU | 1011 |
| 08 | UUUGGAGcaggUUUUcUgacUUcggUcggaaaaccccU | |
| N3-ESSalt2- | UGGGGGGAGGUAGGUAGGUAAGUCACAGUGCAAGGAUUAAAGGUAGA | 1012 |
| 09 | AUUUUUGGAGcaggUUUUcUgacUUcggUcggaaaaccccU | |
| N3-ESSalt2- | UGGGGGGAGGUAGGUAGGUAAGGUAGCAAAAGGGGUAAUACAGUACA | 1013 |
| 10 | AUUUUUGGAGcaggUUUUcUgacUUcggUcggaaaaccccU | |
| N3-ESSalt2- | UGGGGGGAGGUAGGUAGGUACAGUACCCAUAAUAAAGGGCUGAGGGAA | 1014 |
| 11 | UUUUUGGAGcaggUUUUcUgacUUcggUcggaaaaccccU | |
| N3-ESSalt2- | UGGGGGGAGGUAGGUAGGUACAUAAGUCACAGUGCAAGGAUUAAAGGU | 1015 |
| 13 | AGAAUUUUUGGAGcaggUUUUcUgacUUcggUcggaaaaccccU | |
| N3-ESSalt2- | UGGGGGGAGGUAGGUAGGUAUUAAAGGUAGCAAAAGGGGUAAUACAG | 1016 |
| 14 | UACAAUUUUUGGAGcaggUUUUcUgacUUcggUcggaaaaccccU | |
| N3-ESSalt2- | UGGGGGGAGGUAGGUAGGUAAAUACAGUACCCAUAAUAAAGGGCUGAG | 1017 |
| 15 | GGAAUUUUUGGAGcaggUUUUcUgacUUcggUcggaaaaccccU | |
| Scramble-04 | UGGGGGGAGGUAGGUAGGUAGAAAUUUAUGUAUUGAUUAUAAUUUUU | 1018 |
| | GGAGcaggUUUUcUgacUUcggUcggaaaaccccU | |

TABLE 14B
DNA Example U7 snRNA sequences targeting SCN1A mRNA-corresponding with
constructs in Table 2B

| Sequence | | SEQ |
|---|---|---|
| Name | snRNA Sequences (5′ to 3′) | ID NO: |
| N5-01 | AUGAUAGGGACUUAGGGUGAAACCAGCGCUCCACCCCAUCAAUUUUUG | 1019 |
| | GAGcaggUUUUcUgacUUcggUcggaaaaccccU | |
| N5-02 | AUGAUAGGGACUUAGGGUGACAAGUUGGAGCAAGAUUAUCAAUUUUUG | 1020 |
| | GAGcaggUUUUcUgacUUcggUcggaaaaccccU | |
| N5-03 | AUGAUAGGGACUUAGGGUGACUAUACAAAAUAGAAAUAUAAAUUUUU | 1021 |
| | GGAGcaggUUUUcUgacUUcggUcggaaaaccccU | |
| N5-04 | AUGAUAGGGACUUAGGGUGAUAGUUUGUUAUUAGUUAGAAAAUUUUU | 1022 |
| | GGAGcaggUUUUcUgacUUcggUcggaaaaccccU | |
| N5-05 | AUGAUAGGGACUUAGGGUGAGAGGAACCAGCGCUCCACCCCAUCAAUU | 1023 |
| | UUUGGAGcaggUUUUcUgacUUcggUcggaaaaccccU | |
| N5-06 | AUGAUAGGGACUUAGGGUGACAUCCAAGUUGGAGCAAGAUUAUCAAUU | 1024 |
| | UUUGGAGcaggUUUUcUgacUUcggUcggaaaaccccU | |
| N5-07 | AUGAUAGGGACUUAGGGUGAUAUCCUAUACAAAAUAGAAAUAUAAAUU | 1025 |
| | UUUGGAGcaggUUUUcUgacUUcggUcggaaaaccccU | |
| N5-08 | AUGAUAGGGACUUAGGGUGAUAUAUAGUUUGUUAUUAGUUAGAAAAU | 1026 |
| | UUUUGGAGcaggUUUUcUgacUUcggUcggaaaaccccU | |
| N5-09 | AUGAUAGGGACUUAGGGUGAGGGAGGAACCAGCGCUCCACCCCAUCAA | 1027 |
| | UUUUUGGAGcaggUUUUcUgacUUcggUcggaaaaccccU | |
| N5-10 | AUGAUAGGGACUUAGGGUGACCCAUCCAAGUUGGAGCAAGAUUAUCAA | 1028 |
| | UUUUUGGAGcaggUUUUcUgacUUcggUcggaaaaccccU | |
| N5-11 | AUGAUAGGGACUUAGGGUGAAUUAUCCUAUACAAAAUAGAAAUAUAAA | 1029 |
| | UUUUUGGAGcaggUUUUcUgacUUcggUcggaaaaccccU | |
| N5-12 | AUGAUAGGGACUUAGGGUGAAAUAUAUAGUUUGUUAUUAGUUAGAAA | 1030 |
| | AUUUUUGGAGcaggUUUUcUgacUUcggUcggaaaaccccU | |
| N5-13 | AUGAUAGGGACUUAGGGUGAUCAGGGGAGGAACCAGCGCUCCACCCCA | 1031 |
| | UCAAUUUUUGGAGcaggUUUUcUgacUUcggUcggaaaaccccU | |
| N5-14 | AUGAUAGGGACUUAGGGUGACCACCCCAUCCAAGUUGGAGCAAGAUUA | 1032 |
| | UCAAUUUUUGGAGcaggUUUUcUgacUUcggUcggaaaaccccU | |
| N5-15 | AUGAUAGGGACUUAGGGUGACAAGAUUAUCCUAUACAAAAUAGAAAUA | 1033 |
| | UAAAUUUUUGGAGcaggUUUUcUgacUUcggUcggaaaaccccU | |
| N5-16 | AUGAUAGGGACUUAGGGUGAUAGAAAUAUAUAGUUUGUUAUUAGUUA | 1034 |
| | GAAAAUUUUUGGAGcaggUUUUcUgacUUcggUcggaaaaccccU | |
| N3-01 | AUGAUAGGGACUUAGGGUGAAGUGCAAGGAUUAAAGGUAGAAUUUUU | 1035 |
| | GGAGcaggUUUUcUgacUUcggUcggaaaaccccU | |
| N3-02 | AUGAUAGGGACUUAGGGUGACAAAAGGGGUAAUACAGUACAAUUUUUG | 1036 |
| | GAGcaggUUUUcUgacUUcggUcggaaaaccccU | |
| N3-03 | AUGAUAGGGACUUAGGGUGACCAUAAUAAAGGGCUCAGGGAAUUUUUG | 1037 |
| | GAGcaggUUUUcUgacUUcggUcggaaaaccccU | |
| N3-04 | AUGAUAGGGACUUAGGGUGAGAGGAACCAGCGCUCCACCCAAUUUUUG | 1038 |
| | GAGcaggUUUUcUgacUUcggUcggaaaaccccU | |
| N3-05 | AUGAUAGGGACUUAGGGUGAUCACAGUGCAAGGAUUAAAGGUAGAAUU | 1039 |
| | UUUGGAGcaggUUUUcUgacUUcggUcggaaaaccccU | |
| N3-06 | AUGAUAGGGACUUAGGGUGAGUAGCAAAAGGGGUAAUACAGUACAAUU | 1040 |
| | UUUGGAGcaggUUUUcUgacUUcggUcggaaaaccccU | |
| N3-07 | AUGAUAGGGACUUAGGGUGAGUACCCAUAAUAAAGGGCUCAGGGAAUU | 1041 |
| | UUUGGAGcaggUUUUcUgacUUcggUcggaaaaccccU | |
| N3-08 | AUGAUAGGGACUUAGGGUGAAGGGGAGGAACCAGCGCUCCACCCAAUU | 1042 |
| | UUUGGAGcaggUUUUcUgacUUcggUcggaaaaccccU | |
| N3-09 | AUGAUAGGGACUUAGGGUGAAGUCACAGUGCAAGGAUUAAAGGUAGAA | 1043 |
| | UUUUUGGAGcaggUUUUcUgacUUcggUcggaaaaccccU | |
| N3-10 | AUGAUAGGGACUUAGGGUGAAGGUAGCAAAAGGGGUAAUACAGUACAA | 1044 |
| | UUUUUGGAGcaggUUUUcUgacUUcggUcggaaaaccccU | |
| N3-11 | AUGAUAGGGACUUAGGGUGACAGUACCCAUAAUAAAGGGCUCAGGGAA | 1045 |
| | UUUUUGGAGcaggUUUUcUgacUUcggUcggaaaaccccU | |
| N3-12 | AUGAUAGGGACUUAGGGUGAUCAGGGGAGGAACCAGCGCUCCACCCAA | 1046 |
| | UUUUUGGAGcaggUUUUcUgacUUcggUcggaaaaccccU | |
| N3-13 | AUGAUAGGGACUUAGGGUGACAUAAGUCACAGUGCAAGGAUUAAAGGU | 1047 |
| | AGAAUUUUUGGAGcaggUUUUcUgacUUcggUcggaaaaccccU | |
| N3-14 | AUGAUAGGGACUUAGGGUGAUUAAAGGUAGCAAAAGGGGUAAUACAG | 1048 |
| | UACAAUUUUUGGAGcaggUUUUcUgacUUcggUcggaaaaccccU | |
| N3-15 | AUGAUAGGGACUUAGGGUGAAAUACAGUACCCAUAAUAAAGGGCUCAG | 1049 |
| | GGAAUUUUUGGAGcaggUUUUcUgacUUcggUcggaaaaccccU | |
| N3-16 | AUGAUAGGGACUUAGGGUGAGGGCUCAGGGGAGGAACCAGCGCUCCAC | 1050 |
| | CCAAUUUUUGGAGcaggUUUUcUgacUUcggUcggaaaaccccU | |
| N5-ESSalt1- | AUGAUAGGGACUUAGGGUGAAACCAGCGCUCCACCCCAUCAAUUUUUG | 1051 |
| 01 | GAGcaggUUUUcUgacUUcggUcggaaaaccccU | |
| N5-ESSalt1- | UUUGUUCCGUGGGUGGUUUACAAGUUGGAGCAAGAUUAUCAAUUUUUG | 1052 |
| 02 | GAGcaggUUUUcUgacUUcggUcggaaaaccccU | |
| N5-ESSalt1- | UUUGUUCCGUGGGUGGUUUACUAUACAAAAUAGAAAUAUAAAUUUUUG | 1053 |
| 03 | GAGcaggUUUUcUgacUUcggUcggaaaaccccU | |
| N5-ESSalt1- | UUUGUUCCGUGGGUGGUUUAUAGUUUGUUAUUAGUUAGAAAAUUUUU | 1054 |
| 04 | GGAGcaggUUUUcUgacUUcggUcggaaaaccccU | |
| N5-ESSalt1- | UUUGUUCCGUGGGUGGUUUAGAGGAACCAGCGCUCCACCCCAUCAAUU | 1055 |
| 05 | UUUGGAGcaggUUUUcUgacUUcggUcggaaaaccccU | |
| N5-ESSalt1- | UUUGUUCCGUGGGUGGUUUACAUCCAAGUUGGAGCAAGAUUAUCAAUU | 1056 |
| 06 | UUUGGAGcaggUUUUcUgacUUcggUcggaaaaccccU | |
| N5-ESSalt1- | UUUGUUCCGUGGGUGGUUUAUAUCCUAUACAAAAUAGAAAUAUAAAUU | 1057 |
| 07 | UUUGGAGcaggUUUUcUgacUUcggUcggaaaaccccU | |
| N5-ESSalt1- | UUUGUUCCGUGGGUGGUUUAUAUAUAGUUUGUUAUUAGUUAGAAAAU | 1058 |
| 08 | UUUUGGAGcaggUUUUcUgacUUcggUcggaaaaccccU | |
| N5-ESSalt1- | UUUGUUCCGUGGGUGGUUUAGGGAGGAACCAGCGCUCCACCCCAUCAA | 1059 |
| 09 | UUUUUGGAGcaggUUUUcUgacUUcggUcggaaaaccccU | |
| N5-ESSalt1- | UUUGUUCCGUGGGUGGUUUACCCAUCCAAGUUGGAGCAAGAUUAUCAA | 1060 |
| 10 | UUUUUGGAGcaggUUUUcUgacUUcggUcggaaaaccccU | |
| N5-ESSalt1- | UUUGUUCCGUGGGUGGUUUAAUUAUCCUAUACAAAAUAGAAAUAUAAA | 1061 |
| 11 | UUUUUGGAGcaggUUUUcUgacUUcggUcggaaaaccccU | |
| N5-ESSalt1- | UUUGUUCCGUGGGUGGUUUAAAUAUAUAGUUUGUUAUUAGUUAGAAA | 1062 |
| 12 | AUUUUUGGAGcaggUUUUcUgacUUcggUcggaaaaccccU | |
| N5-ESSalt1- | UUUGUUCCGUGGGUGGUUUAUCAGGGGAGGAACCAGCGCUCCACCCCA | 1063 |
| 13 | UCAAUUUUUGGAGcaggUUUUcUgacUUcggUcggaaaaccccU | |
| N5-ESSalt1- | UUUGUUCCGUGGGUGGUUUACCACCCCAUCCAAGUUGGAGCAAGAUUA | 1064 |
| 14 | UCAAUUUUUGGAGcaggUUUUcUgacUUcggUcggaaaaccccU | |
| N5-ESSalt1- | UUUGUUCCGUGGGUGGUUUACAAGAUUAUCCUAUACAAAAUAGAAAUA | 1065 |
| 15 | UAAAUUUUUGGAGcaggUUUUcUgacUUcggUcggaaaaccccU | |
| N5-ESSalt1- | UUUGUUCCGUGGGUGGUUUAUAGAAAUAUAUAGUUUGUUAUUAGUUA | 1066 |
| 16 | GAAAAUUUUUGGAGcaggUUUUcUgacUUcggUcggaaaaccccU | |
| N3-ESSalt1- | UUUGUUCCGUGGGUGGUUUAAGUGCAAGGAUUAAAGGUAGAAUUUUU | 1067 |
| 01 | GGAGcaggUUUUcUgacUUcggUcggaaaaccccU | |
| N3-ESSalt1- | UUUGUUCCGUGGGUGGUUUACAAAAGGGGUAAUACAGUACAAUUUUUG | 1068 |
| 02 | GAGcaggUUUUcUgacUUcggUcggaaaaccccU | |
| N3-ESSalt1- | UUUGUUCCGUGGGUGGUUUACCAUAAUAAAGGGCUCAGGGAAUUUUUG | 1069 |
| 03 | GAGcaggUUUUcUgacUUcggUcggaaaaccccU | |
| N3-ESSalt1- | UUUGUUCCGUGGGUGGUUUAGAGGAACCAGCGCUCCACCCAAUUUUUG | 1070 |
| 04 | GAGcaggUUUUcUgacUUcggUcggaaaaccccU | |
| N3-ESSalt1- | UUUGUUCCGUGGGUGGUUUAUCACAGUGCAAGGAUUAAAGGUAGAAUU | 1071 |
| 05 | UUUGGAGcaggUUUUcUgacUUcggUcggaaaaccccU | |
| N3-ESSalt1- | UUUGUUCCGUGGGUGGUUUAGUAGCAAAAGGGGUAAUACAGUACAAUU | 1072 |
| 06 | UUUGGAGcaggUUUUcUgacUUcggUcggaaaaccccU | |
| N3-ESSalt1- | UUUGUUCCGUGGGUGGUUUAGUACCCAUAAUAAAGGGCUCAGGGAAUU | 1073 |
| 07 | UUUGGAGcaggUUUUcUgacUUcggUcggaaaaccccU | |
| N3-ESSalt1- | UUUGUUCCGUGGGUGGUUUAAGGGGAGGAACCAGCGCUCCACCCAAUU | 1074 |
| 08 | UUUGGAGcaggUUUUcUgacUUcggUcggaaaaccccU | |
| N3-ESSalt1- | UUUGUUCCGUGGGUGGUUUAAGUCACAGUGCAAGGAUUAAAGGUAGAA | 1075 |
| 09 | UUUUUGGAGcaggUUUUcUgacUUcggUcggaaaaccccU | |
| N3-ESSalt1- | UUUGUUCCGUGGGUGGUUUAAGGUAGCAAAAGGGGUAAUACAGUACAA | 1076 |
| 10 | UUUUUGGAGcaggUUUUcUgacUUcggUcggaaaaccccU | |
| N3-ESSalt1- | UUUGUUCCGUGGGUGGUUUACAGUACCCAUAAUAAAGGGCUCAGGGAA | 1077 |
| 11 | UUUUUGGAGcaggUUUUcUgacUUcggUcggaaaaccccU | |
| N3-ESSalt1- | UUUGUUCCGUGGGUGGUUUAUCAGGGGAGGAACCAGCGCUCCACCCAA | 1078 |
| 12 | UUUUUGGAGcaggUUUUcUgacUUcggUcggaaaaccccU | |
| N3-ESSalt1- | UUUGUUCCGUGGGUGGUUUACAUAAGUCACAGUGCAAGGAUUAAAGGU | 1079 |
| 13 | AGAAUUUUUGGAGcaggUUUUcUgacUUcggUcggaaaaccccU | |
| N3-ESSalt1- | UUUGUUCCGUGGGUGGUUUAUUAAAGGUAGCAAAAGGGGUAAUACAGU | 1080 |
| 14 | ACAAUUUUUGGAGcaggUUUUcUgacUUcggUcggaaaaccccU | |
| N3-ESSalt1- | UUUGUUCCGUGGGUGGUUUAAAUACAGUACCCAUAAUAAAGGGCUCAG | 1081 |
| 15 | GGAAUUUUUGGAGcaggUUUUcUgacUUcggUcggaaaaccccU | |
| N3-ESSalt1- | UUUGUUCCGUGGGUGGUUUAGGGCUCAGGGGAGGAACCAGCGCUCCAC | 1082 |
| 16 | CCAAUUUUUGGAGcaggUUUUcUgacUUcggUcggaaaaccccU | |
| N5-ESSalt2- | UUUGUUCCGUGGGUGGUUUAAACCAGCGCUCCACCCCAUCAAUUUUUG | 1083 |
| 01 | GAGcaggUUUUcUgacUUcggUcggaaaaccccU | |
| N5-ESSalt2- | UUUGUUCCGUGGGUGGUUUACAAGUUGGAGCAAGAUUAUCAAUUUUUG | 1084 |
| 02 | GAGcaggUUUUcUgacUUcggUcggaaaaccccU | |
| N5-ESSalt2- | UUUGUUCCGUGGGUGGUUUACUAUACAAAAUAGAAAUAUAAAUUUUUG | 1085 |
| 03 | GAGcaggUUUUcUgacUUcggUcggaaaaccccU | |
| N5-ESSalt2- | UGGGGGGAGGUAGGUAGGUAUAGUUUGUUAUUAGUUAGAAAAUUUUU | 1086 |
| 04 | GGAGcaggUUUUcUgacUUcggUcggaaaaccccU | |
| N5-ESSalt2- | UGGGGGGAGGUAGGUAGGUAGAGGAACCAGCGCUCCACCCCAUCAAUU | 1087 |
| 05 | UUUGGAGcaggUUUUcUgacUUcggUcggaaaaccccU | |
| N5-ESSalt2- | UGGGGGGAGGUAGGUAGGUACAUCCAAGUUGGAGCAAGAUUAUCAAUU | 1088 |
| 06 | UUUGGAGcaggUUUUcUgacUUcggUcggaaaaccccU | |
| N5-ESSalt2- | UGGGGGGAGGUAGGUAGGUAUAUCCUAUACAAAAUAGAAAUAUAAAU | 1089 |
| 07 | UUUUGGAGcaggUUUUcUgacUUcggUcggaaaaccccU | |
| N5-ESSalt2- | UGGGGGGAGGUAGGUAGGUAUAUAUAGUUUGUUAUUAGUUAGAAAAU | 1090 |
| 08 | UUUUGGAGcaggUUUUcUgacUUcggUcggaaaaccccU | |
| N5-ESSalt2- | UGGGGGGAGGUAGGUAGGUAGGGAGGAACCAGCGCUCCACCCCAUCAA | 1091 |
| 09 | UUUUUGGAGcaggUUUUcUgacUUcggUcggaaaaccccU | |
| N5-ESSalt2- | UGGGGGGAGGUAGGUAGGUACCCAUCCAAGUUGGAGCAAGAUUAUCAA | 1092 |
| 10 | UUUUUGGAGcaggUUUUcUgacUUcggUcggaaaaccccU | |
| N5-ESSalt2- | UGGGGGGAGGUAGGUAGGUAAUUAUCCUAUACAAAAUAGAAAUAUAA | 1093 |
| 11 | AUUUUUGGAGcaggUUUUcUgacUUcggUcggaaaaccccU | |
| N5-ESSalt2- | UGGGGGGAGGUAGGUAGGUAUCAGGGGAGGAACCAGCGCUCCACCCCA | 1094 |
| 13 | UCAAUUUUUGGAGcaggUUUUcUgacUUcggUcggaaaaccccU | |
| N5-ESSalt2- | UGGGGGGAGGUAGGUAGGUACCACCCCAUCCAAGUUGGAGCAAGAUUA | 1095 |
| 14 | UCAAUUUUUGGAGcaggUUUUcUgacUUcggUcggaaaaccccU | |
| N5-ESSalt2- | UGGGGGGAGGUAGGUAGGUACAAGAUUAUCCUAUACAAAAUAGAAAUA | 1096 |
| 15 | UAAAUUUUUGGAGcaggUUUUcUgacUUcggUcggaaaaccccU | |
| N3-ESSalt2- | UGGGGGGAGGUAGGUAGGUAAGUGCAAGGAUUAAAGGUAGAAUUUUU | 1097 |
| 01 | GGAGcaggUUUUcUgacUUcggUcggaaaaccccU | |
| N3-ESSalt2- | UGGGGGGAGGUAGGUAGGUACAAAAGGGGUAAUACAGUACAAUUUUU | 1098 |
| 02 | GGAGcaggUUUUcUgacUUcggUcggaaaaccccU | |
| N3-ESSalt2- | UGGGGGGAGGUAGGUAGGUACCAUAAUAAAGGGCUCAGGGAAUUUUUG | 1099 |
| 03 | GAGcaggUUUUcUgacUUcggUcggaaaaccccU | |
| N3-ESSalt2- | UGGGGGGAGGUAGGUAGGUAGAGGAACCAGCGCUCCACCCAAUUUUUG | 1100 |
| 04 | GAGcaggUUUUcUgacUUcggUcggaaaaccccU | |
| N3-ESSalt2- | UGGGGGGAGGUAGGUAGGUAUCACAGUGCAAGGAUUAAAGGUAGAAU | 1101 |
| 05 | UUUUGGAGcaggUUUUcUgacUUcggUcggaaaaccccU | |
| N3-ESSalt2- | UGGGGGGAGGUAGGUAGGUAGUAGCAAAAGGGGUAAUACAGUACAAU | 1102 |
| 06 | UUUUGGAGcaggUUUUcUgacUUcggUcggaaaaccccU | |
| N3-ESSalt2- | UGGGGGGAGGUAGGUAGGUAGUACCCAUAAUAAAGGGCUCAGGGAAUU | 1103 |
| 07 | UUUGGAGcaggUUUUcUgacUUcggUcggaaaaccccU | |
| N3-ESSalt2- | UGGGGGGAGGUAGGUAGGUAAGGGGAGGAACCAGCGCUCCACCCAAUU | 1104 |
| 08 | UUUGGAGcaggUUUUcUgacUUcggUcggaaaaccccU | |
| N3-ESSalt2- | UGGGGGGAGGUAGGUAGGUAAGUCACAGUGCAAGGAUUAAAGGUAGA | 1105 |
| 09 | AUUUUUGGAGcaggUUUUcUgacUUcggUcggaaaaccccU | |
| N3-ESSalt2- | UGGGGGGAGGUAGGUAGGUAAGGUAGCAAAAGGGGUAAUACAGUACA | 1106 |
| 10 | AUUUUUGGAGcaggUUUUcUgacUUcggUcggaaaaccccU | |
| N3-ESSalt2- | UGGGGGGAGGUAGGUAGGUACAGUACCCAUAAUAAAGGGCUCAGGGAA | 1107 |
| 11 | UUUUUGGAGcaggUUUUcUgacUUcggUcggaaaaccccU | |
| N3-ESSalt2- | UGGGGGGAGGUAGGUAGGUACAUAAGUCACAGUGCAAGGAUUAAAGGU | 1108 |
| 13 | AGAAUUUUUGGAGcaggUUUUcUgacUUcggUcggaaaaccccU | |
| N3-ESSalt2- | UGGGGGGAGGUAGGUAGGUAUUAAAGGUAGCAAAAGGGGUAAUACAG | 1109 |
| 14 | UACAAUUUUUGGAGcaggUUUUcUgacUUcggUcggaaaaccccU | |
| N3-ESSalt2- | UGGGGGGAGGUAGGUAGGUAAAUACAGUACCCAUAAUAAAGGGCUCAG | 1110 |
| 15 | GGAAUUUUUGGAGcaggUUUUcUgacUUcggUcggaaaaccccU | |

TABLE 14C
DNA Example U7 snRNA sequences targeting SCN1A mRNA-corresponding with
constructs in Table 3B

| Sequence | | SEQ ID |
|---|---|---|
| Name | snRNA Sequences (5′ to 3′) | NO: |
| 3c5c-24 | AUGAUAGGGACUUAGGGUGAGCACCCCAUCCCCCACUACAAAUUUU | 1111 |
| | UGGAGcaggUUUUcUgacUUcggUcggaaaaccccU | |
| 3c5c-28 | AUGAUAGGGACUUAGGGUGAGUAAUACAGUACCCAUAAUAAAGGAG | 1112 |
| | CAAGAUUAUCCUAUAUAAAAUAAAUUUUUGGAGcaggUUUUcUgacUUc | |
| | ggUcggaaaaccccU | |
| 3c5c-30 | AUGAUAGGGACUUAGGGUGAGGGUAAUACAGUACCCAUAAUAAAGG | 1113 |
| | GCGGAGCAAGAUUAUCCUAUAUAAAAUAGAAAUUUUUGGAGcaggUU | |
| | UUcUgacUUcggUcggaaaaccccU | |
| 3ex5ex-24 | AUGAUAGGGACUUAGGGUGAGGGGUAAUACAGUACCCAUAAUAAAG | 1114 |
| | GGCUUGGAGCAAGAUUAUCCUAUAUAAAAUAGAAAAUUUUUGGAGc | |
| | aggUUUUcUgacUUcggUcggaaaaccccU | |
| 3ex5ex-28 | AUGAUAGGGACUUAGGGUGAAGGGGUAAUACAGUACCCAUAAUAAG | 1115 |
| | AUUAUCCUAUAUAAAAUAGAAAAAUUUUUGGAGcaggUUUUcUgacUUc | |
| | ggUcggaaaaccccU | |
| 3ex5ex-30 | AUGAUAGGGACUUAGGGUGAAAAAGGGGUAAUACAGUACCCAUAAU | 1116 |
| | AAAAGAUUAUCCUAUAUAAAAUAGAAAUAUAAUUUUUGGAGcaggUU | |
| | UUcUgacUUcggUcggaaaaccccU | |
| 3ex5int-24 | UAAACAAGAUUAUCCUAUAUAAAAUAGAAAUAUAAAUUUUUGGAGC | 1117 |
| | aggUUUUcUgacUUcggUcggaaaaccccU | |
| 3ex5int-28 | AUGAUAGGGACUUAGGGUGAUACAGUACCCAUAAUAAAGGGCUGUU | 1118 |
| | GGAGCAAGAUUAUCCUAUAUAAAAUUUUUGGAGcaggUUUUcUgacUUc | |
| | ggUcggaaaaccccU | |
| 3ex5int30 | AUGAUAGGGACUUAGGGUGAAUACAGUACCCAUAAUAAAGGGCUGA | 1119 |
| | GGAAGUUGGAGCAAGAUUAUCCUAUAUAAAAAUUUUUGGAGcaggUU | |
| | UUcUgacUUcggUcggaaaaccccU | |
| 3int5ex-24 | AUGAUAGGGACUUAGGGUGAAAUACAGUACCCAUAAUAAAGGGCUG | 1120 |
| | AGGGCAAGUUGGAGCAAGAUUAUCCUAUAUAAAAAAUUUUUGGAGc | |
| | aggUUUUcUgacUUcggUcggaaaaccccU | |
| 3int5ex-28 | AUGAUAGGGACUUAGGGUGAAGGGGUAAUACAGUACCCAUAAUAUU | 1121 |
| | GGAGCAAGAUUAUCCUAUAUAAAAUUUUUGGAGcaggUUUUcUgacUUc | |
| | ggUcggaaaaccccU | |
| 3int5ex-30 | AUGAUAGGGACUUAGGGUGAAAAAGGGGUAAUACAGUACCCAUAAU | 1122 |
| | AAAAGUUGGAGCAAGAUUAUCCUAUAUAAAAAUUUUUGGAGcaggUU | |
| | UUcUgacUUcggUcggaaaaccccU | |
| 3int5int-24 | AUGAUAGGGACUUAGGGUGACAAAAGGGGUAAUACAGUACCCAUAA | 1123 |
| | UAAACAAGUUGGAGCAAGAUUAUCCUAUAUAAAAAAUUUUUGGAG | |
| | aggUUUUcUgacUUcggUcggaaaaccccU | |
| 3int5int-28 | AUGAUAGGGACUUAGGGUGAUACAGUACCCAUAAUAAAGGGCUGAG | 1124 |
| | AUUAUCCUAUAUAAAAUAGAAAAAUUUUUGGAGcaggUUUUcUgacUUc | |
| | ggUcggaaaaccccU | |
| 3int5int-30 | AUGAUAGGGACUUAGGGUGAAUACAGUACCCAUAAUAAAGGGCUGA | 1125 |
| | GGAAGAUUAUCCUAUAUAAAAUAGAAAUAUAAUUUUUGGAGcaggUU | |
| | UUcUgacUUcggUcggaaaaccccU | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A nucleic acid expression system, comprising:
a regulatory sequence operably coupled to a transcribable region of a nucleic acid, the regulatory sequence comprising a proximal regulatory sequence element (PSE) and a distal regulatory sequence element (DSE);
wherein the PSE comprises a PSE sequence of a first small nuclear RNA (snRNA) of a first organism species, wherein the DSE comprises a DSE sequence of a second small nuclear RNA (snRNA) of a second organism species, and wherein the first and second snRNAs are different or wherein the first and second organism species are different.

2. The system of claim 1, wherein the first snRNA is selected from the group consisting of snRNA U2, snRNA U3, snRNA U12, snRNA U1, snRNA U4, snRNA U5, snRNA U6, snRNA U7, snRNA U11, and snRNA 7SK.

3. The system of claim 1, wherein the second snRNA is selected from the group consisting of snRNA U2 snRNA U3, snRNA U12, snRNAU1, snRNA U4, snRNA U5, snRNA U6, snRNA U1, snRNA U11, and snRNA 7SK.

4. The system of claim 1, wherein the first organism species is selected from the group consisting of human and mouse.

5. The system of claim 1, wherein the second organism species is selected from the group consisting of mouse and human.

6. The system of claim 1, wherein:
the first snRNA is selected from the group consisting of snRNA U2, snRNA U3, snRNA U12, snRNA U1, snRNA U4, snRNA U5, snRNA U6, snRNA U7, snRNA U11, and snRNA 7SK;
the second snRNA is selected from the group consisting of snRNA U2, snRNA U3, snRNA U12, snRNA U1, snRNA U4, snRNA U5, snRNA U6, snRNA U7, snRNA U11, and snRNA 7SK;
the first organism species is selected from the group consisting of human and mouse; and
the second organism species is selected from the group consisting of mouse and human.

7. The system of claim 1, wherein the regulatory sequence comprises a promoter sequence.

8. The system of claim 6, wherein the regulatory sequence comprises a promoter sequence.

9. The system of claim 1, wherein the regulatory sequence comprises a terminator sequence.

10. The system of claim 6, wherein the regulatory sequence comprises a terminator sequence.

11. A nucleic acid expression system, comprising:
a regulatory sequence operably coupled to a transcribable region of a nucleic acid, the regulatory sequence comprising a proximal regulatory sequence element (PSE) and a distal regulatory sequence element (DSE);
wherein the PSE comprises a mouse U3 snRNA (“MmU3”) PSE, mouse U2 snRNA (“MmU2”) PSE, a mouse U11 snRNA (“MmU11”) PSE, a mouse U1 snRNA (“MmU1) PSE, a mouse U4 snRNA (“MmU4”) PSE, a mouse U5 snRNA (“MmU5”) PSE, a mouse U6 snRNA (“MmU6”) PSE, a mouse U7 snRNA (“MmU7”) PSE, a mouse U12 snRNA (“MmU12”) PSE, a mouse U7SK snRNA (“MmU7SK”) PSE, a human U12 snRNA (“HsU12”) PSE, a human U1 snRNA (“HsU1”) PSE, a human U2 snRNA (“HsU2”) PSE, a human U3 snRNA (“HsU3”) PSE, a human U4 snRNA (“HsU4”) PSE, a human U5 snRNA (“HsU5”) PSE, a human U6 snRNA (“HsU6”) PSE, a human U7 snRNA (“HsU7”) PSE, a human U11 snRNA (“HsU11”) PSE, or a human U7SK snRNA (“HsU7SK”) PSE;
wherein the DSE comprises a mouse U3 snRNA (“MmU3”) DSE, mouse U2 snRNA (“MmU2”) DSE, a mouse U11 snRNA (“MmU11”) DSE, a mouse U1 snRNA (“MmU1) DSE, a mouse U4 snRNA (“MmU4”) DSE, a mouse U5 snRNA (“MmU5”) DSE, a mouse U6 snRNA (“MmU6”) DSE, a mouse U7 snRNA (“MmU7”) DSE, a mouse U12 snRNA (“MmU12”) DSE, a mouse U7SK snRNA (“MmU7SK”) DSE, a human U12 snRNA (“HsU12”) DSE, a human U1 snRNA (“HsU1”) DSE, a human U2 snRNA (“HsU2”) DSE, a human U3 snRNA (“HsU3”) DSE, a human U4 snRNA (“HsU4”) DSE, a human U5 snRNA (“HsU5”) DSE, a human U6 snRNA (“HsU6”) DSE, a human U7 snRNA (“HsU7”) DSE, a human U11 snRNA (“HsU11”) DSE, or a human U7SK snRNA (“HsU7SK”) DSE; and
wherein the PSE and the DSE are from a different species as each other, or from a different snRNA as each other.

12. The system of claim 11, wherein the PSE and the DSE are from a different species as each other, and from a different snRNA as each other.

13. The system of claim 11, wherein the regulatory sequence comprises a promoter sequence.

14. The system of claim 11, wherein the regulatory sequence comprises a terminator sequence.

15. A nucleic acid expression system, comprising:
a promoter sequence comprising a promoter proximal sequence element (PSE) and a promoter distal sequence element (DSE);
wherein the promoter PSE comprises a mouse U3 snRNA (“MmU3”) promoter PSE, mouse U2 snRNA (“MmU2”) promoter PSE, a mouse U11 snRNA (“MmU11”) promoter PSE, a mouse U1 snRNA (“MmU1) promoter PSE, a mouse U4 snRNA (“MmU4”) promoter PSE, a mouse U5 snRNA (“MmU5”) promoter PSE, a mouse U6 snRNA (“MmU6”) promoter PSE, a mouse U7 snRNA (“MmU7”) promoter PSE, a mouse U12 snRNA (“MmU12”) promoter PSE, a mouse U7SK snRNA (“MmU7SK”) promoter PSE, a human U12 snRNA (“HsU12”) promoter PSE, a human U1 snRNA (“HsU1”) promoter PSE, a human U2 snRNA (“HsU2”) promoter PSE, a human U3 snRNA (“HsU3”) promoter PSE, a human U4 snRNA (“HsU4”) promoter PSE, a human U5 snRNA (“HsU5”) promoter PSE, a human U6 snRNA (“HsU6”) promoter PSE, a human U7 snRNA (“HsU7”) promoter PSE, a human U11 snRNA (“HsU11”) promoter PSE, or a human U7SK snRNA (“HsU7SK”) promoter PSE;
wherein the promoter DSE comprises a mouse U3 snRNA (“MmU3”) promoter DSE, mouse U2 snRNA (“MmU2”) promoter DSE, a mouse U11 snRNA (“MmU11”) promoter DSE, a mouse U1 snRNA (“MmU1) promoter DSE, a mouse U4 snRNA (“MmU4”) promoter DSE, a mouse U5 snRNA (“MmU5”) promoter DSE, a mouse U6 snRNA (“MmU6”) promoter DSE, a mouse U7 snRNA (“MmU7”) promoter DSE, a mouse U12 snRNA (“MmU12”) promoter DSE, a mouse U7SK snRNA (“MmU7SK”) promoter DSE, a human U12 snRNA (“HsU12”) promoter DSE, a human U1 snRNA (“HsU1”) promoter DSE, a human U2 snRNA (“HsU2”) promoter DSE, a human U3 snRNA (“HsU3”) promoter DSE, a human U4 snRNA (“HsU4”) promoter DSE, a human U5 snRNA (“HsU5”) promoter DSE, a human U6 snRNA (“HsU6”) promoter DSE, a human U7 snRNA (“HsU7”) promoter DSE, a human U11 snRNA (“HsU1”) promoter DSE, or a human U7SK snRNA (“HsU7SK”) promoter DSE; and
wherein the promoter PSE and the promoter DSE are from a different species as each other, or from a different snRNA as each other.

16. The system of claim 15, wherein the promoter PSE and the promoter DSE are from a different species as each other, and from a different snRNA as each other.

17. The system of claim 15, further comprising a transcribable region operably coupled to the promoter sequence.

18. The system of claim 17, further comprising a terminator sequence 3′ to the transcribable region.

19. A nucleic acid expression system, comprising:
a terminator sequence 3′ to a transcribable region, the terminator sequence comprising a terminator proximal sequence element (PSE) and a terminator distal sequence element (DSE);
wherein the PSE comprises a mouse U5 snRNA (“MmU5”) terminator PSE, a mouse U1 snRNA (“MmU1”) terminator PSE, a mouse U11 snRNA (“MmU11”) terminator PSE, a mouse U4 snRNA (“MmU4”) terminator PSE, a mouse U2 snRNA (“MmU2”) terminator PSE, a mouse U6 snRNA (“MmU6”) terminator PSE, a mouse U7 snRNA (“MmU7”) terminator PSE, a mouse U3 snRNA (“MmU3”) terminator PSE, a mouse U12 snRNA (“MmU12”) terminator PSE, a mouse U7SK snRNA (“MmU7SK”) terminator PSE, a human U1 snRNA (“HsU1”) terminator PSE, a human U5 snRNA (“HsU5”) terminator PSE, a human U12 snRNA (“HsU12”) terminator PSE, a human U4 snRNA (“HsU4”) terminator PSE, a human U2 snRNA (“HsU2”) terminator PSE, a human U6 snRNA (“HsU6”) terminator PSE, a human U7 snRNA (“HsU7”) terminator PSE, a human U11 snRNA (“HsU11”) terminator PSE, a human U3 snRNA (“HsU3”) terminator PSE, or a human U7SK snRNA (“HsU7SK”) terminator PSE;
wherein the DSE comprises a mouse U5 snRNA (“MmU5”) terminator DSE, a mouse U1 snRNA (“MmU1”) terminator DSE, a mouse U11 snRNA (“MmU11”) terminator DSE, a mouse U4 snRNA (“MmU4”) terminator DSE, a mouse U2 snRNA (“MmU2”) terminator DSE, a mouse U6 snRNA (“MmU6”) terminator DSE, a mouse U7 snRNA (“MmU7”) terminator DSE, a mouse U3 snRNA (“MmU3”) terminator DSE, a mouse U12 snRNA (“MmU12”) terminator DSE, a mouse U7SK snRNA (“MmU7SK”) terminator DSE, a human U1 snRNA (“HsU1”) terminator DSE, a human U5 snRNA (“HsU5”) terminator DSE, a human U12 snRNA (“HsU12”) terminator DSE, a human U4 snRNA (“HsU4”) terminator DSE, a human U2 snRNA (“HsU2”) terminator DSE, a human U6 snRNA (“HsU6”) terminator DSE, a human U7 snRNA (“HsU7”) terminator DSE, a human U11 snRNA (“HsU11”) terminator DSE, a human U3 snRNA (“HsU3”) terminator DSE, or a human U7SK snRNA (“HsU7SK”) terminator DSE; and
wherein the terminator PSE and the terminator DSE are from a different species as each other, or from a different snRNA as each other.

20. The system of claim 19, wherein the terminator PSE and the terminator DSE are from a different species as each other, and from a different snRNA as each other.

21. The system of claim 19, further comprising a promoter sequence, wherein the transcribable region is operably coupled to the promoter sequence.

22. A nucleic acid expression system, comprising:
a promoter sequence operably coupled to a transcribable region of a nucleic acid, and a terminator sequence coupled to the transcribable region;
wherein the promoter sequence comprises a promoter sequence of a first small nuclear RNA (snRNA) of a first organism species, wherein the terminator sequence comprises a terminator sequence of a second small nuclear RNA (snRNA) of a second organism species, and wherein the first and second snRNAs are different or wherein the first and second organism species are different.

23. The system of claim 22, wherein the first snRNA is selected from the group consisting of snRNA U12, snRNA U3, snRNA U2, snRNA U4, snRNA U5, snRNA U6, snRNA U7, snRNA U11, snRNA U1 and snRNA 7SK.

24. The system of claim 22, wherein the first snRNA is selected from the group consisting of snRNA U12, snRNA U3, snRNA U2, snRNA U4, snRNA U5, snRNA U6, snRNA U7, snRNA U11, snRNA U1 and snRNA 7SK.

25. The system of claim 22, wherein:
the first snRNA is selected from the group consisting of snRNA U12, snRNA U3, snRNA U2, snRNA U4, snRNA U5, snRNA U6, snRNA U7, snRNA U11, snRNA U1 and snRNA 7SK;
the second snRNA is selected from the group consisting of the first snRNA is selected from the group consisting of snRNA U12, snRNA U3, snRNA U2, snRNA U4, snRNA U5, snRNA U6, snRNA U7, snRNA U11, snRNA U1 and snRNA 7SK;
the first organism species is selected from the group consisting of human and mouse; and
the second organism species is selected from the group consisting of mouse and human.

26. The system of claim 22, wherein the promoter comprises a promoter PSE sequence and a promoter DSE sequence, and wherein the either the promoter PSE or the promoter DSE is of a different organism species than the first organism species.

27. The system of claim 22, wherein the promoter comprises a promoter PSE sequence and a promoter DSE sequence, and wherein the either the promoter PSE or the promoter DSE is of a different snRNA than the first snRNA.

28. The system of claim 22, wherein the promoter comprises a promoter PSE sequence and a promoter DSE sequence, and wherein the either the promoter PSE or the promoter DSE is of a different organism species than the first organism species; and
wherein the promoter comprises a promoter PSE sequence and a promoter DSE sequence, and wherein the either the promoter PSE or the promoter DSE is of a different snRNA than the first snRNA.

29. The system of claim 22, wherein the terminator comprises a terminator PSE sequence and a terminator DSE sequence, and wherein the either the terminator PSE or the terminator DSE is of a different organism species than the second organism species.

30. The system of claim 22, wherein the terminator comprises a terminator PSE sequence and a terminator DSE sequence, and wherein the either the terminator PSE or the terminator DSE is of a different snRNA than the second snRNA.

31. The system of claim 22, wherein the terminator comprises a terminator PSE sequence and a terminator DSE sequence, and wherein the either the terminator PSE or the terminator DSE is of a different organism species than the second organism species; and
wherein the terminator comprises a terminator PSE sequence and a terminator DSE sequence, and wherein the either the terminator PSE or the terminator DSE is of a different snRNA than the second snRNA.

32. The system of claim 22, wherein the promoter comprises a promoter PSE sequence and a promoter DSE sequence, and wherein the either the promoter PSE or the promoter DSE is of a different organism species than the first organism species; and
wherein the promoter comprises a promoter PSE sequence and a promoter DSE sequence, and wherein the either the promoter PSE or the promoter DSE is of a different snRNA than the first snRNA;
wherein the terminator comprises a terminator PSE sequence and a terminator DSE sequence, and wherein the either the terminator PSE or the terminator DSE is of a different organism species than the second organism species; and
wherein the terminator comprises a terminator PSE sequence and a terminator DSE sequence, and wherein the either the terminator PSE or the terminator DSE is of a different snRNA than the second snRNA.

33. A nucleic acid expression system, comprising:
a promoter sequence comprising a mouse U3 snRNA (“MmU3”) promoter sequence, a mouse U2 snRNA (“MmU2”) promoter sequence, a mouse U11 snRNA (“MmU11”) promoter sequence, a mouse U4 snRNA (“MmU4”) promoter sequence, a mouse U5 snRNA (“MmU5”) promoter sequence, a mouse U6 snRNA (“MmU6”) promoter sequence, a mouse U7 snRNA (“MmU7”) promoter sequence, a mouse U1 snRNA (“MmU1”) promoter sequence, a mouse U12 snRNA (“MmU12”) promoter sequence, a mouse U7SK snRNA (“MmU7SK”) promoter sequence, a human U12 snRNA (“HsU12”) promoter sequence, a human U1 snRNA (“HsU1”) promoter sequence, a human U2 snRNA (“HsU2”) promoter sequence, a human U4 snRNA (“HsU4”) promoter sequence, a human U5 snRNA (“HsU5”) promoter sequence, a human U6 snRNA (“HsU6”) promoter sequence, a human U7 snRNA (“HsU7”) promoter sequence, a human U11 snRNA (“HsU11”) promoter sequence, a human U3 snRNA (“HsU3”) promoter sequence, or a human U7SK snRNA (“HsU7SK”) promoter sequence, or a fragment or combination of fragments thereof; and
a transcribable region operably coupled to the promoter sequence and to a terminator sequence comprising a mouse U5 snRNA (“MmU5”) terminator sequence, a mouse U1 snRNA (“MmU1”) terminator sequence, a mouse U11 snRNA (“MmU11”) terminator sequence, a mouse U4 snRNA (“MmU4”) terminator sequence, a mouse U2 snRNA (“MmU2”) terminator sequence, a mouse U6 snRNA (“MmU6”) terminator sequence, a mouse U7 snRNA (“MmU7”) terminator sequence, a mouse U3 snRNA (“MmU3”) terminator sequence, a mouse U12 snRNA (“MmU12”) terminator sequence, a mouse U7SK snRNA (“MmU7SK”) terminator sequence, a human U1 snRNA (“HsU1”) terminator sequence, a human U5 snRNA (“HsU5”) terminator sequence, a human U12 snRNA (“HsU12”) terminator sequence, a human U4 snRNA (“HsU4”) terminator sequence, a human U2 snRNA (“HsU2”) terminator sequence, a human U6 snRNA (“HsU6”) terminator sequence, a human U7 snRNA (“HsU7”) terminator sequence, a human U11 snRNA (“HsU11”) terminator sequence, a human U3 snRNA (“HsU3”) terminator sequence, or a human U7SK snRNA (“HsU7SK”) terminator sequence, or a fragment or combination of fragments thereof;
wherein the promoter sequence and the terminator sequence are at least partially of a different organism species as each other, or are at least partially of a different snRNA as each other.

34. The system of claim 33, wherein the promoter sequence and the terminator sequence are at least partially of a different organism species as each other, and are at least partially of a different snRNA as each other.

35. The system of any one claims 1-14 or 17-34, wherein the expression system produces a greater amount of RNA from the transcribable region than an expression system having a wild-type promoter and wild-type terminator operably coupled to the transcribable region.

36. The system of claim 35, wherein the amount of RNA produced is at least 10% greater.

37. A method of producing a ribonucleic acid (RNA), comprising contacting a cell with the expression system of any one claims 1-34.
